(19) United States
(12) Patent Application Publication
(10) Pub. No.: US 2014/0171431 A1
(43) Pub. Date: Jun. 19, 2014

(54) AZOLE HETEROCYCLIC COMPOUND, PREPARATION METHOD, PHARMACEUTICAL COMPOSITION AND USE

(71) Applicants: Jianhua Shen, Shanghai (CN); Yiping Wang, Shanghai (CN); Kai Wang, Shanghai (CN)

(72) Inventors: Jianhua Shen, Shanghai (CN); Yiping Wang, Shanghai (CN); Kai Wang, Shanghai (CN)

(21) Appl. No.: 14/129,199

(22) PCT Filed: May 15, 2012
(86) PCT No.: PCT/CN2012/000661

§ 371 Date: Feb. 28, 2014

(30) Foreign Application Priority Data
Jun. 27, 2011 (CN) PCTCN2011/076402

(51) Int. Cl. C07D 413/06; A61K 45/06; A61K 31/4709; C07D 413/14; A61K 31/194; C07D 403/06; C07D 403/14; A61K 31/513; C07D 401/14; C07C 59/255; A61K 31/517; A61K 31/5377

(52) U.S. Cl. CPC C07D 413/06; A61K 31/517; A61K 45/06; A61K 31/4709; C07D 413/14; A61K 31/5377; C07D 403/06; C07D 403/14; A61K 31/513; C07D 401/14; C07C 59/255; A61K 31/194
USPC 514/234.5; 544/253; 514/258.1; 546/155; 514/312; 544/116; 514/252.16; 544/310; 514/274

(57) ABSTRACT

The present invention relates to the field of pharmaceutical chemistry, and in particular, to a novel class of azole compounds represented by general formula (I), (II) or (III) and a preparation method thereof, a pharmaceutical composition with the compounds as active components, and a use of the azole compounds and the pharmaceutical composition in the preparation of a medicament for treatment of diseases associated with Lp-PLA₂ enzyme activities, wherein each substituent is as defined in the specification.

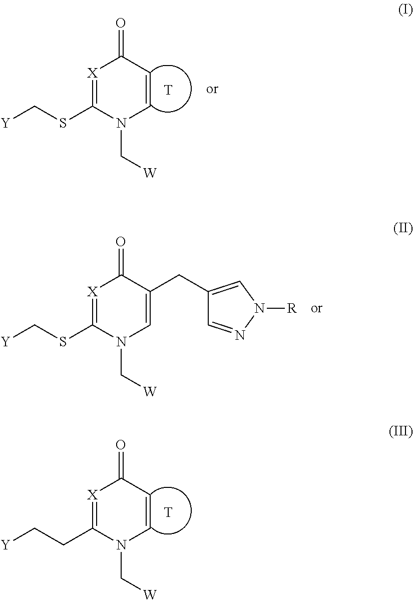

### FIELD OF THE INVENTION

The present invention relates to the field of medicinal chemistry, particularly to novel azole heterocylic compounds, their preparation methods, their pharmaceutical compositions with these compounds as active ingredients and their use in the preparation of medicaments for treating diseases associated with activity of the enzyme Lp-PLA2.

### BACKGROUND OF THE INVENTION

Atherosclerosis is demonstrated to be the pathophysiological basis for the development of cardiovascular diseases. Thrombosis resulting from atherosclerotic plaque rupture is the major cause of cardiovascular events. Hence, it is an important task in urgent need of a solution for modern medicine to prevent or cure atherosclerosis. At present, the standard clinical medication scheme comprises the use of statins for regulating lipid, the use of hypotensors for controlling blood pressure and the concurrent administration of drugs for antiplatelet aggregation. However, a substantial relapse risk still exists in patients despite long-term treatment with such a therapeutic schedule, especially in critical patients.

It is proved that atherosclerosis is a syndrome that relates not only to abnormal lipid level, but also to a variety of inflammatory reactions. Modulation of the related inflammatory factors may be a new approach in treating the disease. Oxidized low density lipoprotein (ox-LDL) is a risk factor in plasma that can accelerate the inflammatory reactions and induces the progression of atherosclerosis.

Furthermore, lipoprotein-associated phospholipase A₂ (Lp-PLA₂) is reported to play an important role in promoting the inflammatory reactions and inducing the atherosclerosis formation, and thus is a crucial enzyme that mediates the above biological effect of ox-LDL(Zalewski A et al, Arterioscler Thromb Vasc Biol, 2005, 25(5):923-931).

Lp-PLA₂, also known as platelet-activating factor (PAF) acetylhydrolase, is a member of the superfamily of PLA₂ and belongs to type VII PLA₂. The enzyme contains 441 amino acids and has a relative molecule weight of 45 kD. In human plasma, 70% Lp-PLA₂ is bound to LDL, and 30% Lp-PLA₂ is bound to high-density lipoprotein (HDL), indicating that Lp-PLA₂ is easily delivered along with LDL to the segments of arterial wall where lesion is formed. Lp-PLA₂ can hydrolyze PAF, PAF-like phospholipids, and oxidatively modified phosphatidylcholines. Its enzymatic activity occurs in the absence of divalent cations such as calcium ions, which is different from many other PLA₂s. Lp-PLA₂ is strongly specific to the short-chain residues of phospholipids at the sn-2 position. When the residue at the sn-2 position is acetyl group, it exhibits the maximum hydrolyzing activity. In contrast, it lacks enzymatic activity for phospholipid substrates in which long-chain fatty acids are located at the sn-2 position. Gene mutagenesis technique has identified that the enzymatic active center is consisted of three residues in Lp-PLA₂, including Ser-273, Asp-296 and His-351.

The atherogenic mechanism of Lp-PLA₂ was first proposed by Macphee and co-workers (Macphee C H, Moores K E, Boyd H F, et al. Biochem J 1999; 338:479-87). As a component of LDL, lecithin is oxidatively modified at sn-2 position to shorten the long chain and then enters the arterial intima as a substrate of Lp-PLA₂. The substrate is rapidly hydrolyzed by Lp-PLA₂ into two products—lysophosphatidylcholine (lyso-LPC) and oxidized free fatty acids (oxNEFAs), both of which have potent proinflammatory properties. The two biological mediators exhibit their pre-atherogenic effects by initiating a variety of inflammation-immune responses in several cells (endothelial cells, smooth muscle cells, monocytes, macrophages, T cells and neutrophils, etc.), for example, up-regulation of adhesion molecules, recruitment of monocytes/macrophages to atherosclerotic plaque region, induction of cytokines (such as interferons) expression, activation of leucocytes, induction of oxidation stress, induction of cell membrane permeability and cellular apoptosis. These effects are deemed to promote atherosclerotic plaque growth and destabilization, and further lead to continuous enlargement of a necrotic core and thinning of a fibrous cap. Thrombus is much likely to be formed once the plaque gets ruptured, ultimately resulting in clinical diseases such as myocardial infarction, coronary heart disease, ischemic stroke and the like.

Therefore, use of Lp-PLA₂ inhibitors is expected to reduce aforementioned inflammatory responses and may represent a novel and non lipid-lowering strategy for atherosclerosis treatment. It was observed in human body that selective inhibition of Lp-PLA₂ was able to notably reduce the generation of ox-NEFA and the apoptosis of macrophage induced by ox-LDL (Rosenson R S, Vracar-Grabar M, Helenowski I. Cardiovasc Drugs Ther 2008; 22:55-8). The effect of Lp-PLA₂ inhibitor was also supported by experimentation on animal models. Wilensky et al. carried out detailed studies on the effects of darapladib as an Lp-PLA₂ inhibitor on atherosclerotic lesion area, composition, and gene expression in diabetic/hypercholesterolemic (DM-HC) swine. It was observed that darapladib treatment remarkably reduced the atherosclerotic plaque progression.

Considerable clinical studies have shown a positive association between the level of Lp-PLA₂ and the incidence of cardiovascular events. In the West of Scotland Coronary Prevention Study (WOSCOPS) a nested case-control study involving 580 patients with established miocardial infarction, ischemic reperfusion or coronary heart diseases and 1,168 matched controls, Caslake et al. first proposed the link of Lp-PLA₂ level with coronary heart disease events (CHD) (Packard C J, O'Reilly D S, Caslake M J, et al. West of Scotland Coronary Prevention Group. N Engl J Med 2000; 343: 1148-55). Lp-PLA₂ was found to be an independent risk factor for predicting future CHD events. Lp-PLA₂ was shown to have positive association with LDL, have little association with Fbg, have no association with CRP, WBC and other risk factors, and was not affected by smoking. The incidence of CHD events increases by 22% for each standard deviation increase in Lp-PLA₂.

Several studies towards apparently healthy patients have also shown that the concentration of Lp-PLA₂ could be used as a diagnostic indicator for early warning to those populations who have relatively low LDL level but have potential risk of CHD events. In the Atherosclerosis Risk in Communities (ARIC) study, Ballantyne et al. examined more than 12,000 middle-aged men and women patients with an LDL level <130 mg/dl. It was found that the incidence of cardiovascular events increased with the elevated level of Lp-PLA₂. Patients in the highest tertile of Lp-PLA₂ level had a hazard ratio of 1.78 for incident CHD events compared to the lowest tertile. But, this association was not evident among those who have an LDL level >130 mg/dl. However, in the Rotterdam Study that involved 7,983 patients over 55 years of age without established CHD, Lp-PLA₂ is proved to be the predictor of CHD events, as well as a risk marker for ischemic stroke, independently of the level of cholesterol.

In the population with established cardiovascular disease (e.g. secondary prevention patients), Lp-PLA₂ also appears to be a risk factor for recurrent cardiovascular events. In a study involving patients who were undergoing clinically indicated coronary angiography, each standard deviation increase in Lp-PLA₂ would lead to 30% increase in the risk of cardiovascular event over a four-year follow-up period, independently of traditional risk factors and C-reactive protein (CRP). Another study towards the patients who were participating in a rehabilitation program demonstrated that, patients in the highest tertile of Lp-PLA₂ concentrations were associated with twice the risk of recurrent events compared with those in the lowest tertile.

The increase of lysophosphatidylchloline (Lyso-PC), one of the hydrolysis products of ox-LDL, was deemed to be connected with endothelial dysfunction in atherosclerosis patients. Thus, Lp-PLA₂ inhibitor is expected to ameliorate such disorder and may have a general application in treating other diseases associated with endothelial dysfunction, for example diabetes, hypertension, angina and ischemic reperfusion.

Lp-PLA₂ is expressed in activated inflammatory cells (such as macrophages, lymphocytes, neutrophils, eosnophils). Therefore, Lp-PLA₂ inhibitors may be of use in treating any disorder that is associated with inflammatory cells. Such conditions include psoriasis and various airway inflammations, such as asthma and chronic bronchitis.

Additionally, Lp-PLA₂ inhibitors may also have a general application in any disorder that involves the hydrolysis of oxidized lipid with the participation of Lp-PLA₂ into two inflammation specific compounds. Such diseases may include atherosclerosis, diabetes, hypertension, angina, rheumatoid arthritis, stroke, myocardial infarction, reperfusion injury, acute and chronic inflammation.

Patent applications WO96/13484, WO96/19451, WO97/02242, WO97/21765, WO97/21766, WO97/41098 and WO97/41099 (SmithKline Beecham plc) disclosed a series of monocyclic beta lactam derivatives which are irreversible acetylation inhibitors of Lp-PLA₂ (Tew et al, Biochemistry, 37, 10087, 1998).

Researchers from SmithKline Beecham plc developed a class of potent, reversible Lp-PLA₂ inhibitors which were characterized by the pyrimidone or pyridone scaffold in the molecules (WO99/24420, WO01/60805, WO02/30911, WO03/016287, WO03/042179, WO03/042206, WO08/048,867, et al). Darapladib (SB480848), a representative inhibitor of this class, is undergoing phase III clinical trials at present.

Korean researchers developed a series of novel O-acyloxime derivatives which displayed micromole activities (U.S. Pat. No. 7,642,291).

We have found various novel azole heterocyclic compounds which are demonstrated by pharmacological experimentation to be potent Lp-PLA₂ inhibitors.

### SUMMARY OF THE INVENTION

A first object of this invention is to provide pharmaceutically valuable azole heterocyclic compounds of Formula (I), (II) or (III), cis-trans isomers, enantiomers, diastereoisomers, racemes, solvates, hydrates, or pharmaceutically acceptable salts thereof.

Another object of this invention is to provide preparation methods of the compounds of Formula (I).

Still another object of this invention is to provide the use of a compound of Formula (I), (II) or (III) as an Lp-PLA₂ inhibitor, and as such the use of the compound in manufacturing a medicament for preventing, curing or ameliorating a disease associated with activity of the enzyme Lp-PLA₂. The mentioned diseases may include atherosclerosis, stroke, myocardial infarction, angina, myocardial ischemia, reperfusion injury, diabetes, asthma, psoriasis, rheumatoid arthritis, or acute and chronic inflammation.

Yet another object of this invention is to provide a pharmaceutical composition that comprises one or more compounds of Formula (I), (II) or (III) in therapeutically effective amount, or a pharmaceutically acceptable salt thereof, and a pharmaceutically acceptable excipient.

Yet another object of this invention is to provide a method of preventing, curing or ameliorating a disease associated with activity of the enzyme Lp-PLA₂, wherein the method involves the use of the azole heterocyclic compounds, cis-trans isomers, enantiomers, diastereoisomers, racemes, solvates, hydrates, or pharmaceutically acceptable salts thereof or the compositions according to this invention.

### DETAILED DESCRIPTION OF THE INVENTION

In the first aspect, the present invention provides azole heterocyclic compounds of Formula (I), (II) or (III), cis-trans isomers, enantiomers, diastereoisomers, racemes, solvates, hydrates, or pharmaceutically acceptable salts thereof:

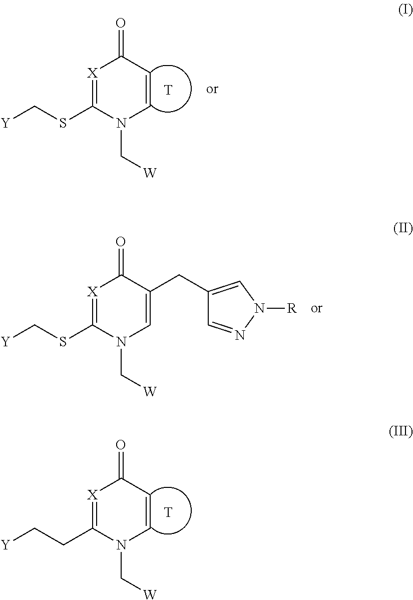

in which:

T is 4 to 6-membered aliphatic ring or benzene ring;

R is C₁-C₆ alkyl;

X is CH or N;

Y is phenyl group, optionally substituted by one or more substituents selected from halogen, C₁-C₆ alkoxyl, C₁-C₆ alkyl or halogenated C₁-C₆ alkyl;

W is selected from 12 structures of Formulae (a-1) as follows:

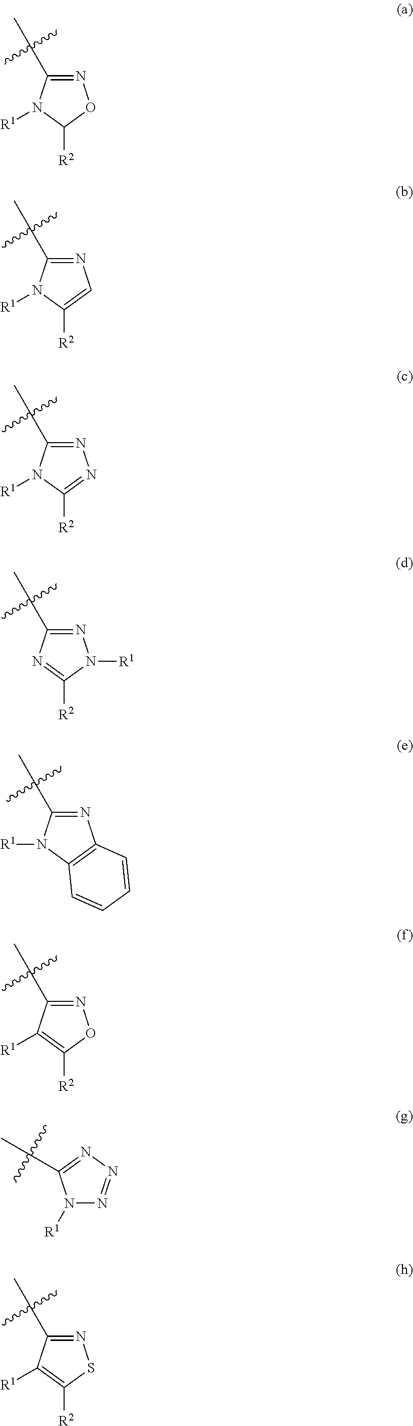

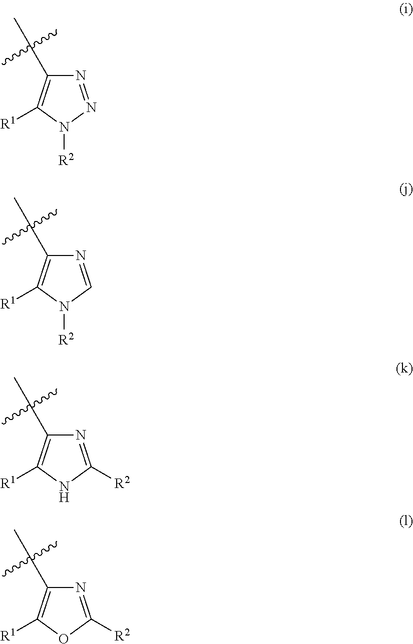

R¹ is selected from H,

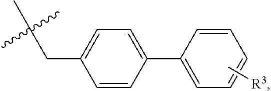

C₃-C₁₂ alkenyl, C₁-C₁₂ alkyl, —NR⁴R⁵ substituted C₂-C₄ alkyl, benzyl or piperidyl which is optionally substituted by —COOR⁴;

R² is selected from H,

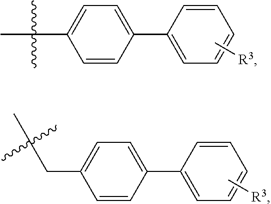

—COR⁴, —COOR⁴, —CONR⁴R⁵, —CH═NNR⁴R⁵, —C(═CH₂)—OC(═O)R⁴, C₁-C₁₂ alkyl, C₃-C₇ cycloalkyl, phenyl, wherein alkyl, cycloalkyl and phenyl are optionally substituted by halogen, —NR⁴R⁵, —OR⁴, —SR⁴, —SO₂R⁴, —NHCOR⁴, —NHSO₂R⁴, —NHCSNHR⁴,

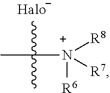

—N₃ or phenyl.

R³, optionally at ortho-, meta- or para-position of the benzene ring, is selected from H, halogen, C₁-C₆ alkyl or partially or fully halogenated C₁-C₆ alkyl;

R⁴ and R⁵ are independently selected from H, C₃-C₇ cycloalkyl, straight or branched C₁-C₆ alkyl, wherein alkyl and cycloalkyl are optionally substituted by —COOR⁹, —NR⁹R¹⁰, —OR⁹, —COR⁹, phenyl, benzyl, aromatic or nonaromatic heterocycle, wherein phenyl, benzyl, aromatic and nonaromatic heterocycle are optionally further substituted by halogen or C₁-C₆ alkyl; or

R⁴ and R⁵ together with the N-atom to which they are attached form 5 to 8-membered nonaromatic heterocycle which may contain another heteroatom selected from the group consisting of N, O and S, and is optionally substituted by halogen, C₁-C₆ alkyl, —NR¹¹R¹², —OR¹¹, ═O, or benzyl, wherein C₁-C₆ alkyl is optionally substituted by —COOR⁴;

R⁶, R⁷ and R⁸ are independently selected from C₁-C₆ alkyl, hydroxyl substituted C₂-C₄ alkyl or benzyl, wherein benzyl is optionally substituted by halogen or C₁-C₆ alkyl;

R⁹ and R¹⁰ are independently selected from H, C₁-C₆ alkyl; or

R⁹, R¹⁰ together with the N-atom to which they are attached form 5 to 8-membered nonaromatic heterocycle which may contain another heteroatom selected from the group consisting of N, O and S;

R¹¹ and R¹² are independently selected from H, C₁-C₆ alkyl;

Halo is an abbreviation of halogen.

In a further preferred embodiment, within the compounds of Formula (I) or (III), T is 5-membered aliphatic ring or benzene ring; X is CH or N.

In a further preferred embodiment, within the compounds of Formula (I), when T is 5-membered aliphatic ring, X is N; when T is benzene ring, X is CH.

In a further preferred embodiment, the compounds of the present invention have the structures of Formula (IA)-(IG), (IIA) or (IIIA):

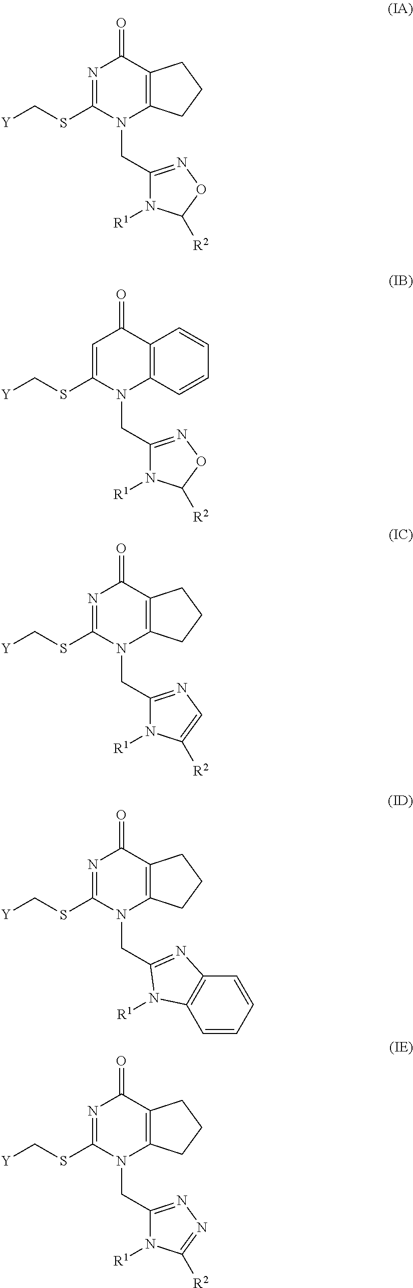

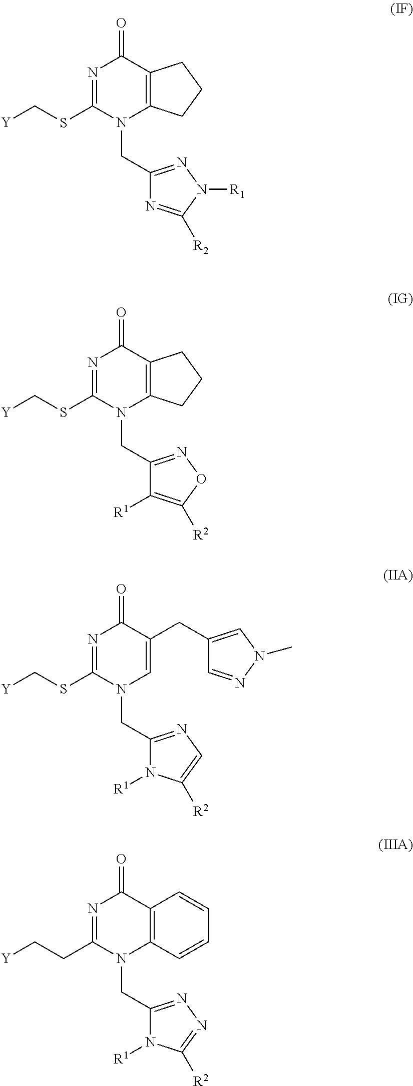

in which:
Y, R¹ and R² are as hereinbefore defined.

In a further preferred embodiment, within the compounds of Formula (I), (II), (III), (IA)-(IG), (IIA) or (IIIA):
Y is phenyl ring which is substituted by fluorine atom.

In a further preferred embodiment, within the compounds of Formula (I), (II), (III), (IA)-(IG), (IIA) or (IIIA):
Y is 4-fluorophenyl or 2,3-difluorophenyl.

In a further preferred embodiment, within the compounds of Formula (I), (II) or (III):
W is selected from 6 structures of Formulae (a-f) as follows:

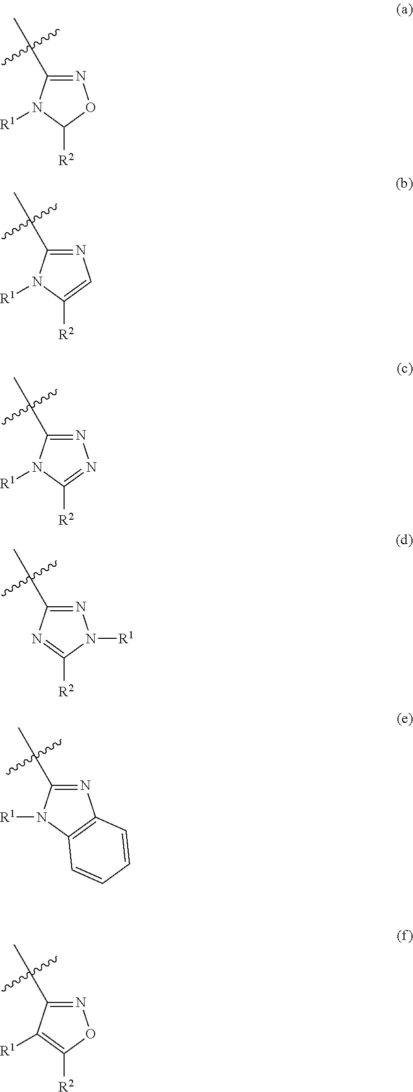

in which:
R¹ and R² are as hereinbefore defined.

In a further preferred embodiment, within the compounds of Formula (I), (II), (III), (IA)-(IG), (IIA) or (IIIA):

R¹ is

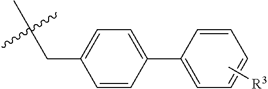

or —NR⁴R⁵ substituted C₂-C₄ alkyl.

In a further embodiment, within the compounds of Formula (I), (II), (III), (IA)-(IG), (IIA) or (IIIA).
R¹ is (4-(trifluoromethyl)biphenyl-4-yl)methyl.

In a further preferred embodiment, within the compounds of Formula (I), (II), (III), (IA)-(IG), (IIA) or (IIIA):
R² is —COR⁴, —CONR⁴R⁵, C₁-C₅ alkyl or C₃-C₅ cycloalkyl, wherein alkyl and cycloalkyl are optionally substituted by —NR⁴R⁵, —OR⁴, —SR⁴, —SO₂R⁴, ═NNR⁴R⁵, —NHCOR⁴, —NHSO₂R⁴, —NHCSNHR⁴ or

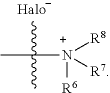

In a further preferred embodiment, within the compounds of Formula (I), (II), (III), (IA)-(IG), (IIA) or (IIIA):

R² is —COR⁴, —CONR⁴R⁵, cyclopropyl or C₁-C₅ alkyl, wherein alkyl is substituted by —NR⁴R⁵, —OR⁴, —SR⁴, —SO₂R⁴, ═NNR⁴R⁵, —NHCOR⁴, —NHSO₂R⁴, —NHCSNHR⁴ or

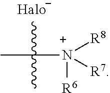

In a further preferred embodiment, within the compounds of Formula (I), (II), (III), (IA)-(IG), (IIA) or (IIIA):

R² is —CONR⁴R⁵, cyclopropyl or C₁-C₅ alkyl, wherein C₁-C₅ alkyl is substituted by —NR⁴R⁵, —OR⁴, —SR⁴, ═NNR⁴R⁵, or

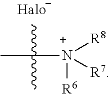

In a further preferred embodiment, within the compounds of Formula (I), (II), (III), (IA)-(IG), (IIA) or (IIIA), R² is selected from: dimethylcarbamoyl, 2-(diethylamino)ethylcarbamoyl, (2-(diethylamino)ethyl)(methyl)carbamoyl, (dimethylamino)methyl, (diethylamino)methyl, pyrrolidin-1-ylmethyl, ((4-fluorobenzyl)(methyl)amino)methyl, isopropyl, cyclopropyl, 3-(diethylamino)propyl, 4-(diethylamino)butyl, hydroxymethyl, 1-hydroxyethyl, (4-fluorobenzylthio)methyl, (isopropyl(methyl)amino)methyl, ((1-ethylpyrrolidin-2-yl)methylamino)methyl, (4-ethylpiperazin-1-yl)methyl, ((2-(dimethylamino)ethyl)(methyl)amino)methyl, ((2-(diethylamino)ethyl)(methyl)amino)methyl, (((2-(dimethylamino)ethyl)(ethyl)amino)methyl, (((3-(dimethylamino)propyl)(methyl)amino)methyl), (methyl(pyridin-2-ylmethyl)amino)methyl, (4-(dimethylamino)piperidin-1-yl)methyl, (2,2-dimethylhydrazono)methyl, (2-hydroxyethoxy)methyl, (2-(diethylamino)ethoxy)methyl, 1-(2-(dimethylamino)ethylamino)ethyl, 1-((2-(dimethylamino) ethyl)(methyl)amino) ethyl, 1-(2-(diethylamino) ethylamino)ethyl, 1-((2-(diethylamino)ethyl)(methyl)amino)ethyl, 1-((3-(dimethylamino)propyl)(methyl)amino) ethyl, ((methyl(2-(pyrrolidin-1-yl)ethyl)amino)methyl, ((methyl(2-(piperidin-1-yl)ethyl)amino)methyl, 3-(pyrrolidin-1-yl)propyl, 3-(piperidin-1-yl)propyl, 4-(pyrrolidin-1-yl)butyl, 4-(piperidin-1-yl)butyl,

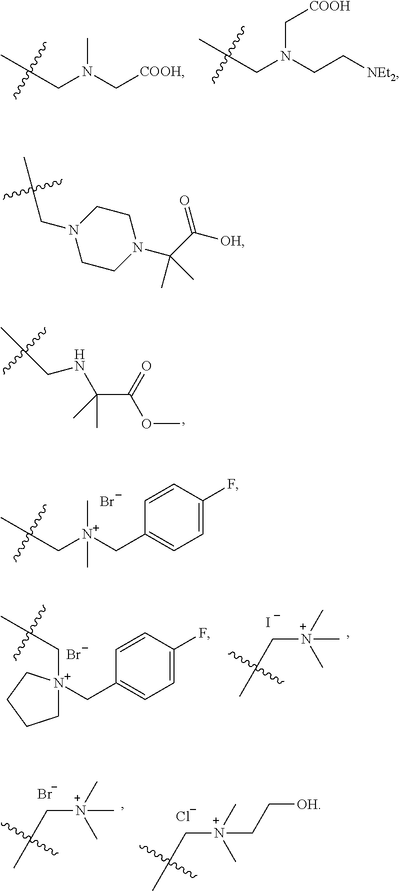

In a further preferred embodiment, the compound is selected from:
2-(4-fluorobenzylthio)-1-((5-n-heptyl-4,5-dihydro-1,2,4-oxadiazol-3-yl)methyl)-6,7-dihydro-1H-cyclopenta[d]pyrimidin-4(5H)-one;
1-((5-n-decyl-4,5-dihydro-1,2,4-oxadiazol-3-yl)methyl)-2-(4-fluorobenzylthio)-6,7-dihydro-1H-cyclopenta[d]pyrimidin-4(5H)-one;
2-(4-fluorobenzylthio)-1-((5-n-heptyl-4,5-dihydro-1,2,4-oxadiazol-3-yl)methyl)quinolin-4(1H)-one;
2-(4-fluorobenzylthio)-1-((5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one;
(E)-2-(4-fluorobenzylthio)-1-((5-n-heptyl-4-(n-oct-1-enyl)-4,5-dihydro-1,2,4-oxadiazol-3-yl)methyl)-6,7-dihydro-1H-cyclopenta[d]pyrimidin-4(5H)-one;
2-(2,3-difluorobenzylthio)-1-((4-(2-morpholinoethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one;
ethyl
4-(3-((2-(2,3-difluorobenzylthio)-4-oxo-4,5,6,7-tetrahydro-1H-cyclopenta[d]pyrimidin-1-yl)methyl)-5-(4′-(trifluoromethyl)biphenyl-4-yl)-1,2,4-oxadiazol-4(5H)-yl)piperidine-1-carboxylate;
ethyl
4-(3-((2-(4-fluorobenzylthio)-4-oxo-4,5,6,7-tetrahydro-1H-cyclopenta[d]pyrimidin-1-yl)methyl)-5-(4′-(trifluoromethyl)biphenyl-4-yl)-1,2,4-oxadiazol-4(5H)-yl)piperidine-1-carboxylate;
2-(4-fluorobenzylthio)-1-((4-(2-(piperidin-1-yl)ethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one;
1-((4-(2-(diethylamino)ethyl)-5-(4′-(trifluoromethyl)biphenyl-4-yl)-4,5-dihydro-1,2,4-oxadiazol-3-yl)methyl)-2-(4-fluorobenzylthio)-6,7-dihydro-1H-cyclopenta[d]pyrimidin-4(5H)-one;
2-(2,3-difluorobenzylthio)-1-((4-(2-(piperidin-1-yl)ethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one;
2-(4-fluorobenzylthio)-1-((5-phenyl-1H-imidazol-2-yl)methyl)-6,7-dihydro-1H-cyclopenta[d]pyrimidin-4(5H)-one;
1-((1H-tetrazol-5-yl)methyl)-2-(4-fluorobenzylthio)-6,7-dihydro-1H-cyclopenta[d]pyrimidin-4(5H)-one;
2-(4-fluorobenzylthio)-1-((1-((4′-(trifluoromethyl)biphenyl-4-yl)methyl)-1H-imidazol-2-yl)methyl)-6,7-dihydro-1H-cyclopenta[d]pyrimidin-4(5H)-one;
1-((1-n-dodecyl-1H-imidazol-2-yl)methyl)-2-(4-fluorobenzylthio)-6,7-dihydro-1H-cyclopenta[d]pyrimidin-4(5H)-one;
1-((1-butyl-1H-imidazol-2-yl)methyl)-2-(4-fluorobenzylthio)-6,7-dihydro-1H-cyclopenta[d]pyrimidin-4(5H)-one;
1-((1-((4′-chlorobiphenyl-4-yl)methyl)-1H-imidazol-2-yl)methyl)-2-(4-fluorobenzylthio)-6,7-dihydro-1H-cyclopenta[d]pyrimidin-4(5H)-one;
1-((1-(biphenyl-4-ylmethyl)-1H-imidazol-2-yl)methyl)-2-(4-fluorobenzylthio)-6,7-dihydro-1H-cyclopenta[d]pyrimidin-4(5H)-one;
1-((1-n-butyl-5-(4-chlorophenyl)-1H-imidazol-2-yl)methyl)-2-(4-fluorobenzylthio)-6,7-dihydro-1H-cyclopenta[d]pyrimidin-4(5H)-one;
1-((1-n-butyl-5-(4-chlorophenyl)-1H-imidazol-2-yl)methyl)-2-(2-nitrobenzylthio)-6,7-dihydro-1H-cyclopenta[d]pyrimidin-4(5H)-one;
1-((5-n-decyl-1-(2-(diethylamino)ethyl)-1H-imidazol-2-yl)methyl)-2-(4-fluorobenzylthio)-6,7-dihydro-1H-cyclopenta[d]pyrimidin-4(5H)-one;
1-((1-(2-(diethylamino)ethyl)-5-(4′-(trifluoromethyl)biphenyl-4-yl)-1H-imidazol-2-yl)methyl)-2-(4-fluorobenzylthio)-6,7-dihydro-1H-cyclopenta[d]pyrimidin-4(5H)-one;
1-((1-benzyl-5-((diethylamino)methyl)-1H-imidazol-2-yl)methyl)-2-(4-fluorobenzylthio)-6,7-dihydro-1H-cyclopenta[d]pyrimidin-4(5H)-one;
2-(4-fluorobenzylthio)-1-((5-methyl-1-((4′-(trifluoromethyl)biphenyl-4-yl)methyl)-1H-imidazol-2-yl)methyl)-6,7-dihydro-1H-cyclopenta[d]pyrimidin-4(5H)-one;
1-((5-((diethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
2-(4-fluorobenzylthio)-1-((5-(pyrrolidin-1-ylmethyl)-1-((4′-(trifluoromethyl)biphenyl-4-yl)methyl)-1H-imidazol-2-yl)methyl)-6,7-dihydro-1H-cyclopenta[d]pyrimidin-4(5H)-one;
1-((54(4-fluorobenzyl)(methyl)amino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
1-((5-((4-benzylpiperazin-1-yl)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
1-((5-((dimethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
N-(4-fluorobenzyl)-1-(2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazol-5-yl)-N,N-dimethylmethanaminium bromide;
1-((5-((dimethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
1-(4-fluorobenzyl)-1-((2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazol-5-yl)methyl)pyrrolidinium bromide;
ethyl
2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxylate;
2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxylic acid;
2-((2-(4-fluorobenzylthio)-4-oxo-4,5,6,7-tetrahydro-1H-cyclopenta[d]pyrimidin-1-yl)methyl)-N,N-dimethyl-1-((4′-(trifluoromethyl)biphenyl-4-yl)methyl)-1H-imidazole-5-carboxamide;
2-(4-fluorobenzylthio)-1-((1-((4′-(trifluoromethyl)biphenyl-4-yl)methyl)-1H-benzo[d]imidazol-2-yl)methyl)-6,7-dihydro-1H-cyclopenta[d]pyrimidin-4(5H)-one;
1-((4-ethyl-5-(4′-(trifluoromethyl)biphenyl-4-yl)-4H-1,2,4-triazol-3-yl)methyl)-2-(4-fluorobenzylthio)-6,7-dihydro-1H-cyclopenta[d]pyrimidin-4(5H)-one;
1-((4-ethyl-5-(4′-(trifluoromethyl)biphenyl-4-yl)-4H-1,2,4-triazol-3-yl)methyl)-2-(4-methoxybenzylthio)-6,7-dihydro-1H-cyclopenta[d]pyrimidin-4(5H)-one;
2-(2,3-difluorobenzylthio)-1-((4-ethyl-5-(4′-(trifluoromethyl)biphenyl-4-yl)-4H-1,2,4-triazol-3-yl)methyl)-6,7-dihydro-1H-cyclopenta[d]pyrimidin-4(5H)-one;
1-((4-ethyl-5-(3-phenylpropyl)-4H-1,2,4-triazol-3-yl)methyl)-2-(4-fluorobenzylthio)-6,7-dihydro-1H-cyclopenta[d]pyrimidin-4(5H)-one;
1-((4-ethyl-5-((4′-(trifluoromethyl)biphenyl-4-yl)methyl)-4H-1,2,4-triazol-3-yl)methy
1)-2-(4-fluorobenzylthio)-6,7-dihydro-1H-cyclopenta[d]pyrimidin-4(5H)-one;
2-(4-fluorobenzylthio)-1-((1-((4′-(trifluoromethyl)biphenyl-4-yl)methyl)-1H-1,2,4-triazol-3-yl)methyl)-6,7-dihydro-1H-cyclopenta[d]pyrimidin-4(5H)-one;
methyl
2-(((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)(methyl)amino)acetate;
2-(((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)(methyl)amino)acetic acid;
2-(4-fluorobenzylthio)-1-((5-methyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-propyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-isopropyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-cyclopropyl-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(benzylsulfonylmethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(3-(diethylamino)propyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(4-(diethylamino)butyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-(5-(hydroxymethyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazole-3-carbaldehyde;
1-((5-((dimethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
N-(4-fluorobenzyl)-1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)-N,N-dimethylmethanaminium bromide;
1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)-N,N,N-trimethylmethanaminium iodide;
1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)-N,N,N-trimethylmethanaminium bromide;
1-(5-((diethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((4-fluorobenzyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((isopropyl(methyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
(±) 1-((5-(((1-ethylpyrrolidin-2-yl)methylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((2S,6R)-2,6-dimethylmorpholino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
methyl-2-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)amino)-2-methylpropanoate;
1-((5-((4-ethylpiperazin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-methoxyethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
methyl-2-(4-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)piperazin-1-yl)-2-methylpropanoate;
2-(4-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)piperazin-1-yl)-2-methylpropanoic acid;
1-((54(2-(dimethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((2-(diethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((2-(diethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one tartrate;
1-((5-(((3-(dimethylamino)propyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-morpholinoethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
(R)-1-((5-((3-(dimethylamino)pyrrolidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(S)-1-((5-((3-(dimethylamino)pyrrolidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-(piperidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((2-(pyrrolidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((2-(diisopropylamino)ethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-((2-(diethylamino)ethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((4-methylpiperazin-1-yl)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((methyl(pyridin-2-ylmethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((cyclopropyl(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-((4-(dimethylamino)piperidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-((3,3-difluoropyrrolidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-((2-(dimethylamino)ethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(S)-1-((5-(((1-ethylpyrrolidin-2-yl)methylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-(2-oxoimidazolidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-(azidomethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(aminomethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
N-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methy1)ethanesulfonamide;
2-(4-fluorobenzylthio)-1-((5-((2-oxoimidazolidin-1-yl)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methy1)-3-methylthiourea;
1-((5-(chloromethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((4-fluorobenzylthio)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
(E)-1-((5-((2,2-dimethylhydrazono)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(E)-1-((5-((2-tert-butylhydrazono)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(E)-2-(4-fluorobenzylthio)-1-((5-((piperidin-1-ylimino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)vinyl acetate;
2-(4-fluorobenzylthio)-1-((5-(1-hydroxyethyl)-4-((4′-(trifluoromethyl)biphenyl-4-yl)methyl)-4H-1,2,4-triazol-3-yl)methyl)-6,7-dihydro-1H-cyclopenta[d]pyrimidin-4 (5H)-one;
1-((5-acetyl-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
N-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)-2-hydroxy-N,N-dimethylethanaminium chloride;
2-(4-fluorobenzylthio)-1-((5-((2-hydroxyethoxy)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((2-(diethylamino)ethoxy)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(n-butoxymethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-(2-(dimethylamino)ethylamino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-(2-(dimethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-(2-(diethylamino)ethylamino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-(2-(diethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-((2-(diethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one tartrate;
1-((5-(1-((3-(dimethylamino)propyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((2-(dimethylamino)ethyl)(ethyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
ethyl-2-((2-(diethylamino)ethyl)((3-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-5-yl)methyl)amino)acetate;
2-((2-(diethylamino)ethyl)((3-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-5-yl)methyl)amino)acetic acid;
N-(2-(diethylamino)ethyl)-2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxamide;
N-(2-(diethylamino)ethyl)-2-((2-(4-fluorobenzylthio)-4-oxo-4,5,6,7-tetrahydro-1H-cyclopenta[d]pyrimidin-1-yl)methyl)-N-methyl-1-((4′-(trifluoromethyl)biphenyl-4-yl)methyl)-1H-imidazole-5-carboxamide;
1-((5-(((2-(diethylamino)ethyl)(methyl)amino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
2-(4-fluorobenzylthio)-1-((5-((methyl(2-(pyrrolidin-1-yl)ethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((methyl(2-(piperidin-1-yl)ethyl)amino)methyl)-4-((4′-(trifluoromethyl)biphenyl-4-yl)methyl)-1H-imidazol-2-yl)methyl)-6,7-dihydro-1H-cyclopenta[d]pyrimidin-4(5H)-one;
2-(4-fluorobenzylthio)-1-((5-(3-(pyrrolidin-1-yl)propyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-(3-(piperidin-1-yl)propyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-(4-(pyrrolidin-1-yl)butyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-(4-(piperidin-1-yl)butyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((methyl(2-(piperidin-1-yl)ethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
methyl-3-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)isoxazole-5-carboxylate;
2-(4-fluorobenzylthio)-1-((5-(hydroxymethyl)-4-((4′-(trifluoromethyl)biphenyl-4-yl)methyl)isoxazol-3-yl)methyl)-6,7-dihydro-1H-cyclopenta[d]pyrimidin-4(5H)-one;
1-((5-(chloromethyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
1-((5-((diethylamino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
1-((5-(((2-(dimethylamino)ethyl)(methyl)amino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
1-((5-(((2-(diethylamino)ethyl)(methyl)amino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
2-(4-fluorobenzylthio)-1-((5-((methyl(2-(piperidin-1-yl)ethyl)amino)methyl)-4-((4′-(trifluoromethyl)biphenyl-4-yl)methyl)isoxazol-3-yl)methyl)-6,7-dihydro-1H-cyclopenta[d]pyrimidin-4(5H)-one;
2-(4-fluorophenethyl)-1-((4-((4′-(trifluoromethyl)biphenyl-4-yl)methyl)-4H-1,2,4-triazol-3-yl)methyl)quinazolin-4(1H)-one.

In the second aspect, the present invention provides a pharmaceutical composition that comprises a therapeutically effective amount of one or more azole heterocylic compounds of the invention, cis-trans isomers, enantiomers, diastereoisomers, racemes, solvates, hydrates, or pharmaceutically acceptable salts thereof, and a pharmaceutically acceptable auxiliary.

In a preferred embodiment, the above mentioned pharmaceutical composition may comprise one or more agents selected from the group consisting of anti-hyperlipidaemic, anti-atherosclerotic, anti-diabetic, anti-anginal, antiinflammatory, anti-hypertension agents and agents for lowering Lp(a).

In the third aspect, the present invention provides the use of said azole heterocylic compounds, cis-trans isomers, enantiomers, diastereoisomers, racemes, solvates, hydrates, or pharmaceutically acceptable salts thereof, in manufacturing a medicament as Lp-PLA₂ inhibitor.

In the fourth aspect, the present invention provides the use of said azole heterocylic compounds, cis-trans isomers, enantiomers, diastereoisomers, racemes, solvates, hydrates, or pharmaceutically acceptable salts thereof, in manufacturing a medicament for preventing, curing or ameliorating diseases associated with activity of the enzyme Lp-PLA₂.

In a preferred embodiment, the above mentioned diseases may include atherosclerosis, stroke, coronary heart disease, diabetes, asthma, psoriasis, rheumatoid arthritis, or acute and chronic inflammation.

In the fourth aspect, the present invention provides a method of preventing, curing or ameliorating a disease associated with activity of the enzyme Lp-PLA₂. The mentioned method involves treating a patient with said azole heterocylic compounds, cis-trans isomers, enantiomers, diastereoisomers, racemes, solvates, hydrates, or pharmaceutically acceptable salts thereof, or a composition of this invention.

In a preferred embodiment, the above mentioned diseases may include atherosclerosis, stroke, coronary heart disease, diabetes, asthma, psoriasis, rheumatoid arthritis, or acute and chronic inflammation.

In the fifth aspect, the present invention provides the preparation methods of the azole heterocyclic compounds of Formula (I), cis-trans isomers, enantiomers, diastereoisomers, racemes, solvates, hydrates, or pharmaceutically acceptable salts thereof. The synthesis of the compounds in this invention is featured by employing one of the processes illustrated by synthetic routes (1-4) as follows:

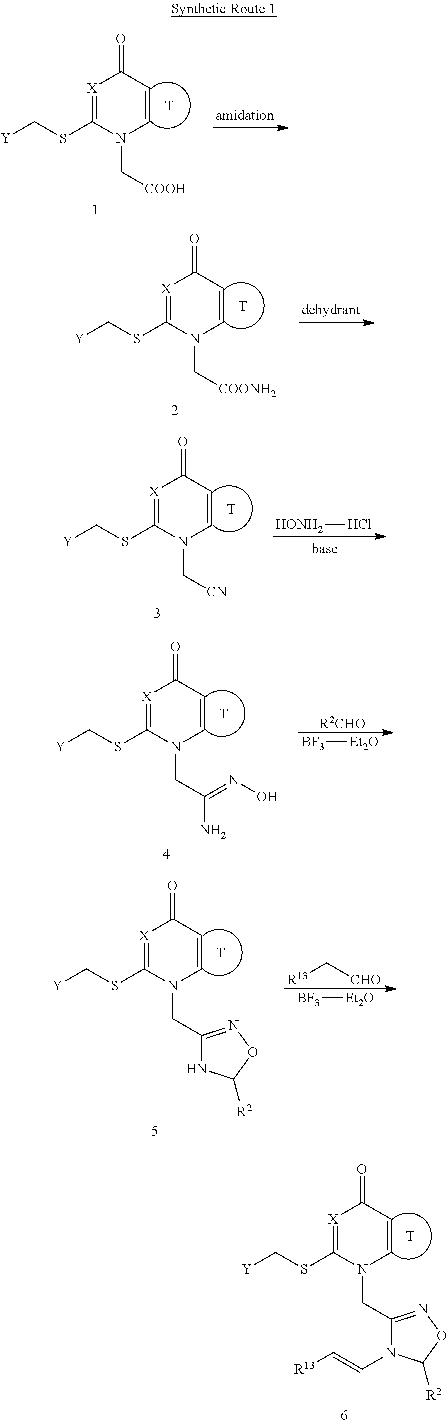

When W is Formula (a) meanwhile R¹ is alkenyl in the compounds of Formula (I), the compounds of Formula (I) (i.e. compound 6 in route 1) can be prepared according to the method illustrated by synthetic route 1, in which: R¹³ is C₁-C₁₀ alkyl; T, X, Y and R² are as hereinbefore defined;

Compound 1 is converted to compound 2 by amidation which is then dehydrated to give compound 3; compound 3 reacts with hydroxylamine hydrochloride in the presence of a base to produce compound 4; catalyzed by boron trifluoride-diethyl ether complex, the reaction of compound 4 with R²CHO yields compound 5 in an aprotic solvent; compound 5 is then transformed into compound 6 by reaction with R¹³CH₂CHO in the presence of boron trifluoride-diethyl ether complex as the catalyst;

or,

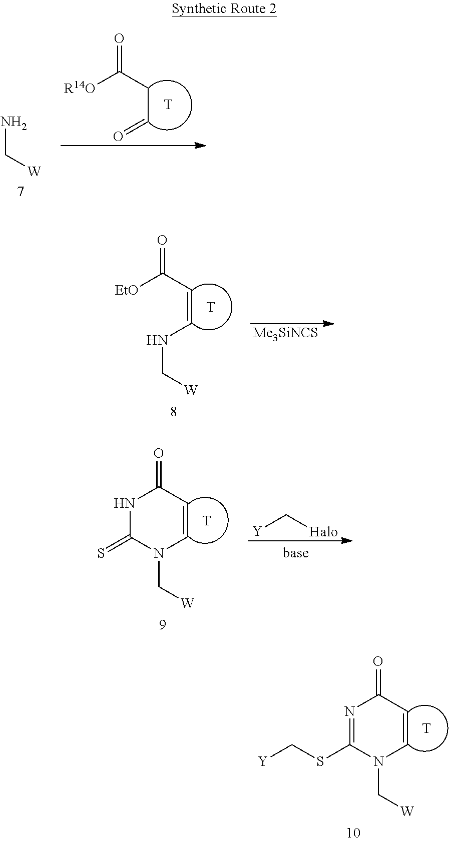

When T is 4 to 6-membered aliphatic ring and X═N, meanwhile R¹ is not alkenyl in the compounds of Formula (I), the compounds of Formula (I) (i.e. compound 10 in route 2) can be prepared according to the method illustrated by synthetic route 2, in which: R¹⁴ is methyl or ethyl; Halo, T, W and Y are as hereinbefore defined;

In a polar solvent, compound 7 is condensed with a cycloalkanone carboxylate

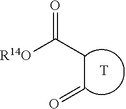

to yield compound 8 in the presence of a dehydrant; compound 8 reacts with Me₃SiNCS to produce compound 9 which is then converted to compound 10 by reaction with

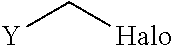

in a polar solvent in the presence of a base;

or,

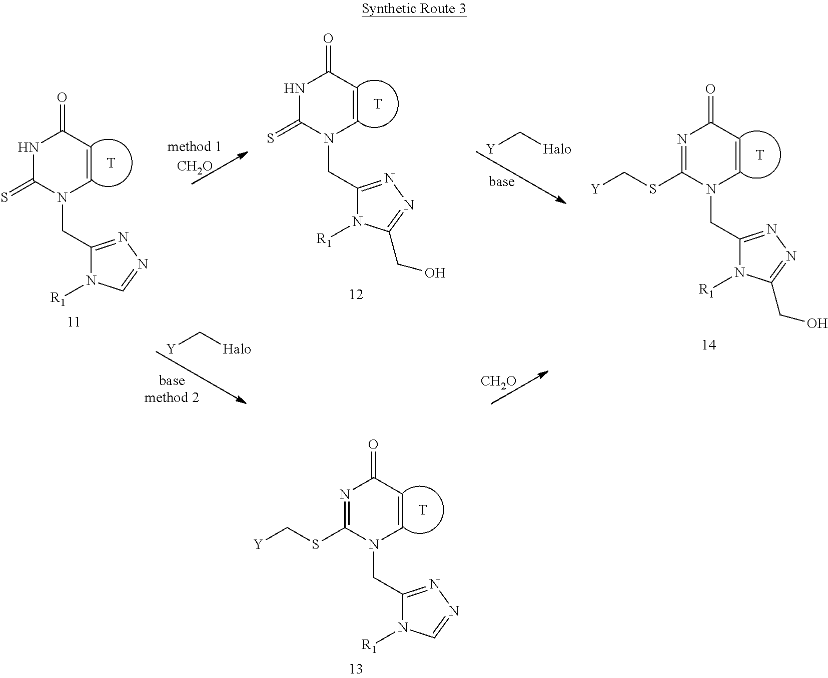

When W is Formula (c) meanwhile R² is hydroxymethyl in the compounds of Formula (I), above mentioned compound 10 can be prepared according to either of the methods illustrated by synthetic route 3, in which Halo, T, Y and R¹ are as hereinbefore defined;

According to the first method, compound 11 is heated with aqueous formaldehyde to afford compound 12 which then reacts in the presence of a base with

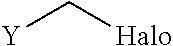

in a polar solvent to give compound 14;

According to the second method, compound 11 first reacts with

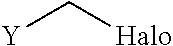

to give compound 13 which is then heated with aqueous formaldehyde to afford compound 14;

or,

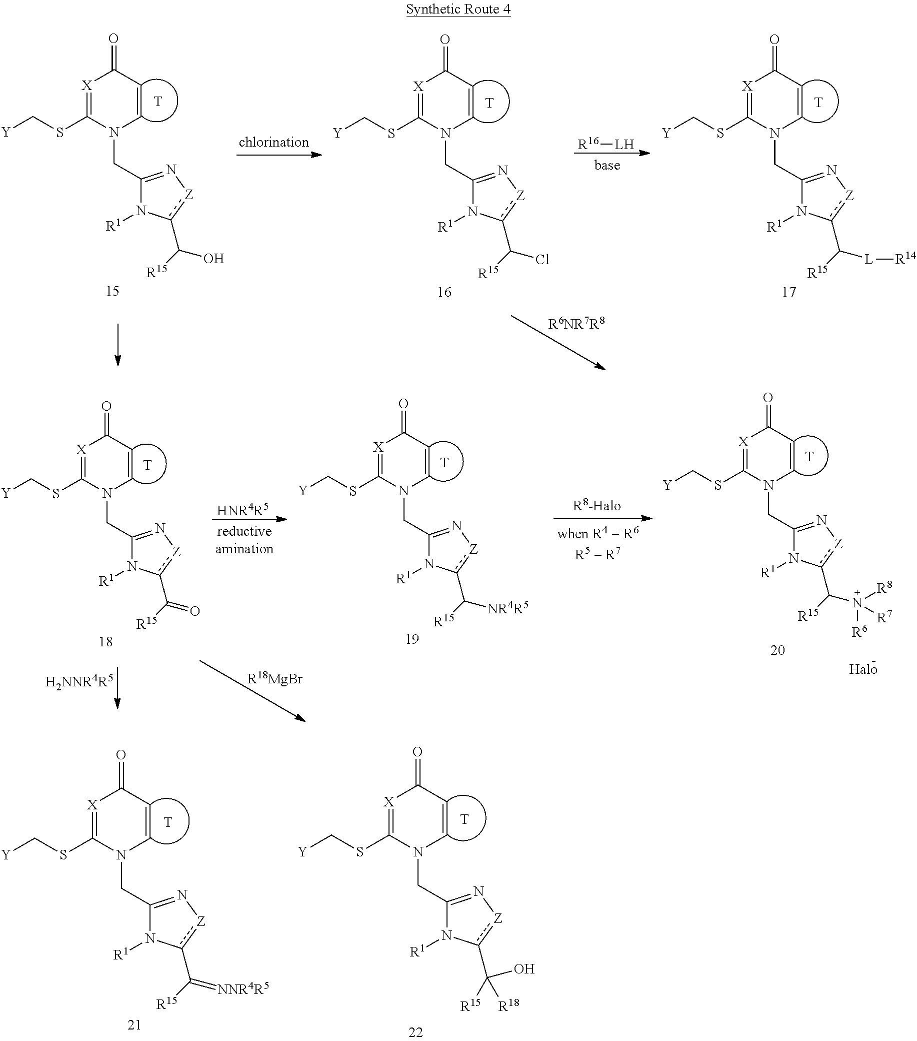

A compound of Formula (I) can be converted into another structure of the compound of Formula (I) by functional transformation, which is illustrated by route 4 as follows:

When R² in Formula (I) is α-hydroxyalkyl, the compound of Formula (I) can be represented by the structure of compound 15, in which R¹⁵ is H or C₁-C₆ alkyl; R¹⁶ is C₁-C₆ alkyl that is optionally substituted by NR⁴R⁵ or phenyl, wherein phenyl is optionally substituted by halogen; R¹⁸ is C₁-C₆ alkyl; L is NR⁴, O or S; Z is CH, N or O; T, X, Y, R¹, R⁴, R⁵, R⁶, R² and R⁸ are as hereinbefore defined;

Compound 15 is chlorinated to afford compound 16 which then reacts with R¹⁶LH to give compound 17 in the presence of a base;

Compound 15 is oxidized to afford compound 18 which then reacts with HNR⁴R⁵ to give compound 19 in the presence of a reductant;

The condensation reaction of compound 16 with R⁶NR⁷R⁸ yields compound 20;

The condensation reaction of compound 19 with R⁸-Halo also yields compound 20;

The condensation reaction of compound 18 with H₂NNR⁴R⁵ yields compound 21;

The reaction of compound 18 with R¹⁸BrMg yields compound 22.

### DETAILED DESCRIPTION OF THE INVENTION

The term “substituted” in the invention means substituted by one or more groups. In the case where more than one group is selected from the same series of candidate substituents, they may be the same or different.

The term “independently” in the invention means that more than one group as defined may be selected from the same series of candidate groups, they do not influence each other, and they may be the same or different.

The term “aliphatic ring” in this invention refers to a cyclic hydrocarbonyl that has 3 to 12 carbon atoms and may have one or more unsaturated bonds. Of particular interest are 4 to 6-membered aliphatic rings. Examples include, but are not limited to cyclopentane or cyclopentene.

As used herein, the term “alkyl” or similar terms such as “alkoxy” includes all straight and branched isomers containing a specified number of carbon atoms. Of particular interest is C₁-C₁₂ alkyl, with C₁-C₆ alkyl preferred. More preferably, it is C₁-C₄ alkyl, most preferably C₁-C₃ alkyl. Examples include, but are not limited to methyl, ethyl, n-propyl, isopropyl, n-butyl, sec-butyl, isobutyl, tert-butyl.

As used herein, the term “alkenyl” refers to all straight and branched isomers that have a specified number of carbon atoms, meanwhile have 1 to 5 double bonds, such as C₃-C₁₂ alkenyl and C₃-C₈ alkenyl. Examples include, but are not limited to vinyl and propenyl.

As used herein, the term “cycloalkyl” refers to a non-aromatic, saturated, cyclic aliphatic groups containing a specified number of carbon atoms. Examples include, but are not limited to cyclopropyl, cyclobutyl, cyclopentyl and cyclohexyl.

Unless otherwise defined, the phrase “nonaromatic heterocycle” refers to a saturated monocyclic ring system that has 2 to 10 carbon atoms and 1 to 4 heteratoms selected from the group consisting of N, O and S. Examples include, but are not limited to aziridine, thiirane, azetidine, tetrahydrofuran, pyrrolidine, piperidine, piperazine and morpholine.

As used herein, the phrase “aromatic heterocycle” refers to a monocyclic ring system that complies with Hilckel's Rule, has 4 to 10 ring atoms and 1 to 4 heteratoms selected from the group consisting of N, O and S. Examples include, but are not limited to pyridine, pyrimidine, pyrazole, furan, thiophene, thiazole and pyrazine.

Unless otherwise indicated, the term “halogen” in this invention includes fluorine, chlorine, bromine and iodine.

Unless otherwise indicated, a substitution on an alkyl, alkenyl or cycloalkyl group in this invention may occur on any carbon atom as long as the substitution on this carbon atom is not saturated.

Unless otherwise indicated, a substitution on a phenyl or a heterocyclic ring in this invention may occur at any position which is not yet occupied by an atom other than hydrogen.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

In certain embodiments, the compounds of Formula (I), (II) or (III) are salts in themselves, for example, when R² is alkyl which is substituted by

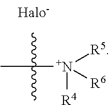

In addition, certain other compounds can form pharmaceutically acceptable salts which are a particularly important portion of the scope of this invention.

The term “pharmaceutically acceptable salts” refers to salts that retain the desired biological activity of the subject compound and exhibit minimal undesired toxicological effects. These pharmaceutically acceptable salts may be obtained directly in the preparation and purification of the compound, or separately by reacting the compound in its free acid or free base form with a suitable base or acid, respectively.

Specifically, certain compounds of this invention may contain a basic group (for example, but without limitation, when R² is alkyl which is substituted by —NR⁴R⁵), and therefore are capable of forming pharmaceutically acceptable salts by treatment with a suitable acid. Suitable acids include both pharmaceutically acceptable inorganic acids and organic acids. Representative examples of pharmaceutically acceptable salts include, but are not limited to hydrochloride, sulfate, hydrobromide, mesylate, nitrate, phosphate, acetate, oxalate, succinate, tartrate, maleate, arginine salts.

Certain compounds of this invention may contain an acid group (for example, but without limitation, when R² is alkyl which is substituted by —COOH), and therefore are capable of forming pharmaceutically acceptable salts by treatment with a suitable base. Suitable bases include both pharmaceutically acceptable inorganic bases and organic bases. Representative examples of pharmaceutically acceptable inorganic basic salts include, but are not limited to sodium, potassium, lithium, calcium, aluminium, zinc, and ammonium salts. Examples of organic basic salts include, but are not limited to the salts formed by treatment of an acid group with methylamine, ethylamine, triethylamine, meglumine, tromethamine

Some compounds or their pharmaceutically acceptable salts in this invention are crystallized or recrystallized from water or organic solvents. The crystalline product may contain solvent molecules that are used. Additionally, different crystallization conditions may result in different polymorphic forms of the crystalline products. Therefore, this invention includes within its scope all crystalline products containing different amounts of solvent as well as all polymorphic forms of the compounds of Formula (I), (II) or (III).

Some compounds of this invention may contain one or more chiral centers. For example, but without limitation, when W is Formula (a), various optical isomers may exist, including racemes, racemic mixtures, enantiomers, diastereoisomers, diastereoisomeric mixtures. This invention includes within its scope all optical isomers of the compounds of Formula (I), (II) or (III). Some compounds of Formula (I), (II) or (III) may be present in the form of cis-trans isomers, for example, but without limitation, when R¹ is alkenyl. Therefore, this invention includes within its scope the individual cis- or trans-isomer as well as the mixture of both isomers of a compound. Some compounds of Formula (I), (II) or (III) may have rotational isomers as a result of rotational restriction of certain group within the molecule. This invention includes within its scope the individual rotational isomer as well as the mixture of several rotational isomers of a compound.

As used herein, the term “solvate” refers to a molecular complex of a compound of this invention with one or more stoichiometric solvent molecules that are pharmaceutically acceptable, such as ethanol. When the solvent is water, the term “hydrate” is used.

### Compounds of Particular Interest

Within the definition of the substituents of the compounds shown by Formula (I), (II) or (III), the following more detailed description of the groups are of particular interest:

Preferably, T is 5-membered aliphatic ring or benzene ring;

Preferably, X is CH or N;

More preferably, within the compounds of Formula (I), when T is 5-membered aliphatic ring, X is N; when T is benzene, X is CH.

Preferably, Y is phenyl which is substituted by halogen, more preferably by fluorine atoms. Most preferably, Y is 4-fluorophenyl or 2,3-difluorophenyl;

W is selected from 6 structures of Formulae (a-f) as follows:

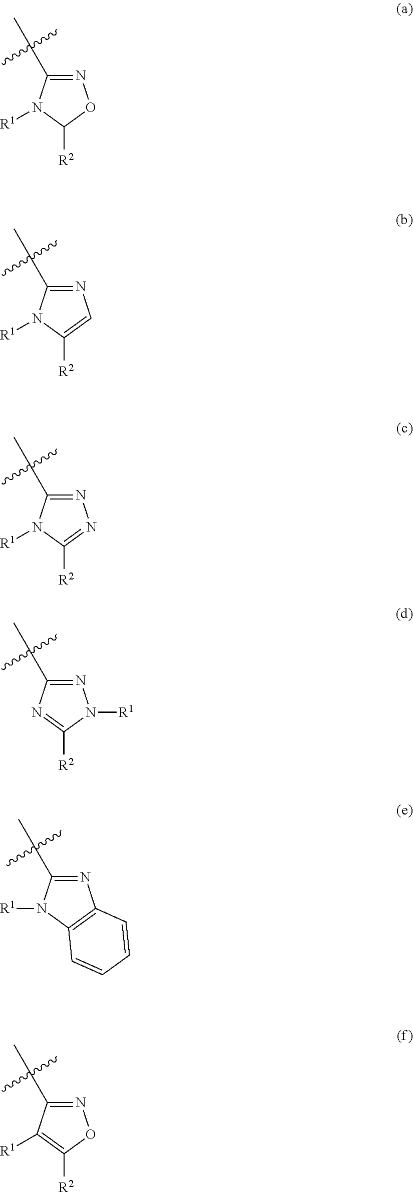

Preferably, R¹ is

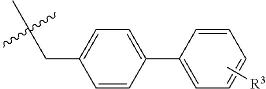

or —NR⁴R⁵ substituted C₂-C₄ alkyl;

More preferably, R¹ is

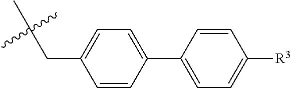

### or —CH₂CH₂NR⁴R⁵;

Most preferably, R¹ is (4-(trifluoromethyl)biphenyl-4-yl)methyl.

Preferably, R² is —COR⁴, —CONR⁴R⁵, C₁-C₅ alkyl or C₃-C₅ cycloalkyl, wherein alkyl or cycloalkyl is optionally substituted by —NR⁴R⁵, —OR⁴, —SR⁴, —SO₂R⁴, ═NNR⁴R⁵, —NHCOR⁴, —NHSO₂R⁴, —NHCSNHR⁴ or

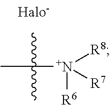

More preferably, R² is —CONR⁴R⁵, cyclopropyl or C₁-C₅ alkyl, wherein the alkyl is substituted by —NR⁴R⁵, —OR⁴, —SR⁴, —SO₂R⁴, ═NNR⁴R⁵, —NHCOR⁴, —NHSO₂R⁴, —NHCSNHR⁴ or

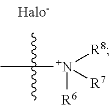

Most preferably, R² is —CONR⁴R⁵, cyclopropyl or C₁-C₅ alkyl, wherein the C₁-C₅ alkyl is substituted by —NR⁴R⁵, —OR⁴, —SR⁴, ═NNR⁴R⁵, or

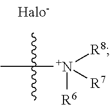

Representative examples of R² include, but are not limited to dimethylcarbamoyl, 2-(diethylamino)ethylcarbamoyl, (2-(di ethylamino)ethyl)(methyl)carbamoyl, (dimethylamino)methyl, (diethylamino)methyl, pyrrolidin-1-ylmethyl, ((4-fluorobenzyl)(methyl)amino)methyl, isopropyl, cyclopropyl, 3-(diethylamino)propyl, 4-(di ethylamino)butyl, hydroxymethyl, 1-hydroxyethyl, (4-fluorobenzylthio)methyl, (isopropyl(methyl)amino)methyl, ((1-ethylpyrrolidin-2-yl)methylamino)methyl, (4-ethylpiperazin-1-yl)methyl, ((2-(dimethylamino)ethyl)(methyl)amino)methyl, ((2-(diethylamino)ethyl)(methyl)amino)methyl, (((2-(dimethylamino)ethyl)(ethyl)amino)methyl, (((3-(dimethylamino)propyl)(methyl)amino)methyl), (methyl(pyridin-2-ylmethyl)amino)methyl, (4-(dimethylamino)piperidin-1-yl)methyl, (2,2-dimethylhydrazono)methyl, (2-hydroxyethoxy)methyl, (2-(diethylamino)ethoxy)methyl, 1-(2-(dimethylamino)ethylamino)ethyl, 1-((2-(dimethylamino)ethyl)(methyl)amino)ethyl, 1-(2-(diethylamino)ethylamino)ethyl, 1-((2-(diethylamino)ethyl)(methyl)amino)ethyl, 1-((3-(dimethylamino)propyl)(methyl)amino)(methyl)ethyl, ((methyl(2-(pyrrolidin-1-yl)ethyl)amino)methyl, ((methyl(2-(piperidin-1-yl)ethyl)amino)methyl, 3-(pyrrolidin-1-yl)propyl, 3-(piperidin-1-yl)propyl, 4-(pyrrolidin-1-yl)butyl, 4-(piperidin-1-yl)butyl,

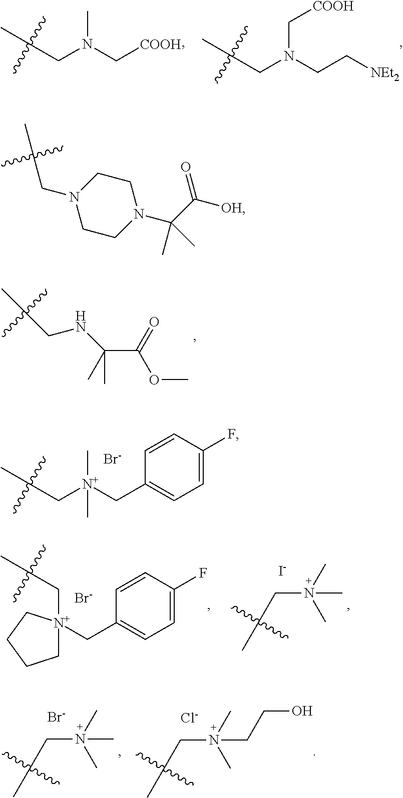

Compounds of Formula (I), (II) or (III) include:
2-(4-fluorobenzylthio)-1-((5-n-heptyl-4,5-dihydro-1,2,4-oxadiazol-3-yl)methyl)-6,7-dihydro-1H-cyclopenta[(1]pyrimidin-4(5H)-one;
1-((5-n-decyl-4,5-dihydro-1,2,4-oxadiazol-3-yl)methyl)-2-(4-fluorobenzylthio)-6,7-dihydro-1H-cyclopenta[d]pyrimidin-4(5H)-one;
2-(4-fluorobenzylthio)-1-((5-n-heptyl-4,5-dihydro-1,2,4-oxadiazol-3-yl)methyl)quinolin-4(1H)-one;
2-(4-fluorobenzylthio)-1-((5-(4′-(trifluoromethyl)biphenyl-4-yl)-4,5-dihydro-1,2,4-oxadiazol-3-yl)methyl)-6,7-dihydro-1H-cyclopenta[d]pyrimidin-4 (5H)-one;
(E)-2-(4-fluorobenzylthio)-1-((5-n-heptyl-4-(n-oct-1-enyl)-4,5-dihydro-1,2,4-oxadiazol-3-yl)methyl)-6,7-dihydro-1H-cyclopenta[d]pyrimidin-4(5H)-one;
2-(2,3-difluorobenzylthio)-1-((4-(2-morpholinoethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one;
ethyl
4-(3-((2-(2,3-difluorobenzylthio)-4-oxo-4,5,6,7-tetrahydro-1H-cyclopenta[d]pyrimidin-1-yl)methyl)-5-(4′-(trifluoromethyl)biphenyl-4-yl)-1,2,4-oxadiazol-4(5H)-yl)piperidine-1-carboxylate;
ethyl
4-(3-((2-(4-fluorobenzylthio)-4-oxo-4,5,6,7-tetrahydro-1H-cyclopenta[d]pyrimidin-1-yl)methyl)-5-(4′-(trifluoromethyl)biphenyl-4-yl)-1,2,4-oxadiazol-4(5H)-yl)piperidine-1-carboxylate;
2-(4-fluorobenzylthio)-1-((4-(2-(piperidin-1-yl)ethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one;
1-((4-(2-(diethylamino)ethyl)-5-(4′-(trifluoromethyl)biphenyl-4-yl)-4,5-dihydro-1,2,4-oxadiazol-3-yl)methyl)-2-(4-fluorobenzylthio)-6,7-dihydro-1H-cyclopenta[d]pyrimidin-4(5H)-one;
2-(2,3-difluorobenzylthio)-1-((4-(2-(piperidin-1-yl)ethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one;
2-(4-fluorobenzylthio)-1-((5-phenyl-1H-imidazol-2-yl)methyl)-6,7-dihydro-1H-cyclopenta[d]pyrimidin-4(5H)-one;
1-((1H-tetrazol-5-yl)methyl)-2-(4-fluorobenzylthio)-6,7-dihydro-1H-cyclopenta[d]pyrimidin-4(5H)-one;
2-(4-fluorobenzylthio)-1-((1-((4′-(trifluoromethyl)biphenyl-4-yl)methyl)-1H-imidazol-2-yl)methyl)-6,7-dihydro-1H-cyclopenta[d]pyrimidin-4(5H)-one;
1-((1-dodecyl-1H-imidazol-2-yl)methyl)-2-(4-fluorobenzylthio)-6,7-dihydro-1H-cyclopenta[d]pyrimidin-4(5H)-one;
1-((1-butyl-1H-imidazol-2-yl)methyl)-2-(4-fluorobenzylthio)-6,7-dihydro-1H-cyclopenta[d]pyrimidin-4(5H)-one;
1-((1-((4′-chlorobiphenyl-4-yl)methyl)-1H-imidazol-2-yl)methyl)-2-(4-fluorobenzylthio)-6,7-dihydro-1H-cyclopenta[d]pyrimidin-4(5H)-one;
1-((1-(biphenyl-4-ylmethyl)-1H-imidazol-2-yl)methyl)-2-(4-fluorobenzylthio)-6,7-dihydro-1H-cyclopenta[d]pyrimidin-4(5H)-one;
1-((1-n-butyl-5-(4-chlorophenyl)-1H-imidazol-2-yl)methyl)-2-(4-fluorobenzylthio)-6,7-dihydro-1H-cyclopenta[d]pyrimidin-4(5H)-one;
1-((1-n-butyl-5-(4-chlorophenyl)-1H-imidazol-2-yl)methyl)-2-(2-nitrobenzylthio)-6,7-dihydro-1H-cyclopenta[d]pyrimidin-4(5H)-one;
1-((5-n-decyl-1-(2-(diethylamino)ethyl)-1H-imidazol-2-yl)methyl)-2-(4-fluorobenzylthio)-6,7-dihydro-1H-cyclopenta[d]pyrimidin-4(5H)-one;
1-((1-(2-(diethylamino)ethyl)-5-(4′-(trifluoromethyl)biphenyl-4-yl)-1H-imidazol-2-yl)methyl)-2-(4-fluorobenzylthio)-6,7-dihydro-1H-cyclopenta[d]pyrimidin-4(5H)-one;
1-((1-benzyl-5-((diethylamino)methyl)-1H-imidazol-2-yl)methyl)-2-(4-fluorobenzylthio)-6,7-dihydro-1H-cyclopenta[d]pyrimidin-4(5H)-one;
2-(4-fluorobenzylthio)-1-((5-methyl-1-((4′-(trifluoromethyl)biphenyl-4-yl)methyl)-1H-imidazol-2-yl)methyl)-6,7-dihydro-1H-cyclopenta[d]pyrimidin-4(5H)-one;
1-((5-((diethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
2-(4-fluorobenzylthio)-1-((5-(pyrrolidin-1-ylmethyl)-1-((4′-(trifluoromethyl)biphenyl-4-yl)methyl)-1H-imidazol-2-yl)methyl)-6,7-dihydro-1H-cyclopenta[d]pyrimidin-4(5H)-one;
1-((5-(((4-fluorobenzyl)(methyl)amino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
1-((5-((4-benzylpiperazin-1-yl)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
1-((5-((dimethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
N-(4-fluorobenzyl)-1-(2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazol-5-yl)-N,N-dimethylmethanaminium bromide;
1-((5-((dimethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
1-(4-fluorobenzyl)-1-((2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazol-5-yl)methyl)pyrrolidinium bromide;
ethyl
2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxylate;
2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxylic acid;
2-((2-(4-fluorobenzylthio)-4-oxo-4,5,6,7-tetrahydro-1H-cyclopenta[d]pyrimidin-1-yl)methyl)-N,N-dimethyl-1-((4′-(trifluoromethyl)biphenyl-4-yl)methyl)-1H-imidazole-5-carboxamide;
2-(4-fluorobenzylthio)-1-((1-((4′-(trifluoromethyl)biphenyl-4-yl)methyl)-1H-benzo[d]imidazol-2-yl)methyl)-6,7-dihydro-1H-cyclopenta[d]pyrimidin-4(5H)-one;
1-((4-ethyl-5-(4′-(trifluoromethyl)biphenyl-4-yl)-4H-1,2,4-triazol-3-yl)methyl)-2-(4-fluorobenzylthio)-6,7-dihydro-1H-cyclopenta[d]pyrimidin-4(5H)-one;
1-((4-ethyl-5-(4′-(trifluoromethyl)biphenyl-4-yl)-4H-1,2,4-triazol-3-yl)methyl)-2-(4-methoxybenzylthio)-6,7-dihydro-1H-cyclopenta[d]pyrimidin-4(5H)-one;
2-(2,3-difluorobenzylthio)-1-((4-ethyl-5-(4′-(trifluoromethyl)biphenyl-4-yl)-4H-1,2,4-triazol-3-yl)methyl)-6,7-dihydro-1H-cyclopenta[d]pyrimidin-4(5H)-one;
1-((4-ethyl-5-(3-phenylpropyl)-4H-1,2,4-triazol-3-yl)methyl)-2-(4-fluorobenzylthio)-6,7-dihydro-1H-cyclopenta[d]pyrimidin-4(5H)-one;
1-((4-ethyl-5-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((1-((4′-(trifluoromethyl)biphenyl-4-yl)methyl)-1H-1,2,4-triazol-3-yl)methyl)-6,7-dihydro-1H-cyclopenta[d]pyrimidin-4(5H)-one;
methyl
2-(((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)(methyl)amino)acetate;
2-(((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)(methyl)amino)acetic acid;
2-(4-fluorobenzylthio)-1-((5-methyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-propyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-isopropyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-cyclopropyl-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(benzylsulfonylmethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(3-(diethyl amino)propyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(4-(diethylamino)butyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-(hydroxymethyl)-4-((4′-(trifluoromethyl)biphenyl-4-yl)methyl)-4H-1,2,4-triazol-3-yl)methyl)-6,7-dihydro-1H-cyclopenta[d]pyrimidin-4 (5H)-one;
2-(4-fluorobenzylthio)-4-((4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazole-3-carbaldehyde;
1-((5-((dimethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
N-(4-fluorobenzyl)-1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)-N,N-dimethylmethanaminium bromide;
1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)-N,N,N-trimethylmethanaminium iodide;
1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)-N,N,N-trimethylmethanaminium bromide;
1-((5-((diethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((4-fluorobenzyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((isopropyl(methyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
(±)1-((5-(((1-ethylpyrrolidin-2-yl)methylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1 H-cyclopenta[d]pyrimidin-4(5H)-one;
1-((5-((2S,6R)-2,6-dimethylmorpholino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
methyl-2-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)amino)-2-methylpropanoate;
1-((5-((4-ethyl)piperazin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-methoxyethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
methyl-2-(4-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)piperazin-1-yl)-2-methylpropanoate;
2-(4-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)piperazin-1-yl)-2-methylpropanoic acid;
1-((5-((2-(dimethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1 H-cyclopenta[d]pyrimidin-4(5H)-one;
1-((5-((2-(diethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-((2-(diethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one tartrate;
1-((5-(((3-(dimethylamino)propyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-morpholinoethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
(R)-1-((5-((3-(dimethylamino)pyrrolidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(S)-1-((5-((3-(dimethylamino)pyrrolidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-(piperidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((2-(pyrrolidin-1-yl)ethylamino)methyl)-4-((4′-(trifluoro methyl)biphenyl-4-yl)methyl)-4H-1,2,4-triazol-3-yl)methyl)-6,7-dihydro-1H-cyclopenta[d]pyrimidin-4(5H)-one;
1-((5-((2-(diisopropylamino)ethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-((2-(diethylamino)ethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((4-methylpiperazin-1-yl)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((methyl(pyridin-2-ylmethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((cyclopropyl(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-((4-(dimethylamino)piperidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-((3,3-difluoropyrrolidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-((2-(dimethylamino)ethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(S)-1-((5-(((1-ethylpyrrolidin-2-yl)methylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-(2-oxoimidazolidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-(azidomethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one
1-((5-(aminomethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
N-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)ethanesulfonamide;
2-(4-fluorobenzylthio)-1-((5-((2-oxoimidazolidin-1-yl)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)-3-methylthiourea;
1-((5-(chloromethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((4-fluorobenzylthio)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
(E)-1-((5-((2,2-dimethylhydrazono)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(E)-1-((5-((2-tert-butylhydrazono)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(E)-2-(4-fluorobenzylthio)-1-((5-((piperidin-1-ylimino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)vinylacetate;
2-(4-fluorobenzylthio)-1-((5-(1-hydroxyethyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-acetyl-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
N-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methy 1)-2-hydroxy-N,N-dimethylethanaminium chloride;
2-(4-fluorobenzylthio)-1-((5-((2-hydroxyethoxy)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((2-(diethylamino)ethoxy)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(n-butoxymethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-(2-(dimethylamino)ethylamino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-(2-(dimethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-(2-(diethylamino)ethylamino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-(((2-(diethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-((2-(diethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one tartrate;
1-((5-(1-((3-(dimethylamino)propyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((2-(dimethylamino)ethyl)(ethyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
ethyl-2-((2-(diethylamino)ethyl)((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)amino)acetate;
2-((2-(diethylamino)ethyl)((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)amino)acetic acid;
N-(2-(diethylamino)ethyl)-2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxamide;
N-(2-(diethylamino)ethyl)-2-((2-(4-fluorobenzylthio)-4-oxo-4,5,6,7-tetrahydro-1H-cyclopenta[d]pyrimidin-1-yl)methyl)-N-methyl-1-((4′-(trifluoromethyl)biphenyl-4-yl)methyl)-1H-imidazole-5-carboxamide;
1-((5-(((2-(diethylamino)ethyl)(methyl)amino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
2-(4-fluorobenzylthio)-1-((5-((methyl(2-(pyrrolidin-1-yl)ethyl)amino)methyl)-4-((4′-(t rifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((methyl(2-(piperidin-1-yl)ethyl)amino)methyl)-4-((4′-(trifluoromethyl)biphenyl-4-yl)methyl)-1H-imidazol-2-yl)methyl)-6,7-dihydro-1H-cyclopenta[d]pyrimidin-4(5H)-one;
2-(4-fluorobenzylthio)-1-((5-(3-(pyrrolidin-1-yl)propyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-(3-(piperidin-1-yl)propyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-(4-(pyrrolidin-1-yl)butyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-(4-(piperidin-1-yl)butyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((methyl(2-(piperidin-1-yl)ethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
methyl-3-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)isoxazole-5-carboxylate;
2-(4-fluorobenzylthio)-1-((5-(hydroxymethyl)-4-((4′-(trifluoromethyl)biphenyl-4-yl)methyl)isoxazol-3-yl)methyl)-6,7-dihydro-1H-cyclopenta[d]pyrimidin-4(5H)-one;

1-((5-(chloromethyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
1-((5-((diethylamino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
1-((5-(((2-(dimethylamino)ethyl)(methyl)amino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
1-((5-(((2-(diethylamino)ethyl)(methyl)amino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
2-(4-fluorobenzylthio)-1-((5-((methyl(2-(piperidin-1-yl)ethyl)amino)methyl)-4-((4′-(trifluoromethyl)biphenyl-4-yl)methyl)isoxazol-3-yl)methyl)-6,7-dihydro-1H-cyclopenta[d]pyrimidin-4(5H)-one;
2-(4-fluorophenethyl)-1-((4-((4′-(trifluoromethyl)biphenyl-4-yl)methyl)-4H-1,2,4-triazol-3-yl)methyl)quinazolin-4(1H)-one;
cis-trans isomers, enantiomers, diastereoisomers, racemes, solvates, hydrates, or pharmaceutically acceptable salts thereof.

The structures of the above most preferred compounds are shown in the following preparation examples.

Compounds of this invention are effective inhibitors of the enzyme lipoprotein associated phospholipase A₂ and are expected to be useful in therapy, especially in the treatment and prevention of acute and chronic coronary events, such as those caused by peripheral vascular atherosclerosis and cerebrovascular atherosclerosis. That is to say, the present invention provides compounds of Formula (I), (II) or (III) that have potential uses in therapy.

Compounds of Formula (I), (II) or (III) according to this invention may inhibit the production of lysophosphatidylchloline (Lyso-PC), and therefore may have a general application in treating diseases associated with endothelial dysfunction, for example atherosclerosis, diabetes, hypertension, angina and ischemic reperfusion. Additionally, the compounds of Formula (I), (II) or (III) may also have a general application in any disorder involving the hydrolysis process of oxidized lipid with the participation of the enzyme Lp-PLA₂. In addition to atherosclerosis and diabetes, such diseases also include ischemia, rheumatoid arthritis, stroke, inflammatory condition of the brain (such as Alzheimer's Disease), myocardial infarction, reperfusion injury, septicemia, acute and chronic inflammation.

The enzyme Lp-PLA₂ is expressed in activated inflammatory cells (such as macrophages, lymphocytes, neutrophils, eosnophils), and therefore the compounds of Formula (I), (II) or (III) according to this invention may be of use in treating any disorder that is associated with activated inflammatory cells. Such conditions include psoriasis and various airway inflammations, such as asthma and chronic bronchitis.

Accordingly, the present invention provides the uses of a compound of Formula (I), (II) or (III) in inhibiting the activity of Lp-PLA₂, and as such in treating diseases associated with activity of the enzyme Lp-PLA₂. Those diseases may be connected with activation of inflammatory cells; with production of phosphatidyl choline and oxidized non-esterified fatty acid; with lipid oxidation with the participation of the enzyme Lp-PLA₂; or with endothelial dysfunction.

A compound of Formula (I), (II) or (III) according to the present invention may be of use in treating the above mentioned diseases in combination with an anti-hyperlipidaemic, anti-atherosclerotic, anti-diabetic, anti-anginal, antiinflammatory, or anti-hypertension agent or an agent for lowering Lp(a). Examples of the above agents include, but are not limited to cholesterol synthesis inhibitors such as statins, anti-oxidants such as probucol, insulin sensitisers, calcium channel antagonists, and non-steroidal anti-inflammatory drugs.

A compound of Formula (I), (II) or (III) according to the present invention may be used in combination with cholesterol lowering agents such as statins. The statins are HMG-CoA reductase inhibitors, including atorvastatin, simvarstatin, pravastatin, cerivastatin, fluvastatin, lovstatin and pitavastatin. The two kinds of agents may be administered at the same time or at different times according to the discretion of the physician.

Up to 30% of the patients with high cholesterol level are negative to statin treatment. A compound of Formula (I), (II) or (III) according to this invention is expected to be applied to this population of patients.

In considering that coronary heart diseases account for majority of the death for diabetic patients, the combination of a compound of Formula (I), (II) or (III) according to this invention with an anti-diabetic agent or an insulin sensitizer is of particular interest. Preferably, the insulin sensitizer is a PPAR gamma activator, such as rosiglitazone or pioglitazone.

In therapeutic use, the compounds of this invention are usually administrated in a standard pharmaceutical composition. The present invention therefore provides a pharmaceutical composition that comprises one or more compounds of Formula (I), (II) or (III) in therapeutically effective amount and a pharmaceutically acceptable auxiliary. The pharmaceutically acceptable auxiliary is a pharmaceutically acceptable carrier, excipient or controlled release agent.

The compounds and pharmaceutical compositions of the present invention can be formulated in various dosage forms, for example tablet, capsule, powder, syrup, solution, suspension or aerosol, and can be incorporated into appropriate solid or liquid carriers or diluents. The pharmaceutical compositions of the present invention may be stored in appropriate injection or instillation disinfectors. The pharmaceutical compositions may also contain an odorant, a flavouring agent, etc.

A pharmaceutical composition of the present invention comprises a safe, effective amount (e.g. 0.1-99.9 parts by weight, preferably 1-90 parts by weight) of a compound of Formula (I), (II) or (III) or pharmaceutically acceptable salt thereof, and a balance of a pharmaceutically acceptable excipient, based on 100 parts by weight of the composition in total. In other words, the pharmaceutical composition of this invention contains 0.1-99.9%, preferably 1-90% by weiht of the compound of Formula (I), or (II) or a pharmaceutically acceptable salt thereof, and a balance of a pharmaceutically acceptable auxiliary, based on 100% by weight of the composition in total.

The preferred proportion of the compound of Formula (I), (II) or (III) as an active ingredient is more than 60% by weight. The pharmaceutically acceptable carrier, excipient or controlled release agent as the rest part accounts for 0-40% by weight, preferably 1-20%, most preferably 1-10%.

The compounds of Formula (I), (II) or (III) according to this invention or pharmaceutical compositions comprising the compounds of Formula (I), (II) or (III) are expected to be administrated to mammal, including human and animal. The routes of administration may include oral, nasal, transdermal, pulmonary or gastrointestinal drug delivery, preferably oral route. Preferably, the composition is in unit dosage form. Such a unit may contain 0.01 mg to 200 mg of the active ingredient, preferably 0.5 mg to 100 mg, in a daily dose or sub-dose. Regardless of the method of administration, the individual optimal dose depends on the condition being treated, and is usually seeked gradually from small dose to high dose.

The pharmaceutical compositions of the present invention may be administrated in oral, intravenous, intramuscular or subcutaneous route. In consideration of the convenience for preparation and administration, a solid composition is preferred, especially tablet and capsule that is packed with solid or liquid materials. The preferred route of administration of the pharmaceutical composition is p.o.

The solid carriers may include starch, lactose, calcium dihydrogen phosphate, microcrystalline cellulose, sucrose and white clay. The liquid carriers may include sterile water, polyethylene glycol (PEG), nonionic surfactant and cooking oil (such as corn oil, peanut oil and sesame oil) as far as the carrier is in accordance with the property of active ingredient and the route of administration. The commonly used adjuvants for preparing the pharmaceutical compositions include spices, pigments, preservatives and antioxidants such as vitamin E, vitamin C, BHT and BHA.

The injections include, but are not limited to sterile, injectable, aqueous or non-aqueous solution, suspension and emulsion. Those injections can be prepared with appropriate parenteral diluent, dispersant, wetting agent or suspending agent. The injections can be sterilized with biofilter that is capable of retaining bacteria, or with bactericide which is dissolved or dispersed in injectable medium, or prepared by the other methods well known in the pharmacy art.

### PREPARATION METHODS

### Abbreviations

DUB—1,8-diazabicyclo[5.4.0]undec-7-ene
DCE—dichloroethane
DCM—dichloromethane

### DIPEA—N,N-diisopropylethylamine

DMAP—4-dimethylaminopyridine
DME—dimethoxyethane
DMF—dimethyl formamide
DMSO—dimethylsulfoxide
DPPA—diphenyl phosphoryl azide
EA—ethyl acetate
EDCI—1-(3-dimethylaminopropyl)-3-ethylcarbodiimide hydrochloride
Et₂O—diethyl ether
EtOH—ethanol
HOBt—1-hydroxybenzotriazole
HOBu-t—tert-butanol
i-PrOH—isopropanol
KOBu-t—potassium tert-butoxide
LDA—lithium diisopropylamide
m-CPBA—meta-chloroperoxybenzoic acid
MeOH—methanol
NaH—sodium hydride
NaHMDS—sodium bis(trimethylsilyl)amide
n-BuLi—n-butyllithium

### NCS—N-chlorosuccinimide

PE—petroleum ether
s-BuLi—isobutyllithium
TFFA—trifluoro acetic anhydride
THF—tetrahydrofuran

### Synthetic Routes

The compounds of this invention can be prepared according to the methods illustrated by the following synthetic route 1, 2, 3 or 4. Unless otherwise indicated, the substituents appeared in the synthetic routes are as hereinbefore defined for Formula (I).

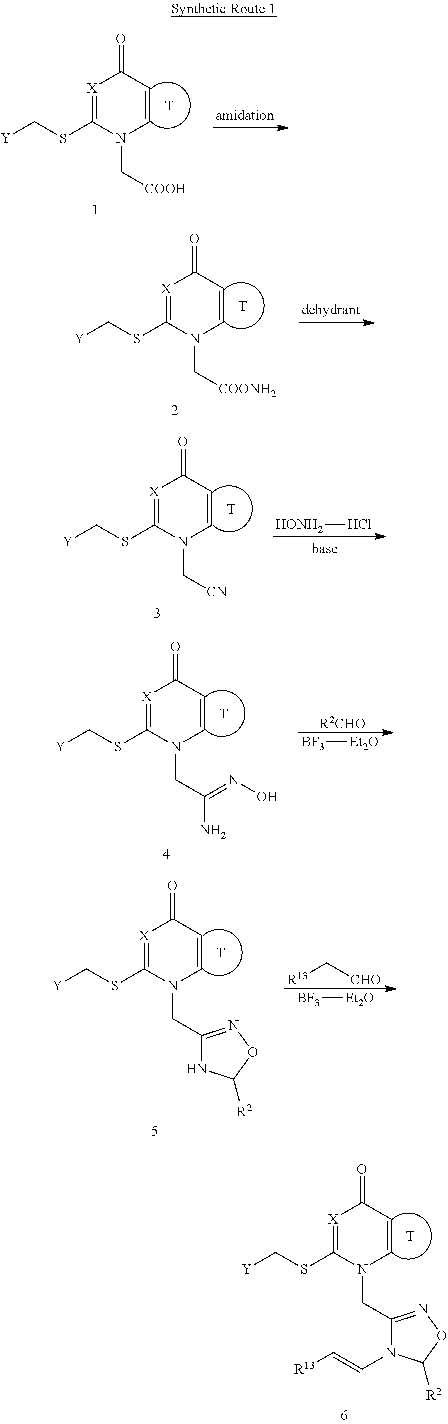

When W is Formula (a) meanwhile R¹ is alkenyl in the compounds of Formula (I), the compounds of Formula (I) (i.e. compound 6 in route 1) can be prepared according to the method illustrated by synthetic route 1, in which: R¹³ is C₁-C₁₀ alkyl; T, X, Y and R² are as hereinbefore defined:

Compound 1 may be purchased from suppliers or prepared according to the literature methods (such as those disclosed in PCT patent applications WO03016287 and WO03042206, the published contents of which are cited in their entity as references in this invention).

Compound 1 is converted to compound 2 by amidation. In one embodiment, this process may be accomplished by chlorination of the acid compound 1 and subsequent amination of the resulting acyl chloride. The chlorinating agents include thionyl chloride and oxalyl chloride. The amination agents can be excessive concentrated aqueous ammonia or ammonia-methanol solution. The reaction occurs in aprotic solvent, such as DCM, DCE, acetonitrile and THF. The reaction temperature is between −15° C. and 0° C. In another embodiment, this process may be accomplished by direct reaction of compound 1 with an ammonium salt in the presence of a condensing agent, wherein the condensing agent includes dicyclohexylcarbodiimide (DCC), diethyl azodicarboxylate/Ph3P, carbonyldiimidazole, 1-(3-dimethylaminopropyl)-3-ethylcarbodiimide hydrochloride/1-hydroxybenzotriazole (EDCI/HOBt), O-(Benzotriazol-1-yl)-N,N,N′,N′-tetramethylisouronium tetrafluoroborate (TBTU), and the inorganic ammonium salts include, for example, ammonium chloride, ammonium carbonate and ammonium bicarbonate. The reaction occurs in the presence of an organic base, such as triethylamine, DIPEA and DBU. The reaction occurs in a non-alcohol solvent, such as DCM, DCE, acetonitrile, THF, toluene and DMF; preferably in DCM, acetonitrile or DMF. The reaction temperature is between 0° C. and room temperature (rt).

Compound 2 is dehydrated to give compound 3. The preferred dehydrant is TFFA or POCl₃. An organic base, such as pyridine and triethylamine, is optionally added to the reaction solution. The reaction occurs in an aprotic solvent, such as DCM, DCE and THF; preferably in DCM or DMF. The reaction temperature is between −40° C. and rt.

Compound 3 reacts with hydroxylamine hydrochloride in the presence of an excessive base to produce compound 4, wherein the mentioned base is an organic base or an inorganic base, such as triethylamine, potassium carbonate and sodium hydroxide. The reaction occurs in a polar solvent, such as methanol, ethanol, DMF and water or a mixed solvent thereof. The reaction temperature is between 0° C. and 60° C., preferably room temperature.

Catalyzed by boron trifluoride-diethyl ether complex (BF₃-Et₂O), the reaction of compound 4 with 1 equivalent of R²CHO yields compound 5. More than 1 equivalent (equiv) of BF₃-Et₂O is required, typically 2 equiv. The reaction solvents include, for example, Et₂O or THF. The reaction temperature is between 0° C. and 40° C., preferably room temperature.

Compound 5 is transformed into compound 6 by reaction with R¹³CH₂CHO in the presence of BF₃-Et₂O. More than 1 equiv of BF₃-Et₂O is required; typically 2 equiv. The reaction solvent is Et₂O or THF. The reaction temperature is between 0° C. and 40° C., preferably room temperature.

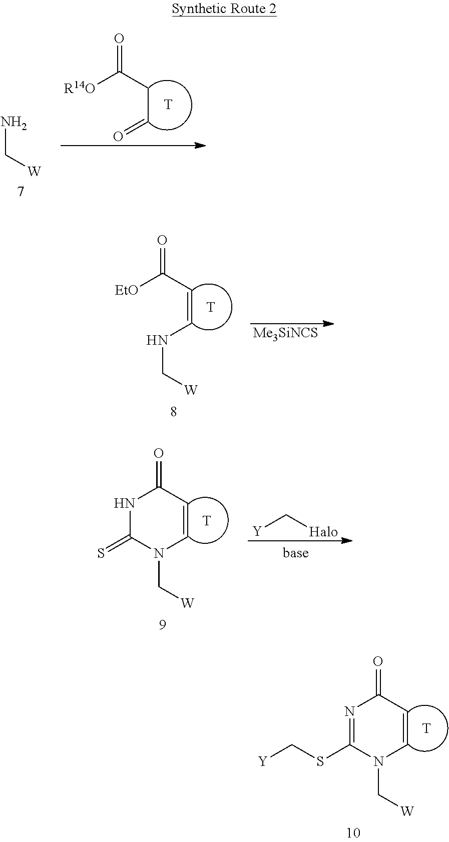

When T is 4 to 6-membered aliphatic ring and X═N, meanwhile R¹ is not alkenyl in the compounds of Formula (I), the compounds of Formula (I) (i.e. compound 10) can be prepared according to the method illustrated by synthetic route 2, in which: R¹⁴ is methyl or ethyl; Halo, T, W and Y are as hereinbefore defined:

Compound 7 or its hydrochloride form is condensed with 1 equiv of a cycloalkanone carboxylate

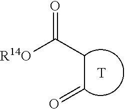

to yield compound 8. When compound 7 is in hydrochloride form, at least 1 equiv of an organic base is necessary, such as triethylamine and DIPEA. The reaction occurs in the presence of a dehydrant, including such as molecular sieve, azeotropic toluene and Si(OEt)₄. The reaction solvents include such as methanol, ethanol, toluene and acetic acid. The reaction temperature is between 0° C. and 140° C., preferably the reflux temperature of the solvent.

Compound 8 reacts with isocyanate via condensation to produce compound 9, wherein the mentioned isocyanate may include Me₃SiNCS,

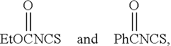

etc., preferably Me₃SiNCS, which is usually added at 3.5 equiv of that of compound 8. The reaction occurs in solvent-free condition or in DMF. The reaction temperature is between 100° C. and 160° C., most preferably 140° C.

Compound 9 is converted to compound 10 by reaction with

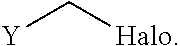

More than 1 equiv of an organic or inorganic base is required, such as triethylamine, DBU, DIPEA, potassium carbonate and sodium carbonate. A catalyst is optionally added, such as potassium iodide and tetrabutylammonium iodide. The reaction solvents include acetonitrile, acetone, DME, DCM, DCE, EA, ethanol, methanol, THF, etc. The reaction temperature is between 0° C. and 80° C., preferably the reflux temperature of the solvent.

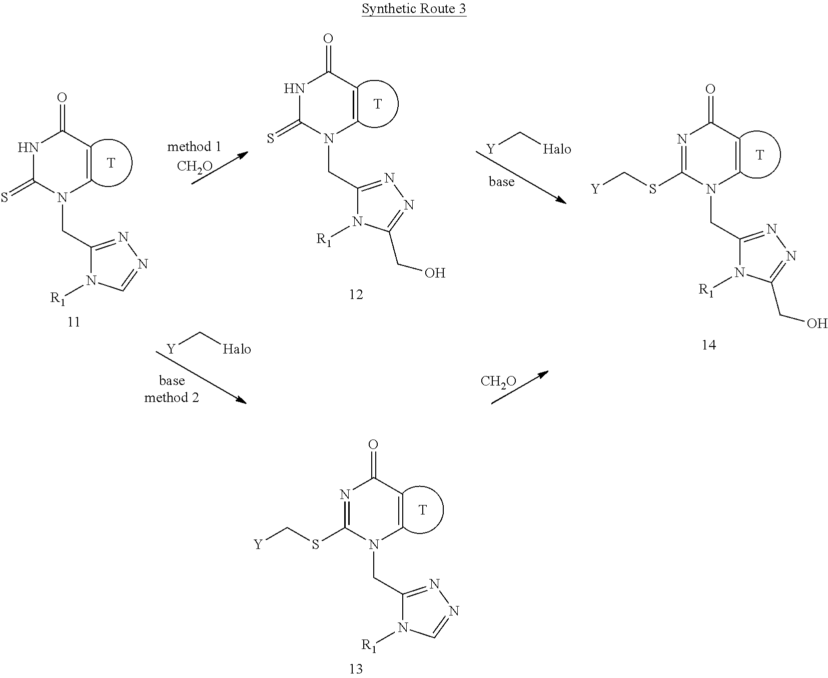

When W is Formula (c) meanwhile R² is hydroxymethyl in the above mentioned compound 10, the compound (i.e. compound 14) can be prepared according to either of the methods illustrated by synthetic route 3, in which Halo, T, Y and R¹ are as hereinbefore defined;

Compound 11 is prepared according to a method analogous to that described for compound 9;

According to the first method, compound 11 is heated with excessive aqueous formaldehyde to afford compound 12. The reaction occurs in a pressure-proof sealed container or a reflux condenser. The reaction is solvent-free or occurs in dioxane. The reaction temperature is between 100° C. and 160° C. Compound 12 then reacts with

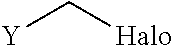

to give compound 14 under a condition analogous to that described for the preparation of compound 10 from compound 9.

According to the second method, compound 11 first reacts with

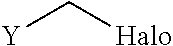

to give compound 13 under a condition analogous to that described for the preparation of compound 10 from compound 9. Compound 13 is then heated with excessive aqueous formaldehyde to afford compound 14 under a condition analogous to that described for the preparation of compound 14 from compound 12.

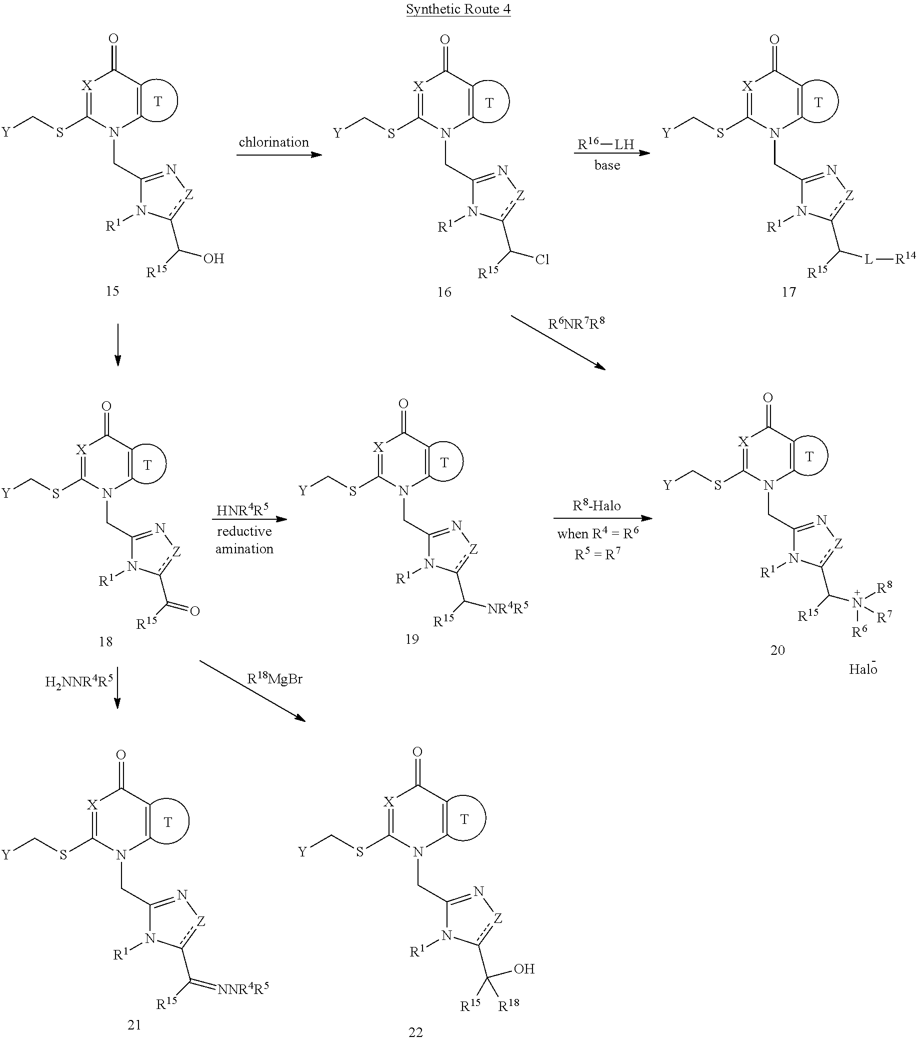

In certain circumstances, a compound of Formula (I) can be converted into another compound of Formula (I) by functional transformation, some examples of which are illustrated by synthetic route 4. It would be specially mentioned that these examples represent only part of the functional transformations of this invention, and therefore are not intended to limit the scope of synthetic methods of the invention in any way. More functional transformation examples will be given in the preparation methods of the specific examples.

The functional transformation methods in route 4 include:

When R² is α-hydroxyalkyl, a compound of Formula (I) can be represented by the structure of compound 15, in which R¹⁵ is H or C₁-C₆ alkyl; R¹⁶ is C₁-C₆ alkyl that is optionally substituted by NR⁴R⁵ or phenyl, wherein phenyl is optionally substituted by halogen; R¹⁸ is C₁-C₆ alkyl; L is NR⁴, O or S; Z is CH, N or O; T, X, Y, R¹, R⁴, R⁵, R⁶, R⁷ and R⁸ are as hereinbefore defined:

Compound 15 is chlorinated to afford compound 16. The chlorinating agent may be SOCl₂ or PCl₃. The reaction occurs in an aprotic solvent, such as DCM, CCl₄ and Et₂O. The reaction temperature is between 0° C. and room temperature;

Compound 16 reacts with R¹⁶LH to give compound 17 in the presence of a base, wherein the base may be an inorganic base, such as NaH, n-BuLi, KOBu-t and K₂CO₃, or an organic base, such as triethylamine, DIPEA, DBU and DMAP. The reaction solvent is selected from the group consisting of THF, CH₃CN, DME, DMF, EtOH, MeOH and HOBu-t, in accordance with the base used. The reaction temperature is between −80° C. and 80° C.

Compound 15 is oxidized to afford compound 18 under reaction conditions which have been adequately described in reference books, such as Comprehensive Org. Syn., Vol. 7, pp 251-327. In the present invention, the preferred oxidant is activated MnO₂, which is usually added at least 5 equiv of that of compound 15. The reaction solvent is dioxane or chloroform. The reaction temperature is between room temperature and 80° C., preferably 50-70° C.

Compound 18 reacts with HNR⁴R⁵ to give compound 19 in the presence of a reductant under reaction conditions which have been adequately described in reference books, such as Comprehensive Org. Trans., 2^{nd} ed., Wiley-VCH, NY, 1999, pp. 835-840. In the present invention, the preferred reductant is borohydride, such as NaBH₄, NaBH₃CN/Ti(OPr-i)₄ or NaBH(OAc)₃. The reaction solvents include DCM, DCE, THF, EtOH and MeOH, etc. The reaction temperature is between 0° C. and 80° C., preferably room temperature.

The condensation reaction of compound 16 with R⁶NR⁷R⁸ yields compound 20. When R⁴═R⁶ and R⁵═R⁷, the reaction of compound 19 with R⁸-Halo in an appropriate solvent also yields compound 20. The reaction solvents may include acetone, DCM, THF, DMF and acetonitrile. The reaction temperature is between −20° C. and room temperature.

The condensation reaction of compound 18 with H₂NNR⁴R⁵ in an appropriate solvent yields compound 21. The reaction solvents may include DCM, DCE, THF, EtOH and MeOH. A dehydant such as molecular sieve or anhydrous MgSO₄ is optionally added. The reaction temperature is between 0° C. and room temperature.

The reaction of compound 18 with R¹⁸BrMg yields compound 22; the reaction occurs in anhydrous THF, Et₂O or DME; the reaction temperature is between −20° C. and rt.

The above mentioned intermediate compound 7 can be prepared according to either of the methods illustrated by synthetic route 5 and 6:

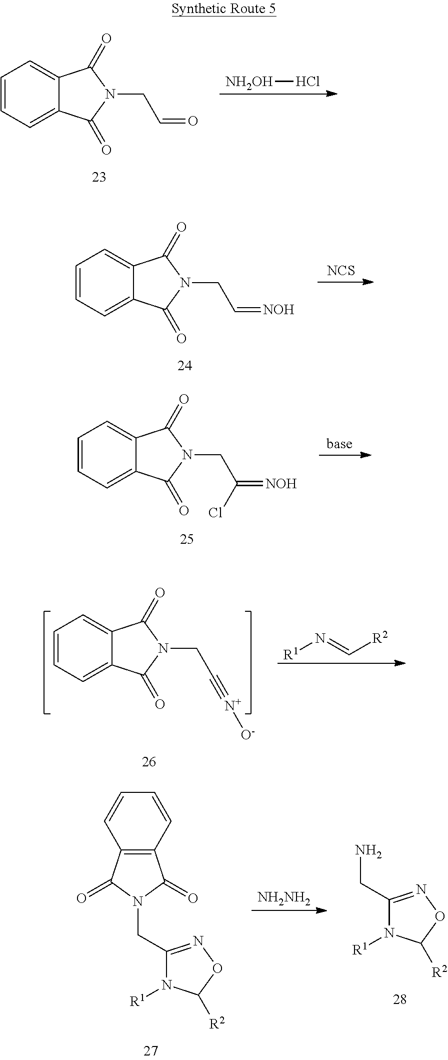

When W is Formula (a) in intermediate compound 7, this compound (i.e. compound 28) can be prepared according to the method illustrated by synthetic route 5, in which: R¹ and R² are as hereinbefore defined;

Compound 23 may be purchased from suppliers or prepared according to the literature method (Tetrahedron Letters 2001, 42, 315-317). Compound 23 reacts with hydroxylamine hydrochloride via condensation in the presence of a base to produce compound 24, wherein the mentioned bases include organic bases and inorganic bases, such as triethylamine, potassium carbonate and sodium hydroxide. The reaction occurs in a polar solvent, such as methanol, ethanol, DMF, water or a mixed solvent thereof. The reaction temperature is between 0° C. and 40° C.

Compound 24 is chlorinated with equivalent NCS to afford compound 25. The reaction solvents may include THF and DMF, etc. The reaction temperature is between 0° C. and 60° C.

Dehydrochloration of compound 25 in the presence of a base yields intermediate 26, followed by addition reaction with

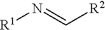

to form compound 27. The base used is an organic base, such as triethylamine, DIPEA or DBU. The reaction solvents may include THF and DMF, etc. The reaction temperature is between −40° C. and room temperature.

Compound 27 reacts with hydrazine hydrate to give compound 28. The reaction occurs in a polar solvent, such as THF, MeOH and EtOH. The reaction temperature is between 0° C. and 80° C., preferably 50-60° C.

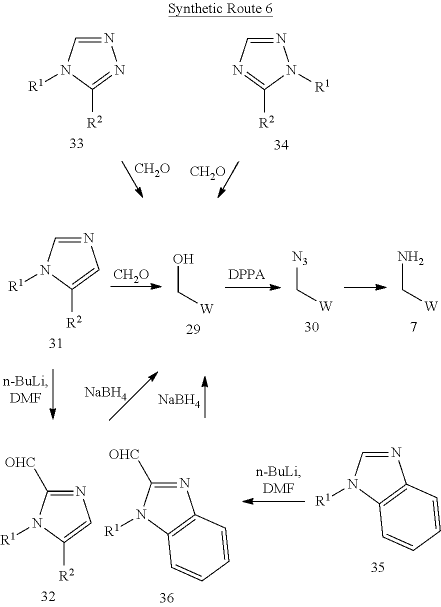

Except for the case where W is Formula (a) in intermediate compound 7 as mentioned above, compound 7 can be prepared by the methods shown in route 6, in which R¹ and R² are as hereinbefore defined;

Compound 29 reacts with DPPA to give compound 30 in the presence of a base, wherein the base is an organic base, such as triethylamine, DIPEA, DBU or DMAP, most preferably DBU and DMAP; the reaction solvent is a polar aprotic solvent, such as THF, CH₃CN and DME, etc., most preferably THF; the reaction temperature is between 0° C. and 100° C., most preferably reflux temperature of the solvent.

Compound 30 is converted to compound 7 by hydrogenation reduction or Staudinger reaction (Gololobov, Y. G. Sixty years of Staudinger reaction. Tetrahedron 1981, 37: 437). The hydrogenation reduction is catalyzed by Pd/C in a suitable solvent such as methanol, ethanol, EA or THF. The reaction temperature is between 0° C. and 80° C., most preferably room temperature. The Staudinger reaction occurs in the mixed solvent of THF with H₂O in the presence of excessive Ph₃P at a temperature between 0° C. and 50° C.

The above mentioned intermediate compound 29 can be prepared according to one of the methods as described below:

Compound 31 is heated with excessive aqueous formaldehyde to afford compound 29 under a condition analogous to that described for the preparation of compound 14 from compound 12 in; or

Compound 31 reacts with a formic acid derivative to afford compound 32 in the presence of a strong base, followed by reduction reaction with NaBH₄ to form compound 29. The mentioned strong bases include n-BuLi, s-BuLi, LDA or NaHMDS, etc., most preferably n-BuLi. The mentioned formic acid derivatives include DMF, ethyl formate or methyl formate, etc. The solvent herein used is THF and the reaction temperature is between −80° C. and room temperature.

Compound 33 is heated with excessive aqueous formaldehyde to afford compound 29 under a condition analogous to that described for the preparation of compound 14 from compound 12.

Compound 34 is heated with excessive aqueous formaldehyde to afford compound 29 under a condition analogous to that described for the preparation of compound 14 from compound 12.

Compound 35 reacts with a formic derivative to afford compound 36 in the presence of a strong base, followed by reduction reaction with NaBH₄ to form compound 29. The mentioned strong bases include n-BuLi, s-BuLi, LDA or NaHMDS, etc., most preferably n-BuLi. The mentioned formic derivatives include DMF, ethyl formate or methyl formate. The solvent herein used is THF and the reaction temperature is between −80° C. and room temperature.

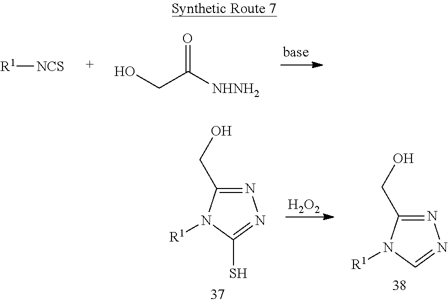

When W is Formula (c) meanwhile R² is H in intermediate compound 29, the compound (i.e. compound 38) can be prepared according to the method illustrated by synthetic route 7, in which: R¹ is as hereinbefore defined;

R¹—NCS is condensed with

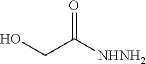

in the presence of a base to produce compound 37, wherein the mentioned base is an inorganic base, such as potassium carbonate, sodium carbonate or sodium hydroxide, etc.; the reaction occurs in a strong polar protic solvent, such as water, ethanol, etc.; and the reaction temperature is the reflux temperature of the solvent used.

The oxidative desulfurization reaction of compound 37 gives rise to compound 38. The oxidants include, for example, hydrogen peroxide, HNO₃/NaNO₂ or Fe(III) salts, most preferably aqueous hydrogen peroxide solution (30% w/w). AcOH is optionally added as a catalyst. The solvent may be a H₂O-DCM mixture or a H₂O—AcOH mixture.

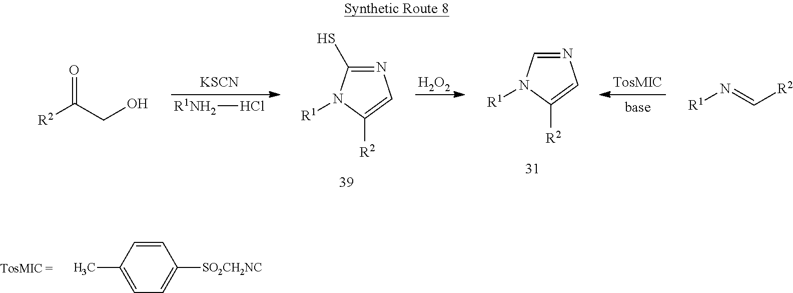

The above mentioned intermediate compound 31 can be prepared according to the method illustrated by synthetic route 8, in which: R¹ and R² are as hereinbefore defined;

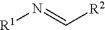

reacts with p-tosylmethyl isocyanide (TosMIC) via condensation in the presence of a base to produce compound 31, wherein the mentioned base may be an inorganic base such as NaH, KOBu-t, K₂CO₃ and Cs₂CO₃, etc., or an organic bases such as pyridine, triethylamine, DIPEA and DBU. Alternatively, R¹NH₂ can be used as the base directly. The reaction solvents include THF, CH₃CN, DME, DMF, EtOH, MeOH, HOBu-t and mixed solvents thereof. The reaction temperature is between 0° C. and 80° C.; or

The mixture of

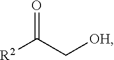

R¹NH₂—HCl and KSCN is first heated and condensed to produce compound 39, followed by oxidative desulfurization reaction to yield compound 31. The condensation reaction occurs in acetonitrile or HOBu-t in the presence of acetic acid or propionic acid as a catalyst at a temperature between 50 and 100° C. The oxidative desulfurization condition is analogous to that described for the preparation of compound 38 from compound 37.

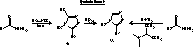

The above mentioned intermediate compound 33 can be prepared according to the methods illustrated by synthetic route 9, in which: R¹ and R² are as hereinbefore defined;

The mixture of

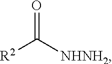

1,1-dimethoxy-N,N-dimethylethylamine and R¹NH₂ is heated and condensed to produce compound 33. The condensation reaction occurs in the mixed solvent of CH₃CN and AcOH at a temperature between 60 and 120° C.; or

The condensation reaction of

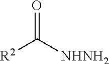

with R¹NCS in the presence of a base yields compound 40, followed by oxidative desulfurization reaction to form compound 33. The condensation condition is the same as described for the preparation of compound 37. The oxidative desulfurization condition is analogous to that described for the preparation of compound 38 from compound 37.

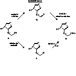

When R² is specified as

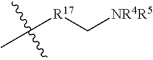

in the above intermediate compound 31 or compound 33, the compound (i.e. compound 44) can be prepared according to both methods illustrated by synthetic route 10, in which: R¹⁷ is a chemical bond, straight or branched C₁-C₄ alkylene; U is CH or N; R¹, R², R⁴ and R⁵ are as hereinbefore defined;

According to method A, compound 41 is first oxidized to afford compound 42; the preferred oxidizing condition is activated MnO₂ or Swern Oxidation (A. J. Mancuso, S-L. Huang, D. Swern. J. Org. Chem., 1978, 43, 2480); and the use of MnO₂ is analogous to that described for the preparation of compound 18 from compound 15. In this invention, the preferred reagent for Swern Oxidation is the combination of oxalyl chloride, DMSO and NEt₃. The reductive amination reaction of compound 42 with HNR⁴R⁵ affords compound 44. The reaction condition is analogous to that described for the preparation of compound 19 from compound 18.

According to method B, compound 41 is first chlorinated to afford compound 43; the reaction condition is analogous to that described for the preparation of compound 16 from compound 15; compound 43 then reacts with HNR⁴R⁵ to give compound 44 in the presence of a base, such as triethylamine, DIPEA, DBU or K₂CO₃; KI is optionally added as a catalyst; the reaction solvents include CH₃CN, DCM, DCE, THF or acetone; and the reaction temperature is between room temperature and 90° C., most preferably the reflux temperature of the solvent.

In addition to the preparation methods illustrated by synthetic routes 5 to 10 for the intermediate compounds in the invention, other novel intermediates or raw materials can be prepared according to similar literature methods and will be described in detail with reference to the methods for preparing the compounds in the specific examples. The already known intermediates or raw materials can be purchased from suppliers or prepared according to literature methods.

### Specific Examples

The following synthetic examples are provided to more specifically illustrate the invention. These examples are not intended to limit the scope of the invention, but to give more detailed description to the invention. Unless otherwise indicated, all the parameters and other instructions provided in the invention are based on quality. In general, chromatographic column is packed with silica gel unless otherwise indicated. An experimental method, the condition of which is not specifically described in the invention, is carried out under conventional condition or condition recommended by the manufacturer.

Unless otherwise defined, all professional and scientific terms are in accordance with what are familiarized by the skilled artisan in this area. In addition, any similar or equivalent method and material to that recorded in this invention can be used herein. The preferred examples, methods and materials are used for demonstration purposes only.

### Synthesis Examples

The number of intermediate in this invention was started with a letter selected from “M, A, B, C, D, E, F”, such as intermediate “M1” or “A8”; the number of final compound was started with the word “example”, such as “example 18”.

### Intermediate M1—4′-(trifluoromethyl)biphenyl-4-carbaldehyde

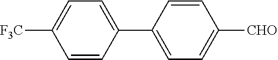 [SMILES: O=Cc1ccc(-c2ccc(C(F)(F)F)cc2)cc1]

A mixture of 4-(trifluoromethyl)phenylboronic acid (3.80 g, 1 equiv), 4-bromobenzaldehyde (3.7 g, 1 equiv), palladium acetate (0.225 g, 0.05 equiv), aq. Na₂CO₃ (20 ml, 2M solution, 2 equiv) solution in DME (40 ml) was purged with nitrogen and then evacuated, then refluxed under nitrogen for 3 h. TLC detection showed that the reaction was complete. The insoluble substance was filtered while hot, and then washed with ethyl acetate. The liquid phase was transferred to a separatory funnel and the organic phase was separated and the aqueous phase was extracted with ethyl acetate twice. The organic phases were combined, washed with brine twice, dried over MgSO₄ and evaporated under reduced pressure. The residue was purified by column chromatography or recrystallized from petroleum ether and diethyl ether to give the title compound.

### Intermediate M2—methyl 2-(4-(trifluoromethylsulfonyloxy)phenyl)acetate

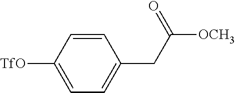 [SMILES: COC(=O)Cc1ccc(OS(=O)(=O)C(F)(F)F)cc1]

A solution of triflumethanesulfonic anhydride (4.3 ml, 26 mmol, 1.3 equiv) in dichloromethane (100 ml) was placed in a low-temperature reactor of −40° C. under nitrogen. Pyridine (2.5 ml, 30 mmol, 1.5 equiv) was added dropwise over 10 min via a syringe, during the addition, a precipitate appeared and the mixture was stirred vigorously. After the addition was complete, the mixture was further stirred for 15 min before a solution of methyl 4-hydroxyphenylacetate (3.32 g, 20 mmol, 1 equiv) in dichloromethane (40 ml) was added dropwise via a syringe over 5 min. The refrigeration was stopped and the mixture was stirred for a further 30 min, then saturated ammonium chloride solution was added. The organic layer was separated, washed successively with diluted hydrochloric acid, brine and saturated sodium bicarbonate solution, dried over MgSO₄, and then evaporated in vacuum to give 3.15 g of oil, which turned to be a crystalline upon standing (3.15 g). ¹H-NMR (CDCl₃, 300 MHz) δ 3.65 (s, 2H), 3.71 (s, 3H), 7.24 (d, 2H, J=8.7), 7.37 (d, 2H, J=9.0).

### Intermediate M3—methyl 2-(4′-(trifluoromethyl)biphenyl-4-yl)acetate

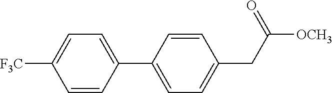 [SMILES: COC(=O)Cc1ccc(-c2ccc(C(F)(F)F)cc2)cc1]

The title compound was prepared by a procedure similar to that described for intermediate M1 except that M2 was used in place of 4-bromobenzaldehyde. ¹H-NMR (CDCl₃, 300 MHz) δ 3.69 (s, 2H), 3.72 (s, 3H), 7.39 (d, 2H, J=8.1), 7.56 (m, 2H, J=7.8), 7.68 (m, 4H)

### Intermediate M4—biphenyl-4-carbaldehyde

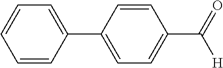 [SMILES: O=Cc1ccc(-c2ccccc2)cc1]

The title compound was prepared by a procedure similar to that described for intermediate M1 except that phenylboronic acid was used in place of 4-(trifluoromethyl)phenylboronic acid. ¹H-NMR (CDCl₃, 400 MHz) δ 7.42 (m, 1H), 7.49 (m, 2H), 7.64 (m, 2H), 7.76 (d, 2H, J=8.4), 7.96 (d, 2H, J=8.4), 10.06 (s, 1H).

### Intermediate M5—4′-methylbiphenyl-4-carbaldehyde

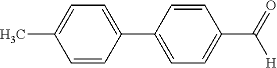

The title compound was prepared by a procedure similar to that described for intermediate M1 except that p-tolylboronic acid was used in place of 4-(trifluoromethyl)phenylboronic acid. ¹H-NMR (CDCl₃, 400 MHz) δ 2.42 (s, 3H), 7.29 (d, 2H, J=8.0), 7.54 (d, 2H, J=8.4), 7.79 (d, 2H, J=8.0), 7.94 (d, 2H, J=8.0), 10.04 (s, 1H).

### Intermediate M6—4′-chlorobiphenyl-4-carbaldehyde

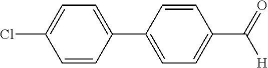 [SMILES: O=Cc1ccc(-c2ccc(Cl)cc2)cc1]

The title compound was prepared by a procedure similar to that described for intermediate M1 except that 4-chlorophenylboronic acid was used in place of 4-(trifluoromethyl)phenylboronic acid. ¹H-NMR (CDCl₃, 300 MHz) δ 7.46 (d, 2H, J=8.4), 7.58 (d, 2H, J=8.4), 7.73 (d, 2H, J=8.1), 7.94 (d, 2H, J=8.1), 10.07 (s, 1H). The following intermediates were prepared by a procedure similar to that described for intermediate M1.

| Name | Structure | Reactant |
|---|---|---|
| M7: methyl 4′-(trifluoromethyl)biphenyl- 4-carboxylate | 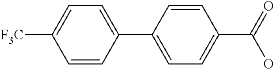 [SMILES: O=C(O)c1ccc(-c2ccc(C(F)(F)F)cc2)cc1] | 4-(trifluoromethyl)phenyl boronic acid, methyl 4-bromobenzoate |
| M8: methyl biphenyl-4-carboxylate | 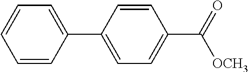 [SMILES: COC(=O)c1ccc(-c2ccccc2)cc1] | phenylboronic acid, methyl 4-bromobenzoate |
| M9: methyl 2-(biphenyl-4-yl)acetate | 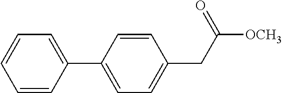 [SMILES: COC(=O)Cc1ccc(-c2ccccc2)cc1] | phenylboronic acid, M2 |

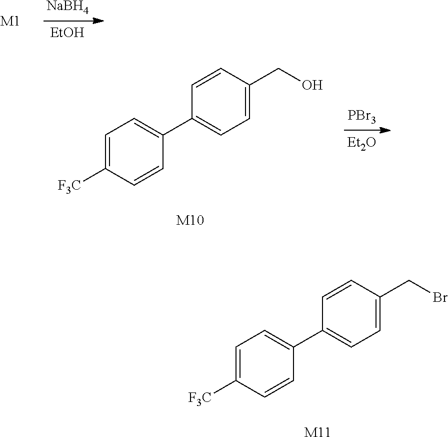

### Intermediate M11—4-(bromomethyl)-4′-(trifluoromethyl)biphenyl

To an ice cold solution of intermediate M1 (2.5 g, 1 equiv) in absolute ethanol (20 ml) was added NaBH₄ (190 mg) in batches, and then the mixture was stirred at room temperature for 1 h. After the reaction was complete, solvent was evaporated and the residue was diluted with water. Concentrated hydrochloric acid was added dropwise to the solution in an ice bath until no more bubbles were generated. Then, sodium bicarbonate solution was added and the mixture was extracted with dichloromethane three times. The combined organic phase was washed with brine twice, dried over MgSO₄, and then filtered. The filtrate was evaporated in vacuo to give intermediate M10 as a white solid (2.5 g). ¹H-NMR (CDCl₃, 300 MHz) δ 4.77 (s, 2H), 7.48 (d, 2H, J=8.4), 7.61 (d, 2H, J=8.1), 7.70 (s, 4H).

To an ice cold solution of intermediate M10 (2.02 g, 1 equiv) in anhydrous diethyl ether (20 ml) protected with a CaCl₂ drying tube to insulate moisture was added phosphorus tribromide (0.38 ml, 0.5 equiv). The mixture was stirred at room temperature for 1.5 h to obtain a clear solution. TLC detection showed that the reaction was complete and then the mixture was quenched with saturated sodium bicarbonate solution. The resulting precipitate was filtered off and the filtrate was extracted with dichloromethane twice (40 ml), dried over MgSO₄, filtered and then evaporated in vacuo to give intermediate M11 (1.77 g) as a white solid which could be used in next step without further purification.

The following intermediates were prepared by a procedure similar to that described for intermediate M11.

| Name | Structure | Precusor |
|---|---|---|
| M12: 4-(bromometh- yl)biphenyl | 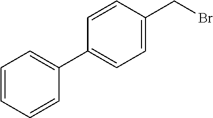 [SMILES: BrCc1ccc(-c2ccccc2)cc1] | M4 |
| M13: 4-(bromomethyl)- 4′-methylbiphenyl | 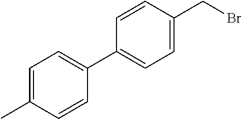 | M5 |
| M14: 4-(bromomethyl)- 4′-chlorobiphenyl | 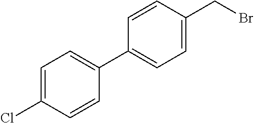 [SMILES: Clc1ccc(-c2ccc(CBr)cc2)cc1] | M6 |

### Intermediate M15—(E)-3-(1-methyl-1H-pyrazol-4-yl)acrylic acid

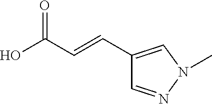

Methylpyrazole (4.1 g, 50 mmol, 1 equiv) and anhydrous DMF (11.6 ml, 3 equiv) were placed in a 100 ml of two-neck flask which was equipped with a Allihn condenser (a CaCl₂ drying tube was connected thereto) and a constant-pressure dropping funnel, and the flask was placed in the oil bath of 90° C. Phosphorus oxychloride (5.6 ml, 1.2 equiv) was added dropwise over a period of 1 h. After the addition was completed, the mixture was stirred for a further 2 h. Then the solution was cooled and poured into a lot of ice water. The resulting mixture was brought to pH 4-5 with a solution of 10% NaOH solution and then extracted with dichloromethane several times until the product in the aqueous phase is little. The combined organic phase was washed with a small amount of brine twice, dried over MgSO₄, filtered and then evaporated in vacuo to give the crude product of 1-methyl-1H-pyrazole-4-carbaldehyde, which can be directly used in the next step without further separation.

1-methyl-1H-pyrazole-4-carbaldehyde previously obtained, malonic acid (4.68 g, 45 mmol), pyridine (4 ml, 50 mmol) and piperidine (0.09 ml, 1 mmol) were placed in a 250 ml flask and heated at 110° C. for 6 h under nitrogen, then the reaction was stopped and cooled. The resulting solution was added with water (100 ml), followed by addition of strong aqua dropwise to dissolve all the solids. The solution was then brought to pH˜1 with concentrated hydrochloric acid. The precipitate thus obtained was collected by filtration, washed with water several times and then dried in vacuo to give the title compound (2.9 g). ¹H-NMR (d₆-DMSO, 300 MHz) δ3.83 (s, 3H), 6.17 (d, 1H, J=16.2), 7.45 (d, 1H, J=15.9), 7.83 (s, 1H), 8.07 (s, 1H), 12.07 (s, 1H).

### Intermediate M16—(E)-methyl 3-(1-methyl-1H-pyrazol-4-yl)acrylate

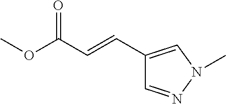

To a stirred slurry of intermediate M15 (2.9 g, 19.1 mmol) in methanol (30 ml) was added concentrated sulfuric acid dropwise (1.9 ml). Heat was released and the mixture was refluxed for 2.5 h before it was evaporated in vacuo to remove most of the solvent. Ice water was added to the residue and the resulting solution was neutralized with 10% aqueous sodium hydroxide solution and then extracted with dichloromethane three times. The dichloromethane layer was dried over MgSO₄, filtered and then evaporated to give the title compound as a solid (3.08 g). ¹H-NMR (CDCl₃, 300 MHz) δ3.75 (s, 3H), 3.89 (s, 3H), 6.14 (d, 1H, J=15.9), 7.53 (s, 1H), 7.54 (d, 1H, J=16.2), 7.67 (s, 1H).

### Intermediate M17—methyl 3-(1-methyl-1H-pyrazol-4-yl)propanoate

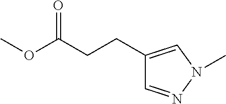

To a solution of M16 (3.08 g, 18.55 mmol) in absolute methanol (20 ml) was added 10% palladium on charcoal (300 mg) and the mixture was stirred at 50° C. and normal pressure for 3.5 h. TLC detection showed that the reaction was complete. The catalyst was filtered off and the filtrate was evaporated to give the title compound as a colorless oil (3.06 g). ¹H-NMR (CDCl₃, 300 MHz) δ2.54 (t, 2H, J=7.2), 2.77 (t, 2H, J=7.2), 3.65 (s, 3H), 3.83 (s, 3H), 7.16 (s, 1H), 7.30 (s, 1H).

### Intermediate M18—methyl 3-hydroxy-2-((1-methyl-1H-pyrazol-4-yl)methyl)acrylate

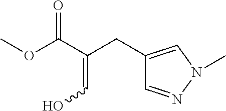

To a solution of intermediate M17 (2.53 g, 15.06 mmol, 1 equiv) in anhydrous DME (20 ml) was added sodium hydride (55-65% m/m, suspended in mineral oil, 0.75 g, 1.25 equiv) under nitrogen at room temperature and the mixture was stirred for 1 h. Methyl formate (2.74 ml, 3 equiv) was added and the mixture was stirred at room temperature for a further 15 h. Then, anhydrous diethyl ether (100 ml) was added and the mixture was stirred for 15 min. The solid thus obtained was collected by filtration, washed with diethyl ether and then the filter cake was poured into saturated ammonium chloride solution. The resulting mixture was acidified with concentrated hydrochlolic acid to pH 4-5 in an ice bath and then extracted with dichloromethane three times. The combined organic phase was washed with brine twice, dried over MgSO₄, filtered and then evaporated to give the title compound as a solid (0.51 g) which could be used in next step without further purification.

### Intermediate M19—tert-butyl 4-(1-methoxy-2-methyl-1-oxopropan-2-yl)piperazine-1-carboxylate

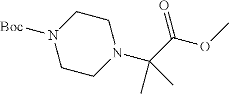 [SMILES: COC(=O)C(C)(C)N1CCN(C(=O)OC(C)(C)C)CC1]

A mixture of tert-butyl piperazine-1-carboxylate (3.72 g, 2 equiv), methyl 2-bromo-2-methylpropanoate (2.58 ml, 2 equiv), anhydrous K₂CO₃ (5.52 g, 4 equiv) and KI (166 mg, 0.1 equiv) in acetonitrile (40 ml) was refluxed for 8 h. Additional methyl 2-bromo-2-methylpropanoate (1.29 ml, 1 equiv) was added and the mixture was refluxed for a further 12 h before it was cooled and filtered to remove the insoluble inorganic substances. The filtrate was evaporated and then purified by silicagel column chromatography (petroleum ether/ethyl acetate=2:1) to give the title compound as an oil (2.82 g). ¹H-NMR (CDCl₃, 400 MHz) δ1.31 (s, 6H), 1.44 (s, 9H), 2.52 (vbrs, 4H), 3.42 (vbrs, 4H), 3.69 (s, 3H).

### Intermediate M20—methyl 2-methyl-2-(piperazin-1-yl)propanoate hydrochloride

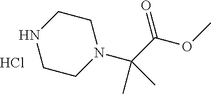

To an ice cold solution of intermediate M19 (2.82 g, 1 equiv) in methanol (20 ml) was added acetyl chloride (3.5 ml, 5 equiv) dropwise and the mixture was stirred for 1 h before it was evaporated to remove part of the solvent. White solid precipitated. Diethyl ether (20 ml) was added to the residue and the mixture was stirred for a few minutes. The precipitate thus obtained was collected by filtration and dried in vacuo to give the title compound (2.33 g). ¹H-NMR (d₆-DMSO, 300 MHz) δ1.49 (s, 6H), 3.33 (vbrs, 8H), 3.73 (s, 3H), 9.52 (s, 1H).

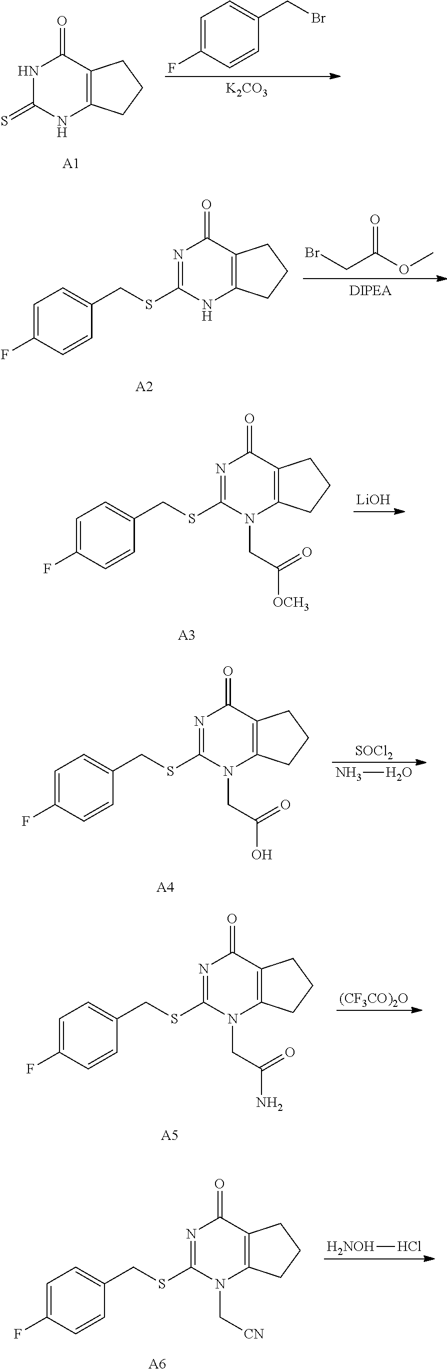

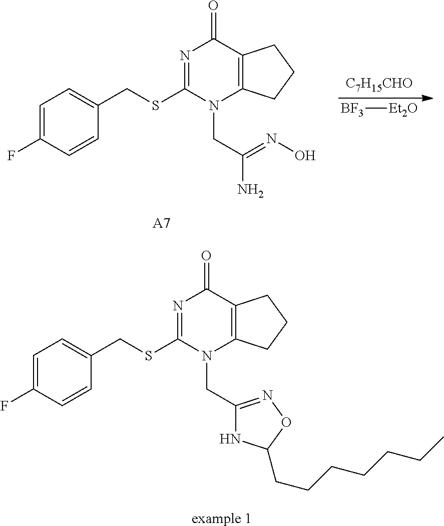

### Intermediate A1—2-thioxo-2,3,6,7-tetrahydro-1H-cyclopenta[d]pyrimidin-4(5H)-one

Prepared according to reference method [J. Amer Chem. Soc., 81, 3108 (1959)].

### Intermediate A2—A2-2-(4-fluorobenzylthio)-6,7-dihydro-1H-cyclopenta[d]pyrimidin-4(5H)-one

A mixture of intermediate A1 (4.06 g, 1 equiv), 1-(bromomethyl)-4-fluorobenzene (3.2 ml, 1.05 equiv), anhydrous K₂CO₃ (5 g, 1.5 equiv), KI (0.4 g, 0.1 equiv) in acetone (40 ml) was refluxed for 2 h. Then most of the solvent was removed by evaporation in reduced pressure and the residue was poured into water. The resulting mixture was neutralized with con. HCl. The solid thus obtained was collected by filtration, washed with water several times and then the moisture was evacuated. The solid was transferred into a flask, to which methanol (30 ml) was added. The mixture was refluxed for 0.5 h, cooled and filtered and dried to get the title compound (4.62 g) as a solid.

### Intermediate A3—Methyl 2-(2-(4-fluorobenzylthio)-4-oxo-4,5,6,7-tetrahydro-1H-cyclopenta[d]pyrimidin-1-yl)acetate

A solution of intermediate A2 (2.67 g, 10 mmol), methyl 2-bromoacetate (1 ml, 11 mmol) and DIPEA (1.82 ml, 11 mmol) in THF (20 ml) was refluxed overnight under nitrogen and then quenched with saturated NH₄Cl solution. Then it was extracted with EA twice. The organic phase was washed with water twice, dried over MgSO₄, filtered, and then concentrated. The residue was purified by column chromatography (DCM/MeOH=30:1) to give the title compound as a jelly (0.5 g). ¹H-NMR (CDCl₃, 300 MHz) δ2.10 (m, 2H), 2.81 (m, 4H), 2.98 (t, 2H, J=7.5), 3.78 (s, 3H), 4.49 (s, 2H), 4.57 (s, 2H), 6.97 (t, 2H, J=8.4), 7.35 (dd, 2H, J=7.8, 6.0).

### Intermediate A4—2-(2-(4-fluorobenzylthio)-4-oxo-4,5,6,7-tetrahydro-1H-cyclopenta[d]pyrimidin-1-yl)acetic acid

A solution of intermediate A3 (6.9 g, 1 equiv), LiOH.H₂O (2.5 g, 3 equiv) in isopropanol/water (1:2, 60 ml) was stirred at room temperature for 2 h to obtain a clear solution. The solution was acidified with concd. HCl to pH<3 in an ice bath, the solid thus obtained was collected by filtration, washed with water several times and the moisture was evacuated as much as possible. The solid was transferred to a 100 mL flask and 30 ml acetone was added to obtain a mixture, which was refluxed for 0.5 h. Then the mixture was placed in refrigerator for several hours until the recrystallization was complete. The solid obtained was filtered and washed by acetone, then dried to afford the title compound as a white solid (4.3 g). ¹H-NMR (d₆-DMSO, 300 MHz) δ1.96 (m, 2H), 2.58 (t, 2H, J=7.2), 2.82 (t, 2H, J=7.5), 4.42 (s, 2H), 4.69 (s, 2H), 7.13 (t, 2H, J=9.0), 7.47 (dd, 2H, J=8.9, 5.6), 13.5 (vbrs, 1H); MS (ESI): 333 (M−H).

### Intermediate A5—2-(2-(4-fluorobenzylthio)-4-oxo-4,5,6,7-tetrahydro-1H-cyclopenta[d]pyrimidin-1-yl)acetamide

To an ice cold suspension of intermediate A4 (2.0 g, 1 equiv) in dichloromethane (15 ml) (wherein a drying tube was used to insulate moisture) were added thionyl chloride (0.47 ml) and DMF (2 drops, catalyst). The mixture was stirred for 0.5 h to get a brown clear solution and then added slowly to a flask containing strong aqua (20 ml) over 10 min in an ice bath with vigorous stirring. The solid thus formed was collected by filtration, washed to white with water and ethanol, and then dried in vacuo to give the title compound (1.0 g). MS (ESI): 334 (M+H).

### Intermediate A6—2-(2-(4-fluorobenzylthio)-4-oxo-4,5,6,7-tetrahydro-1H-cyclopenta[d]pyrimidin-1-yl)acetonitrile

To an ice-salt cold (−15° C.) suspension of intermediate A5 (10 mmol) in anhydrous THF (20 ml) was added trifluoroacetic anhydride (20 mmol) dropwise via a syringe under nitrogen over 10 min. The reaction mixture was allowed to warm to room temperature, quenched with saturated NaHCO₃ solution and extracted with EA twice. The combined organic phase was washed with brine twice, dried and purified by column chromatography (DCM/MeOH=30:1) to give the title compound. ¹H-NMR (CDCl₃, 300 MHz) δ2.16 (m, 2H), 2.82 (t, 2H, J=7.2), 2.98 (t, 2H, J=7.5), 4.53 (s, 2H), 4.76 (s, 2H), 5.67 (t, 1H, J=4.8), 7.00 (t, 2H, J=8.7), 7.39 (dd, 2H, J=8.3, 5.3);

MS (ESI): 316 (M+H).

### Intermediate A7—(Z)-2-(2-(4-fluorobenzylthio)-4-oxo-4,5,6,7-tetrahydro-1H-cyclopenta[d]pyrimidin-1-yl)-N′-hydroxyacetimidamide

To a solution of intermediate A6 (10 mmol) in absolute ethanol (20 ml) were added hydroxylamine hydrochloride (13 mmol) and K₂CO₃ (15 mmol). The mixture was stirred at room temperature for 12 h. TLC detection showed that the reaction was complete. Then the mixture was evaporated to remove most of the solvent. Water (10 ml) and EA (10 ml) was added to the residue and the resulting mixture was stirred for a 1 h. The solid thus obtained was collected by filtration, washed with water and EA, and then dried in vacuo to give the title compound. ¹H-NMR (d₆-DMSO, 300 MHz) δ96 (m, 2H), 2.59 (t, 2H, J=7.3), 2.90 (t, 2H, J=7.3), 4.43 (s, 2H), 4.55 (s, 2H), 5.72 (s, 2H), 7.17 (t, 2H, J=8.8), 7.49 (dd, 2H, J=8.8, 5.6), 9.44 (s, 1H); MS (ESI): 349 (M+H).

### Example 1

### 2-(4-fluorobenzylthio)-1-((5-n-heptyl-4,5-dihydro-1,2,4-oxadiazol-3-yl)methyl)-6,7-dihydro-1H-cyclopenta[d]pyrimidin-4(5H)-one

To a mixture of intermediate A7 (1 mmol) and octyl aldehyde (1.1 mmol) in anhydrous THF (5 ml) was added BF₃-Et₂O solution (2 mmol) under nitrogen. The solution was stirred at room temperature until completion of the reaction monitored by TLC. The reaction mixture was then poured into saturated NaHCO₃ solution and extracted with EA. The EA solution was dried over MgSO₄ and then purified by column chromatography to give the title compound. ¹H-NMR (CDCl₃, 300 MHz) δ0.84 (t, 3H, J=6.9), 1.22-1.32 (m, 10H), 1.70 (m, 2H), 2.13 (m, 2H), 2.69 (t, 2H, J=7.5), 3.00 (m, 2H), 4.52 (s, 2H), 4.76 (2×d, 2H, J=16.5), 5.67 (t, 1H, J=4.8), 6.93 (t, 2H, J=9.0), 7.10 (s, 1H), 7.48 (dd, 2H, J=8.7, 5.1); MS (ESI): 459 (M+H).

### Example 2

### 1-((5-n-decyl-4,5-dihydro-1,2,4-oxadiazol-3-yl)methyl)-2-(4-fluorobenzylthio)-6,7-dihydro-1H-cyclopenta[d]pyrimidin-4(5H)-one

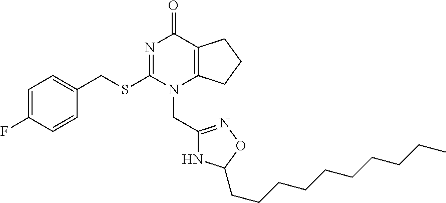 [SMILES: CCCCCCCCCCC1NC(Cn2c(SCc3ccc(F)cc3)nc(=O)c3c2CCC3)=NO1]

Following a procedure similar to that described for the preparation of example 1 except that n-undecylic aldehyde was used in place of octyl aldehyde. ¹H-NMR (CD₃OD, 400 MHz) δ0.89 (t, 3H, J=6.6), 1.27 (m, 16H), 1.60 (m, 2H), 2.12 (m, 2H), 2.75 (t, 2H, J=7.2), 3.00 (t, 2H, J=7.6), 4.52 (s, 2H), 4.86 (s, 2H), 5.56 (t, 1H, J=4.8), 7.01 (t, 2H, J=8.4), 7.48 (dd, 2H, J=8.0, 5.6); MS (ESI): 501 (M+H).

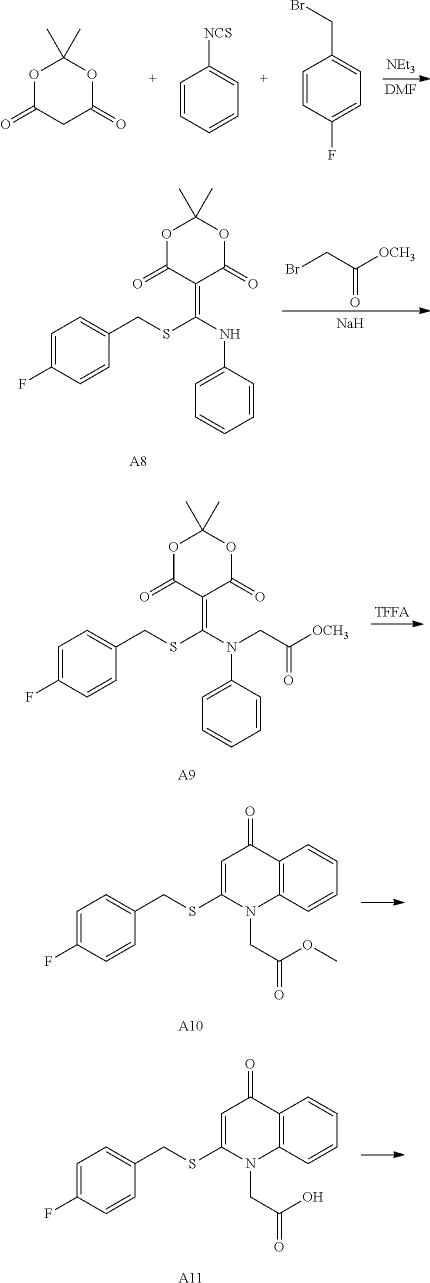

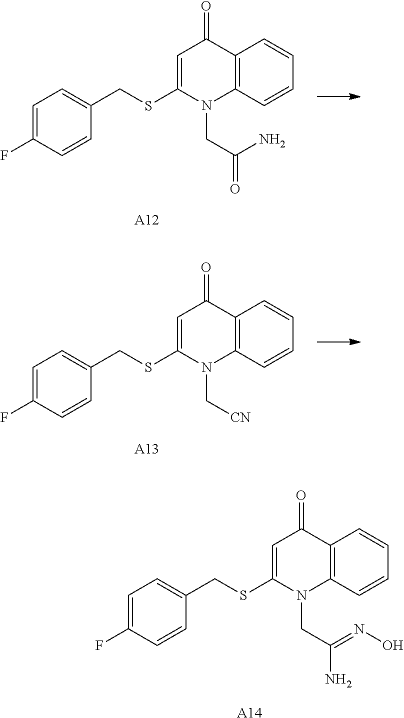 [SMILES: NC(=O)Cn1c(SCc2ccc(F)cc2)cc(=O)c2ccccc21 ; "A12" ; N#CCn1c(SCc2ccc(F)cc2)cc(=O)c2ccccc21 ; "A13" ; N/C(Cn1c(SCc2ccc(F)cc2)cc(=O)c2ccccc21)=N\O ; "A14"]

### Intermediate A8—5-((4-fluorobenzylthio)(phenylamino)methylene)-2,2-dimethyl-1,3-dioxane-4,6-dione

To a solution of 2,2-dimethyl-1,3-dioxane-4,6-dione (1.5 g, 1 equiv) in DMF (10 ml) was added triethylamine (2.7 ml, 2 equiv) and the mixture was stirred at room temperature for 0.5 h to obtain a light yellow solution before phenyl isocyanate (1.2 ml, 1 equiv) was added. The resulting mixture was stirred for a further 5 h at 40-45° C. and then placed in an ice bath. 1-(bromomethyl)-4-fluorobenzene (1.25 ml, 1 equiv) was added and the mixture was stirred at room temperature overnight. After the reaction was complete, the mixture was poured into water, extracted with EA three times. The combined organic phase was washed with brine several times, dried over MgSO₄ and concentrated. The solid thus obtained was collected by filtration and dried to give the title compound (2.2 g). ¹H-NMR (d₆-DMSO, 300 MHz) δ1.63 (s, 6H), 4.09 (s, 2H), 7.14 (t, 2H, J=8.7), 7.29 (dd, 2H, J=8.9, 5.6), 7.44 (m, 5H), 12.13 (s, 1H); MS (ESI): 388 (M+H).

### Intermediate A9—methyl 2-(((2,2-dimethyl-4,6-dioxo-1,3-dioxan-5-ylidene)(4-fluorobenzylthio)methyl)(phenyl)amino)acetate

To an ice cold solution of intermediate A8 (13.55 g, 1 equiv) in anhydrous DMF (40 ml) was added NaH (2.8 g, 2 equiv) in batches under nitrogen and the mixture was stirred for 20 min. Methyl 2-bromoacetate (6.7 ml, 2 equiv) was added and the solution was stirred at room temperature for 2 h and then at 60° C. overnight. After the reaction was complete as monitored by TLC, the mixture was poured into saturated NH₄Cl solution and extracted with 200 ml of EA three times. The organic phase was washed with brine five times, dried over MgSO₄, and purified by column chromatography (PE/EA=2:1) to give the title compound as a jelly (8.2 g). ¹H-NMR (CDCl₃, 400 MHz) δ56 (s, 6H), 3.72 (s, 3H), 4.31 (s, 2H), 4.82 (s, 2H), 6.98 (t, 2H, J=8.6), 7.21 (m, 2H), 7.39 (m, 5H).

### Intermediate A10—methyl 2-(2-(4-fluorobenzylthio)-4-oxoquinolin-1(4H)-yl)acetate

A mixture of intermediate A9 (8.2 g, 17.86 mmol) and trifluoroacetic anhydride (10 ml) was stirred at room temperature overnight. Excess trifluoroacetic anhydride was removed under reduced pressure. The residue was quenched with saturated aq. NaHCO₃ solution (20 ml) and extracted with dichloromethane (3*10 ml). The combined organic phase was dried over MgSO₄, and then purified by column chromatography (PE/EA=1:3) to give the title compound (5.5 g). ¹H-NMR (CDCl₃, 400 MHz) δ3.79 (s, 3H), 4.23 (s, 2H), 5.09 (s, 2H), 6.44 (s, 1H), 7.02 (t, 2H, J=8.6), 7.18 (d, 1H, J=8.7), 7.33 (dd, 2H, J=8.8, 5.2), 7.39 (t, 1H, J=7.4), 7.63 (ddd, 1H, J=8.8, 7.2, 1.6), 8.41 (dd, 1H, J=8.2, 1.4).

### Intermediate A11—2-(2-(4-fluorobenzylthio)-4-oxoquinolin-1 (4H)-yl)acetic acid

Following a procedure similar to that described for the preparation of intermediate A4 except that intermediate A10 was used as a starting material. ¹H-NMR (d₆-DMSO, 300 MHz) δ4.45 (s, 2H), 5.18 (s, 2H), 6.25 (s, 1H), 7.19 (t, 2H, J=8.7), 7.37 (t, 1H, J=7.2), 7.50 (dd, 2H, J=8.7, 5.4), 7.60 (d, 1H, J=8.7), 7.69 (ddd, 1H, J=8.7, 7.2, 1.7), 8.13 (dd, 1H, J=8.0, 1.8).

### Intermediate A12—2-(2-(4-fluorobenzylthio)-4-oxoquinolin-1 (4H)-yl)acetamide

To an ice cold suspension of intermediate A11 (1.37 g, 1 equiv) in anhydrous DMF (6 ml) were added EDCI (1.15 g, 1.5 equiv) and HOBt (0.81 g, 1.5 equiv) under nitrogen and the mixture was stirred for 0.5 h to obtain a yellow clear solution. Then, DIPEA (2.64 ml, 4 equiv) and NH₄Cl (0.43 g, 2 equiv) were added and the mixture was stirred at room temperature overnight. Then the mixture was diluted with water (20 ml) and EA (20 ml), stirred for 1 h. The solid thus obtained was collected by filtration, washed with water and EA, and dried to obtain the title compound (0.54 g). Aqueous phase of the filtrate was separated and acidified with concd. HCl to recycle the starting materials. ¹H-NMR (d₆-DMSO, 300 MHz) δ4.42 (s, 2H), 5.04 (s, 2H), 6.23 (s, 1H), 7.19 (t, 2H, J=8.9), 7.36 (t, 1H, J=7.4), 7.49 (m, 4H), 7.68 (t, 1H, J=7.4), 7.79 (s, 2H), 8.13 (d, 1H, J=7.8); MS (ESI) found (M+H)=343.

### Intermediate A13—2-(2-(4-fluorobenzylthio)-4-oxoquinolin-1(4H)-yl)acetonitrile

Following a procedure similar to that described for the preparation of intermediate A6 except that intermediate A12 was used as a starting material. ¹H-NMR (CDCl₃, 300 MHz) δ4.27 (s, 2H), 5.22 (s, 2H), 6.43 (s, 1H), 7.03 (t, 2H, J=8.6), 7.33 (dd, 2H, J=8.7, 5.1), 7.41 (d, 1H, J=8.7), 7.45 (dd, 1H, J=7.5, 7.5), 7.75 (ddd, 1H, J=8.7, 7.2, 1.5), 8.41 (dd, 1H, J=8.0, 1.7).

### Intermediate A14—(Z)-2-(2-(4-fluorobenzylthio)-4-oxoquinolin-1 (4H)-yl)-N′-hydroxyacetimidamide

Following a procedure similar to that described for the preparation of intermediate A7 except that intermediate A13 was used as a starting material. ¹H-NMR (d₆-DMSO, 300 MHz) δ4.42 (s, 2H), 5.04 (s, 2H), 5.68 (s, 2H), 6.21 (s, 1H), 7.19 (t, 2H, J=8.7), 7.33 (t, 1H, J=7.4), 7.47-7.55 (m, 3H), 7.68 (m, 1H), 8.09 (dd, 1H, J=7.8, 1.5).

### Example 3

### 2-(4-fluorobenzylthio)-1-((5-n-heptyl-4,5-dihydro-1,2,4-oxadiazol-3-yl)methyl)quinolin-4(1H)-one

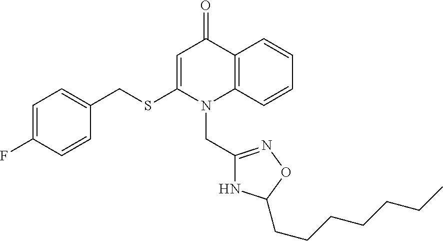 [SMILES: CCCCCCCC1NC(Cn2c(SCc3ccc(F)cc3)cc(=O)c3ccccc32)=NO1]

Following a procedure similar to that described for the preparation of example 1 except that intermediate A14 and octyl aldehyde were used as starting materials. ¹H-NMR (CDCl₃, 400 MHz) δ0.83 (t, 3H, J=7.0), 1.18-1.30 (m, 10H), 1.70 (m, 2H), 4.08 (s, 2H), 5.21 (s, 2H), 5.59 (t, 1H, J=5.2), 5.71 (s, 1H), 6.04 (s, 1H), 7.03 (t, 2H, J=8.6), 7.33 (m, 3H), 7.67 (m, 2H), 8.25 (d, 1H, J=7.6).

### Example 4

### 1-((5-n-butyl-4,5-dihydro-1,2,4-oxa diazol-3-yl)methyl)-2-(4-fluorobenzylthio)-6,7-dihydro-1H-cyclopenta[d]pyrimidin-4 (5H)-one

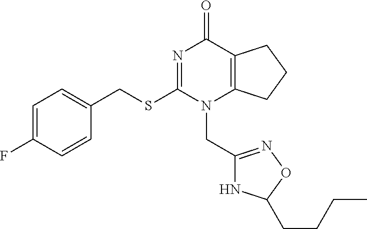 [SMILES: CCCCC1NC(Cn2c(SCc3ccc(F)cc3)nc(=O)c3c2CCC3)=NO1]

Following a procedure similar to that described for the preparation of example 1 except that n-valeraldehyde was used in place of octyl aldehyde. ¹H-NMR (CDCl₃, 400 MHz) δ 0.85 (t, 3H, J=7.2), 1.30 (m, 4H), 1.68 (m, 2H), 2.13 (m, 2H), 2.70 (t, 2H, J=7.2), 3.00 (t, 2H), 4.45 (s, 2H), 4.75 (2×d, 2H, J=16.4), 5.66 (t, 1H, J=4.8), 6.94 (t, 2H, J=8.4), 7.29 (dd, 2H, J=8.4, 4.8).

### Example 5

### 1-((5-n-decyl-4,5-dihydro-1,2,4-oxadiazol-3-yl)methyl)-2-(4-fluorobenzylthio)quinolin-4(1H)-one

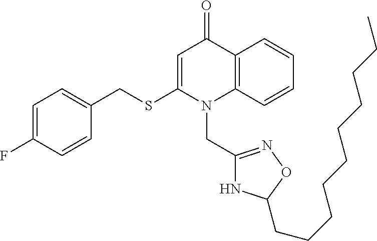 [SMILES: CCCCCCCCCCC1NC(Cn2c(SCc3ccc(F)cc3)cc(=O)c3ccccc32)=NO1]

Following a procedure similar to that described for the preparation of example 1 except that intermediate A14 and n-undecanal was used as staring materials. ¹H-NMR (CDCl₃, 400 MHz) δ0.86 (t, 3H), 1.20 (m, 16H), 1.71 (m, 2H), 4.11 (s, 2H), 5.21 (s, 2H), 5.58 (t, 1H), 5.71 (s, 1H), 6.09 (s, 1H), 7.03 (t, 2H), 7.34 (m, 3H), 7.67 (m, 2H), 8.27 (d, 1H).

The following compounds were prepared by a procedure similar to that described for example 1.

| Name | Structure | Precusor |
|---|---|---|
| Example 6: 2-(4-fluorobenzylthio)-1-((5- n-propyl-4,5-dihydro-1,2,4- oxadiazol-3-yl)methyl)-6,7- dihydro-1H-cyclo- penta[d]pyrimidin- 4(5H)-one | 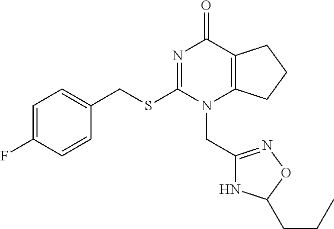 [SMILES: CCCC1NC(Cn2c(SCc3ccc(F)cc3)nc(=O)c3c2CCC3)=NO1] | A7,n-butanal |
| Example 7: 2-(4-fluorobenzylthio)-1-((5- (4′-(trifluoromethyl)biphen- yl-4-yl)-4,5-dihydro-1,2,4- oxadiazol-3-yl)methyl)-6,7- dihydro-1H-cyclo- penta[d]pyrimidin- 4(5H)-one | 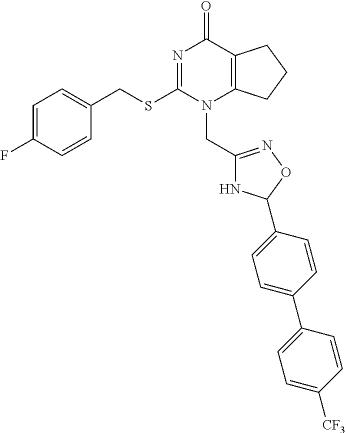 [SMILES: O=c1nc(SCc2ccc(F)cc2)n(CC2=NOC(c3ccc(-c4ccc(C(F)(F)F)cc4)cc3)N2)c2c1CCC2] | A7,M1 |

### Example 8

### (E)-2-(4-fluorobenzylthio)-1-((5-n-heptyl-4-(n-oct-1-enyl)-4,5-dihydro-1,2,4-oxadiazol-3-yl)methyl)-6,7-dihydro-1H-cyclopenta[d]pyrimidin-4 (5H)-one

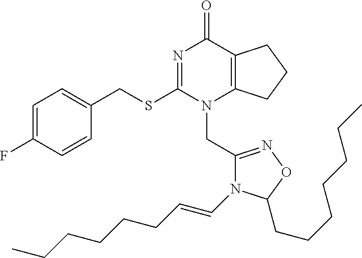 [SMILES: CCCCCC/C=C/N1C(Cn2c(SCc3ccc(F)cc3)nc(=O)c3c2CCC3)=NOC1CCCCCCC]

To a mixture of example 1 (1 equiv) and octyl aldehyde (2 equiv) in anhydrous THF was added boron trifluoride-ether complex (3 equiv) under nitrogen. The mixture was stirred for 2 h at room temperature. About a half of the starting materials were converted and there was no significant conversion of starting materials as time increases. The mixture was quenched with saturated aq. NaHCO₃ solution and extracted with EA. The organic phase was dried over MgSO₄, purified by column chromatography to give the title compound. ¹H-NMR (d₆-DMSO, 400 MHz) δ0.84 (m, 6H), 1.19-1.32 (m, 18H), 1.68 (m, 2H), 1.96 (m, 4H), 2.57 (t, 2H, J=7.2), 2.88 (M, 2H), 4.42 (2×d, 2H, J=13.4), 4.75 (dt, 1H, J=13.6, 6.8), 5.07 (s, 2H), 5.87 (m, 1H), 7.13 (t, 2H, J=8.8), 7.47 (dd, 2H, J=8.4, 5.6); MS (ESI): 569 (M+H).

The following compounds were prepared by a procedure similar to that described for example 8.

| Name | Structure | Precursor |
|---|---|---|
| Example 9: (E)-2-(4-fluorobenzylthio)- 1-((4-(n-pent-1-enyl)-5-n- propyl-4,5-dihydro-1,2,4- oxadiazol-3-yl)methyl)-6,7- dihydro-1H-cyclo- pypenta[d]primidin-4(5H)- one | 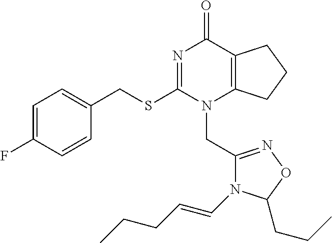 [SMILES: CCC/C=C/N1C(Cn2c(SCc3ccc(F)cc3)nc(=O)c3c2CCC3)=NOC1CCC] | Example 4,n-valeraldehyde |
| Example 10: (E)-1-((4-(n-but-1-enyl)-5- n-propyl-4,5-dihydro-1,2,4- oxadiazol-3-yl)methyl)-2- (4-fluorobenzylthio)-6,7- dihydro-1H-cyclo- penta[d]pyrimidin- 4(5H)-one | 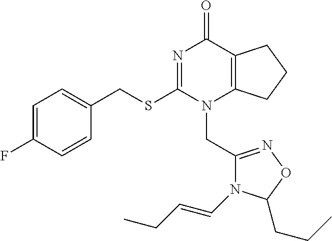 [SMILES: CC/C=C/N1C(Cn2c(SCc3ccc(F)cc3)nc(=O)c3c2CCC3)=NOC1CCC] | Example 4,n-butyl aldehyde |
| Example 11: (E)-1-((5-n-butyl-4-(n-hex- 1-enyl)-4,5-dihydro-1,2,4- oxadiazol-3-yl)methyl)-2- (4-fluorobenzylthio)-6,7- dihydro-1H-cyclo- penta[d]pyrimidin-4(5H)- one | 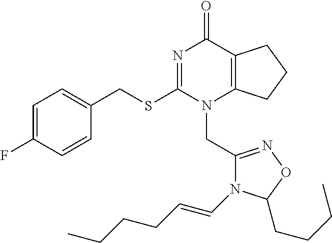 [SMILES: CCCC/C=C/N1C(Cn2c(SCc3ccc(F)cc3)nc(=O)c3c2CCC3)=NOC1CCCC] | Example 5,n-hexanal |
| Example 12: (E)-1-((4-(n-but-1-enyl)-5- n-butyl-4,5-dihydro-1,2,4- oxadiazol-3-yl)methyl)-2- (4-fluorobenzylthio)-6,7- dihydro-1H-cyclo- penta[d]pyrimidin- 4(5H)-one | 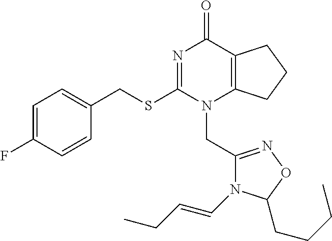 [SMILES: CC/C=C/N1C(Cn2c(SCc3ccc(F)cc3)nc(=O)c3c2CCC3)=NOC1CCCC] | Example 5,n-butyl aldehyde |
| Example 13: (E)-2-(4-fluorobenzylthio)- 1-((4-(n-hex-1-enyl)-5-pro- pyl-4,5-dihydro-1,2,4-oxa- diazol-3-yl)methyl)-6,7-di- hydro-1H-cyclopenta[d]py- rimidin-4(5H)-one | 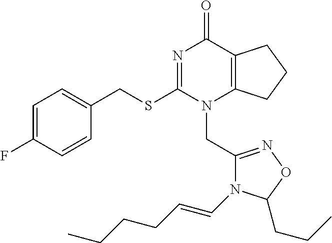 [SMILES: CCCC/C=C/N1C(Cn2c(SCc3ccc(F)cc3)nc(=O)c3c2CCC3)=NOC1CCC] | Example 4,n-hexanal |
| Example 14: (E)-1-((5-n-butyl-4-(n-pent- 1-enyl)-4,5-dihydro-1,2,4- oxadiazol-3-yl)methyl)-2- (4-fluorobenzylthio)-6,7- dihydro-1H-cyclopenta[d]py- rimidin-4(5H)-one | 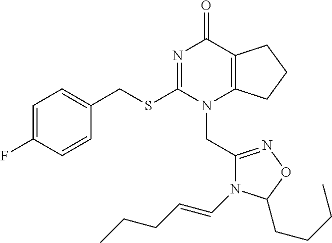 [SMILES: CCC/C=C/N1C(Cn2c(SCc3ccc(F)cc3)nc(=O)c3c2CCC3)=NOC1CCCC] | Example 4,n-valeraldehyde |
| Example 15: (E)-2-(4-fluorobenzylthio)- 1-((4-(n-oct-1-enyl)-5-pro- pyl-4,5-dihydro-1,2,4-oxa- diazol-3-yl)methyl)-6,7- dihydro-1H-cyclopenta[d]py- rimidin-4(5H)-one | 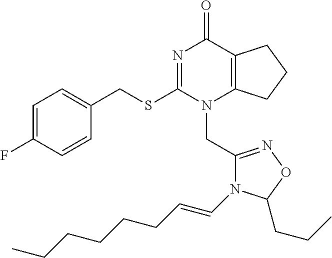 [SMILES: CCCCCC/C=C/N1C(Cn2c(SCc3ccc(F)cc3)nc(=O)c3c2CCC3)=NOC1CCC] | Example 4, octyl aldehyde |
| Example 16: (E)-2-(4-fluorobenzylthio)- 1-((5-n-heptyl-4-(n-pent-1- enyl)-4,5-dihydro-1,2,4- oxadiazol-3-yl)methyl)-6,7- dihydro-1H-cyclo- penta[d]pyrimidin-4(5H)- one | 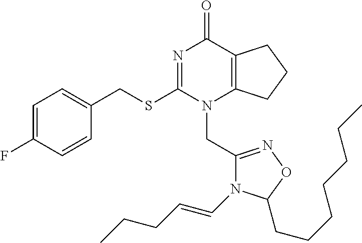 [SMILES: CCC/C=C/N1C(Cn2c(SCc3ccc(F)cc3)nc(=O)c3c2CCC3)=NOC1CCCCCCC] | Example 1, n- valeraldehyde |
| Example 17: (E)-1-((4-(n-but-1-enyl)-5- n-heptyl-4,5-dihydro-1,2,4- oxadiazol-3-yl)methyl)-2- (4-fluorobenzylthio)-6,7- dihydro-1H-cyclopenta[d]py- rimidin-4(5H)-one | 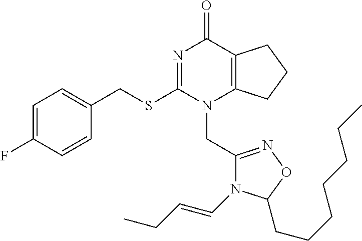 [SMILES: CC/C=C/N1C(Cn2c(SCc3ccc(F)cc3)nc(=O)c3c2CCC3)=NOC1CCCCCCC] | Example 1, n- butyl aldehyde |
| Example 18: (E)-2-(4-fluorobenzylthio)- 1-((5-n-heptyl-4-(n-hex-1- enyl)-4,5-dihydro-1,2,4-ox- adiazol-3-yl)methyl)-6,7-di- hydro-1H-cyclopenta[d]py- rimidin-4(5H)-one | 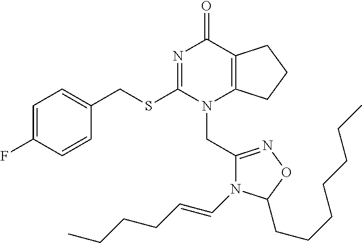 [SMILES: CCCC/C=C/N1C(Cn2c(SCc3ccc(F)cc3)nc(=O)c3c2CCC3)=NOC1CCCCCCC] | Example 1, n-hexanal |

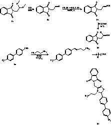

### Intermediate B1—2-(2,2-dimethoxyethyl)isoindoline-1,3-dione

Refer to [Eur. J. Org. Chem., 2008, 895-913.]

### Intermediate B2—2-(1,3-dioxoisoindolin-2-yl)acetaldehyde

To a suspension of intermediate B1 (4.18 g) in water (20 ml) was added concd.HCl (4 ml). The mixture was refluxed for 1 h. TLC detection showed that the reaction was complete. The mixture was extracted with dichloromethane three times. The combined organic phase was washed with brine twice, saturated aq.NaHCO₃ solution once, dried over MgSO₄, decolored with active carbon and purified by column chromatography (PE/EA=2:1) to give the title compound as a white solid (2.93 g). ¹H-NMR (CDCl₃, 300 MHz) δ4.55 (s, 2H), 7.75 (m, 2H), 7.88 (m, 2H), 9.65 (s, 1H).

### Intermediate B3—2-(1,3-dioxoisoindolin-2-yl)acetaldehyde oxime

A mixture of intermediate B2 (1.46 g, 1 equiv), hydroxylamine hydrochloride (0.64 g, 1.2 equiv), anhydrous K₂CO₃ (1.6 g, 1.5 equiv) in absolute methanol (10 ml) was stirred at room temperature overnight. The solvent was evaporated under reduced pressure and the residue was diluted with water, extracted with dichloromethane three times. The organic phase was combined and washed with brine, dried and solvent evaporated to give the title compound as a white solid (0.75 g). ¹H-NMR (d₆-DMSO, 400 MHz, ca 1:1 cis-trans isomer) 64.30/4.37 (2×d, 2H, J=4.0), 6.81/7.35 (2×t, 1H, J=4.0), 7.81-7.88 (m, 4H), 10.86/11.34 (2×s, 1H); MS (EI) m/z: 204 (M⁺).

### Intermediate B4—2-(1,3-dioxoisoindolin-2-yl)-N-hydroxyacetimidoyl chloride

A mixture of intermediate B3 (0.43 g, 1 equiv) and NCS (0.28 g, 1 equiv) in DMF (5 ml) was heated at 60° C. for 2 h. After cooled, the mixture was diluted with EA (20 ml), washed with brine five times to wash out DMF, dried and evaporated to dryness to give the title compound as white powder (0.42 g). ¹H-NMR (d₆-DMSO, 300 MHz) δ4.60 (s, 2H), 7.89-7.97 (m, 4H), 12.02 (s, 1H).

### Intermediate B5—2-((4-(2-(diethylamino)ethyl)-5-(4′-(trifluoromethyl)biphenyl-4-yl)-4,5-dihydro-1,2,4-oxadiazol-3-yl)methyl)isoindoline-1,3-dione

4 Å molecular sieves (3 g) was added to a mixture of intermediate M1 (10 mmol), 2-diethylaminoethylamine (10 mmol) in dichloromethane (10 ml). The mixture was heated under reflux for 2 h, using CaCl₂ drying tube to insulate moisture, to afford the intermediate imine solution. Heating was stopped and the mixture was cooled for use. To an ice-salt cold solution of intermediate B4 (10 mmol) in anhydrous THF (15 ml) was added triethylamine (13 mmol) dropwise over 5 min under nitrogen and vigorous stirring. After the addition, the mixture was further stirred for 15 min. Then, the solution of intermediate imine in DCM was added rapidly and the mixture was stirred at room temperature for 2 h. Then the reaction mixture was poured into saturated aq. NH₄Cl solution and extracted with dichloromethane twice. The combined organic phase was washed with brine twice, dried and then purified by column chromatography to give the title compound as a jelly. ¹H-NMR (CDCl₃, 400 MHz) δ1.02 (t, 6H, J=7.2), 2.44-2.53 (m, 6H), 3.14 (m, 1H), 3.25 (m, 1H), 4.75 (2×d, 2H, J=16.0), 6.38 (s, 1H), 7.63 (2×d, 4H, J=8.4), 7.71 (s, 4H), 7.77 (m, 2H), 7.93 (m, 2H); MS (ESI): 551 (M+H).

### Intermediate B6—2-((4-(2-morpholinoethyl)-5-(4′-(trifluoromethyl)biphenyl-4-yl)-4,5-dihydro-1,2,4-oxadiazol-3-yl)methyl)isoindoline-1,3-dione

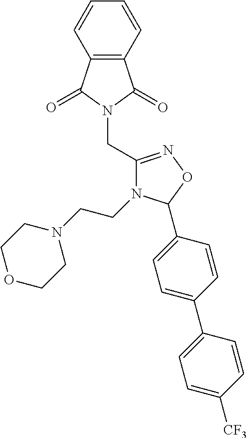 [SMILES: O=C1c2ccccc2C(=O)N1CC1=NOC(c2ccc(-c3ccc(C(F)(F)F)cc3)cc2)N1CCN1CCOCC1]

Following a procedure similar to that described for the preparation of intermediate B5 except that 2-morpholinoethanamine was used in place of 2-diethylaminoethylamine ¹H-NMR (CDCl₃, 300 MHz) δ 2.40 (m, 6H), 3.18 (dt, 1H, J=15.0, 6.0), 3.29 (dt, 1H, J=15.0, 6.3), 3.68 (t, 4H, J=4.7), 4.72 (2×d, 2H, J=15.9), 6.35 (s, 1H), 7.56 (d, 2H, J=8.4), 7.62 (d, 2H, J=8.4), 7.68 (m, 4H), 7.75 (m, 2H), 7.90 (m, 2H).

The following intermediates were prepared by a procedure similar to that described for intermediate B5.

| Name | Structure | Starting material |
|---|---|---|
| Intermediate B7: ethyl 4-(3-((1,3-dioxoisoindolin- 2-yl)methyl)- 5-(4′-(trifluoromethyl)bi- phenyl-4-yl)-1,2,4-oxadiazol- 4(5H)-yl)piperidine-1-car- boxylate | 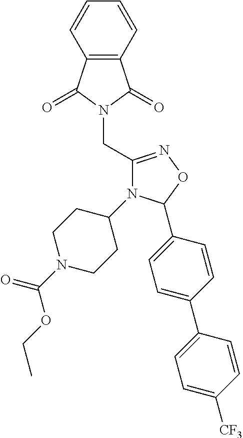 [SMILES: CCOC(=O)N1CCC(N2C(CN3C(=O)c4ccccc4C3=O)=NOC2c2ccc(-c3ccc(C(F)(F)F)cc3)cc2)CC1] | 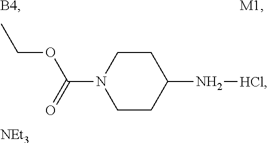 |
| Intermediate B8: 2-((4-(2-(piperidin-1-yl)eth- yl)-5-(4′-(trifluoromethyl)bi- phenyl-4-yl)-4,5-dihydro- 1,2,4-oxadiazol-3-yl)meth- yl)isoindoline-1,3-dione | 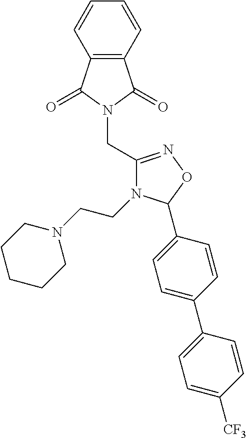 [SMILES: O=C1c2ccccc2C(=O)N1CC1=NOC(c2ccc(-c3ccc(C(F)(F)F)cc3)cc2)N1CCN1CCCCC1] | 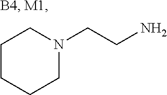 |

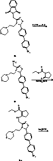

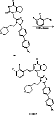

Intermediate B10—ethyl 2-((4-(2-morpholinoethyl)-5-(4′-(trifluoromethyl)biphenyl-4-yl)-4,5-dihydro-1,2,4-oxadiazol-3-yl)methylamino)cyclopent-1-enecarboxylate

To a solution of intermediate B6 (2.57 g, 1 equiv) in THF (15 ml) was added hydrazine hydrate (85%, 0.78 ml, 3 equiv) and the mixture was stirred at 50° C. for 5 h. TLC detection showed that the reaction was complete. Then the mixture was cooled and filtered to remove the insoluble white solid. The filtrate was evaporated to dryness unfrt reduced pressure. Then toluene was added to remove the trace amount of water and this procedure was repeated twice to give intermediate B9 (1.82 g) as an oil. A mixture of intermediate B9 (1.82 g, 1 equiv), ethyl 2-oxocyclopentanecarboxylate (0.65 ml, 1.05 equiv), Si(OEt)₄ (1.86 ml, 2 equiv) in absolute ethanol was refluxed for 4 h under nitrogen until the reaction was complete. The solution was mixed with an amount of silica gel, evaporated to dryness and purified by column chromatography to give the title compound as a jelly (1.37 g). ¹H-NMR (CDCl₃, 300 MHz) δ 1.26 (t, 3H, J=7.2), 1.87 (m, 2H), 2.34 (m, 6H), 2.54 (t, 2H, J=7.2), 2.72 (t, 2H, J=7.2), 3.20 (m, 2H), 3.63 (t, 4H, J=4.5), 4.13 (q, 2H, J=7.2), 4.18 (d, 2H, J=6.6), 6.37 (s, 1H), 7.54 (d, 2H, J=8.4), 7.63 (d, 2H, J=8.4), 7.70 (m, 5H).

### Intermediate B11—1-((4-(2-morpholinoethyl)-5-(4′-(trifluoromethyl)biphenyl-4-yl)-4,5-dihydro-1,2,4-oxa diazol-3-yl)methyl)-2-thioxo-2,3,6,7-tetrahydro-1H-cyclopenta[d]pyrimidin-4(5H)-one

A mixture of intermediate B10 (1.36 g, 1 equiv), Me₃SiNCS (1.33 ml, 4 equiv) and anhydrous DMF (2 ml) was heated at 140° C. for 4 h under nitrogen. TLC detection showed that the reaction was complete. Then, the solution was cooled in an ice bath, quenched with saturated aq. NaHCO₃ solution and then diluted with EA (20 ml). The mixture was stirred for 0.5 h before transferred into a separating funnel. The organic layer was separated, washed with brine twice, dried over MgSO₄, decolored with active carbon, and then the solution was concentrated to a small amount and then silica gel was added and purified by column chromatography to collect the target product and then recrystallized from PE-EA to give the title compound as a white solid (100 mg). ¹H-NMR (d₆-DMSO, 400 MHz) δ 2.05 (m, 2H), 2.32 (m, 6H), 2.62 (t, 2H, J=7.6), 3.04 (m, 3H), 3.35 (m, 1H), 3.56 (m, 4H), 5.38 (s, 2H), 6.44 (s, 1H), 7.64 (d, 2H, J=8.4), 7.83 (d, 2H, J=8.4), 7.93 (d, 2H, J=8.4), 12.65 (s, 1H); MS (ESI): 586 (M+H).

### Example 19

### 2-(2,3-difluorobenzylthio)-1-((4-(2-morpholinoethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one

A mixture of intermediate B11 (30 mg, 1 equiv), 1-(bromomethyl)-2,3-difluorobenzene (7.2 μl, 1.1 equiv), anhydrous K₂CO₃ (11 mg, 1.5 equiv) in acetone (2 ml) was heated to reflux for 0.5 h. Then the mixture was filtered to remove the inorganic salts and the filtrate was purified by preparative TLC (DCM/MeOH=15:1) and the target product portion was collected to give the title compound as a solid (20 mg). ¹H-NMR (CDCl₃, 300 MHz) δ 2.13 (m, 2H), 2.38 (m, 6H), 2.83 (t, 2H, J=7.2), 3.07 (m, 4H), 3.64 (m, 4H), 4.65 (2×d, 2H, J=12.9), 4.84 (s, 2H), 6.37 (s, 1H), 7.05 (m, 2H), 7.38 (m, 1H), 7.52 (d, 2H, J=6.9), 7.63 (d, 2H, J=7.5), 7.70 (4H, m); MS (ESI): 712 (M+H).

### Example 20

### 2-(4-fluorobenzylthio)-1-((4-(2-(piperidin-1-yl)ethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one

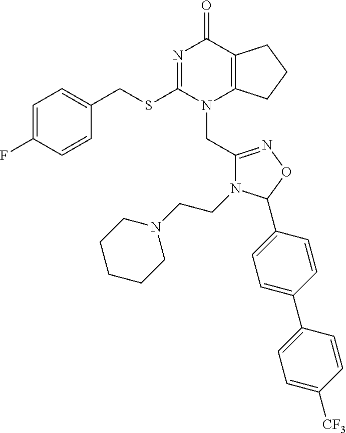 [SMILES: O=c1nc(SCc2ccc(F)cc2)n(CC2=NOC(c3ccc(-c4ccc(C(F)(F)F)cc4)cc3)N2CCN2CCCCC2)c2c1CCC2]

Following a procedure similar to that described for the preparation of example 19 except that intermediate B8 and 1-(bromomethyl)-4-fluorobenzene was used in place of intermediate B6 and 1-(bromomethyl)-2,3-difluorobenzene. ¹H-NMR (CDCl₃, 300 MHz) δ 1.45 (m, 2H), 1.62 (m, 4H), 2.12 (m, 2H), 2.47 (m, 6H), 2.83 (t, 2H, J=6.9), 3.03 (t, 2H, J=7.2), 3.30 (m, 2H), 4.55 (2×d, 2H, J=12.9), 5.00 (2×d, 2H, J=18.0), 6.31 (s, 1H), 6.99 (d, 2H, J=8.7), 7.40 (dd, 2H, J=7.2, 5.7), 7.52 (d, 2H, J=8.1), 7.59 (d, 2H, J=8.1), 7.66 (d, 2H, J=8.4), 7.72 (d, 2H, J=8.4); MS (ESI): 692 (M+H).

### Example 21

### ethyl 4-(3-((2-(2,3-difluorobenzylthio)-4-oxo-4,5,6,7-tetrahydro-1H-cyclopenta[d]pyrimidin-1-yl)methyl)-5-(4′-(trifluoromethyl)biphenyl-4-yl)-1,2,4-oxadiazol-4(5H)-yl)piperidine-1-carboxylate

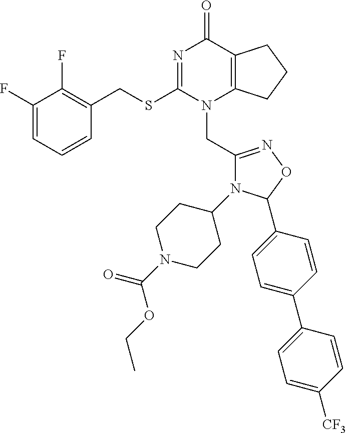 [SMILES: CCOC(=O)N1CCC(N2C(Cn3c(SCc4cccc(F)c4F)nc(=O)c4c3CCC4)=NOC2c2ccc(-c3ccc(C(F)(F)F)cc3)cc2)CC1]

Following a procedure similar to that described for the preparation of example 19 except that intermediate B7 and 1-(bromomethyl)-4-fluorobenzene was used in place of intermediate B6 and 1-(bromomethyl)-2,3-difluorobenzene. ¹H-NMR (CDCl₃, 300 MHz) δ 1.16 (m, 2H), 1.23 (t, 3H, J=6.9), 1.57 (m, 1H), 1.84 (m, 1H), 2.09 (m, 2H), 2.53 (t, 1H, J=13.5), 2.70 (t, 1H, J=12.6), 2.80 (t, 2H, J=6.9), 2.96 (m, 2H), 3.41 (t, 1H, J=12.3), 4.10 (m, 3H), 4.30 (m, 1H), 4.65 (2×d, 2H, J=13.8), 4.80 (2×d, 2H J=17.4), 6.39 (s, 1H), 7.05 (m, 2H), 7.38 (t, 1H, J=6.3), 7.47 (d, 2H, J=8.1), 7.59 (d, 2H, J=8.1), 7.69 (4H, m); MS (ESI): 754 (M+H)⁺.

### Example 22

### ethyl 4-(3-(2-(4-fluorobenzylthio)-4-oxo-4,5,6,7-tetrahydro-1H-cyclopenta[d]pyrimidin-1-yl)methyl)-5-(4′-(trifluoromethyl)biphenyl-4-yl)-1,2,4-oxadiazol-4(5H)-yl)piperidine-1-carboxylate

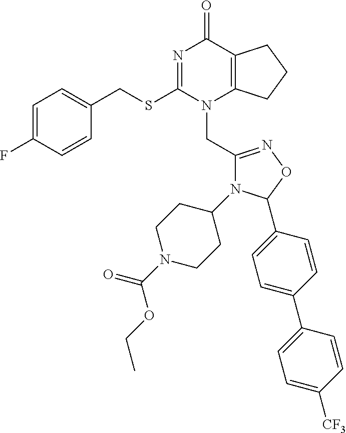 [SMILES: CCOC(=O)N1CCC(N2C(Cn3c(SCc4ccc(F)cc4)nc(=O)c4c3CCC4)=NOC2c2ccc(-c3ccc(C(F)(F)F)cc3)cc2)CC1]

Following a procedure similar to that described for the preparation of example 19 except that intermediate B7 was used in place of intermediate B6. ¹H-NMR (CDCl₃, 300 MHz) δ 1.26 (m, 5H), 1.54 (m, 1H), 1.82 (m, 1H), 2.10 (m, 2H), 2.50 (t, 1H, J=12.0), 2.66 (t, 1H, J=13.2), 2.83 (t, 2H, J=7.2), 2.97 (m, 2H), 3.39 (t, 1H, J=12.3), 4.11 (m, 3H), 4.30 (m, 1H), 4.52 (d, 1H, J=13.5), 4.65 (d, 1H, J=13.5), 4.80 (2×d, 2H J=17.4), 6.40 (s, 1H), 7.00 (t, 2H, J=8.4), 7.40 (dd, 2H, J=8.4, 5.1), 7.47 (d, 2H, J=8.9), 7.57 (d, 2H, J=8.4), 7.67 (d, 2H, J=8.4), 7.71 (d, 2H, J=8.7); MS (ESI): 736 (M+H)⁺.

### Example 23

### 2-(2,3-difluorobenzylthio)-1-((4-(2-(piperidin-1-yl)ethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one

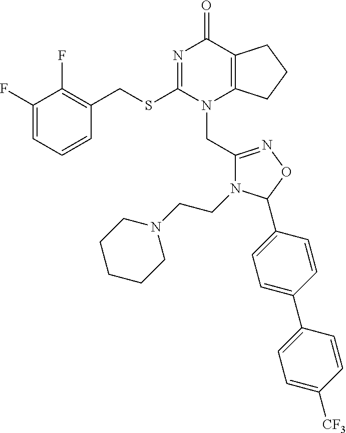 [SMILES: O=c1nc(SCc2cccc(F)c2F)n(CC2=NOC(c3ccc(-c4ccc(C(F)(F)F)cc4)cc3)N2CCN2CCCCC2)c2c1CCC2]

Following a procedure similar to that described for the preparation of example 19 except that intermediate B8 and 1-(bromomethyl)-4-fluorobenzene was used in place of intermediate B6 and 1-(bromomethyl)-2,3-difluorobenzene. ¹H-NMR (CDCl₃, 300 MHz) δ 1.46 (m, 2H), 1.61 (m, 4H), 2.13 (m, 2H), 2.46 (m, 6H), 2.84 (t, 2H), 3.01 (t, 2H), 3.25 (m, 2H), 4.63 (2×d, 2H), 4.94 (2×d, 2H), 6.32 (s, 1H), 7.04 (m, 2H), 7.38 (m, 1H), 7.55 (d, 2H), 7.63 (d, 2H), 7.71 (4H, m); MS (ESI): 710 (M+H)⁺.

The following compounds were prepared by a procedure similar to that described for example 19 from B6.

| Name | Structure | Precusor |
|---|---|---|
| Example 24: 1-((4-(2-(diethylamino)eth- yl)-5-(4′-(trifluorometh- yl)biphenyl-4-yl)-4,5-di- hydro-1,2,4-oxadiazol-3-yl )methyl)-2-(4-fluorobenzyl- thio)-6,7-dihydro-1H- cyclopenta[d]pyrimidin- 4(5H)-one | 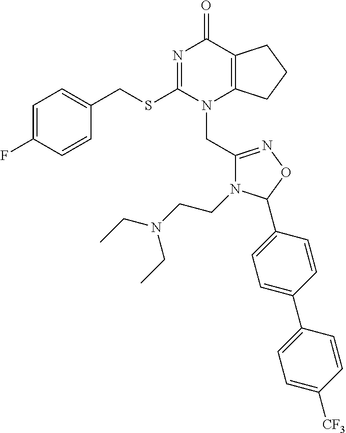 [SMILES: CCN(CC)CCN1C(Cn2c(SCc3ccc(F)cc3)nc(=O)c3c2CCC3)=NOC1c1ccc(-c2ccc(C(F)(F)F)cc2)cc1] | B5, 1-(bromomethyl)- 4-fluorobenzene |
| Example 25: 2-(4-fluorobenzylthio)-1- ((4-(2-morpholinoethyl)- 5-(4′-(trifluoromethyl)bi- phenyl-4-yl)-4,5-dihydro- 1,2,4-oxadiazol-3-yl)meth- yl)-6,7-dihydro-1H-cy- clopenta[d]pyrimidin- 4(5H)-one | 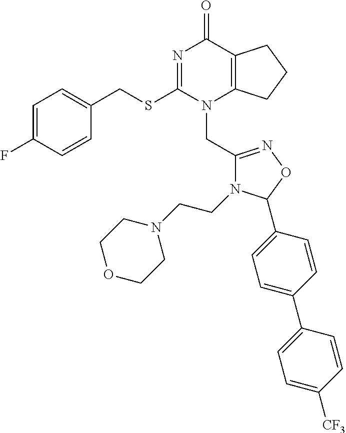 [SMILES: O=c1nc(SCc2ccc(F)cc2)n(CC2=NOC(c3ccc(-c4ccc(C(F)(F)F)cc4)cc3)N2CCN2CCOCC2)c2c1CCC2] | B6, 1-(bromomethyl)- 4-fluorobenzene |

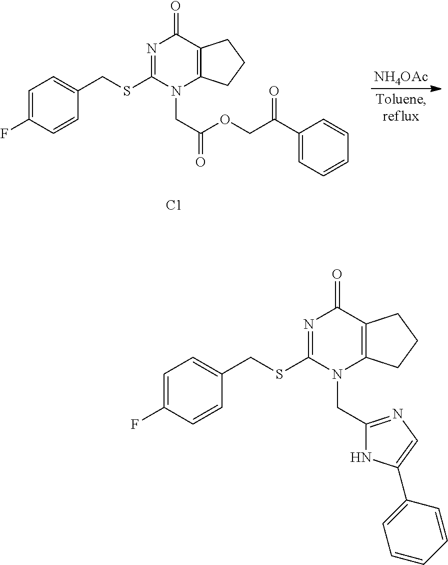 [SMILES: O=C(Cn1c(SCc2ccc(F)cc2)nc(=O)c2c1CCC2)OCC(=O)c1ccccc1 ; "C1" ; O=c1nc(SCc2ccc(F)cc2)n(Cc2ncc(-c3ccccc3)[nH]2)c2c1CCC2 ; "NH4OAc"]

Intermediate C1—2-oxo-2-phenylethyl 2-(2-(4-fluorobenzylthio)-4-oxo-4,5,6,7-tetrahydro-1H-cyclopenta[d]pyrimidin-1-yl)acetate

A mixture of intermediate A4 (1.67 g, 1 equiv), 2-bromo-1-phenylethanone (1 g, 1 equiv), anhydrous K₂CO₃ (1.38 g, 2 equiv) in acetone (20 ml) was stirred at room temperature for 6 h. TLC detection showed that the reaction was complete. Solvent was evaporated under reduced pressure and the residue was diluted with water and EA. The mixture was stirred for 10 min and the solid so obtained was collected by filtration, washed with water and EA, and then dried to give the title compound as a white solid (1.14 g). Then organic layer of the filtrate was separated and evaporated to remove most of the solvent to give a further batch of the title compound (0.75 g). ¹H-NMR (d₆-DMSO, 300 MHz) δ1.99 (m, 2H), 2.60 (t, 2H, J=7.3), 2.95 (t, 2H, J=7.5), 4.45 (s, 2H), 5.02 (s, 2H), 5.66 (s, 2H), 7.15 (t, 2H, J=8.8), 7.49 (dd, 2H, J=8.8, 5.6), 7.56 (t, 2H, J=7.3), 7.69 (t, 1H, J=7.2), 7.95 (d, 1H, J=6.6); MS (ESI): 453 (M+H).

### Example 26

### 2-(4-fluorobenzylthio)-1-((5-phenyl-1H-imidazol-2-yl)methyl)-6,7-dihydro-1H-cyclopenta[d]pyrimidin-4(5H)-one

A mixture of intermediate C1 (665 mg, 1.47 mmol) and ammonium acetate (560 mg, 5 equiv) in toluene was refluxed under nitrogen in a Dean-Stark trap for 1.5 h. Toluene was evaporated and the residue was dissolved in methanol, mixed with silica gel, concentrated to dryness and purified by column chromatography. The column was first eluted with DCM/MeOH (30:1) to recycle material and then eluted with DCM/MeOH (15:1) to obtain the title compound (67 mg). ¹H-NMR (d₆-DMSO, 300 MHz) δ2.00 (m, 2H), 2.61 (t, 2H, J=7.5), 3.02 (t, 2H, J=6.9), 4.41 (s, 2H), 5.17 (s, 2H), 7.11 (t, 2H, J=9.0), 7.34 (m, 3H), 7.45 (dd, 2H, J=8.6, 5.6), 7.68 (d, 2H, J=6.0); MS (ESI): 433 (M+H).

### Example 27

### 1-((1H-tetrazol-5-yl)methyl)-2-(4-fluorobenzylthio)-6,7-dihydro-1H-cyclopenta[d]pyrimidin-4(5H)-one

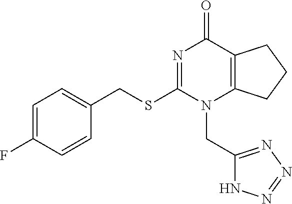 [SMILES: O=c1nc(SCc2ccc(F)cc2)n(Cc2nnn[nH]2)c2c1CCC2]

A mixture of intermediate A6 (95 mg, 1 equiv), sodium azide (21 mg, 1.06 equiv) and ammonium chloride (18 mg, 1.1 equiv) in DMF (2 ml) was heated at 90-100° C. for 2 h. The mixture was diluted with water, acidified to pH 3-4 with concd. HCl and extracted with EA three times. The combined organic phase was washed with brine to neutral, dried over MgSO₄ and solvent evaporated. The residue was recrystallized from MeOH-EA to give the title compound (11 mg) as a solid. ¹H-NMR (d₆-DMSO, 300 MHz) δ1.97 (m, 2H), 2.58 (t, 2H, J=7.4), 2.91 (t, 2H, J=7.6), 4.36 (s, 2H), 5.46 (s, 2H), 7.10 (t, 2H, J=9.0), 7.40 (dd, 2H, J=8.8, 5.6); MS (ESI): 359 (M+H).

### Intermediate C2—1H-imidazole-2-carbaldehyde

Refer to [J. Het. Chem., 32, 611 (1995).]

### Intermediate C3—1-((4′-(trifluoromethyl)biphenyl-4-yl)methyl)-1H-imidazole-2-carbaldehyde

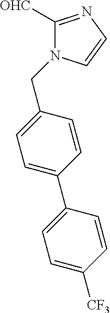 [SMILES: O=Cc1nccn1Cc1ccc(-c2ccc(C(F)(F)F)cc2)cc1]

A mixture of intermediate M11 (1.26 g, 1 equiv), intermediate C2 (396 mg, 1 equiv) and anhydrous K₂CO₃ (830 mg, 1.5 equiv) in acetonitrile (10 ml) was refluxed for 1 h. TLC detection showed that the reaction was complete. The mixture was filtered to remove the insoluble substances and washed with EA. The filtrate was concentrated and then purified by column chromatography (PE/EA=2:1) to give the title compound as a white solid (1.14 g). ¹H-NMR (CDCl₃, 300 MHz) δ5.67 (s, 2H), 7.20 (s, 1H), 7.30 (d, 2H, J=8.4), 7.33 (s, 1H), 7.57 (d, 2H, J=8.4), 7.67 (q, 4H), 9.86 (s, 1H); MS (ESI): 331 (M+H).

The following intermediates were prepared by a procedure similar to that described for intermediate C3.

| Name | Structure | Precusor |
|---|---|---|
| Intermediate C4: 1-n-dodecyl-1H-imidazole- 2-carbaldehyde | 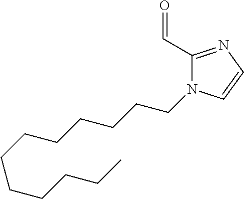 [SMILES: CCCCCCCCCCCCn1ccnc1C=O] | C2, bromododecane |
| Intermediate C5: 1-n-butyl-1H-imidazole-2- carbaldehyde | 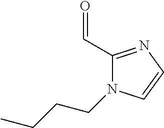 | C2, 1-bromobutane |
| Indtermediate C6: 1-(biphenyl-4-ylmethyl)-1H- imidazole-2-carbalde- hyde | 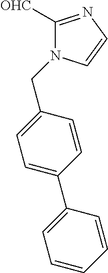 [SMILES: O=Cc1nccn1Cc1ccc(-c2ccccc2)cc1] | C2, M4 |
| Intermediate C7: 1-((4′-methylbiphenyl-4- yl)methyl)-1H-imidazole-2- carbaldehyde | 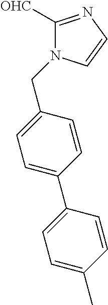 [SMILES: Cc1ccc(-c2ccc(Cn3ccnc3C=O)cc2)cc1] | C2, M5 |
| Intermediate C8: 1-((4′-chlorobiphenyl-4-yl) methyl)-1H-imidazole-2- carbaldehyde | 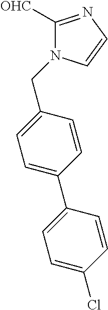 [SMILES: O=Cc1nccn1Cc1ccc(-c2ccc(Cl)cc2)cc1] | C2, M6 |

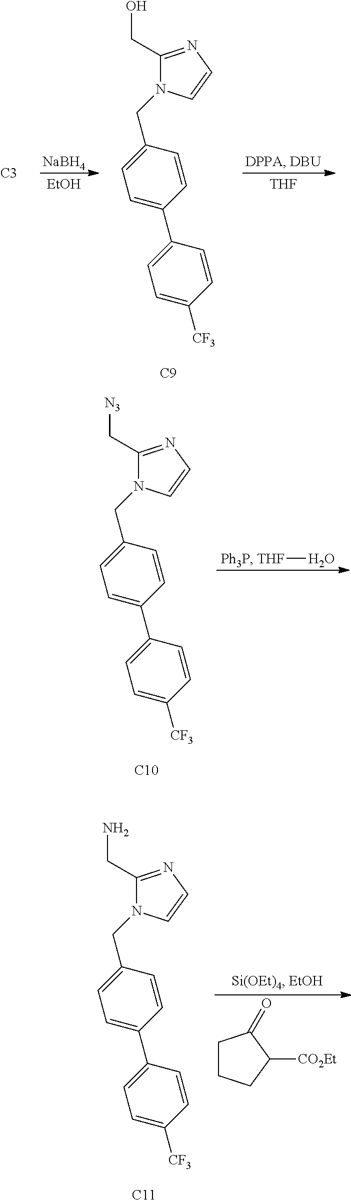

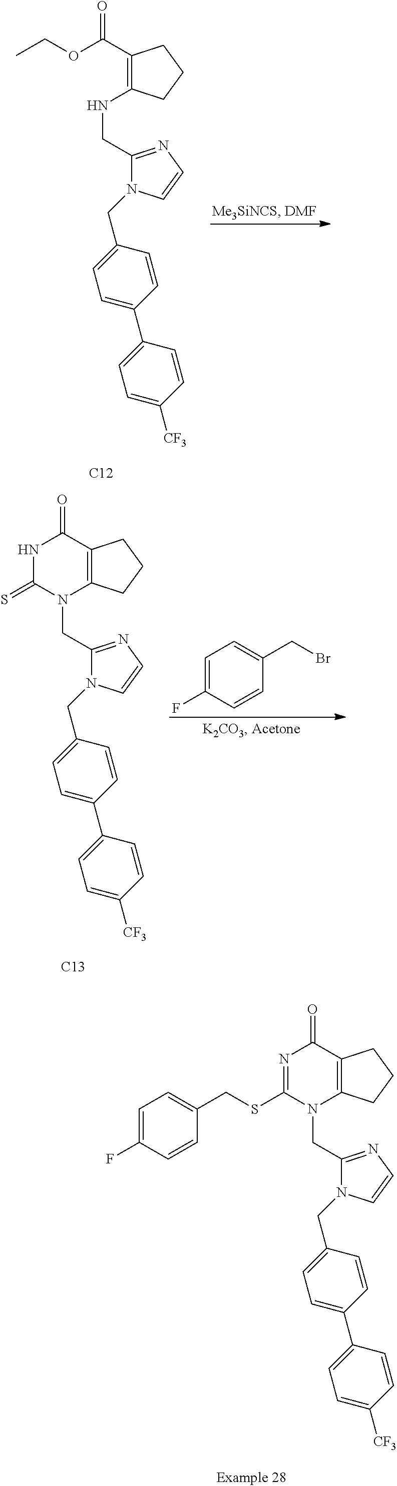

### Intermediate C9—(1-((4′-(trifluoromethyl)biphenyl-4-yl)methyl)-1H-imidazol-2-yl)methanol

Following a procedure similar to that described for the preparation of intermediate M10 except that intermediate C3 was used in place of intermediate M1. ¹H-NMR (CDCl₃, 300 MHz) δ4.68 (s, 2H), 5.29 (s, 2H), 6.88 (s, 1H), 6.97 (s, 1H), 7.25 (d, 2H, J=8.1), 7.57 (d, 2H, J=8.1), 7.65 (d, 2H, J=9.0), 7.69 (d, 2H, J=9.0); MS (ESI): 333 (M+H).

### Intermediate C10—2-(azidomethyl)-1-((4′-(trifluoromethyl)biphenyl-4-yl)methyl)-1H-imidazole

A mixture of intermediate C9 (1.05 g, 1 equiv), DPPA (0.89 ml, 1.3 equiv) and DBU (0.66 ml, 1.4 equiv) in THF (10 ml) was refluxed for 3 h under nitrogen. TLC detection showed that the reaction was complete. After cooled, the solution was poured into saturated NH₄Cl solution and extracted with EA. The organic phase was washed with brine, dried and then purified by column chromatography (PE/EA=1:2) to give the title compound as a white solid (1.10 g). ¹H-NMR (CDCl₃, 300 MHz) δ4.41 (s, 2H), 5.23 (s, 2H), 6.97 (d, 1H, J=1.5), 7.08 (d, 1H, J=0.9), 7.20 (d, 2H, J=8.4), 7.59 (d, 2H, J=8.4), 7.68 (m, 4H).

### Intermediate C11—(1-((4′-(trifluoromethyl)biphenyl-4-yl)methyl)-1H-imidazol-2-yl)methanamine

To a solution of intermediate C10 (0.50 g, 1 equiv) in THF—H₂O (5-0.5 ml) was added Ph₃P (550 mg, 1.5 equiv) and the mixture was stirred at room temperature for 4 h. Then the solution was mixed with silica gel, evaporated to dryness and loaded on a short silica column. The column was first eluted with EA to get rid of Ph₃P and Ph₃P═O and then eluted with methanol to afford the target product portion. The methanol solution so collected was evaporated to dryness and the residue was added a mixed solvent of DCM/MeOH (15:1). Insoluble silica gel was removed by filtration and the filtrate was evaporated to dryness to give the title compound as a yellow solid (0.45 g), which can be used directly in the next reaction.

Intermediate C12—ethyl 2-((1-((4′-(trifluoromethyl)biphenyl-4-yl)methyl)-1H-imidazol-2-yl)methylamino)cyclopent-1-enecarboxylate

To a solution of intermediate C11 (0.45 g, 1 equiv) in absolute ethanol (5 ml) were added ethyl 2-oxocyclopentanecarboxylate (210 μl, 1.05 equiv) and tetraethyl orthosilicate (610 μl, 2 equiv). The mixture was refluxed under nitrogen for 3 h until the completion of the reaction monitored by TLC. Silica gel was added to the solution and the mixture was evaporated to dryness, and then purified by column chromatography (PE/EA=1:3) to give the title compound as a jelly (0.56 g). ¹H-NMR (CDCl₃, 300 MHz) δ1.22 (t, 3H, J=7.1) 1.77 (m, 2H), 2.48 (m, 4H), 4.08 (q, 2H, J=7.2), 4.37 (d, 2H, J=5.7), 5.21 (s, 2H), 6.93 (d, 1H, J=1.2), 7.03 (d, 1H, J=0.9), 7.15 (d, 2H, J=8.4), 7.55 (d, 2H, J=8.1), 7.67 (m, 5H); MS (ESI) found (M+H)=470.

### Intermediate C13—2-thioxo-1-((1-((4′-(trifluoromethyl)biphenyl-4-yl)methyl)-1H-imidazol-2-yl)methyl)-2,3,6,7-tetrahydro-1H-cyclopenta[d]pyrimidin-4(5H)-one

A mixture of intermediate C12 (0.55 g, 1 equiv), isothiocyanatotrimethylsilane (620 μl, 3.5 equiv) in anhydrous DMF (1 ml) was heated at 140° C. for 4 h under nitrogen. Then, the solution was cooled in an ice bath, quenched with saturated NaHCO₃ solution, and stirred with EA (15 ml) and water (10 ml) for 10 minutes. The organic layer was separated and the aqueous layer was extracted with 10 ml of EA again. The combined organic phase was washed with brine twice, dried over anhydrous MgSO₄, decolored with active carbon and filtered to remove the insoluble substances one hour later. The filtrate was concentrated to about 3 ml and stayed overnight after cooled. The white solid so obtained was collected by filtration and dried to give the title compound (100 mg). If no solid precipitated out from EA, the crude product could be purified by column chromatography. ¹H-NMR (d₆-DMSO, 300 MHz) δ1.93 (m, 2H), 2.52 (t, 2H), 2.88 (t, 2H, J=7.6), 5.43 (s, 2H), 5.52 (s, 2H), 6.90 (d, 1H, J=0.6), 7.27 (d, 3H), 7.71 (d, 2H, J=8.4), 7.81 (d, 2H, J=8.4), 7.87 (d, 2H, J=8.1), 12.50 (s, 1H); MS (ESI) found (M+H)=483.

### Example 28

### 2-(4-fluorobenzylthio)-1-((1-((4′-(trifluoromethyl)biphenyl-4-yl)methyl)-1H-imid azol-2-yl)methyl)-6,7-dihydro-1H-cyclopenta[d]pyrimidin-4(5H)-one

A mixture of intermediate C13 (30 mg, 1 equiv), 1-(bromomethyl)-4-fluorobenzene (9 μl, 1.1 equiv) and anhydrous K₂CO₃ (13 mg, 1.5 equiv) in acetone (2 ml) was refluxed for 0.5 h. TLC detection showed that the reaction was complete. The inorganic salt was removed by filtration and the filtrate was purified by column chromatography or preparative TLC (spread with DCM/MeOH=20:1) to collect the target product portion, obtaining the title compound as a solid (20 mg). ¹H-NMR (CDCl₃, 300 MHz) δ1.97 (m, 2H), 2.68 (m, 4H), 4.47 (s, 2H), 5.01 (s, 2H), 5.19 (s, 2H), 6.90-7.03 (m, 5H), 7.10 (s, 1H), 7.33 (t, 2H), 7.48 (d, 2H, J=7.8), 7.63 (d, 2H, J=7.5), 7.48 (d, 2H, J=8.1); MS (ESI): 591 (M+H).

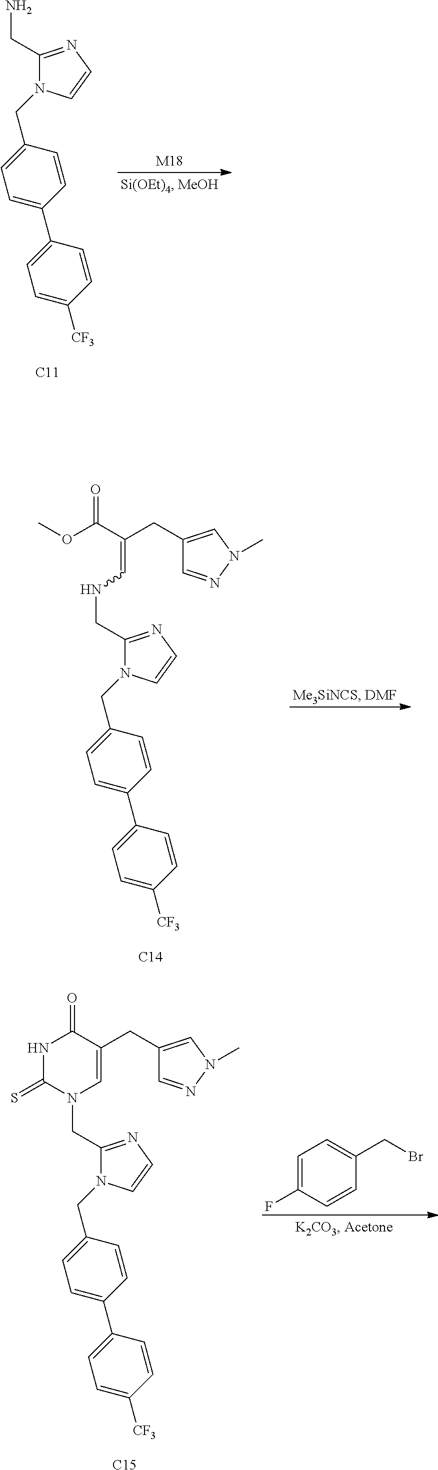

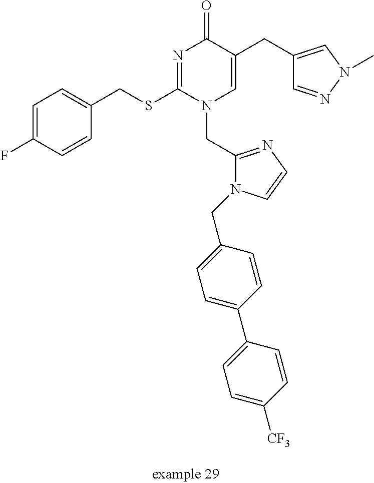 [SMILES: Cn1cc(Cc2cn(Cc3nccn3Cc3ccc(-c4ccc(C(F)(F)F)cc4)cc3)c(SCc3ccc(F)cc3)nc2=O)cn1]

### Intermediate C14—methyl 2-((1-methyl-1H-pyrazol-4-yl)methyl)-3-((1-((4′-(trifluoromethyl)biphenyl-4-yl)methyl)-1H-imidazol-2-yl)methylamino)acrylate

To a solution of intermediate C11 (0.23 g, 1 equiv) in anhydrous methanol (3 ml) were added intermediate M18 (200 mg, 1.43 equiv) and tetraethyl orthosilicate (445 μl, 2 equiv) and the mixture was refluxed under nitrogen for 3 h. TLC detection showed that the reaction was complete. The solution was mixed with silica gel, evaporated to dryness and purified by column chromatography (eluted with DCM/MeOH=30:1) to give the title compound as a jelly (0.35 g). ¹H-NMR (CDCl₃, 300 MHz, ca 2.3:1 cis-trans isomer) 63.18/3.24 (2×s, 2H), 3.62/3.64 (2×s, 3H), 3.80/3.77 (2×s, 3H), 4.33/4.34 (2×d, 2H, J=5.4), 5.17/5.13 (2×s, 2H), 6.56/7.32 (2×d, 1H, J=12.9), 6.95/6.97 (2×d, 1H, J=1.2), 7.06-7.13 (m, 4H), 7.22/7.23 (2×s, 1H), 7.55/7.57 (2×d, 2H, J=8.1), 7.64 (m, 4H), 7.84 (m, 1H).

### Intermediate C15—5-((1-methyl-1H-pyrazol-4-yl)methyl)-2-thioxo-1-((1-((4′-(trifluoromethyl)biphenyl-4-yl)methyl)-1H-imidazol-2-yl)methyl)-2,3-dihydropyrimidin-4(1H)-one

A mixture of intermediate C14 (0.34 g, 1 equiv), isothiocyanatotrimethylsilane (340 μl, 3.5 equiv) in anhydrous DMF (0.6 ml) was heated at 140° C. for 3 h under nitrogen. Then, the solution was cooled in an ice bath, quenched with saturated NaHCO₃ solution, diluted with EA (5 ml) and water (5 ml), and then stirred for a further 20 min to dissolve all the product (if the product could not be dissolved, a small amount of methanol may be added) before it was stored in refrigerator. The precipitate so obtained was collected by filtration and dried to give the title compound (130 mg). ¹H-NMR (d6-DMSO, 300 MHz) δ3.29 (s, 2H), 3.74 (s, 3H), 5.39 (s, 2H), 5.45 (s, 2H), 6.94 (d, 1H, J=0.9), 7.20 (s, 1H), 7.22 (d, 2H, J=8.7), 7.33 (d, 1H, J=0.9), 7.43 (s, 1H), 7.47 (s, 1H), 7.66 (d, 2H, J=8.4), 7.81 (d, 2H, J=8.7), 7.85 (d, 2H, J=8.4), 12.60 (s, 1H); MS (ESI): 537 (M+H).

### Example 29

### 2-(4-fluorobenzylthio)-5-((1-methyl-1H-pyrazol-4-yl)methyl)-1-((1-((4′-(trifluoromethyl)biphenyl-4-yl)methyl)-1H-imidazol-2-yl)methyl)pyrimidin-4(1H)-one

Following a procedure similar to that described for the preparation of example 28 except that intermediate C15 was used in place of intermediate C13. ¹H-NMR (CDCl₃, 300 MHz) δ3.38 (s, 2H), 3.79 (s, 3H), 4.44 (s, 2H), 4.92 (s, 2H), 5.11 (s, 2H), 6.90-6.98 (m, 5H), 7.05 (s, 1H), 7.12 (s, 1H), 7.19 (s, 1H), 7.22 (s, 1H), −7.37 (m, 4H), 7.62 (d, 2H, J=8.4), 7.70 (d, 2H, J=8.1); MS (ESI): 645 (M+H).

### Example 30

### 1-((1-n-dodecyl-1H-imidazol-2-yl)methyl)-2-(4-fluorobenzylthio)-6,7-dihydro-1H-cyclopenta[d]pyrimidin-4(5H)-one

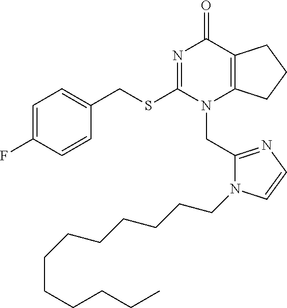 [SMILES: CCCCCCCCCCCCn1ccnc1Cn1c(SCc2ccc(F)cc2)nc(=O)c2c1CCC2]

Following a procedure similar to that described for the preparation of example 28 except that intermediate C4 was used in place of intermediate C3. ¹H-NMR (CDCl₃, 300 MHz) δ 0.85 (t, 3H), 1.23 (m, 18H), 1.69 (m, 2H), 2.08 (m, 2H), 2.86 (m, 4H), 3.86 (t, 2H), 4.48 (s, 2H), 5.05 (s, 2H), 6.88 (s, 1H), 6.96 (t, 2H), 6.99 (s, 1H), 7.30 (dd, 2H); MS (ESI): 525 (M+H).

### Example 31

### 1-((1-n-butyl-1H-imidazol-2-yl)methyl)-2-(4-fluorobenzylthio)-6,7-dihydro-1H-cyclopenta[d]pyrimidin-4(5H)-one

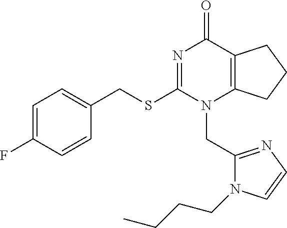 [SMILES: CCCCn1ccnc1Cn1c(SCc2ccc(F)cc2)nc(=O)c2c1CCC2]

Following a procedure similar to that described for the preparation of example 28 except that intermediate C5 was used in place of intermediate C3. ¹H-NMR (CDCl₃, 300 MHz) δ0.89 (t, 3H, J=7.5), 1.28 (m, 2H), 1.66 (m, 2H), 2.08 (m, 2H), 2.82 (t, 2H, J=7.5), 2.89 (t, 2H, J=7.8), 3.87 (t, 2H, J=7.5), 4.48 (s, 2H), 5.05 (s, 2H), 6.88 (s, 1H), 6.95 (t, 2H, J=9.0), 6.98 (s, 1H), 7.32 (dd, 2H, J=8.7, 5.8); MS (ESI): 413 (M+H).

### Example 32

### 1-((1-((4′-chlo robiphenyl-4-yl)methyl)-1H-imidazol-2-yl)methyl)-2-(4-fluorobenzylthio)-6,7-dihydro-1H-cyclopenta[d]pyrimidin-4(5H)-one

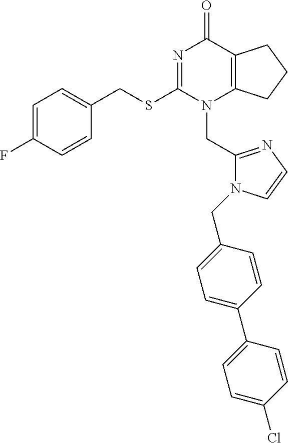 [SMILES: O=c1nc(SCc2ccc(F)cc2)n(Cc2nccn2Cc2ccc(-c3ccc(Cl)cc3)cc2)c2c1CCC2]

Following a procedure similar to that described for the preparation of example 28 except that intermediate C8 was used in place of intermediate C3. ¹H-NMR (CDCl₃, 300 MHz) δ1.96 (m, 2H), 2.68 (m, 4H), 4.46 (s, 2H), 5.00 (s, 2H), 5.17 (s, 2H), 6.91˜7.03 (m, 5H), 7.09 (s, 1H), 7.32 (dd, 2H, J=8.1, 5.4), 7.43 (m, 6H); MS (ESI): 557 (M+H).

### Example 33

### 1-((1-(biphenyl-4-ylm ethyl)-1H-imidazol-2-yl)methyl)-2-(4-fluorobenzylthio)-6,7-dihydro-1H-cyclopenta[d]pyrimidin-4 (5H)-one

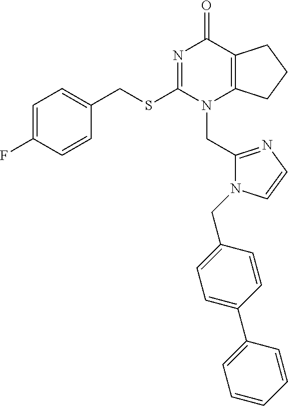 [SMILES: O=c1nc(SCc2ccc(F)cc2)n(Cc2nccn2Cc2ccc(-c3ccccc3)cc2)c2c1CCC2]

Following a procedure similar to that described for the preparation of example 28 except that intermediate C6 was used in place of intermediate C3. ¹H-NMR (CDCl₃, 300 MHz) δ1.98 (m, 2H), 2.71 (m, 4H), 4.46 (s, 2H), 4.98 (s, 2H), 5.17 (s, 2H), 6.92 (t, 2H, J=8.4), 7.02 (d, 2H, J=8.1), 7.04 (s, 1H), 7.09 (s, 1H), 7.30 (dd, 2H, J=8.4, 5.4), 7.39 (m, 1H), 7.50 (m, 6H); MS (ESI): 523 (M+H).

### Example 34

### 2-(4-fluorobenzylthio)-5-((1-methyl-1H-pyrazol-4-yl)methyl)-1-((1-((4′-methylb ip henyl-4-yl)methyl)-1H-imidazol-2-yl)methyl)pyrimidin-4(1H)-one

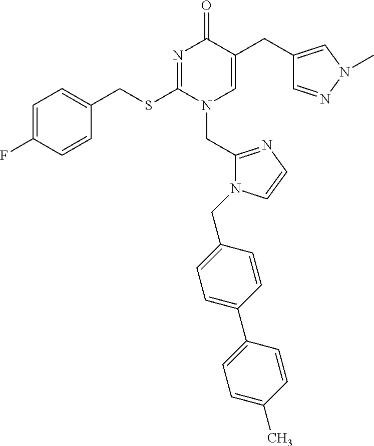 [SMILES: Cc1ccc(-c2ccc(Cn3ccnc3Cn3cc(Cc4cnn(C)c4)c(=O)nc3SCc3ccc(F)cc3)cc2)cc1]

Following a procedure similar to that described for the preparation of example 29 except that intermediate C7 was used in place of intermediate C3. ¹H-NMR (CDCl₃, 300 MHz) δ2.40 (s, 3H), 3.43 (s, 2H), 3.80 (s, 3H), 4.46 (s, 2H), 4.90 (s, 2H), 5.08 (s, 2H), 6.90-6.93 (m, 5H), 7.04 (s, 1H), 7.11 (s, 1H), 7.22-7.30 (m, 4H), −7.34 (m, 2H), 7.43 (m, 4H); MS (ESI): 591 (M+H).

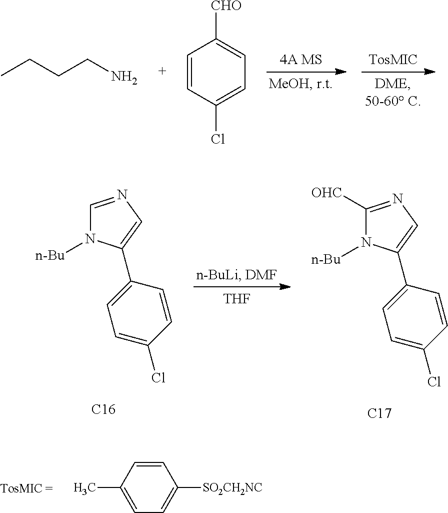

### Intermediate C16—1-n-butyl-5-(4-chlorophenyl)-1H-imidazole

To a mixture of n-butylamine (2.2 ml, 2.2 equiv) and 4-chlorobenzaldehyde (1.4 g, 1 equiv) in anhydrous methanol (10 ml) was added 4 Å molecular sieve (5 g) and the mixture was stayed for 3 h with occasional shake of the flask equipped with CaCl₂ drying tube. Then, DME (10 ml) and 1-(isocyanomethylsulfonyl)-4-methylbenzene (2.34 g, 1.2 equiv) were added and the mixture was heated at 5060° C. for 12 h. After cooled, the mixture was filtered to remove molecular sieve and the filtrate was mixed with silica gel, and evaporated to dryness and then purified by column chromatography to give the title compound as a jelly (0.98 g). ¹H-NMR (CDCl₃, 300 MHz) δ0.83 (t, 3H, J=7.4), 1.22 (m, 2H), 1.59 (m, 2H), 3.93 (t, 2H, J=7.4), 7.04 (d, 1H, J=1.2), 7.29 (d, 2H, J=9.0), 7.40 (d, 2H, J=8.7), 7.55 (d, 1H, J=0.9); MS (EI) m/z: 234 (M⁺).

### Intermediate C17—1-n-butyl-5-(4-chlorophenyl)-1H-imidazole-2-carbaldehyde

A two-necked flask containing a solution of intermediate C16 (0.97 g, 1 equiv) in anhydrous THF (5 ml) was backfilled with nitrogen and then placed in a −80° C. low-temperatuer reactor. n-Butyl lithium (1.72 ml, 1.6M solution in hexane, 1.2 equiv) was added dropwise over 5 min and then the mixture was stirred for 1 h before anhydrous DMF (0.64 ml, 2 equiv) was added dropwise. After the addition, the solution was allowed to warm to room temperature slowly and stirred for a few hours. The reaction was quenched with saturated NH₄Cl solution and extracted with EA twice. The combined organic phase was washed with brine, dried over MgSO₄ and purified by column chromatography to give the title compound as an oil (0.78 g). ¹H-NMR (CDCl₃, 300 MHz) δ0.82 (t, 3H, J=7.4), 1.21 (m, 2H), 1.62 (m, 2H), 4.34 (t, 2H, J=7.6), 7.29 (s, 1H), 7.33 (d, 2H, J=8.4), 7.48 (d, 2H, J=8.7), 9.85 (s, 1H); MS (EI) m/z: 262 (M⁺).

### Intermediate C18—5-n-decyl-1-(2-(diethylamino)ethyl)-1H-imidazole-2-carb aldehyde

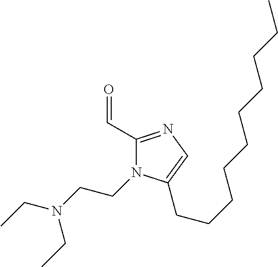 [SMILES: CCCCCCCCCCc1cnc(C=O)n1CCN(CC)CC]

Following a procedure similar to that described for the preparation of intermediate C17 except that decyl aldehyde and N¹,N¹-diethylethane-1,2-diamine were used as starting materials. ¹H-NMR (CDCl₃, 300 MHz) δ 0.87 (t, 3H), 1.03 (t, 6H), 1.26 (m, 14H), 1.69 (m, 2H), 2.66 (m, 8H), 4.40 (t, 2H), 7.08 (s, 1H), 9.70 (s, 1H).

### Intermediate C19—1-(2-(diethylamino)ethyl)-5-(4′-(trifluoromethyl)biphenyl-4-yl)-1H-imidazole-2-carbaldehyde

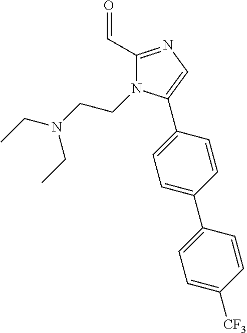 [SMILES: CCN(CC)CCn1c(-c2ccc(-c3ccc(C(F)(F)F)cc3)cc2)cnc1C=O]

Following a procedure similar to that described for the preparation of intermediate C17 except that intermediate M1 and N¹,N¹-diethylethane-1,2-diamine was used as starting materials. ¹H-NMR (CDCl₃, 300 MHz) δ0.84 (t, 6H, J=7.2), 2.42 (q, 4H, J=7.2), 2.65 (t, 2H, J=6.9), 4.51 (t, 2H, J=6.9), 7.36 (s, 1H), 7.58 (d, 2H, J=8.4), 7.72 (d, 2H, J=8.1), 7.73 (s, 4H), 9.85 (s, 1H); MS (ESI): 416 (M+H).

### Example 35

### 1-((1-n-butyl-5-(4-chlorophenyl)-1H-imidazol-2-yl)methyl)-2-(4-fluorobenzylthio)-6,7-dihydro-1H-cyclopenta[d]pyrimidin-4(5H)-one

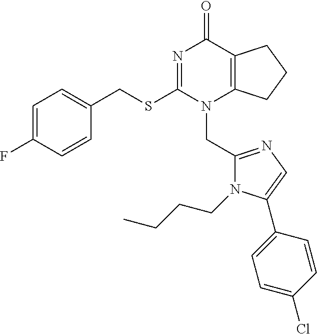 [SMILES: CCCCn1c(-c2ccc(Cl)cc2)cnc1Cn1c(SCc2ccc(F)cc2)nc(=O)c2c1CCC2]

Following a procedure similar to that described for the preparation of example 28 except that intermediate C17 was used in place of intermediate C3. ¹H-NMR (CDCl₃, 300 MHz) δ0.77 (t, 3H, J=7.2), 1.14 (m, 2H), 1.48 (m, 2H), 2.12 (m, 2H), 2.85 (t, 2H, J=7.5), 2.95 (t, 2H, J=7.5), 3.89 (t, 2H, J=7.6), 4.50 (s, 2H), 5.10 (s, 2H), 6.95 (t, 2H, J=8.7), 6.97 (s, 1H), 7.24 (d, 2H, J=8.7), 7.33 (dd, 2H, J=8.7, 5.3), 7.41 (d, 2H, J=8.1); MS (ESI): 523 (M+H).

### Example 36

### 1-((1-n-butyl-5-(4-chlorophenyl)-1H-imidazol-2-yl)methyl)-2-(2-nitrobenzylthio)-6,7-dihydro-1H-cyclopenta[d]pyrimidin-4(5H)-one

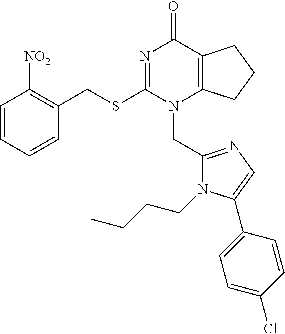 [SMILES: CCCCn1c(-c2ccc(Cl)cc2)cnc1Cn1c(SCc2ccccc2[N+](=O)[O-])nc(=O)c2c1CCC2]

Following a procedure similar to that described for the preparation of example 28 except that intermediate C17 and 1-(chloromethyl)-2-nitrobenzene were used as starting materials in place of C3 and 1-(bromomethyl)-4-fluorobenzene, respectively. ¹H-NMR (CDCl₃, 300 MHz) δ0.81 (t, 3H, J=7.2), 1.16 (m, 2H), 1.50 (m, 2H), 2.12 (m, 2H), 2.86 (t, 2H, J=7.4), 2.97 (t, 2H, J=7.2), 3.92 (t, 2H, J=7.6), 4.89 (s, 2H), 5.05 (s, 2H), 6.92 (s, 1H), 7.27 (d, 2H, J=8.1), 7.42 (m, 3H), 7.56 (t, 1H, J=7.6), 7.99 (m, 2H); MS (ESI): 550 (M+H).

### Example 37

### 1-((5-n-decyl-1-(2-(diethylamino)ethyl)-1H-imid azol-2-yl)methyl)-2-(4-fluorobenzylthio)-6,7-dihydro-1H-cyclopenta[d]pyrimidin-4(5H)-one

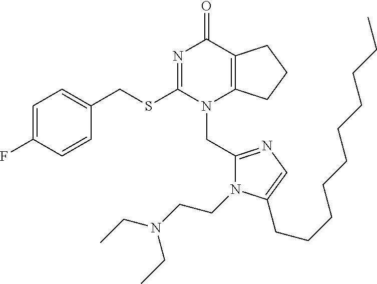 [SMILES: CCCCCCCCCCc1cnc(Cn2c(SCc3ccc(F)cc3)nc(=O)c3c2CCC3)n1CCN(CC)CC]

Following a procedure similar to that described for the preparation of example 28 except that intermediate C18 was used in place of intermediate C3. The product is an oil. ¹H-NMR (CDCl₃, 400 MHz) δ0.90 (m, 9H), 1.26 (m, 14H), 1.63 (m, 2H), 2.14 (m, 2H), 2.46 (m, 6H), 2.56 (t, 2H, J=7.6), 2.84 (t, 2H, J=7.4), 2.91 (t, 2H, J=7.5), 3.84 (t, 2H, J=7.5), 4.47 (s, 2H), 5.10 (s, 2H), 6.72 (s, 1H), 6.95 (t, 2H, J=8.6), 7.32 (dd, 2H, J=8.6, 5.6); MS (ESI): 596 (M+H).

### Example 38

### 1-((1-(2-(diethylamino)ethyl)-5-(4′-(trifluoromethyl)biphenyl-4-yl)-1H-imidazol-2-yl)methyl)-2-(4-fluorobenzylthio)-6,7-dihydro-1H-cyclopenta[d]pyrimidin-4(5H)-one

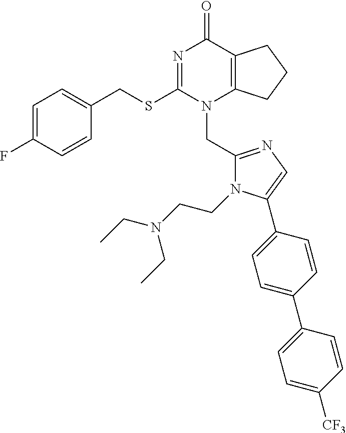 [SMILES: CCN(CC)CCn1c(-c2ccc(-c3ccc(C(F)(F)F)cc3)cc2)cnc1Cn1c(SCc2ccc(F)cc2)nc(=O)c2c1CCC2]

Following a procedure similar to that described for the preparation of example 28 except that intermediate C19 was used in place of intermediate C3. ¹H-NMR (CDCl₃, 300 MHz) δ0.83 (t, 6H, J=6.9), 2.13 (m, 2H), 2.43 (m, 6H), 2.88 (t, 2H, J=6.9), 3.00 (t, 2H, J=7.2), 4.05 (t, 2H, J=5.7), 4.51 (s, 2H), 5.22 (s, 2H), 6.96 (t, 2H, J=8.6), 7.06 (s, 1H), 7.35 (dd, 2H, J=8.1, 5.7), 7.45 (d, 2H, J=8.1), 7.71 (m, 6H); MS (ESI): 676 (M+H).

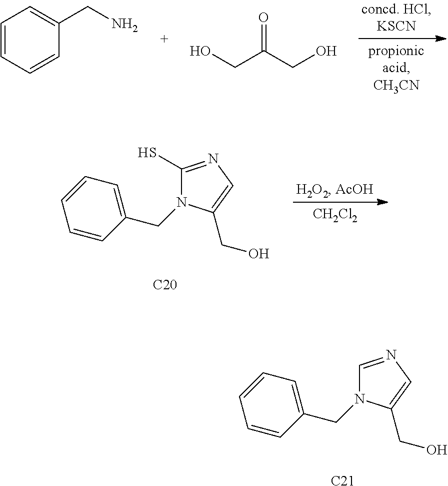

### Intermediate C20—(1-benzyl-2-mercapto-1H-imidazol-5-yl)methanol

To an ice cold solution of phenylmethanamine (3.3 ml, 30 mmol) in acetonitrile (30 ml) was added concd.HCl (2.55 ml, 30 mmol) over 3 min with vigorous stirring, white solid was formed. Then propionic acid (2.5 ml), 1,3-dihydroxypropan-2-one (4.5 g, 25 mmol) and KSCN (2.9 g, 30 mmol) were added and the mixture was stirred at 70° C. for 2 h. After cooled, the mixture was diluted with water (100 ml) and stirred for 20 min. The precipitate thus obtained was collected by filtration, washed with water and cold industrial ethanol, and then dried to obtain the title compound (4.72 g). ¹H-NMR (d6-DMSO, 300 MHz) δ4.13 (s, 2H), 5.26 (s, 1H, —OH), 5.32 (s, 2H), 6.85 (s, 1H), 7.27 (m, 5H), 12.17 (s, 1H).

### Intermediate C21—(1-benzyl-1H-imidazol-5-yl)methanol

To a suspension of intermediate C20 (4.72 g, 21.45 mmol, 1 equiv) in DCM (30 ml) were added AcOH (2.5 ml) and an aqueous solution of hydrogen peroxide (0.5 ml, 30% wt). The mixture was heated slightly to initiate the reaction. Then, the rest aqueous solution of hydrogen peroxide (5.5 ml) was added dropwise (>2.5 equiv in total) at such a rate that the solution boiled mildly. After the addition, the mixture was stirred at room temperature for 0.5 h and then the reaction flask was placed in an ice bath and the mixture was adjusted to pH>10 with a solution of NaOH (10% m/v). The mixture was stayed for a few hours and the precipitate thus obtained was collected by filtration, washed with water and DCM, and then dried in vacuo to give the title compound. Organic layer of the filtrate was then separated and concentrated and solid precipitated and was collected by filtration to give a further batch of product (3.25 g in total). ¹H-NMR (CDCl₃, 300 MHz) δ4.49 (s, 2H), 5.22 (s, 2H), 6.92 (s, 1H), 7.12 (m, 2H), 7.32 (m, 3H), 7.44 (s, 1H); MS (EI) m/z: 188 (M⁺).

### Intermediate C22—(1-(4-bromobenzyl)-1H-imidazol-5-yl)methanol

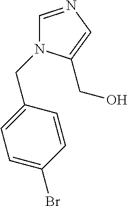 [SMILES: OCc1cncn1Cc1ccc(Br)cc1]

Following a procedure similar to that described for the preparation of intermediate C21 except that (4-bromophenyl)methanamine was used in place of phenylmethanamine ¹H-NMR (d₆-DMSO, 300 MHz) δ4.30 (d, 2H, J=4.8), 5.13 (t, 1H, J=5.1, —OH), 5.21 (s, 2H), 6.83 (s, 1H), 7.11 (d, 2H, J=8.1), 7.55 (d, 2H, J=8.4), 7.69 (s, 1H); MS (ESI): 267/269 (M+H).

### Intermediate C23—1-(4-bromobenzyl)-5-methyl-1H-imidazole

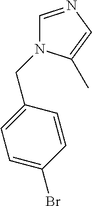

Following a procedure similar to that described for the preparation of intermediate C21 except that 2-hydroxy acetone was used in place of 1,3-dihydroxypropan-2-one. ¹H-NMR (CDCl₃, 300 MHz) δ2.06 (s, 3H), 5.01 (s, 2H), 6.83 (s, 1H), 6.90 (d, 2H, J=8.4), 7.45 (d, 2H, J=8.1), 7.50 (s, 1H); MS (ESI): 251/253 (M+H).

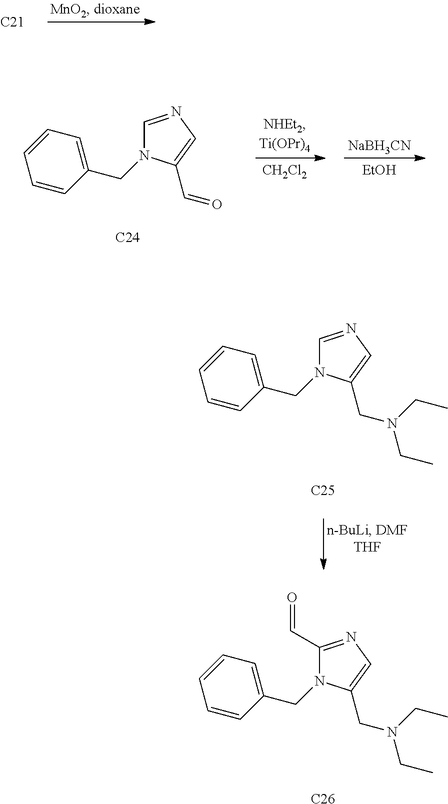

### Intermediate C24—1-benzyl-1H-imidazole-5-carbaldehyde

To a solution of intermediate C21 (11.5 g, 1 equiv) in dioxane (60 ml) was added activated MnO₂ (32 g, 6 equiv) and the mixture was stirred at 60° C. for 3 h (TLC detection showed that the reaction was complete) before it was filtered to remove MnO₂ and washed with EA. The solvent was evaporated to give the title compound as a solid (10.38 g). ¹H-NMR (CDCl₃, 300 MHz) δ5.52 (s, 2H), 7.20 (m, 2H), 7.33 (m, 3H), 7.71 (s, 1H), 7.83 (s, 1H), 9.76 (s, 1H); MS (EI) m/z: 186 (M⁺).

### Intermediate C25—N-((1-benzyl-1H-imidazol-5-yl)methyl)-N-ethylethanamine

A mixture of intermediate C24 (1.5 g, 1 equiv), diethylamine (0.875 ml, 1.05 equiv), tetrapropoxytitanium (3.1 ml, 1.3 equiv) in DCM (8 ml) was stirred at room temperature for 4 h under nitrogen before absolute ethanol (8 ml) and sodium cyanoborohydride (507 mg, 1 equiv) was added. The mixture was stirred at room temperature for 16 h and then quenched with 20 ml water. The precipitated white solid was removed by filtration and washed with 20 ml EA. The filtrate was transferred to a seperatory funnel and the EA layer was separated. The aqueous phase was extracted with 20 ml of EA once. The combined organic phase was washed with brine twice, dried over MgSO₄ and purified by column chromatography (EA/EtOH=4:1) to give the title compound as an oil (1.32 g). ¹H-NMR (CDCl₃, 300 MHz) δ0.94 (t, 6H, J=6.9), 2.46 (q, 4H, J=7.2), 3.39 (s, 2H), 5.30 (s, 2H), 6.95 (s, 1H), 7.08 (m, 2H), 7.29 (m, 3H), 7.53 (s, 1H); MS (ESI): 244 (M+H).

### Intermediate C26—1-benzyl-5-((diethylamino)methyl)-1H-imidazole-2-carbaldehyde

Following a procedure similar to that described for the preparation of intermediate C17 except that intermediate C25 was used in place intermediate C16. ¹H-NMR (CDCl₃, 300 MHz) δ0.96 (t, 6H, J=7.1), 2.49 (q, 4H, J=7.2), 3.46 (s, 2H), 5.88 (s, 2H), 6.97 (m, 2H), 7.27 (m, 4H), 9.80 (s, 1H).

### Example 39

### 1-((1-benzyl-5-((diethylamino)methyl)-1H-imidazol-2-yl)methyl)-2-(4-fluorobenzylthio)-6,7-dihydro-1H-cyclopenta[d]pyrimidin-4(5H)-one

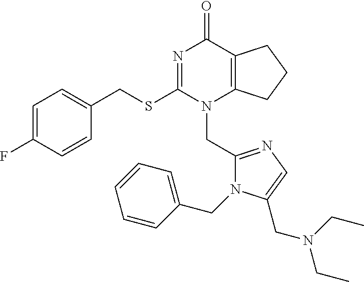 [SMILES: CCN(CC)Cc1cnc(Cn2c(SCc3ccc(F)cc3)nc(=O)c3c2CCC3)n1Cc1ccccc1]

Following a procedure similar to that described for the preparation of example 28 except that intermediate C26 was used in place intermediate C3. ¹H-NMR (CDCl₃, 300 MHz) δ0.93 (t, 6H, J=7.2), 2.00 (m, 2H), 2.48 (m, 4H), 2.72 (q, 4H, J=7.4), 3.47 (s, 2H), 4.42 (s, 2H), 4.85 (s, 2H), 5.39 (s, 2H), 6.87 (m, 2H), 6.96 (m, 3H), 7.23-7.33 (m, 5H); MS (ESI): 532 (M+H).

### Intermediate C27—5-methyl-1-((4′-(trifluoromethyl)biphenyl-4-yl)methyl)-1H-imidazole

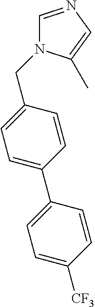 [SMILES: Cc1cncn1Cc1ccc(-c2ccc(C(F)(F)F)cc2)cc1]

A mixture of intermediate C23 (1.07 g, 4.26 mmol, 1 equiv), 4-(trifluoromethyl)phenylboronic acid (0.85 g, 1.05 equiv), Cs₂CO₃ (2.8 g, 2 equiv) and Pd(PPh)₃ (246 mg, 0.05 equiv) in dioxane (10 ml) was refluxed for 6 h in a nitrogen atmosphere and then filtered to remove the insoluble substances while hot. The filtrate was mixed with silica gel, evaporated and purified by column chromatography (DCM/MeOH=15:1) to give the title compound as a solid (1.11 g). ¹H-NMR (CDCl₃, 300 MHz) δ2.13 (s, 3H), 5.13 (s, 2H), 6.87 (s, 1H), 7.15 (d, 2H, J=7.8), 7.57 (d, 2H, J=7.8), 7.58 (s, 1H), 7.65 (d, 2H, J=9.0), 7.70 (d, 2H, J=9.3); MS (ESI): 317 (M+H).

### Intermediate C28—(5-methyl-1-((4′-(trifluoromethyl)biphenyl-4-yl)methyl)-1H-imidazol-2-yl)methanol

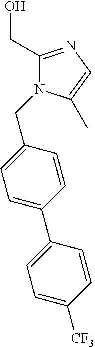 [SMILES: Cc1cnc(CO)n1Cc1ccc(-c2ccc(C(F)(F)F)cc2)cc1]

A mixture of intermediate C27 (1.53 g, 4.84 mmol) and formaldehyde (10 ml, 37% aqueous solution) in dioxane (3 ml) was heated in a pressure tube at 140-150° C. for 36 h (the plug was screwed tightly). Then, the heating was stopped and TLC detection showed that the reaction was complete. The reaction mixture was poured into 20 ml of saturated brine and extracted with DCM (60 mL) three times. The combined organic phase was washed with brine three times, dried over MgSO₄ and purified by column chromatography to give the title compound as a white solid (0.9 g). ¹H-NMR (CDCl₃, 300 MHz) δ2.10 (s, 3H), 4.62 (s, 2H), 5.29 (s, 2H), 6.73 (s, 1H), 7.10 (d, 2H, J=8.4), 7.54 (d, 2H, J=8.4), 7.67 (m, 4H); MS (ESI): 347 (M+H).

### Example 40

### 2-(4-fluorobenzylthio)-1-((5-methyl-1-((4′-(trifluoromethyl)biphenyl-4-yl)methyl)-1H-imidazol-2-yl)methyl)-6,7-dihydro-1H-cyclopenta[d]pyrimidin-4 (5H)-one

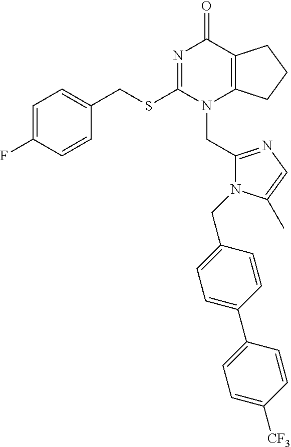 [SMILES: Cc1cnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 28 except that intermediate C28 was used in place intermediate C9. ¹H-NMR (CDCl₃, 300 MHz) δ1.93 (m, 2H), 2.22 (s, 3H), 2.60 (t, 2H, J=7.2), 2.69 (t, 2H, J=7.5), 4.46 (s, 2H), 5.01 (s, 2H), 5.14 (s, 2H), 6.84 (d, 2H, J=8.1), 6.88 (s, 1H), 6.95 (t, 2H, J=8.4), 7.34 (dd, 2H, J=5.4, 8.4), 7.44 (d, 2H, J=8.4), 7.63 (d, 2H, J=8.4), 7.70 (d, 2H, J=8.1); MS (ESI): 605 (M+H).

### Example 41

### 2-(4-fluorobenzylthio)-1-((5-methyl-1-((4′-(trifluoromethyl)biphenyl-4-yl)methyl)-1H-imidazol-2-yl)methyl)-5-((1-methyl-1H-pyrazol-4-yl)methyl)pyrimidin-4(1H)-one

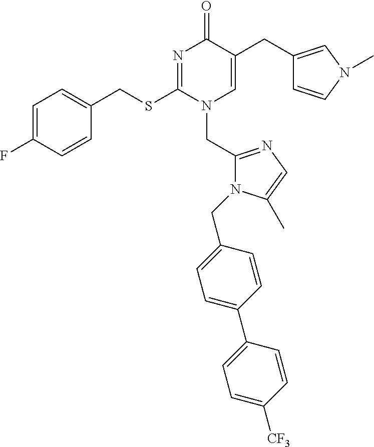 [SMILES: Cc1cnc(Cn2cc(Cc3ccn(C)c3)c(=O)nc2SCc2ccc(F)cc2)n1Cc1ccc(-c2ccc(C(F)(F)F)cc2)cc1]

Following a procedure similar to that described for the preparation of example 29 except that intermediate C28 was used in place intermediate C9. ¹H-NMR (CDCl₃, 300 MHz) δ2.20 (s, 3H), 3.39 (s, 2H), 3.80 (s, 3H), 4.40 (s, 2H), 4.89 (s, 2H), 5.09 (s, 2H), 6.74 (d, 2H, J=8.1), 6.96 (m, 4H), 7.23 (s, 1H), 7.33 (m, 5H), 7.62 (d, 2H, J=8.1), 7.70 (d, 2H, J=8.7); MS (ESI): 659 (M+H).

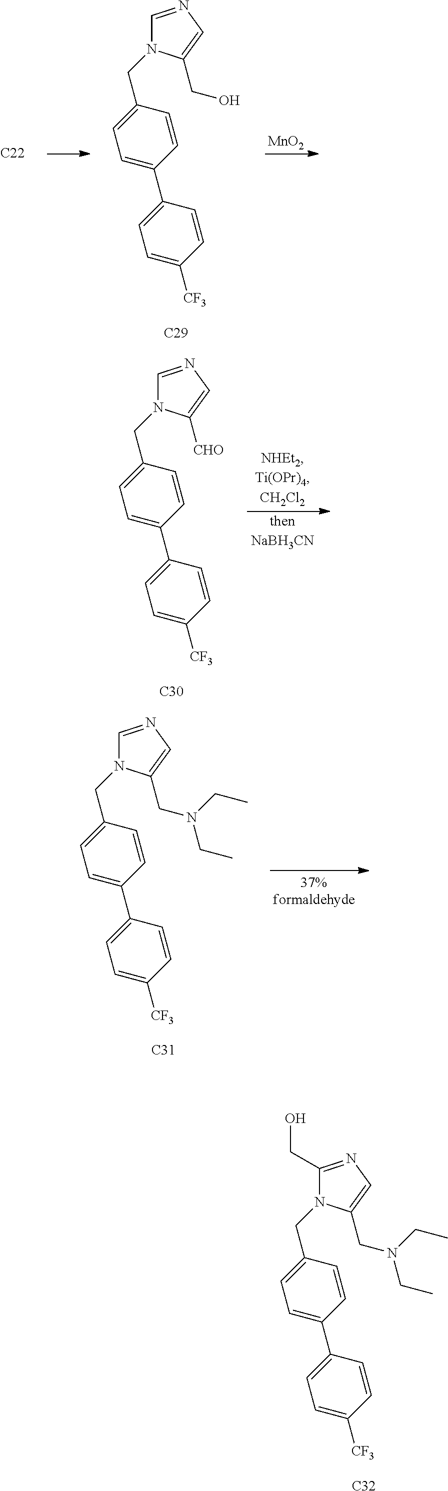

Intermediate C29—(1-((4′-(trifluoromethyl)biphenyl-4-yl)methyl)-1H-imid azol-5-yl)methanol

A mixture of intermediate C22 (10 mol, 1 equiv), 4-(trifluoromethyl)phenylboronic acid (1.1 equiv), Cs₂CO₃ (2 equiv) and Pd(PPh₃)₄ (0.05 equiv) in 30 ml dioxane was backfilled with nitrogen several times and heated under reflux for 15 h. Then, it was filtered to remove the inorganic salts and washed with dioxane. The filtrate was evaporated under reduced pressure to dryness to give a brown oil or solid which was then dissolved in EA (20 ml). The solution was stayed for a few hours to wait for an adequate precipitation of the product. The solid thus obtained was filtered off and dried. The above operation was repeated for the mother liquor. ¹H-NMR (d₆-DMSO, 300 MHz) δ4.35 (s, 2H), 5.30 (s, 2H), 6.85 (s, 1H), 7.29 (d, 2H, J=8.1), 7.73 (d, 2H, J=7.8), 7.74 (s, 1H), 7.80 (d, 2H, J=8.1), 7.88 (d, 2H, J=8.4).

### Intermediate C30—1-((4′-(trifluoromethyl)biphenyl-4-yl)methyl)-1H-imidazole-5-carbaldehyde

Following a procedure similar to that described for the preparation of intermediate C24. ¹H-NMR (CDCl₃, 300 MHz) δ5.58 (s, 2H), 7.30 (d, 2H, J=8.1), 7.57 (d, 2H, J=8.1), 7.67 (m, 4H), 7.78 (s, 1H), 7.84 (s, 1H), 9.78 (s, 1H).

### Intermediate C31—N-ethyl-N-((1-((4′-(trifluoromethyl)biphenyl-4-yl)methyl)-1H-imidazol-5-yl)methyl)ethanamine

Following a procedure similar to that described for the preparation of intermediate C25. ¹H-NMR (CDCl₃, 300 MHz) δ0.99 (t, 6H, J=7.2), 2.53 (q, 4H, J=6.9), 3.47 (s, 2H), 5.39 (s, 2H), 7.00 (s, 1H), 7.17 (d, 2H, J=7.8), 7.55 (d, 2H, J=8.1), 7.63 (s, 1H), 7.67 (m, 4H); MS (ESI): 388 (M+H).

### Intermediate C32—(5-((diethylamino)methyl)-1-((4′-(trifluoromethyl)biphenyl-4-yl)methyl)-1H-imidazol-2-yl)methanol

Following a procedure similar to that described for the preparation of intermediate C28. ¹H-NMR (CDCl₃, 300 MHz) δ0.93 (t, 6H, J=7.2), 2.46 (q, 4H, J=7.2), 3.37 (s, 2H), 4.62 (s, 2H), 5.53 (s, 2H), 6.87 (s, 1H), 7.10 (d, 2H, J=8.4), 7.53 (d, 2H, J=8.4), 7.67 (m, 4H).

### Intermediate C33—(5-(pyrrolidin-1-ylmethyl)-1-((4′-(trifluoromethyl)biphenyl-4-yl)methyl)-1H-imidazol-2-yl)methanol

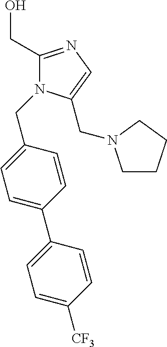 [SMILES: OCc1ncc(CN2CCCC2)n1Cc1ccc(-c2ccc(C(F)(F)F)cc2)cc1]

Following a procedure similar to that described for the preparation of intermediate C32 from C30 except that pyrrolidine was used in place of diethylamine ¹H-NMR (CDCl₃, 300 MHz) δ1.72 (m, 4H), 2.42 (m, 4H), 3.41 (s, 2H), 4.62 (s, 2H), 5.49 (s, 2H), 6.83 (s, 1H), 7.14 (d, 2H, J=8.4), 7.53 (d, 2H, J=8.1), 7.67 (m, 4H).

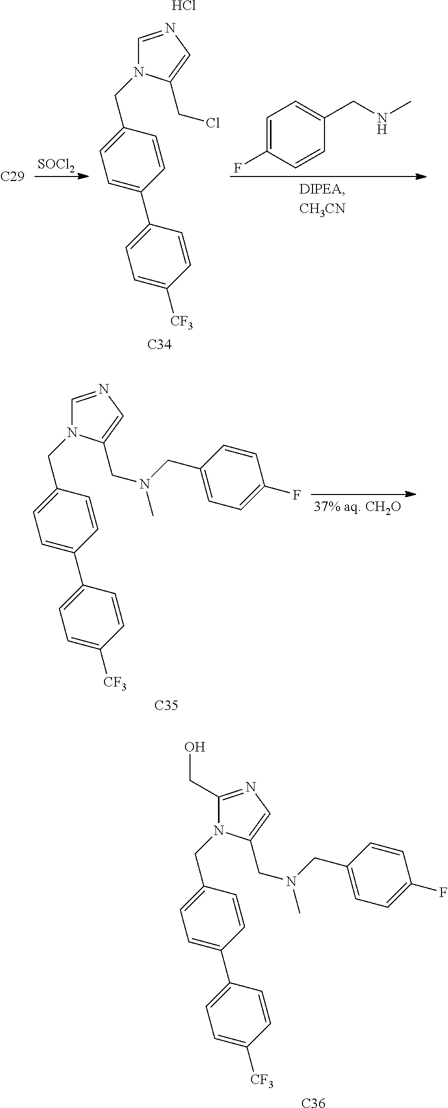

### Intermediate C34—5-(chlo romethyl)-1-((4′-(trifluoromethyl)biphenyl-4-yl)methyl)-1H-imidazole Hydrochloride

To an ice cold mixture of intermediate C29 (5 mmol) and DMF (2 drops, catalyst) in DCM (20 ml) in a 100 ml of flask was added a solution of thionyl chloride (0.42 ml, 6 mmol) in DCM (5 ml) dropwise via a constant pressure funnel over 10 min. The mixture was stirred at room temperature for 2 h until the completion of the reaction monitored by TLC. The solid so formed was collected by filtration with a buchner funnel, washed with DCM three times and then dried at 80° C. to give the title compound as white or light yellow powder. ¹H-NMR (d₆-DMSO, 300 MHz) δ4.96 (s, 2H), 5.61 (s, 2H), 7.52 (d, 2H, J=8.4), 7.82 (m, 3H), 7.89 (m, 4H), 9.39 (s, 1H).

### Intermediate C35—N-(4-fluorobenzyl)-N-methyl-1-(1-((4′-(trifluoromethyl)biphenyl-4-yl)methyl)-1H-imidazol-5-yl)methanamine

A mixture of intermediate C34 (1.38 g, 3.57 mmol, 1 equiv), 1-(4-fluorophenyl)-N-methylmethanamine (0.5 g, 1 equiv) and DIPEA (1.47 ml, 2.5 equiv) in acetonitrile (10 ml) was heated at 70-80° C. overnight until the completion of the reaction monitored by TLC. Solvent was evaporated and aq. NH₄Cl solution (40 ml) was added to the residue. The resulting mixture was extracted with DCM twice and the combined organic phase was washed with brine twice, dried and then purified by column chromatography to give the title compound as an oil (0.9 g). ¹H-NMR (CDCl₃, 400 MHz) δ2.16 (s, 3H), 3.38 (s, 2H), 3.46 (s, 2H), 5.31 (s, 2H), 6.97 (t, 2H, J=8.4), 7.03 (s, 1H), 7.08 (d, 2H, J=8.4), 7.21 (dd, 2H, J=8.4, 5.6), 7.54 (d, 2H, J=8.0), 7.60 (s, 1H), 7.68 (d, 2H, J=8.8), 7.72 (d, 2H, J=8.0).

### Intermediate C36—(5-(((4-fluorobenzyl)(methyl)amino)methyl)-1-((4′-(trifluoromethyl)biphenyl-4-yl)methyl)-1H-imidazol-2-yl)methanol

Following a procedure similar to that described for the preparation of intermediate C28 except that intermediate C35 was used in place of intermediate C27. ¹H-NMR (CDCl₃, 300 MHz) δ2.07 (s, 3H), 3.26 (s, 2H), 3.37 (s, 2H), 4.60 (s, 2H), 5.45 (s, 2H), 6.83 (s, 1H), 6.92 (t, 2H, J=8.4), 7.01 (d, 2H, J=8.4), 7.12 (dd, 2H, J=8.4, 5.4), 7.49 (d, 2H, J=8.1), 7.64 (d, 2H, J=8.7), 7.68 (d, 2H, J=8.4).

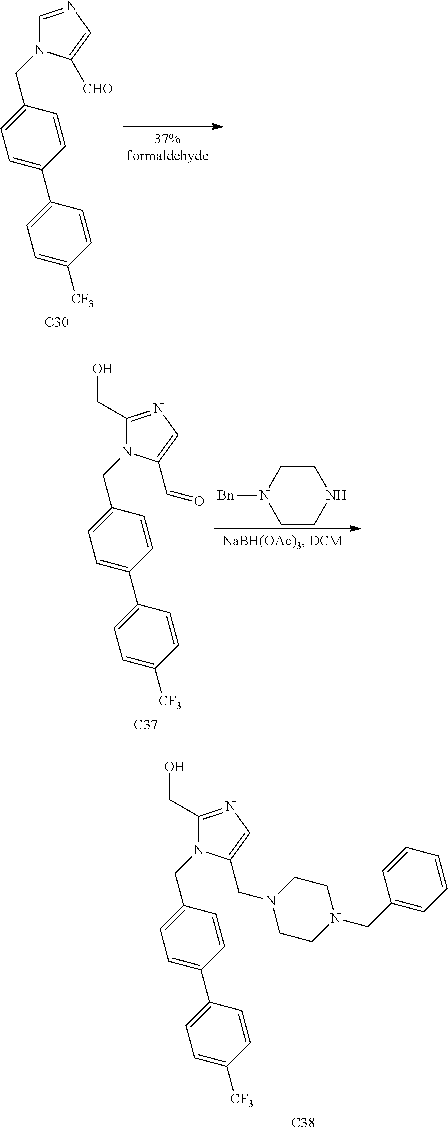

### Intermediate C37—2-(hydroxymethyl)-1-((4′-(trifluoromethyl)biphenyl-4-yl)methyl)-1H-imidazole-5-carbaldehyde

Following a procedure similar to that described for the preparation of intermediate C28 except that intermediate C30 was used in place of intermediate C27. ¹H-NMR (CDCl₃, 300 MHz) δ4.75 (s, 2H), 5.74 (s, 2H), 7.22 (d, 2H, J=8.1), 7.54 (d, 2H, J=7.8), 7.63 (d, 2H, J=8.7), 7.69 (d, 2H, J=8.7), 7.77 (s, 1H), 9.46 (s, 1H).

### Intermediate C38—(5-((4-benzylpiperazin-1-yl)methyl)-1-((4′-(trifluoromethyl)biphenyl-4-yl)methyl)-1H-imidazol-2-yl)methanol

A mixture of intermediate C37 (0.56 g, 1 equiv), 1-benzylpiperazine (300 μl, 1.1 equiv), NaBH(OAc)₃ (495 mg, 1.5 equiv) and AcOH (180 μl, 2 equiv) in 5 ml DCM was stirred at room temperature overnight until the completion of the reaction monitored by TLC. The reaction mixture was washed with brine twice, with saturated NaHCO₃ solution twice, dried over Na₂SO₄ and then purified by column chromatography to give the title compound as a solid (0.5 g). ¹H-NMR (CDCl₃, 400 MHz) δ2.43 (m, 8H), 3.45 (s, 2H), 3.52 (s, 2H), 4.75 (s, 2H), 5.50 (s, 2H), 7.26 (m, 8H), 7.55 (d, 2H, J=8.4), 7.65 (d, 2H, J=8.4), 7.69 (d, 2H, J=8.4).

### Intermediate C39—(5-((dimethylamino)methyl)-1-((4′-(trifluoromethyl)biphenyl-4-yl)methyl)-1H-imidazol-2-yl)methanol

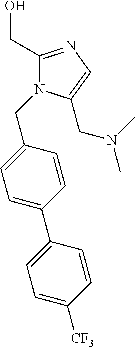 [SMILES: CN(C)Cc1cnc(CO)n1Cc1ccc(-c2ccc(C(F)(F)F)cc2)cc1]

Following a procedure similar to that described for the preparation of intermediate C38 from C37 except that dimethylamine hydrochloride and DIPEA (2 equiv) was used in place of 1-benzylpiperazine. ¹H-NMR (CDCl₃, 300 MHz) δ2.15 (s, 6H), 3.19 (s, 2H), 4.62 (s, 2H), 4.63 (vbrs, 1H), 5.49 (s, 2H), 6.83 (s, 1H), 7.14 (d, 2H, J=8.1), 7.54 (d, 2H, J=8.1), 7.67 (m, 4H); MS (ESI): 390 (M+H).

### Example 42

### 1-((5-((diethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one

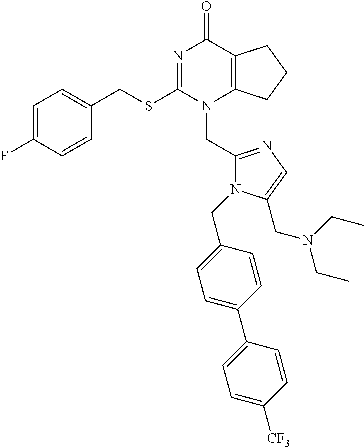 [SMILES: CCN(CC)Cc1cnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 28 except that intermediate C32 was used in place of intermediate C9. ¹H-NMR (CDCl₃, 300 MHz) δ0.93 (t, 6H, J=6.9), 1.97 (m, 2H), 2.47 (q, 4H, J=6.9), 2.70 (m, 4H), 3.48 (s, 2H), 4.45 (s, 2H), 4.92 (s, 2H), 5.44 (s, 2H), 6.95 (m, 5H), 7.31 (m, 2H), 7.45 (d, 2H, J=7.8), 7.62 (d, 2H, J=7.8), 7.70 (d, 2H, J=8.1); MS (ESI): 676 (M+H).

### Example 43

### 2-(4-fluorobenzylthio)-1-((5-(pyrrolidin-1-ylmethyl)-1-((4′-(trifluoromethyl)biphenyl-4-yl)methyl)-1H-imidazol-2-yl)methyl)-6,7-dihydro-1H-cyclopenta[d]pyrimidin-4(5H)-one

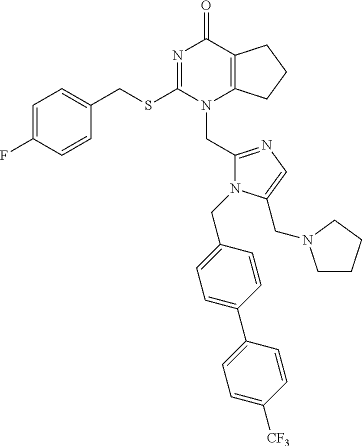 [SMILES: O=c1nc(SCc2ccc(F)cc2)n(Cc2ncc(CN3CCCC3)n2Cc2ccc(-c3ccc(C(F)(F)F)cc3)cc2)c2c1CCC2]

Following a procedure similar to that described for the preparation of example 28 except that intermediate C33 was used in place of intermediate C9. ¹H-NMR (CDCl₃, 300 MHz) δ1.79 (m, 4H), 1.96 (m, 4H), 2.53 (m, 2H), 2.68 (m, 4H), 3.61 (s, 2H), 4.44 (s, 2H), 4.95 (s, 2H), 5.45 (s, 2H), 6.93 (m, 4H), 6.99 (s, 1H), 7.30 (dd, 2H, J=7.8, 5.4), 7.43 (d, 2H, J=7.8), 7.60 (d, 2H, J=7.8), 7.70 (d, 2H, J=7.8).

### Example 44

### 1-((5-(((4-fluorobenzyl)(methypamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopentaidlpyrimidin-4(5H)-one

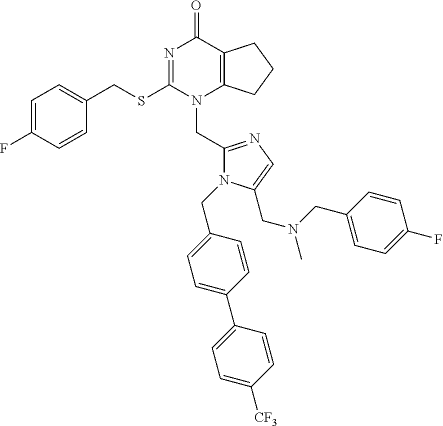 [SMILES: CN(Cc1ccc(F)cc1)Cc1cnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 28 except that intermediate C36 was used in place of intermediate C9. ¹H-NMR (CDCl₃, 400 MHz) δ1.98 (m, 2H), 2.18 (s, 3H), 2.70 (m, 4H), 3.43 (s, 2H), 3.45 (s, 2H), 4.45 (s, 2H), 4.94 (s, 2H), 5.29 (s, 2H), 6.84-6.96 (m, 6H), 7.02 (s, 1H), 7.11 (m, 2H), 7.31 (dd, 2H, J=8.4, 6.4), 7.44 (d, 2H, J=8.0), 7.64 (d, 2H, J=8.4), 7.73 (d, 2H, J=8.0); MS (ESI): 742 (M+H).

### Example 45

### 1-((5-((4-benzylpiperazin-1-yl)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one

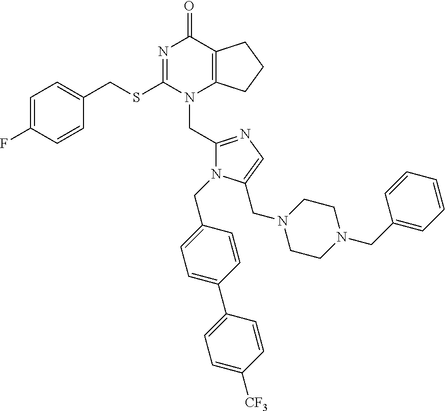 [SMILES: O=c1nc(SCc2ccc(F)cc2)n(Cc2ncc(CN3CCN(Cc4ccccc4)CC3)n2Cc2ccc(-c3ccc(C(F)(F)F)cc3)cc2)c2c1CCC2]

Following a procedure similar to that described for the preparation of example 28 except that intermediate C38 was used in place of intermediate C9. ¹H-NMR (CDCl₃, 400 MHz) δ1.99 (m, 2H), 2.40 (m, 4H), 2.53 (m, 4H), 2.66 (t, 2H), 2.78 (t, 2H), 3.50 (s, 2H), 3.74 (s, 2H), 4.47 (s, 2H), 5.05 (s, 2H), 5.45 (s, 2H), 6.96 (m, 4H), 7.31 (m, 8H), 7.48 (d, 2H), 7.65 (d, 2H), 7.74 (d, 2H).

### Example 46

### 1-((5-((dimethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one

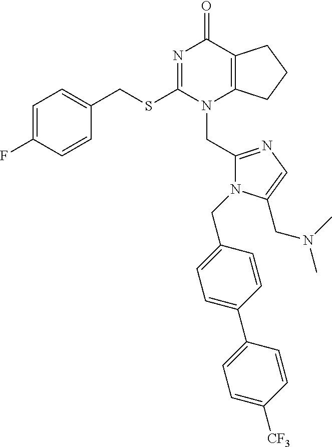 [SMILES: CN(C)Cc1cnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 28 except that intermediate C39 was used in place of intermediate C9. ¹H-NMR (CDCl₃, 300 MHz) δ1.96 (m, 2H), 2.19 (s, 6H), 2.68 (m, 4H), 3.33 (s, 2H), 4.45 (s, 2H), 4.93 (s, 2H), 5.39 (s, 2H), 6.93 (m, 5H), 7.31 (m, 2H), 7.45 (d, 2H, J=7.8), 7.62 (d, 2H, J=7.8), 7.70 (d, 2H, J=7.5); MS (ESI): 648 (M+H).

### Example 47

### N-(4-fluorobenzyl)-1-(2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazol-5-yl)-N,N-dimethylmethanaminium bromide

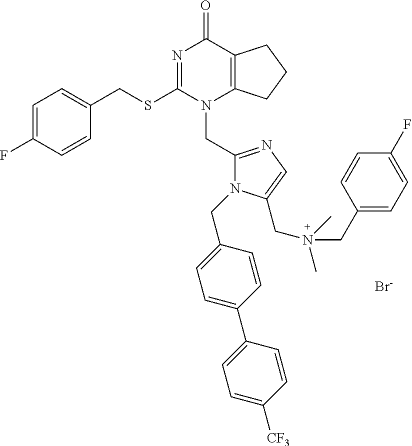

A mixture of example 46 (25 mg), 1-(bromomethyl)-4-fluorobenzene (6 μl) in acetone (1.5 ml) was refluxed for 1 h. TLC showed that most of the reaction was complete. The mixture was then stored in refrigerator for a few hours. The precipitate thus obtained was collected by filtration, dried to give the title compound as white powder (10 mg). ¹H-NMR (CDCl₃, 300 MHz) δ2.06 (m, 2H), 2.71 (t, 2H, J=6.6), 2.85 (t, 2H, J=6.6), 2.98 (s, 6H), 4.36 (s, 2H), 4.62 (s, 2H), 4.80 (s, 2H), 5.25 (s, 2H), 5.57 (s, 2H), 6.86 (t, 2H, J=8.7), 7.07 (d, 2H, J=7.8), 7.27 (m, 4H), 7.74 (s, 1H), 7.59 (m, 4H), 7.71 (d, 2H, J=8.1), 7.75 (d, 2H, J=8.4); MS (ESI): 756(M-Br).

### Example 48

### 1-((5-((dimethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one hydrochloride

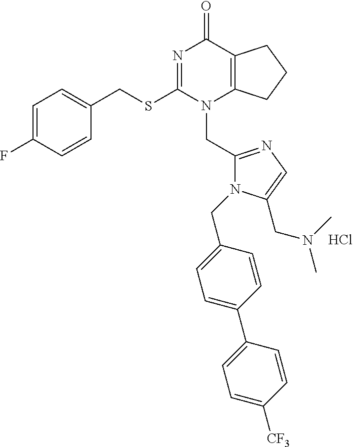 [SMILES: CN(C)Cc1cnc(Cn2c(SCc3ccc(F)cc3)nc(=O)c3c2CCC3)n1Cc1ccc(-c2ccc(C(F)(F)F)cc2)cc1 ; Cl]

To an ice cold solution of example 46 (100 mg, 1 equiv) in isopropanol (1.5 ml) was added concd. HCl (15 μl, 1.1 equiv) and the mixture was stirred for 20 min before ethyl ether (5 ml) was added. Then the mixture was stored in refrigerator. The solid thus obtained was collected by filtration, washed with ethyl ether, and then dried to give the title compound (80 mg).

### Example 49

### 1-(4-fluorobenzyl)-1-(2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazol-5-yl)methyl)pyrrolidinium bromide

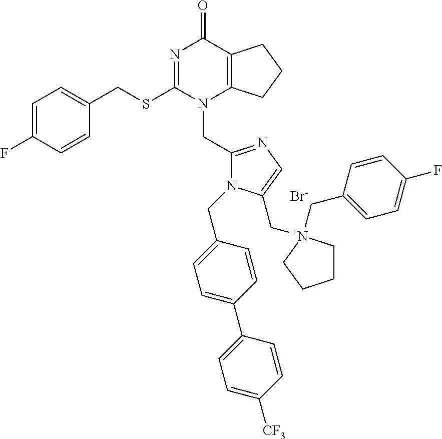 [SMILES: O=c1nc(SCc2ccc(F)cc2)n(Cc2ncc(C[N+]3(Cc4ccc(F)cc4)CCCC3)n2Cc2ccc(-c3ccc(C(F)(F)F)cc3)cc2)c2c1CCC2 ; [Br-]]

Following a procedure similar to that described for the preparation of example 47 except that example 43 was used in place of example 46.

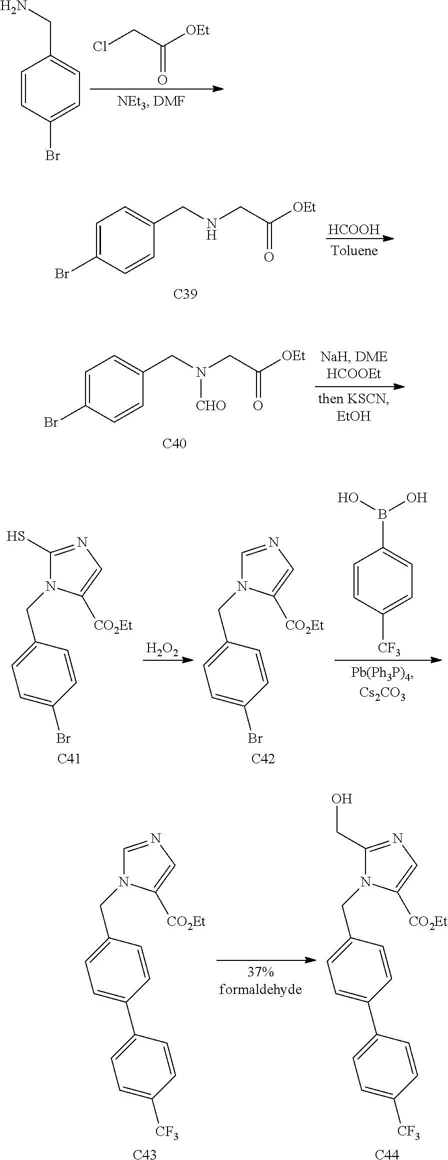

### Intermediate C39—ethyl 2-(4-bromobenzylamino)acetate

To a mixture of (4-bromophenyl)methanamine (20 mmol), triethylamine (20 mmol) in DMF (30 ml) in a flask placed in a oil bath of 40˜50° C. was added a solution of ethyl 2-chloroacetate (20 mmol) in DMF (10 ml) dropwise over 1.5 h via a constant pressure funnel. After the addition, the mixture was stirred for 1.5 h before it was diluted with EA (150 ml). The resulting solution was washed with brine six times (30-40 ml each time), dried over anhydrous NaSO₄ and then solvent evaporated to give the title compound as a colorless oil which could be used in next step without further purification. ¹H-NMR (CDCl₃, 300 MHz) δ1.28 (t, 3H, J=7.2), 2.17 (s, 1H), 3.39 (s, 2H), 3.77 (s, 2H), 4.19 (q, 2H, J=7.2), 7.21 (d, 2H, J=8.4), 7.44 (d, 2H, J=8.4).

### Intermediate C40—ethyl 2-(N-(4-bromobenzyl)formamido)acetate

A mixture of intermediate C39 (3.4 g, 13.6 mmol, 1 equiv) and formic acid (1.5 ml, 2.5 equiv) in toluene was refluxed in a Dean-Stark trap until no more water could be separated. Toluene was evaporated and the residue was mixed with silica gel and evaporated before purified by column chromatography (PE/EA=2:1) to give the title compound as an oil (2.87 g). ¹H-NMR (CDCl₃, 400 MHz, ca 2:1 rotational isomer) 61.27 (t, 3H, J=7.2), 3.96/3.85 (2×s, 2H), 4.19 (m, 2H), 4.51/4.59 (2×s, 2H), 7.12/7.14 (2×d, 2H, J=8.0), 7.52/7.47 (2×d, 2H, J=8.0), 8.36/8.19 (2×s, 1H).

### Intermediate C41—ethyl 1-(4-bromobenzyl)-2-mercapto-1H-imidazole-5-carboxylate

To an ice cold solution of intermediate C40 (6.21 g, 20.7 mmol, 1 equiv) in DME (50 ml) was added NaH (1.0 g, 55-60% m/m, suspended in mineral oil) in batches under nitrogen and the mixture was stirred for 20 min. Then, ethyl formate (5 ml) was added and the mixture was stirred for a further 4 h at room temperature. DME was evaporated to obtain a yellow solid, which was dissolved with ice water (40 ml), acidified to pH˜1 with concd. HCl. Then, KSCN (3.0 g) and EtOH (40 ml) were added and the mixture was refluxed for 15 h. After the solution was cooled in an ice bath, it was adjusted to pH 6-7 with a solution of 10% NaOH (m/v) in an ice bath. The precipitate thus obtained was collected by filtration and dried to give the title compound (3.11 g). ¹H-NMR (d₆-DMSO, 400 MHz) δ1.17 (t, 3H, J=7.2), 4.15 (q, 2H, J=7.2), 5.51 (s, 2H), 7.14 (d, 2H, J=8.4), 7.50 (d, 2H, J=8.4), 7.88 (s, 1H), 13.10 (s, 1H).

### Intermediate C42—ethyl 1-(4-bromobenzyl)-1H-imidazole-5-carboxylate

To a slurry of intermediate C41 (3.10 g, 9.09 mmol, 1 equiv) in DCM (20 ml) was added

AcOH (1 ml) and hydrogen peroxide (0.2 ml, 30% aqueous solution), and the mixture was heated slightly to initiate the reaction. Then, the rest aqueous solution of hydrogen peroxide (2.5 ml) was added dropwise (>2.5 equiv in total) at such a rate that the solution boiled mildly. After the addition, the mixture was stirred at room temperature for 0.5 h and then reaction flask was place in an ice bath and adjusted to pH 6˜7 with a solution of NaOH (10% m/v). The resulting mixture was poured into saturated NaHCO₃ solution (15 ml) and the DCM layer was separated. The aqueous phase was extracted with DCM once. The combined organic phase was washed with brine twice, dried over anhydrous NaSO₄ and purified by flash chromatography to obtain the title compound as a solid (1.85 g). ¹H-NMR (CDCl₃, 400 MHz) δ1.31 (t, 3H, J=7.2), 4.25 (q, 2H, J=7.2), 5.48 (s, 2H), 7.02 (d, 2H, J=8.4), 7.44 (d, 2H, J=8.8), 7.63 (s, 1H), 7.76 (s, 1H).

### Intermediate C43—ethyl 1-((4′-(trifluoromethyl)biphenyl-4-yl)methyl)-1H-imidazole-5-carboxylate

Following a procedure similar to that described for the preparation of intermediate C27 except that intermediate C42 was used in place of intermediate C23. ¹H-NMR (CDCl₃, 400 MHz) δ1.33 (t, 3H, J=7.2), 4.29 (q, 2H, J=6.8), 5.58 (s, 2H), 7.26 (d, 2H, J=8.4), 7.57 (d, 2H, J=8.4), 7.68 (m, 5H), 7.79 (s, 1H).

### Intermediate C44—ethyl 2-(hydroxymethyl)-1-((4′-(trifluoromethyl)biphenyl-4-yl)methyl)-1H-imidazole-5-carboxylate

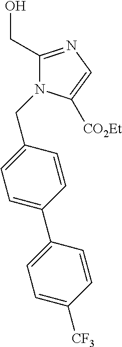 [SMILES: CCOC(=O)c1cnc(CO)n1Cc1ccc(-c2ccc(C(F)(F)F)cc2)cc1]

Following a procedure similar to that described for the preparation of intermediate C28 except that intermediate C43 was used in place of intermediate C27. ¹H-NMR (CDCl₃, 300 MHz) δ1.30 (t, 3H, J=7.2), 4.27 (q, 2H, J=7.2), 4.70 (s, 2H), 5.76 (s, 2H), 7.16 (d, 2H, J=7.8), 7.53 (d, 2H, J=8.4), 7.66 (m, 5H).

### Example 50

### ethyl 2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxylate

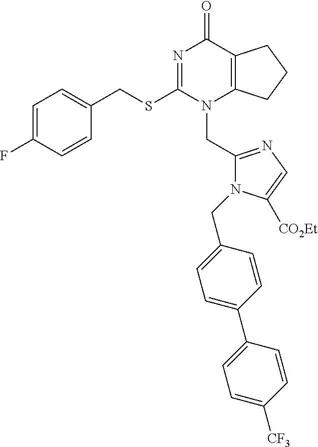 [SMILES: CCOC(=O)c1cnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 28 except that intermediate C44 was used in place of intermediate C9. ¹H-NMR (CDCl₃, 300 MHz) δ1.35 (t, 3H, J=6.9), 2.02 (m, 2H), 2.71 (m, 4H), 4.32 (q, 2H, J=7.2), 4.41 (s, 2H), 5.03 (s, 2H), 5.74 (s, 2H), 6.89 (t, 2H, J=8.4), 7.05 (d, 2H, J=7.8), 7.27 (dd, 2H, J=8.1, 5.4), 7.46 (d, 2H, J=7.8), 7.59 (d, 2H, J=8.1), 7.69 (d, 2H, J=8.1), 7.75 (s, 1H).

### Example 51

### 2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxylic acid

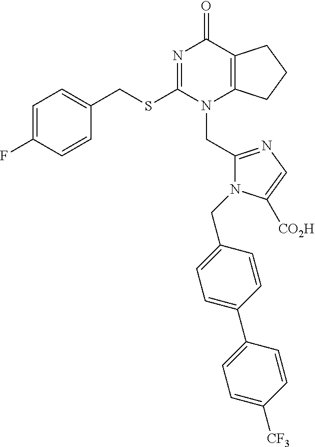 [SMILES: O=C(O)c1cnc(Cn2c(SCc3ccc(F)cc3)nc(=O)c3c2CCC3)n1Cc1ccc(-c2ccc(C(F)(F)F)cc2)cc1]

To an ice cooled solution of example 50 (110 mg) in EtOH (3 ml) was added NaOH (0.5 ml, 10% aq. solution) and the mixture was stirred at room temperature for 2 h. TLC detection showed that the reaction was complete. Then ice water (15 ml) was added and the solution was acidified to pH 5˜6 with concd, HCl. The precipitate thus obtained was collected by filtration and dried to give the title compound (60 mg). ¹H-NMR (d₆-DMSO, 300 MHz) δ1.91 (m, 2H), 2.56 (t, 2H, J=7.2), 2.73 (t, 2H, J=7.2), 4.29 (s, 2H), 5.21 (s, 2H), 5.72 (s, 2H), 7.01 (t, 2H, J=8.7), 7.14 (d, 2H, J=8.1), 7.31 (dd, 2H, J=8.4, 5.7), 7.62 (d, 2H, J=7.8), 7.83 (m, 5H), 12.98 (s, 1H).

### Example 52

### 2-((2-(4-fluorobenzylthio)-4-oxo-4,5,6,7-tetrahydro-1H-cyclopenta[d]pyrimidin-1-yl)methyl)-N,N-dimethyl-1-((4′-(trifluoromethyl)biphenyl-4-yl)methyl)-1H-imid azole-5-carboxamide

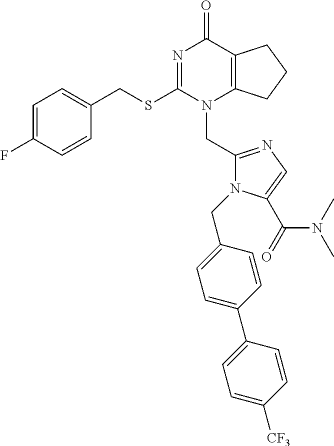 [SMILES: CN(C)C(=O)c1cnc(Cn2c(SCc3ccc(F)cc3)nc(=O)c3c2CCC3)n1Cc1ccc(-c2ccc(C(F)(F)F)cc2)cc1]

A mixture of example 51 (40 mg), EDCI (18 mg), HOBt (13 mg), dimethylamine hydrochloride (8 mg) and DIPEA (17 μl) in DCM (5 ml) was stirred at room temperature for 4 h. Then, the solution was washed with saturated NH₄Cl solution twice, dried over anhydrous NaSO₄ and purified by preparative TLC to give the title compound as a white solid (20 mg). ¹H-NMR (CDCl₃, 300 MHz) δ2.01 (m, 2H), 2.75 (m, 4H), 3.07 (s, 3H), 3.09 (s, 3H), 4.44 (s, 2H), 4.96 (s, 2H), 5.49 (s, 2H), 6.89 (t, 2H, J=8.4), 7.11 (d, 2H, J=7.8), 7.27 (m, 3H), 7.46 (d, 2H, J=7.8), 7.59 (d, 2H, J=8.1), 7.70 (d, 2H, J=8.7).

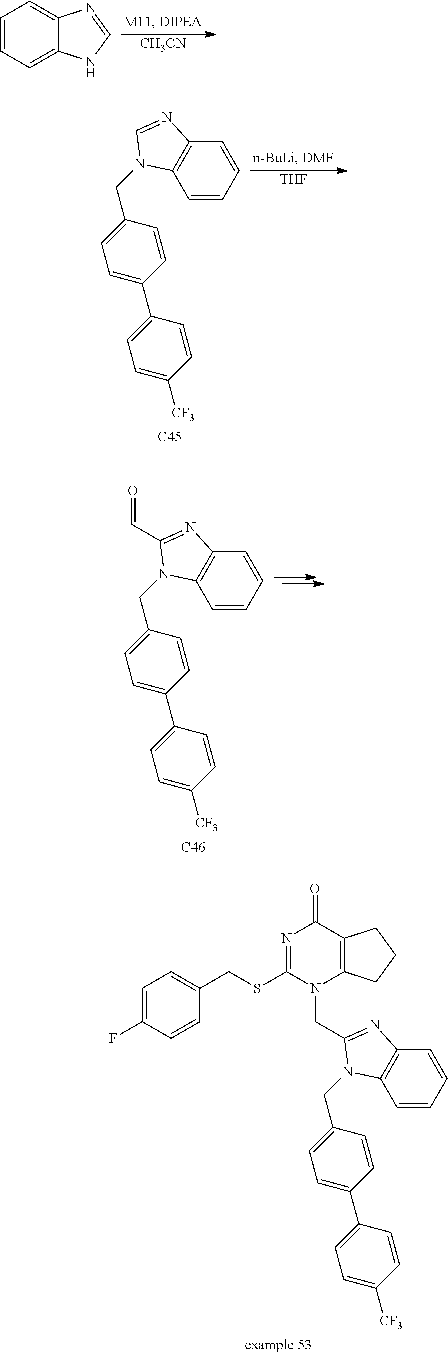

### Intermediate C45—1-((4′-(trifluoromethyl)biphenyl-4-yl)methyl)-1H-benzo[d]imidazole

A mixture of 1H-benzo[d]imidazole (519 mg, 4.4 mmol, 1.1 equiv), intermediate M11 (1.26 g, 4 mmol, 1 equiv) and DIPEA (730 mg, 1.1 equiv) in acetonitrile (10 ml) was refluxed for 4 h under nitrogen. TLC detection showed that the reaction was complete. The mixture was then filtered to remove the insoluble substances and the filtrate was mixed with silica gel, evaporated and then purified by column chromatography (PE/EA=1:2) to give the title compound as a white solid (590 mg). ¹H-NMR (CDCl₃, 300 MHz) δ5.43 (s, 2H), 7.29 (m, 5H), 7.57 (d, 2H, J=8.1), 7.67 (m, 4H), 7.86 (m, 1H), 8.00 (s, 1H); MS (ESI): 353 (M+H).

### Intermediate C46—1-((4′-(trifluoromethyl)biphenyl-4-yl)methyl)-1H-benzo[d]imidazole-2-carbaldehyde

Following a procedure similar to that described for the preparation of intermediate C17 except that intermediate C45 was used in place of intermediate C16. ¹H-NMR (CDCl₃, 300 MHz) δ5.92 (s, 2H), 7.27 (d, 2H, J=8.1), 7.42-7.53 (m, 5H), 7.62 (d, 2H, J=8.7), 7.67 (d, 2H, J=8.4), 7.98 (m, 1H), 10.17 (s, 1H).

### Example 53

### 2-(4-fluorobenzylthio)-1-((1-((4′-(trifluoromethyl)biphenyl-4-yl)methyl)-1H-benzo[d]imidazol-2-yl)methyl)-6,7-dihydro-1H-cyclopenta[d]pyrimidin-4(5H)-one

Following a procedure similar to that described for the preparation of example 28 except that intermediate C46 was used in place of intermediate C3. ¹H-NMR (CDCl₃, 300 MHz) δ1.95 (m, 2H), 2.68 (m, 4H), 4.46 (s, 2H), 5.22 (s, 2H), 5.44 (s, 2H), 6.90 (t, 2H, J=8.7), 7.00 (d, 2H, J=7.8), 7.26-7.37 (m, 5H), 7.43 (d, 2H, J=7.8), 7.60 (d, 2H, J=8.4), 7.69 (d, 2H, J=8.4), 7.82 (m, 2H); MS (ESI) found (M+H)=641.

### Example 54

### 2-(4-fluorobenzylthio)-5-((1-methyl-1H-pyrazol-4-yl)methyl)-1-((1-((4′-(trifluoromethyl)biphenyl-4-yl)methyl)-1H-benzo[d]imidazol-2-yl)methyl)pyrimidin-4 (1H)-one

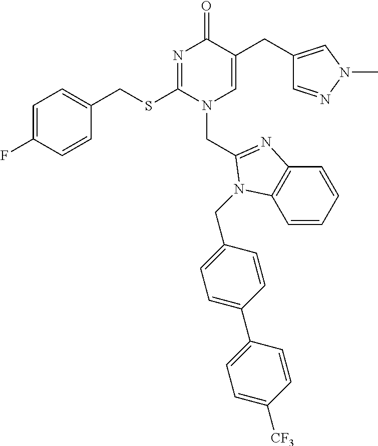 [SMILES: Cn1cc(Cc2cn(Cc3nc4ccccc4n3Cc3ccc(-c4ccc(C(F)(F)F)cc4)cc3)c(SCc3ccc(F)cc3)nc2=O)cn1]

Following a procedure similar to that described for the preparation of example 29 except that intermediate C46 was used in place of intermediate C3. ¹H-NMR (CDCl₃, 300 MHz) δ3.40 (s, 2H), 3.74 (s, 3H), 4.45 (s, 2H), 5.11 (s, 2H), 5.40 (s, 2H), 6.91-6.99 (m, 5H), 7.19 (s, 1H), 7.22 (s, 1H), 7.35 (m, 7H), 7.60 (d, 2H, J=7.5), 7.70 (d, 2H, J=7.5), 7.84 (m, 1H); MS (ESI): 695 (M+H).

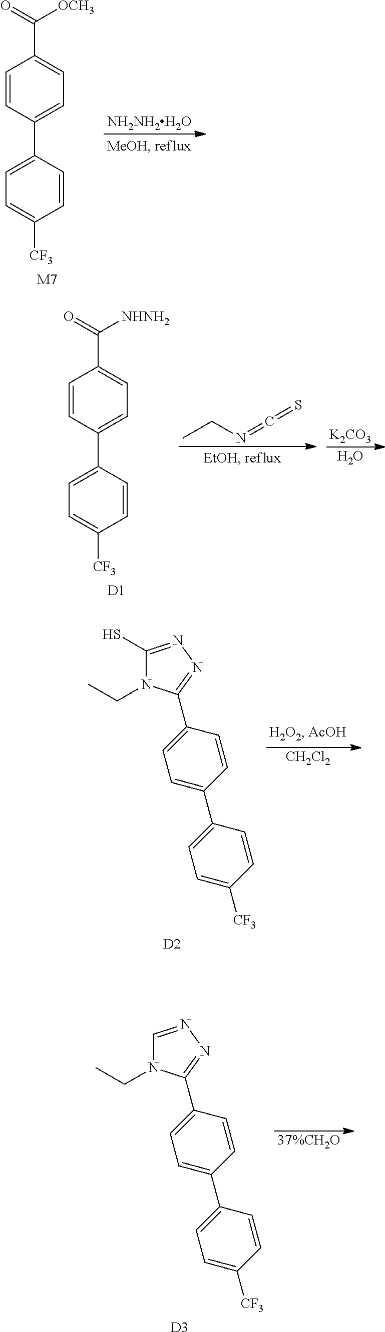

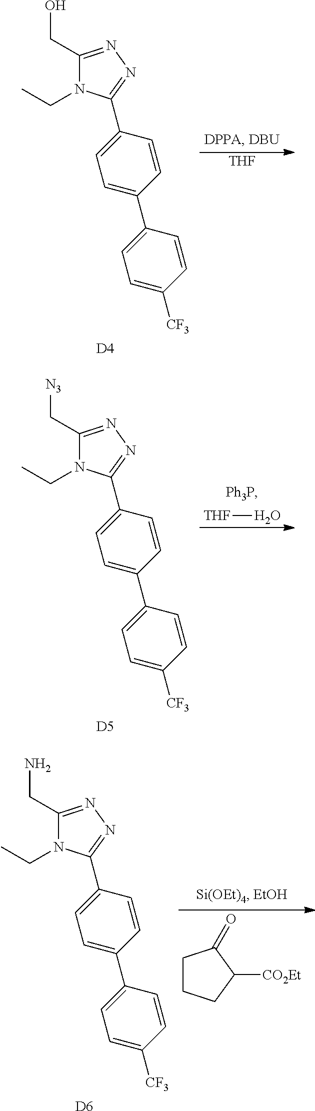

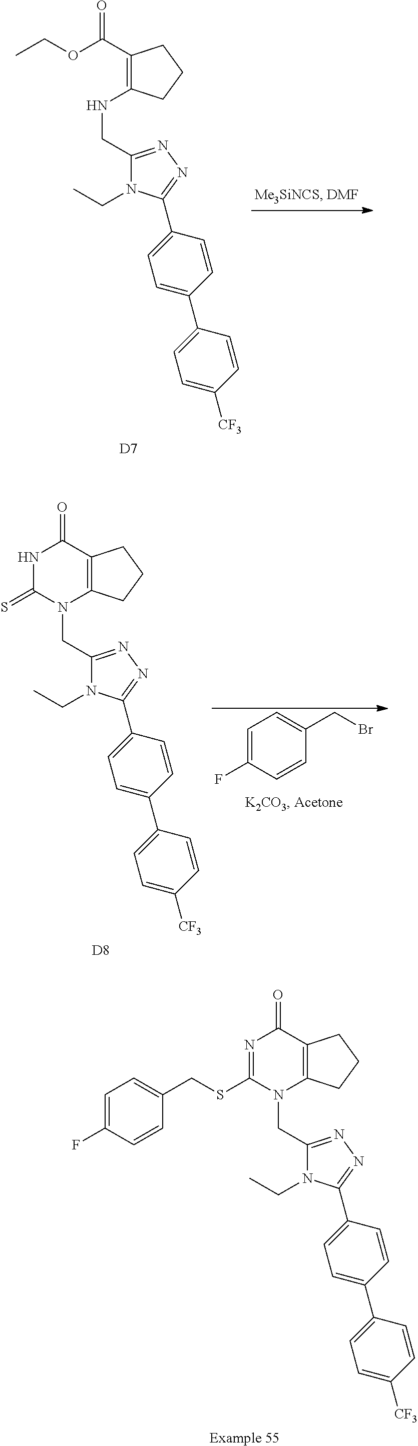

### Intermediate D1—4′-(trifluoromethyl)biphenyl-4-carbohydrazide

To a solution of intermediate M7 (10.5 g) in methanol (30 ml) was added hydrazine hydrate solution (20 ml, 85% m/m) and the mixture was refluxed for 4 h before it was evaporated to dryness. Toluene was added and evaporated again to remove the residual water to give the title compound as a white solid. ¹H-NMR (CDCl₃, 300 MHz) δ 4.15 (s, 2H), 7.53 (s, 1H), 7.67 (d, 2H, J=8.1), 7.71 (s, 4H), 7.85 (d, 2H, J=8.1); MS (EI) m/z: 280 (M⁺).

### Intermediate D2—4-ethyl-5-(4′-(trifluoromethyl)biphenyl-4-yl)-4H-1,2,4-triazole-3-thiol

A mixture of intermediate D1 (5.6 g, 1 equiv) and isothiocyanatoethane (1.76 ml, 1 equiv) in absolute ethanol (30 ml) was refluxed for 2.5 h. TLC detection showed that the starting material disappeared. Then most of the solvent was evaporated under reduced pressure, followed by addition of K₂CO₃ (3.26 g, 1.1 equiv) and water (30 ml). The resulting mixture was refluxed for a further 2 h and then neutralized with concd.HCl in an ice bath. The white precipitate thus obtained was collected by filtration, washed with water and dried to give the title compound (6.3 g). ¹H-NMR (d6-DMSO, 300 MHz) δ1.90 (t, 3H, J=7.2), 4.09 (q, 2H, J=7.2), 7.82 (d, 2H, J=8.4), 7.85 (d, 2H, J=9.3), 7.94 (d, 2H, J=8.4), 7.98 (d, 2H, J=8.1), 13.99 (s, 1H); MS (EI) m/z: 349 (M⁺).

### Intermediate D3—4-ethyl-3-(4′-(trifluoromethyl)biphenyl-4-yl)-4H-1,2,4-triazole

To a slurry of intermediate D2 (6.3 g, 1 equiv) in DCM (30 ml) was added AcOH (1.5 ml) and the mixture was heated in an oil bath (35° C.). An aqueous solution of hydrogen peroxide (5.1 ml, 30% wt, >2.5 equiv) was added slowly over 0.5 h. After the addition, the mixture was stirred at room temperature for a further 0.5 h. Then, the solution was adjusted to pH>10 with a solution of NaOH (10%) in an ice bath and extracted with DCM twice. The combined organic phase was washed with brine twice, dried over anhydrous MgSO₄ and purified by column chromatography to give the title compound as a solid (4.5 g). ¹H-NMR (CDCl₃, 300 MHz) δ1.47 (t, 3H, J=7.4), 4.15 (q, 2H, J=7.2), 7.73 (d, 8H), 8.30 (s, 1H); MS (EI) m/z: 317 (M⁺).

### Intermediate D4—(4-ethyl-5-(4′-(trifluoromethyl)biphenyl-4-yl)-4H-1,2,4-triazol-3-yl)methanol

A solution of intermediate D3 (4.5 g) in formalin (20 ml, 37% wt) was refluxed for 12 h to obtain a clear solution. After cooled, the solution was diluted with saturated brine (20 ml) and extracted with DCM three times (totally 100 ml). The combined organic phase was washed with brine four times, dried over MgSO₄, filtered and solvent concentrated to let a batch of product precipitate. The solid so obtained was collected by filtration and dried to give the title compound (2.0 g). The mother liquid was purified by column chromatography to give another batch of product (2.3 g) as a white solid. ¹H-NMR (CD₃OD, 300 MHz) δ1.33 (t, 3H, J=7.2), 4.28 (q, 2H, J=7.2), 4.85 (s, 2H), 7.78 (d, 4H, J=8.1), 7.90 (d, 4H, J=7.8); MS (EI) m/z: 347 (M⁺).

### Intermediate D5—3-(azido methyl)-4-ethyl-5-(4′-(trifluoromethyl)biphenyl-4-yl)-4H-1,2,4-triazole

A mixture of intermediate D4 (1.04 g, 1 equiv), DPPA (0.77 ml, 1.2 equiv) and DBU (0.67 ml, 1.5 equiv) in THF (10 ml) was refluxed for 3 h. TLC detection showed that the reaction was complete. After cooled, the mixture was then poured into saturated NH₄Cl solution and extracted with EA. The combined organic phase was washed with brine twice, dried and then purified by column chromatography (DCM/MeOH=20:1) to give the title compound as a solid (0.95 g). ¹H-NMR (CDCl₃, 300 MHz) δ1.38 (t, 3H, J=7.4), 4.15 (q, 2H, J=7.4), 4.64 (s, 2H), 7.74 (d, 8H); MS (EI) m/z: 372 (M⁺).

### Intermediate D6—(4-ethyl-5-(4′-(trifluoromethyl)biphenyl-4-yl)-4H-1,2,4-triazol-3-yl)methanamine

To a solution of D5 (0.94 g, 1 equiv) in THF—H₂O (8-0.5 ml) was added Ph₃P (990 mg, 1.5 equiv) and the mixture was stirred at room temperature overnight. After the reaction was complete, the mixture was then mixed with silica gel, evaporated to dryness and loaded on a short silica column. The column was first eluted with EA to get rid of Ph₃P and Ph₃P═O and then eluted with methanol to afford the target product portion. The methanol solution so collected was evaporated to dryness and the residue was dissolved in a mixed solvent of DCM/MeOH (15:1). Insoluble silica gel was removed by filtration and the filtrate was evaporated to dryness to give the title compound as a yellow solid (0.68 g). ¹H-NMR (CDCl₃, 300 MHz) δ1.35 (t, 3H, J=7.5), 1.75 (s, 2H), 4.13 (s, 2H), 4.15 (q, 2H, J=7.4), 7.72 (s, 8H); MS (EI) m/z: 346 (M⁺).

### Intermediate D7—ethyl 2-((4-ethyl-5-(4′-(trifluoromethyl)biphenyl-4-yl)-4H-1,2,4-triazol-3-yl)methylamino)cyclopent-1-enecarboxylate

To a solution of D6 (0.67 g, 1 equiv) in absolute ethanol (10 ml) were added ethyl 2-oxocyclopentanecarboxylate (320 μl, 1.1 equiv) and tetraethyl orthosilicate (860 μl, 2 equiv). The mixture was refluxed under nitrogen for 5 h until the completion of the reaction monitored by TLC. Silica gel was added to the solution and the mixture was evaporated to dryness, then purified by column chromatography (DCM/MeOH=30:1) to give the title compound as a solid (0.88 g). ¹H-NMR (CDCl₃, 300 MHz) δ1.25 (t, 3H, J=7.2), 1.34 (t, 3H, J=7.2), 1.86 (m, 2H), 2.52 (t, 2H, J=7.2), 2.74 (t, 2H, J=7.6), 4.13 (q, 2H, J=7.2), 4.65 (d, 2H, J=6.6), 7.73 (m, 9H); MS (EI) m/z: 484 (M⁺).

### Intermediate D8—1-((4-ethyl-5-(4′-(trifluoromethyl)biphenyl-4-yl)-4H-1,2,4-triazol-3-yl)methyl)-24 hioxo-2,3,6,7-tetrahydro-1H-cyclopenta[d]pyrimidin-4(5H)-one

A mixture of D7 (0.80 g, 1 equiv), isothiocyanatotrimethylsilane (0.9 ml, 4 equiv) in anhydrous DMF (2 ml) was heated at 140° C. for 8 h under nitrogen. The flask was cooled in an ice bath and the mixture was quenched by addition of saturated NaHCO₃ solution, diluted with EA (15 ml) and water (10 ml), and then stirred for 2 h. The precipitate thus obtained was collected and dried to give the title compound (0.53 g). ¹H-NMR (d₆-DMSO, 300 MHz) δ1.35 (t, 3H, J=7.2), 2.06 (m, 2H), 2.67 (t, 2H, J=7.5), 3.07 (t, 2H, J=7.2), 4.20 (q, 2H, J=7.2), 5.68 (s, 2H), 7.79 (d, 2H, J=8.4), 7.86 (d, 2H, J=8.7), 7.94 (d, 2H, J=8.4), 7.99 (d, 2H, J=8.1); MS (EI) m/z: 497 (M).

### Example 55

### 1-((4-ethyl-5-(4′-(trifluoromethyl)biphenyl-4-yl)-4H-1,2,4-triazol-3-yl)methyl)-2-(4-fluorobenzylthio)-6,7-dihydro-1H-cyclopenta[d]pyrimidin-4(5H)-one

A mixture of D8 (100 mg, 1 equiv), 1-(bromomethyl)-4-fluorobenzene (28 μl, 1.1 equiv) and anhydrous K₂CO₃ (40 mg, 1.5 equiv) in acetone (3 ml) was refluxed for 1 h. Inorganic salts were removed by filtration and the filtrate was purified by column chromatography (DCM/MeOH=10:1) to give the title compound as a solid (60 mg). ¹H-NMR (CDCl₃, 300 MHz) δ1.30 (t, 3H, J=7.2), 2.13 (m, 2H), 2.86 (t, 2H, J=7.4), 3.05 (t, 2H, J=7.6), 4.10 (q, 2H, J=7.2), 4.53 (s, 2H), 5.24 (s, 2H), 6.97 (t, 2H, J=8.7), 7.35 (dd, 2H, J=8.7, 5.3), 7.67-7.76 (m, 8H); MS (ESI): 606 (M+H).

### Example 56

### 1-((4-ethyl-5-(4′-(trifluoromethyl)biphenyl-4-yl)-4H-1,2,4-triazol-3-yl)methyl)-2-(4-methoxybenzylthio)-6,7-dihydro-1H-cyclopenta[d]pyrimidin-4(5H)-one

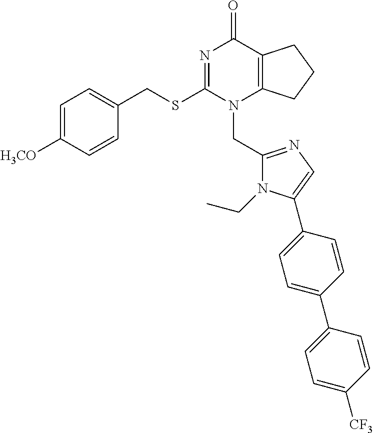 [SMILES: CCn1c(-c2ccc(-c3ccc(C(F)(F)F)cc3)cc2)cnc1Cn1c(SCc2ccc(OC)cc2)nc(=O)c2c1CCC2]

Following a procedure similar to that described for the preparation of example 55 except that 1-(chloromethyl)-4-methoxybenzene was used in place of 1-(bromomethyl)-4-fluorobenzene. ¹H-NMR (CDCl₃, 300 MHz) δ1.28 (t, 3H, J=7.2), 2.12 (m, 2H), 2.85 (t, 2H, J=7.4), 3.05 (t, 2H, J=7.6), 3.76 (s, 3H), 4.10 (q, 2H, J=7.2), 4.51 (s, 2H), 5.24 (s, 2H), 6.81 (d, 2H, J=8.4), 7.29 (d, 2H, J=8.7), 7.73 (m, 8H); MS (ESI): 618 (M+H).

### Example 57

### 2-(2,3-difluorobenzylthio)-1-((4-ethyl-5-(4′-(trifluoromethyl)biphenyl-4-yl)-4H-1,2,4-triazol-3-yl)methyl)-6,7-dihydro-1H-cyclopenta[d]pyrimidin-4 (5H)-one

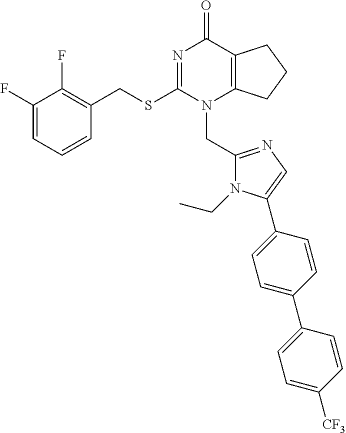 [SMILES: CCn1c(-c2ccc(-c3ccc(C(F)(F)F)cc3)cc2)cnc1Cn1c(SCc2cccc(F)c2F)nc(=O)c2c1CCC2]

Following a procedure similar to that described for the preparation of example 55 except that 1-(bromomethyl)-2,3-difluorobenzene was used in place of 1-(bromomethyl)-4-fluorobenzene. ¹H-NMR (CDCl₃, 300 MHz) δ 1.31 (t, 3H), 2.13 (m, 2H), 2.86 (t, 2H), 3.06 (t, 2H), 4.13 (q, 2H), 4.61 (s, 2H), 5.23 (s, 2H), 7.04 (m, 2H), 7.35 (t, 1H), 7.74 (m, 8H).

### Example 58

### 1-((4-ethyl-5-(4′-(trifluoromethyl)biphenyl-4-yl)-4H-1,2,4-triazol-3-yl)methyl)-2-(4-fluorobenzylthio)-5-((1-methyl-1H-pyrazol-4-yl)methyl)pyrimidin-4 (1H)-one

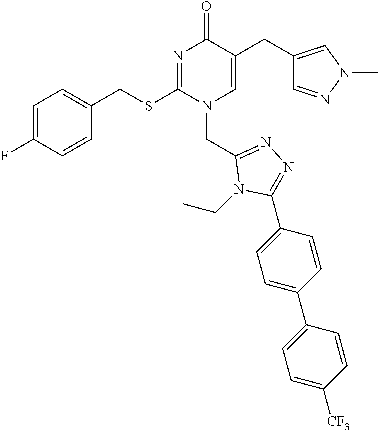 [SMILES: CCn1c(Cn2cc(Cc3cnn(C)c3)c(=O)nc2SCc2ccc(F)cc2)nnc1-c1ccc(-c2ccc(C(F)(F)F)cc2)cc1]

Following a procedure similar to that described for the preparation of example 29 except that intermediate D6 was used in place of intermediate C3. ¹H-NMR (CDCl₃, 300 MHz) δ 1.16 (t, 3H, J=7.2), 3.56 (s, 2H), 3.83 (s, 3H), 4.02 (q, 2H, J=7.2), 4.55 (s, 2H), 5.16 (s, 2H), 6.99 (t, 2H, J=8.4), 7.21 (s, 1H), 7.30 (s, 1H), 7.32 (s, 1H), 7.40 (m, 2H), 7.72 (m, 8H); MS (ESI): 660 (M+H).

### Intermediate D9—methyl 4-phenylbutanoate

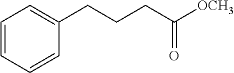 [SMILES: COC(=O)CCCc1ccccc1]

To a stirred solution of 4-phenylbutanoic acid (3.28 g, 20 mmol) in absolute methanol (20 ml) was added concd. H₂SO₄ (2 ml) dropwise (heat was released) and the mixture was refluxed for 4 h. Then, most of the solvent was evaporated and ice water (30 ml) was added to the residue. The resulting mixture was neutralized with a solution of NaOH (10%) and extracted with DCM (60 ml) three times. The combined organic phase was dried over MgSO₄ and solvent evaporated to dryness to give the title compound as an oil which could be used in next step without further purification. ¹H-NMR (CDCl₃, 300 MHz) δ 1.96 (m, 2H), 2.33 (t, 2H, J=7.5), 2.65 (t, 2H, J=7.5), 3.66 (s, 3H), ˜7.25 (m, 5H).

### Intermediate D10—4-phenylbutanehydrazide

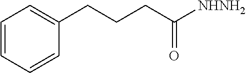 [SMILES: NNC(=O)CCCc1ccccc1]

Following a procedure similar to that described for the preparation of intermediate D1 except that intermediate D9 was used in place of intermediate M7. H-NMR (CD₃OD, 300 MHz) δ1.97 (m, 2H), 2.14 (t, 2H, J=7.2), 2.64 (t, 2H, J=7.2), 3.89 (vbrs, 2H), 6.80 (vbrs, 1H), 7.17 (m, 3H), 7.27 (m, 2H).

### Intermediate D11—2-(4′-(trifluoromethyl)biphenyl-4-yl)acetohydrazide

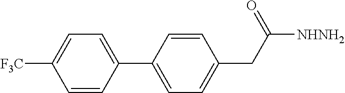 [SMILES: NNC(=O)Cc1ccc(-c2ccc(C(F)(F)F)cc2)cc1]

Following a procedure similar to that described for the preparation of intermediate D1 except that intermediate M3 was used in place of intermediate M7. ¹H-NMR (d6-DMSO, 300 MHz) δ 3.41 (s, 2H), 4.26 (s, 2H, —NH₂), 7.39 (d, 2H), 7.67 (d, 2H), 7.80 (d, 2H), 7.87 (d, 2H), 9.26 (s, 1H)

### Example 59

### 1-((4-ethyl-5-(3-phenylpropyl)-4H-1,2,4-triazol-3-yl)methyl)-2-(4-fluorobenzylthio)-6,7-dihydro-1H-cyclopenta[d]pyrimidin-4(5H)-one

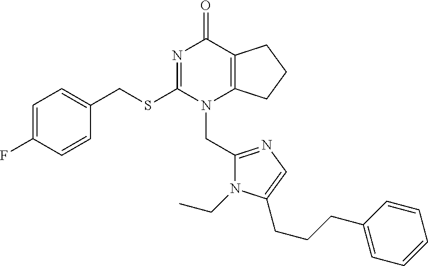 [SMILES: CCn1c(CCCc2ccccc2)cnc1Cn1c(SCc2ccc(F)cc2)nc(=O)c2c1CCC2]

Following a procedure similar to that described for the preparation of example 55 except that intermediate D10 was used in place of intermediate D1. ¹H-NMR (CDCl₃, 300 MHz) δ 1.13 (t, 3H), 2.13 (m, 4H), 2.66 (t, 2H), 2.78 (m, 4H), 2.95 (t, 2H), 3.79 (q, 2H), 4.49 (s, 2H), 5.12 (s, 2H), 6.95 (t, 2H), 7.18 (m, 3H), 7.28 (m, 2H), 7.33 (dd, 2H); MS (ESI): 504 (M+H).

### Example 60

### 1-((4-ethyl-5-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

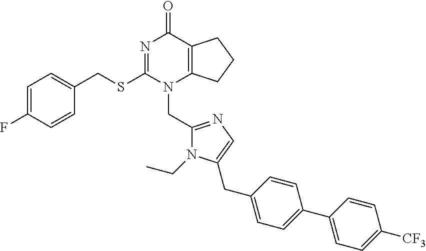 [SMILES: CCn1c(Cc2ccc(-c3ccc(C(F)(F)F)cc3)cc2)cnc1Cn1c(SCc2ccc(F)cc2)nc(=O)c2c1CCC2]

Following a procedure similar to that described for the preparation of example 55 except that intermediate D11 was used in place of intermediate D1. ¹H-NMR (CDCl₃, 300 MHz) δ1.02 (t, 3H, J=7.2), 2.11 (m, 2H), 2.83 (t, 2H, J=7.5), 2.98 (t, 2H, J=7.5), 3.82 (q, 2H, J=7.2), 4.23 (s, 2H), 4.49 (s, 2H), 5.11 (s, 2H), 6.95 (t, 2H, J=8.7), 7.31 (m, 4H), 7.53 (d, 2H, J=8.1), 7.64 (d, 2H, J=8.4), 7.69 (d, 2H, J=8.7); MS (ESI): 620 (M+H).

### Intermediate D12—1-((4′-trifluoromethyfibinhenyl-4-yl)methyl)-1H-1,2,4-triazole

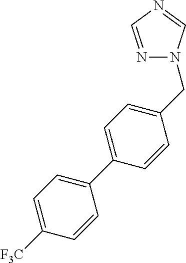 [SMILES: FC(F)(F)c1ccc(-c2ccc(Cn3cncn3)cc2)cc1]

A mixture of 4-(bromomethyl)-4′-(trifluoromethyl)biphenyl (1.25 g), 1H-1,2,4-triazole (0.4 g) and DIPEA (0.9 ml) in acetonitrile (15 ml) was refluxed for 3 h. Then insoluble substance was filtered off and the filtrate was mixed with silica gel and evaporated t dryness and then purified by column chromatography to give the title compound as a solid (0.54 g). ¹H-NMR (CD₃OD, 300 MHz) δ5.49 (s, 2H), 7.42 (d, 2H, J=8.1), 7.68 (d, 2H, J=8.1), 7.72 (d, 2H, J=8.4), 7.79 (d, 2H, J=8.4), 8.02 (s, 1H), 8.60 (s, 1H).

### Intermediate D13—(1-((4′-(trifluoromethyl)biphenyl-4-yl)methyl)-1H-1,2,4-triazol-3-yl)methanol

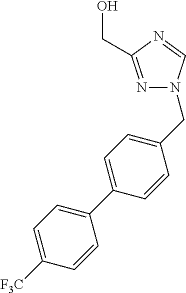 [SMILES: OCc1ncn(Cc2ccc(-c3ccc(C(F)(F)F)cc3)cc2)n1]

Following a procedure similar to that described for the preparation of intermediate D4. ¹H-NMR (CDCl₃, 300 MHz) δ3.91 (s, 1H), 4.79 (s, 2H), 5.48 (s, 2H), 7.37 (d, 2H, J=8.4), 7.58 (d, 2H, J=8.4), 7.64 (d, 2H, J=8.7), 7.69 (d, 2H, J=8.7), 7.84 (s, 1H).

### Example 61

### 2-(4-fluorobenzylthio)-1-((1-((4′-(trifluoromethyl)biphenyl-4-yl)methyl)-1H-1,2,4-triazol-3-yl)methyl)-6,7-dihydro-1H-cyclopenta[d]pyrimidin-4(5H)-one

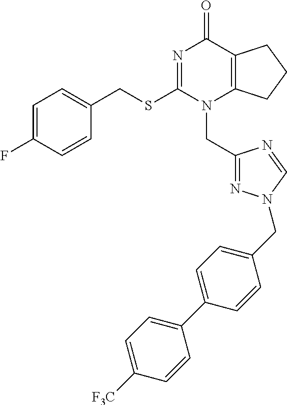 [SMILES: O=c1nc(SCc2ccc(F)cc2)n(Cc2ncn(Cc3ccc(-c4ccc(C(F)(F)F)cc4)cc3)n2)c2c1CCC2]

Following a procedure similar to that described for the preparation of example 55 except that intermediate D13 was used in place of intermediate D4. ¹H-NMR (CDCl₃, 300 MHz) δ1.99 (m, 2H), 2.71 (m, 4H), 4.45 (s, 2H), 5.10 (s, 2H), 5.46 (s, 2H), 6.92 (t, 2H, J=7.8), 7.18 (d, 2H, J=8.1), 7.29 (m, 2H), 7.50 (d, 2H, J=7.8), 7.62 (d, 2H, J=7.8), 7.70 (d, 2H, J=7.8), 7.92 (s, 1H).

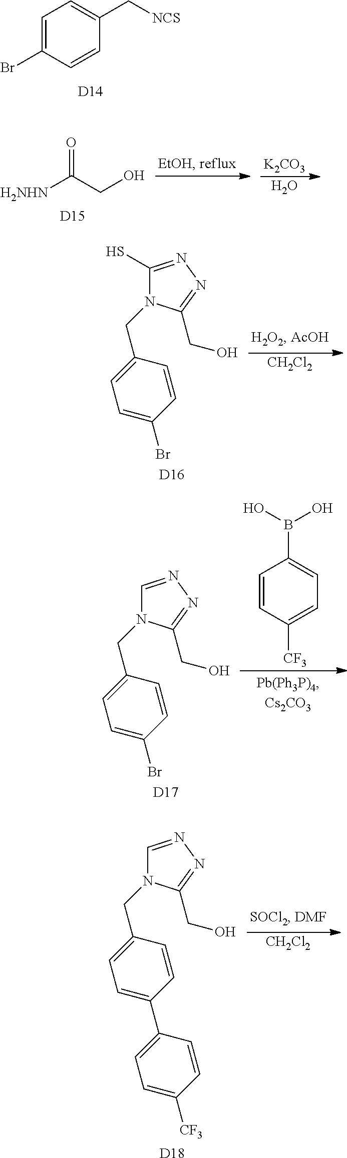

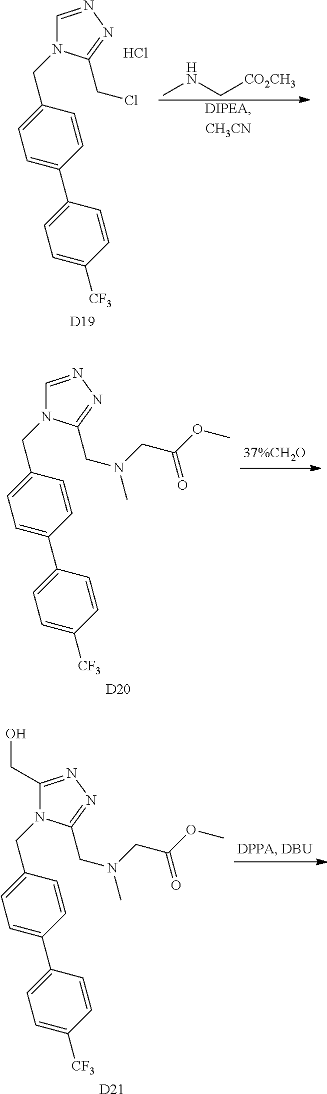

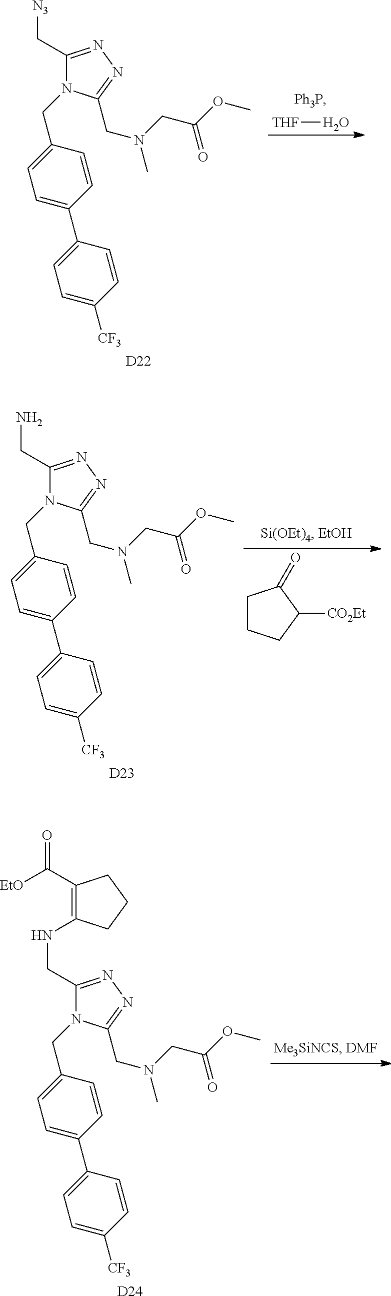

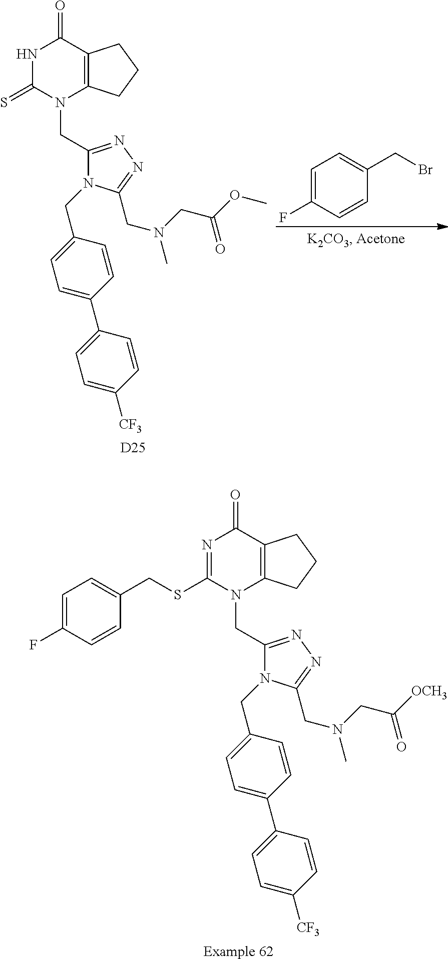

### Intermediate D14—1-bromo-4-(isothiocyanatomethyl)benzene

To an ice cold mixture of (4-bromophenyl)methanamine (22.8 g, 0.1 mol, 1 equiv) and triethylamine (17.5 ml, 3.3 equiv) in THF (100 ml) in a flask equipped with a drying tube for insulating moisture was added a solution of CS₂ (6 ml, 1 equiv) in THF (25 ml) over 30 min to obtain a white slurry. The mixture was stirred at room temperature for 1 h and then placed in ice bath again. Tosyl chloride (20.9 g, 1.1 equiv) was added in batches and the mixture was stirred for a further 1 h before it was diluted with ice water. The solution was adjusted to pH˜5 with concd. HCl and extracted with EA. The combined organic phase was washed with brine, dried and evaporated to give the title compound as a yellow oil which could be used in next step without further purification.

### Intermediate D15—2-hydroxyacetohydrazide

A mixture of methyl 2-hydroxyacetate (9 g, 0.1 mol, 1 equiv) and hydrazine hydrate (9.6 ml, 1.5 equiv, 85%) in methanol (100 ml) was refluxed for 8 h before methanol and exceesive hydrazine hydrate were evaporated. Toluene was added and evaporated again to remove the residual water to give the title compound as a white solid which could be used in next step without further purification.

### Intermediate D16—(4-(4-bromobenzyl)-5-mercapto-4H-1,2,4-triazol-3-yl)methanol

A mixture of D14 (0.1 mol) and D15 (0.1 mol) from the previous steps in ethanol (200 ml) was refluxed for 2 h and a lot of white precipitates separated out. Then most of the solvent was evaporated. Water (200 ml) and K₂CO₃ (20.7 g, 1.5 equiv) was added to the residue and the mixture was refluxed for a further 1 h. After cooled, the solution was placed in an ice bath and adjusted to pH 8-9 with conc. HCl. A lot of white solid precipitated (if a yellow solid was present, a small amount of DCM could be added) and was collected by filtration, washed with water several times and DCM several times to wash off the color and dried in vacuo to give the title compound as a white solid (2.5 g). ¹H-NMR (d₆-DMSO, 300 MHz) δ4.37 (s, 2H), 5.24 (s, 2H), 5.73 (s, 1H, —OH), 7.25 (d, 2H, J=8.4), 7.54 (d, 2H, J=8.7), 13.80 (s, 1H).

### Intermediate D17(4-(4-bromobenzyl)-4H-1,2,4-triazol-3-yl)methanol

To a suspension of D16 (25 g, 1 equiv) in DCM (100 ml) was added AcOH (6 ml) and the mixture was heated in an oil bath (35° C.). An aqueous solution of hydrogen peroxide (24 ml, >2.5 equiv) was added slowly over 1 h. After the addition, the mixture was stirred at room temperature for a further 0.5 h before it was adjusted to pH>10 with a solution of NaOH (10%) in an ice bath. The mixture was stayed for 0.5 h and the precipitate so formed was collected by filtration, washed with water and DCM. Organic layer of the filtrate was separated and concentrated. Solids precipitated and was collected by filtration and dried to obtain an off white powder (20.4 g in total). ¹H-NMR (d₆-DMSO, 300 MHz) δ4.55 (s, 2H), 5.27 (s, 2H), 5.64 (s, 1H, —OH), 7.22 (d, 2H, J=8.4), 7.57 (d, 2H, J=8.4), 8.53 (s, 1H).

### Intermediate D18—(4-((4′-(trifluoromethyl)biphenyl-4-yl)methyl)-4H-1,2,4-triazol-3-yl)methanol

A mixture of intermediate C17 (13.4 g, 50 mmol, 1 equiv), 4-(trifluoromethyl)phenylboronic acid (10.4 g, 1.1 equiv), Cs₂CO₃ (32.6 g, 2 equiv) and Pd(PPh)₃ (2.9 g, 0.05 equiv) in dioxane (100 ml) was refluxed for 18 h in a nitrogen atmosphere. The inorganic salt was filtered off, washed with dioxane and the filtrate was evaporated to dryness to give a residue that was recrystallized from DCM/MeOH to give the title compound as a yellow powder (13 g). ¹H-NMR (d₆-DMSO, 300 MHz) δ4.59 (d, 2H, J=5.4), 5.37 (s, 2H), 5.69 (t, 1H, J=5.4), 7.40 (d, 2H, J=8.1), 7.75 (d, 2H, J=8.4), 7.81 (d, 2H, J=8.4), 7.89 (d, 2H, J=8.4), 8.59 (s, 1H); MS (ESI): 334 (M+H).

### Intermediate D19—3-(chloromethyl)-4-((4′-(trifluoromethyl)biphenyl-4-yl)methyl)-4H-1,2,4-triazole Hydrochloride

To an ice cold mixture of D18 (5 mmol) and DMF (2 drops, catalyst) in DCM (20 ml) in a 100 ml of flask was added a solution of thionyl chloride (0.42 ml, 6 mmol) in DCM (5 ml) over 10 min and the mixture was stirred at room temperature. TLC monitored the progress of the reaction and showed that the reaction was complete about 2 hours later. (Handling Method 1) The solid thus formed was collected by filtration, washed with DCM and dried AT 80° C. to give the title compound as a white or light yellow solid. (Handling Method 2) The filtrate was evaporated to dryness to give the title compound as a yellow solid which could be used in next step without further purification. ¹H-NMR (d₆-DMSO, 300 MHz) δ5.06 (s, 2H), 5.46 (s, 2H), 7.47 (d, 2H, J=8.0), 7.77 (d, 2H, J=8.4), 7.80 (d, 2H, J=8.8), 7.89 (d, 2H, J=8.0), 9.12 (s, 1H).

### Intermediate D20—methyl 2-(methyl((4-((4′-(trifluoromethyl)biphenyl-4-yl)methyl)-4H-1,2,4-triazol-3-yl)methyl)amino)acetate

A mixture of D19 (2.0 g, 1 equiv), methyl 2-(methylamino)acetate hydrochloride (0.86 g, 1.2 equiv) and DIPEA (3 ml, 3 equiv) in acetonitrile (20 ml) was heated at 7080° C. until the completion of the reaction monitored by TLC. Acetonitrile was evaporated and saturated NH₄Cl solution (40 ml) was added to the residue. The mixture was extracted with DCM twice and the combined organic phase was washed with brine twice, dried, and then purified by column chromatography to give the title compound as a jelly (1.13 g). ¹H-NMR (CDCl₃, 300 MHz) δ2.38 (s, 3H), 3.32 (s, 2H), 3.70 (s, 3H), 3.86 (s, 2H), 5.51 (s, 2H), 7.31 (d, 2H, J=8.4), 7.60 (d, 2H, J=8.1), 7.68 (m, 4H), 8.16 (s, 1H).

### Intermediate D21—methyl 2-(((5-(hydroxymethyl)-4-((4′-(trifluoromethyl)biphenyl-4-yl)methyl)-4H-1,2,4-triazol-3-yl)methyl)(methyl)amino)acetate

A solution of D₂O (1.13 g) in formalin (15 ml, 37% wt) was refluxed for 5 h. TLC detection showed that the reaction was complete. The solution was poured into saturated brine (40 ml) and extracted with DCM three times (60 ml). The combined organic phase was washed with brine three times, dried over MgSO₄, and then purified by column chromatography to give the title compound as a jelly (1.0 g). ¹H-NMR (CDCl₃, 400 MHz) δ2.33 (s, 3H), 3.26 (s, 2H), 3.66 (s, 3H), 3.75 (s, 2H), 4.74 (s, 2H), 5.62 (s, 2H), 7.26 (d, 2H, J=8.0), 7.56 (d, 2H, J=8.0), 7.65 (d, 2H, J=8.8), 7.69 (d, 2H, J=8.8).

### Intermediate D22—methyl 2-(((5-(azidomethyl)-4-((4′-(trifluoromethyl)biphenyl-4-yl)methyl)-4H-1,2,4-triazol-3-yl)methyl)(methyl)amino)acetate

A mixture of D21 (1.0 g, 1 equiv), DPPA (0.58 ml, 1.2 equiv) and DBU (0.44 ml, 1.3 equiv) in THF (10 ml) was refluxed for 3 h under nitrogen. TLC detection showed that the reaction was complete. After cooled, the mixture was then poured into saturated NH₄Cl solution and extracted with EA. The combined organic phase was washed with brine twice, dried and then purified by flash chromatography to give the title compound as a jelly (0.97 g). ¹H-NMR (CDCl₃, 400 MHz) δ2.44 (s, 3H), 3.36 (s, 2H), 3.70 (s, 3H), 3.88 (s, 2H), 4.47 (s, 2H), 5.63 (s, 2H), 7.22 (d, 2H, J=8.0), 7.61 (d, 2H, J=8.0), 7.69 (d, 2H, J=8.4), 7.72 (d, 2H, J=8.8).

### Intermediate D23—methyl 2-(((5-(aminomethyl)-4-((4′-(trifluoromethyl)biphenyl-4-yl)methyl)-4H-1,2,4-triazol-3-yl)methyl)(methyl)amino)acetate

To a solution of D22 (0.97 g, 1 equiv) in THF—H₂O (8-0.5 ml) was added Ph₃P (806 mg, 1.5 equiv) and the mixture was stirred at room temperature overnight. After the reaction was complete, the mixture was then mixed with silica gel, evaporated to dryness and loaded on a short silica column. The column was first eluted with EA to get rid of Ph₃P and Ph₃P═O and then eluted with methanol to afford the target product portion. The methanol solution so collected was evaporated to dryness and the residue was dissolved in a mixed solvent of DCM/MeOH (15:1). Insoluble silica gel was removed by filtration and the filtrate was evaporated to dryness to give the title compound as a jelly (0.8 g). ¹H-NMR (CDCl₃, 400 MHz) δ2.37 (s, 2H+3H), 3.30 (s, 2H), 3.69 (s, 3H), 3.81 (s, 2H), 4.01 (s, 2H), 5.62 (s, 2H), 7.23 (d, 2H, J=8.4), 7.59 (d, 2H, J=8.0), 7.69 (m, 4H).

### Intermediate D24—ethyl 2-((5-(((2-methoxy-2-oxoethyl)(methyl)amino)methyl)-4-((4′-(trifluoromethyl)biphenyl-4-yl)methyl)-4H-1,2,4-triazol-3-yl)methylamino)cyclopent-1-enecarboxylate

To a solution of D23 (0.8 g, 1 equiv) in absolute ethanol (10 ml) were added ethyl 2-oxocyclopentanecarboxylate (260 μl, 1 equiv) and tetraethyl orthosilicate (800 μl, 2 equiv). The mixture was refluxed under nitrogen for 3 h until the completion of the reaction monitored by TLC. Silica gel was added to the solution and the mixture was evaporated to dryness, then purified by column chromatography (DCM/MeOH=40:1) to give the title compound as a jelly (0.89 g). ¹H-NMR (CDCl₃, 400 MHz) δ1.21 (t, 3H, J=7.2), 1.75 (m, 2H), 2.36 (s, 3H), 2.50 (m, 4H), 3.29 (s, 2H), 3.65 (s, 3H), 3.82 (s, 2H), 4.06 (q, 2H, J=7.5), 4.36 (d, 2H, J=6.0), 5.56 (s, 2H), 7.15 (d, 2H, J=8.1), 7.56 (m, 3H), 7.68 (m, 4H); MS (ESI): 586 (M+H).

### Intermediate D25—methyl 2-(methyl((5-((4-oxo-2-thioxo-2,3,4,5,6,7-hexahydro-1H-cyclopenta[d]pyrimidin-1-yl)methyl)-4-((4′-(trifluoromethyl)biphenyl-4-yl)methyl)-4H-1,2,4-triazol-3-yl)methyl)amino)acetate

A mixture of D24 (0.89 g, 1 equiv), isothiocyanatotrimethylsilane (0.77 ml, 4 equiv) in anhydrous DMF (1 ml) was heated at 140° C. for 6 h under nitrogen. After cooled, the mixture was placed in an ice bath and quenched by addition of saturated NaHCO₃ solution, and then diluted with EA (15 ml) and water (10 ml), and then stirred for 2 h. The precipitate thus obtained was collected and dried to give the title compound (0.12 g). ¹H-NMR (d₆-DMSO, 300 MHz) δ1.94 (m, 2H), 2.24 (s, 3H), 2.50 (t, 2H), 2.83 (t, 2H, J=6.9), 3.35 (s, 2H), 3.57 (s, 3H), 3.81 (s, 2H), 5.50 (s, 2H), 5.61 (s, 2H), 7.26 (d, 2H, J=8.1), 7.71 (d, 2H, J=8.1), 7.81 (d, 2H, J=8.4), 7.87 (d, 2H, J=9.0), 12.55 (s, 1H).

### Example 62

### methyl 2-(((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)(methyl)amino)acetate

A mixture of D25 (120 mg, 1 equiv), 1-(bromomethyl)-4-fluorobenzene (281.11, 1.1 equiv) and anhydrous K₂CO₃ (55 mg, 2 equiv) in acetone (3 ml) was refluxed for 1 h. Inorganic salts were removed by filtration and the filtrate was purified by flash chromatography to give the title compound as a solid (65 mg). ¹H-NMR (CDCl₃, 400 MHz) δ1.98 (m, 2H), 2.43 (s, 3H), 2.67 (t, 2H, J=7.2), 2.76 (t, 2H, J=7.2), 3.38 (s, 2H), 3.66 (s, 3H), 3.95 (s, 2H), 4.43 (s, 2H), 5.03 (s, 2H), 5.64 (s, 2H), 6.92 (t, 2H, J=8.0), 7.00 (d, 2H, J=7.6), 7.30 (m, 2H), 7.46 (d, 2H, J=7.6), 7.61 (d, 2H, J=8.0), 7.71 (d, 2H, J=8.0).

### Example 63

### 2-(((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)(methyl)amino)acetic acid

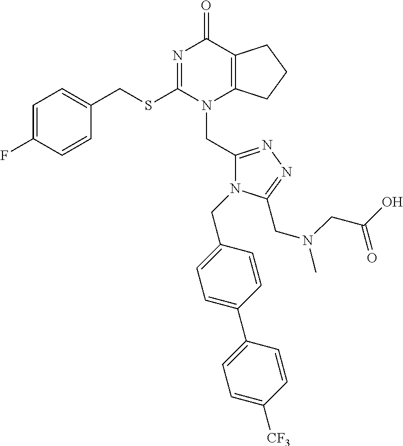 [SMILES: CN(CC(=O)O)Cc1nnc(Cn2c(SCc3ccc(F)cc3)nc(=O)c3c2CCC3)n1Cc1ccc(-c2ccc(C(F)(F)F)cc2)cc1]

To an ice cold solution of example 62 (40 mg) in the solvent of i-PrOH—CH₂Cl₂—H₂O (1 ml-0.5 ml-3 ml) was added 10% NaOH solution (0.1 ml) and the mixture was stirred for 1 h. Then it was diluted with water (5 ml), transferred into a separating funnel and washed with DCM (3 ml). The aqueous phase was collected and neutralized with a diluted solution of AcOH. The precipitate thus obtained was collected by filtration, washed with an aqueous acetone solution (50%, v/v) twice and dried to give the title compound as a white solid (12 mg). ¹H-NMR (CD₃OD, 300 MHz) δ2.06 (m, 2H), 2.41 (s, 3H), 2.70 (t, 2H, J=7.2), 2.84 (t, 2H, J=6.9), 3.38 (s, 2H), 4.00 (s, 2H), 4.35 (s, 2H), 5.26 (s, 2H), 5.67 (s, 2H), 6.86 (t, 2H, J=8.7), 7.23-7.31 (m, 4H), 7.56 (d, 2H, J=7.8), 7.73 (m, 4H); MS (ESI): 691(M−H).

### Intermediate D26—4-(4-bromobenzyl)-3-methyl-4H-1,2,4-triazole

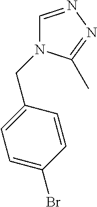

A mixture of 1,1-dimethoxy-N,N-dimethylmethanamine (2.6 ml) and acethydrazide (2 g) in acetonitrile (3 ml) was heated at 60° C. for 0.5 h before (4-bromophenyl)methanamine (3.7 g) and AcOH (4.5 ml) were added. The mixture was stirred at 120° C. for a further 3 h. After cooled, the mixture was poured into water and extracted with EA twice. The combined organic phase was dried over MgSO₄ and then purified by column chromatography to give the title compound as a white solid (1.1 g). ¹H-NMR (CDCl₃, 300 MHz) δ2.36 (s, 3H), 5.04 (s, 2H), 6.97 (d, 2H, J=8.4), 7.50 (d, 2H, J=8.4), 8.08 (s, 1H).

### Intermediate D27—3-methyl-4-((4′-(trifluoromethyl)biphenyl-4-yl)methyl)-4H-1,2,4-triazole

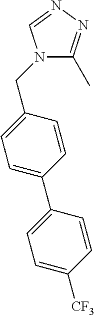 [SMILES: Cc1nncn1Cc1ccc(-c2ccc(C(F)(F)F)cc2)cc1]

A mixture of intermediate D26 (2.0 g, 1 equiv), 4-(trifluoromethyl)phenylboronic acid (1.6 g, 1.05 equiv), Cs₂CO₃ (5.2 g, 2 equiv) and Pd(PPh)₃ (0.5 g, 0.05 equiv) in dioxane (15 ml) was refluxed for 12 h in a nitrogen atmosphere. The inorganic salts were filtered off and washed with dioxane. The filtrate was concentrated and the precipitate thus obtained was collected, and then dried to give the title compound (1.65 g). ¹H-NMR (CDCl₃, 300 MHz) δ2.42 (s, 3H), 5.15 (s, 2H), 7.21 (d, 2H, J=8.4), 7.61 (d, 2H, J=8.1), 7.66 (d, 2H, J=8.7), 7.70 (d, 2H, J=9.0), 8.13 (s, 1H).

### Intermediate D28—(5-methyl-4-((4′-(trifluoromethyl)biphenyl-4-yl)methyl)-4H-1,2,4-triazol-3-yl)methanol

Following a procedure similar to that described for the preparation of intermediate D21 except that intermediate D27 was used in place of intermediate D20. ¹H-NMR (CDCl₃, 300 MHz) δ2.34 (s, 3H), 4.75 (s, 2H), 5.32 (s, 2H), 7.17 (d, 2H), 7.57 (d, 2H), 7.66 (d, 2H), 7.69 (d, 2H).

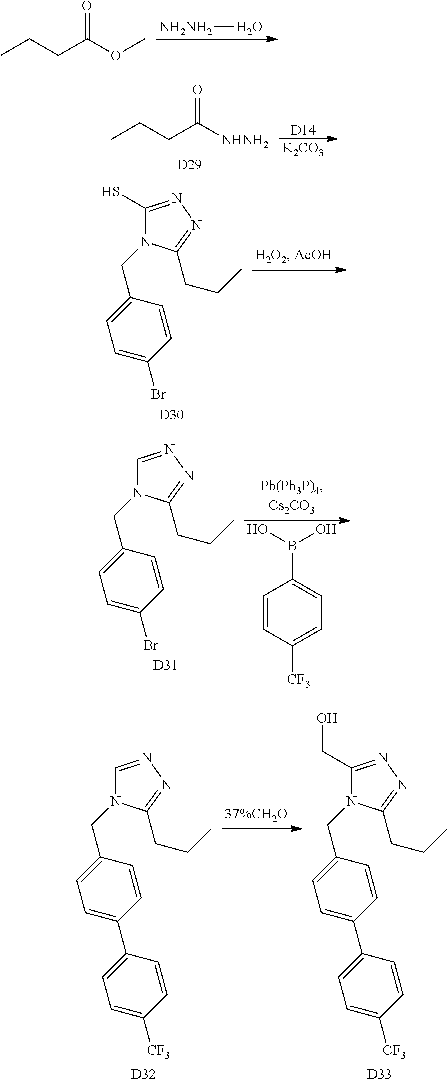

### Intermediate D29—Butyrohydrazide

Following a procedure similar to that described for the preparation of intermediate D15 except that methyl butyrate was used in place of methyl 2-hydroxyacetate.

### Intermediate D30—4-(4-bromobenzyl)-5-propyl-4H-1,2,4-triazole-3-thiol

Following a procedure similar to that described for the preparation of intermediate D16 except that D29 was used in place of D15. H-NMR (d₆-DMSO, 300 MHz) δ0.83 (t, 3H, J=7.2), 1.49 (m, 2H), 2.48 (t, 2H, J=7.2), 5.21 (s, 2H), 7.22 (d, 2H, J=8.7), 7.56 (d, 2H, J=8.7), 13.69 (s, 1H).

### Intermediate D31—4-(4-bromobenzyl)-3-n-propyl-4H-1,2,4-triazole

To a slurry of intermediate D30 (1.2 g, 1 equiv) in DCM (30 ml) was added AcOH (0.3 ml) and the mixture was heated in an oil bath (35° C.). A solution of hydrogen peroxide (1.1 ml, >2.5 equiv) was added slowly over 10 minutes. After the addition, the mixture was stirred at room temperature for a further 0.5 h. Then, the flask was placed in an ice bath and the solution was adjusted to pH>10 with a solution of NaOH (10%) and extracted with DCM twice. The combined organic phase was washed with brine twice, dried over anhydrous MgSO₄ and purified by column chromatography to give the title compound as a jelly (630 mg). H-NMR (CDCl₃, 300 MHz) δ0.98 (t, 3H, J=7.2), 1.77 (m, 2H), 2.62 (t, 2H, J=7.2), 5.04 (s, 2H), 6.97 (d, 2H, J=8.7), 7.51 (d, 2H, J=8.7), 8.06 (s, 1H).

Intermediate D32—3-n-propyl-4-((4′-(trifluoromethyl)biphenyl-4-yl)methyl)-4H-1,2,4-triazole

A mixture of intermediate D31 (0.62 g, 1 equiv), 4-(trifluoromethyl)phenylboronic acid (0.466 g, 1.1 equiv), Cs₂CO₃ (1.45 g, 2 equiv) and Pd(PPh)₃ (0.13 g, 0.05 equiv) in dioxane (8 ml) was refluxed for 12 h in a nitrogen atmosphere. The inorganic salts were filtered off and washed with dioxane. The filtrate was concentrated and mixed with silica gel and then purified by column chromatography to give a solid (0.62 g). H-NMR (CDCl₃, 300 MHz) δ 1.00 (t, 3H), 1.81 (m, 2H), 2.68 (t, 2H), 5.15 (s, 2H), 7.20 (d, 2H), 7.61 (d, 2H), 7.69 (m, 4H), 8.13 (s, 1H).

### Intermediate D33—(5-n-propyl-4-((4′-(trifluoromethyl)biphenyl-4-yl)methyl)-4H-1,2,4-triazol-3-yl)methanol

Following a procedure similar to that described for the preparation of intermediate D21 except that D32 was used in place of D20. H-NMR (CDCl₃, 400 MHz) δ0.97 (t, 3H), 1.76 (m, 2H), 2.62 (t, 2H), 4.66 (s, 1H), 4.76 (s, 2H), 5.35 (s, 2H), 7.18 (d, 2H), 7.60 (d, 2H), 7.68 (d, 2H), 7.72 (d, 2H).

### Intermediate D34—(5-isopropyl-4-((4′-(trifluoromethyl)biphenyl-4-yl)methyl)-4H-1,2,4-triazol-3-yl)methanol

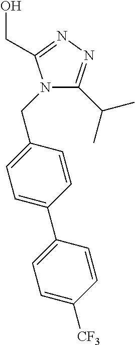 [SMILES: CC(C)c1nnc(CO)n1Cc1ccc(-c2ccc(C(F)(F)F)cc2)cc1]

Following a procedure similar to that described for the preparation of intermediate D33 except that ethyl isobutyrate was used in place of methyl butyrate. H-NMR (CDCl₃, 400 MHz) δ1.30 (d, 6H), 2.30 (s, 1H), 2.92 (m, 1H), 4.67 (s, 2H), 5.34 (s, 2H), 7.13 (d, 2H), 7.59 (d, 2H), 7.68 (m, 4H).

### Intermediate D35—(5-cyclopropyl-4-((4′-(trifluoromethyl)biphenyl-4-yl)methyl)-4H-1,2,4-triazol-3-yl) methanol

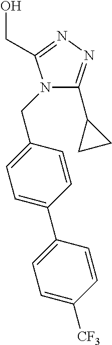 [SMILES: OCc1nnc(C2CC2)n1Cc1ccc(-c2ccc(C(F)(F)F)cc2)cc1]

Following a procedure similar to that described for the preparation of intermediate D33 except that ethyl cyclopropanecarboxylate was used in place of methyl butyrate.

### Example 64

### 2-(4-fluorobenzylthio)-1-((5-methyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

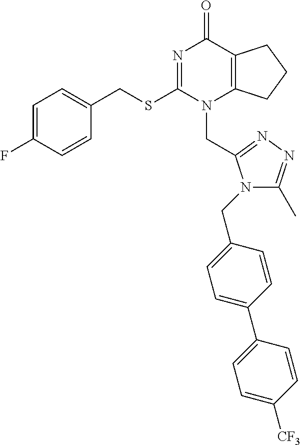 [SMILES: Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 62 except that D28 was used in place of D21. ¹H-NMR (CDCl₃, 300 MHz) δ1.92 (m, 2H), 2.50 (s, 3H), 2.56 (t, 2H, J=7.2), 2.73 (t, 2H, J=7.2), 4.46 (s, 2H), 5.11 (s, 2H), 5.18 (s, 2H), 6.82 (d, 2H, J=7.8), 6.95 (t, 2H, J=8.7), 7.34 (dd, 2H, J=5.4, 8.7), 7.44 (d, 2H, J=8.1), 7.62 (d, 2H, J=8.1), 7.70 (d, 2H, J=8.7); MS (ESI): 606 (M+H).

### Example 65

### 2-(4-fluorobenzylthio)-5-((1-methyl-1H-pyrazol-4-yl)methyl)-1-((5-methyl-4-((4′-(trifluoromethyl)biphenyl-4-yl)methyl)-4H-1,2,4-triazol-3-yl)methyl)pyrimidin-4(1H)-one

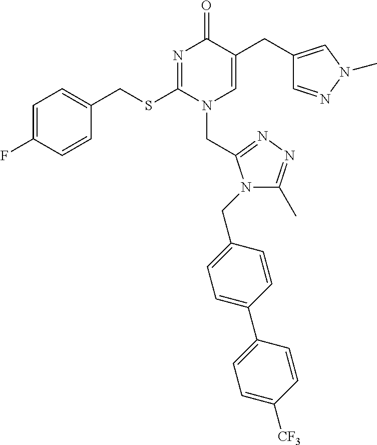 [SMILES: Cc1nnc(Cn2cc(Cc3cnn(C)c3)c(=O)nc2SCc2ccc(F)cc2)n1Cc1ccc(-c2ccc(C(F)(F)F)cc2)cc1]

Following a procedure similar to that described for the preparation of example 29 except that D28 was used in place of C3. ¹H-NMR (CDCl₃, 300 MHz) δ2.46 (s, 3H), 3.37 (s, 2H), 3.78 (s, 3H), 4.39 (s, 2H), 5.01 (s, 2H), 5.13 (s, 2H), 6.77 (d, 2H, J=8.1), 6.97 (m, 3H), 7.34 (m, 6H), 7.65 (m, 4H); MS (ESI): 660 (M+H).

### Example 66

### 2-(4-fluorobenzylthio)-1-((5-propyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

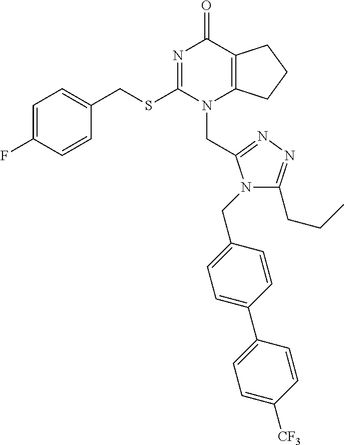 [SMILES: CCCc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 62 except that D33 was used in place of D21. ¹H-NMR (CDCl₃, 300 MHz) δ1.04 (t, 3H, J=7.5), 1.82-2.00 (m, 4H), 2.56 (t, 2H, J=7.2), 2.75 (m, 4H), 4.42 (s, 2H), 5.16 (s, 2H), 5.25 (s, 2H), 6.86 (d, 2H, J=8.1), 6.93 (t, 2H, J=8.7), 7.32 (dd, 2H, J=5.7, 8.4), 7.45 (d, 2H, J=7.8), 7.61 (d, 2H, J=8.1), 7.71 (d, 2H, J=8.4).

### Example 67

### 2-(4-fluorobenzylthio)-1-((5-isopropyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

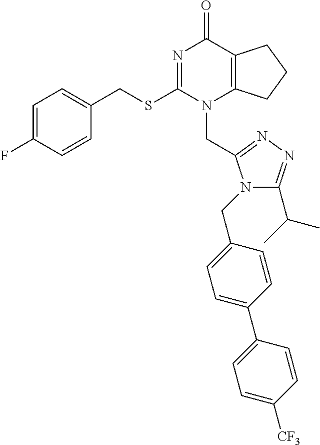 [SMILES: CC(C)c1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 62 except that D34 was used in place of D21. ¹H-NMR (CDCl₃, 300 MHz) δ1.42 (d, 6H, J=6.9), 1.93 (m, 2H), 2.57 (t, 2H, J=7.5), 2.76 (t, 2H, J=7.5), 2.99 (m, 1H), 4.43 (s, 2H), 5.10 (s, 2H), 5.25 (s, 2H), 6.82 (d, 2H, J=8.1), 6.94 (t, 2H, J=8.7), 7.33 (dd, 2H, J=8.4, 5.4), 7.44 (d, 2H, J=7.8), 7.61 (d, 2H, J=8.4), 7.70 (d, 2H, J=8.7).

### Example 68

### 1-((5-cyclopropyl-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5 H)-one

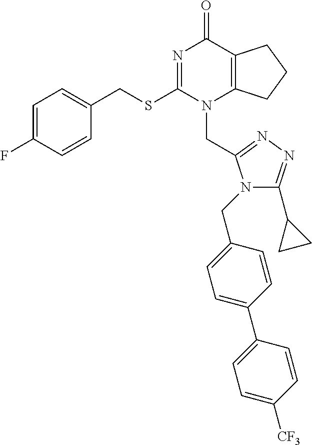 [SMILES: O=c1nc(SCc2ccc(F)cc2)n(Cc2nnc(C3CC3)n2Cc2ccc(-c3ccc(C(F)(F)F)cc3)cc2)c2c1CCC2]

Following a procedure similar to that described for the preparation of example 62 except that D35 was used in place of D21. ¹H-NMR (CDCl₃, 300 MHz) δ1.07 (m, 2H), 1.23 (m, 2H), 1.71 (m, 2H), 1.90 (m, 2H), 2.54 (t, 2H), 2.70 (t, 2H), 2.99 (m, 1H), 4.47 (s, 2H), 5.10 (s, 2H), 5.31 (s, 2H), 6.87 (d, 2H), 6.96 (t, 2H), 7.35 (dd, 2H), 7.45 (d, 2H), 7.63 (d, 2H), 7.71 (d, 2H).

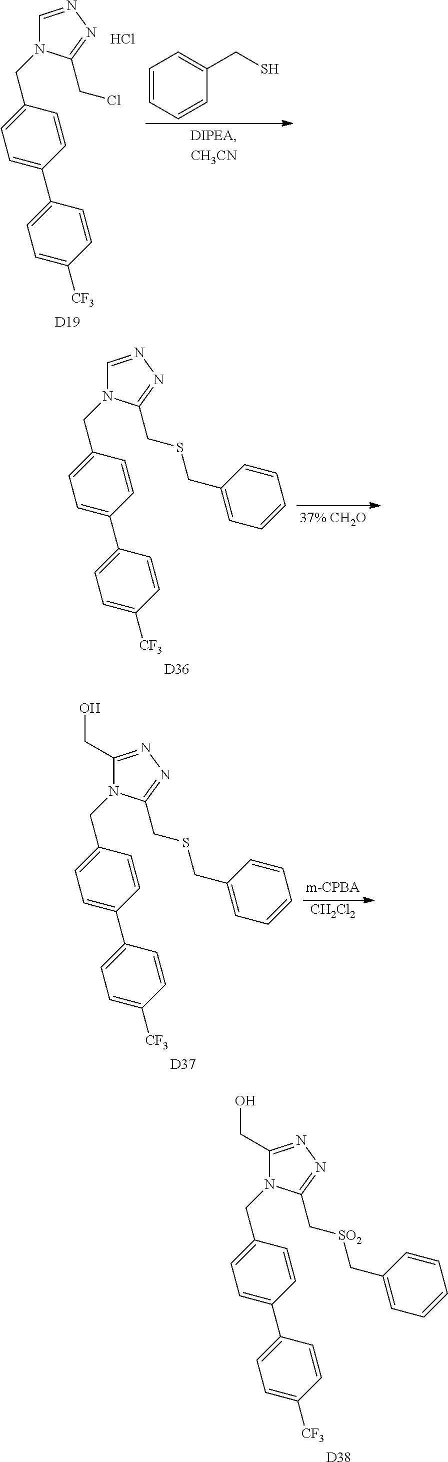

Intermediate D36—3-(benzylthiomethyl)-4-((4′-(trifluoromethyl)biphenyl-4-yl)methyl)-4H-1,2,4-tria zole

A mixture of D19 (610 mg), phenylmethanethiol (0.18 ml) and DIPEA (0.65 ml) in acetonitrile (10 ml) was heated at 60° C. for 3 h. Acetonitrile was evaporated and the residue was dissolved in EA, washed with brine three times, dried over anhydrous Na₂SO₄ and purified by flash chromatography to give the title compound as a light yellow solid (630 mg). ¹H-NMR (CDCl₃, 300 MHz) δ3.70 (s, 2H), 3.71 (s, 2H), 5.22 (s, 2H), 7.21 (d, 2H, J=8.4), 7.33 (m, 5H), 7.65 (d, 2H, J=8.4), 7.70 (d, 2H, J=8.4), 8.16 (s, 1H).

### Intermediate D37—(5-(benzylthiomethyl)-4-((4′-(trifluoromethyl)biphenyl-4-yl)methyl)-4H-1,2,4-triazol-3-yl)methanol

Following a procedure similar to that described for the preparation of D21 except that D36 was used in place of D20. ¹H-NMR (d₆-DMSO, 300 MHz) δ3.65 (s, 2H), 3.70 (s, 2H), 4.52 (d, 2H, J=5.7), 5.36 (s, 2H), 5.64 (t, 1H, J=5.7), 7.21 (d, 2H, J=8.1), 7.29 (m, 5H), 7.70 (d, 2H, J=8.4), 7.80 (d, 2H, J=8.4), 7.87 (d, 2H, J=8.7).

### Intermediate D38—(5-(benzylsulfonylmethyl)-4-((4′-(trifluoromethyl)biphenyl-4-yl)methyl)-4H-1,2,4-triazol-3-yl)methanol

To an ice cold suspension of D37 (0.7 g, 1 equiv) in DCM (15 ml) was added m-CPBA (1.07 g, 70-75% pure, 3 equiv) and the mixture was stirred at room temperature for 2 h before it was quenched with saturated NaHCO₃ solution until no more bubbles was generated. The organic layer was separated and the aqueous phase was extracted with DCM again. The combined organic phase was washed with saturated brine, dried over anhydrous Na₂SO₄ and purified by flash chromatography to give the title compound as a white solid (470 mg). ¹H-NMR (CDCl₃, 300 MHz) δ2.90 (s, 1H), 4.18 (s, 2H), 4.37 (s, 2H), 4.87 (s, 2H), 5.54 (s, 2H), 7.19 (d, 2H, J=7.8), 7.36 (m, 3H), 7.53 (d, 2H, J=8.1), 7.58 (m, 4H), 7.67 (d, 2H, J=8.7).

### Example 69

### 1-((5-(benzylsulfonylmethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

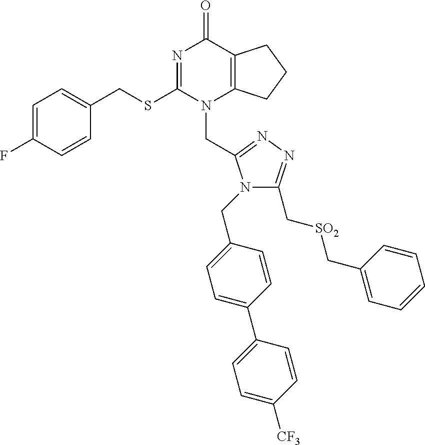 [SMILES: O=c1nc(SCc2ccc(F)cc2)n(Cc2nnc(CS(=O)(=O)Cc3ccccc3)n2Cc2ccc(-c3ccc(C(F)(F)F)cc3)cc2)c2c1CCC2]

Following a procedure similar to that described for the preparation of example 62 except that D38 was used in place of D21. ¹H-NMR (CDCl₃, 400 MHz) δ2.04 (m, 2H), 2.71 (t, 2H, J=7.2), 2.83 (t, 2H, J=7.2), 4.28 (s, 2H), 4.40 (s, 2H), 4.44 (s, 2H), 5.08 (s, 2H), 5.42 (s, 2H), 6.91 (t, 2H, J=8.4), 6.96 (d, 2H, J=8.0), 7.29 (dd, 2H, J=8.4, 5.6), 7.43 (m, 5H), 7.58 (d, 2H, J=8.4), 7.66 (m, 2H), 7.70 (d, 2H, J=8.0).

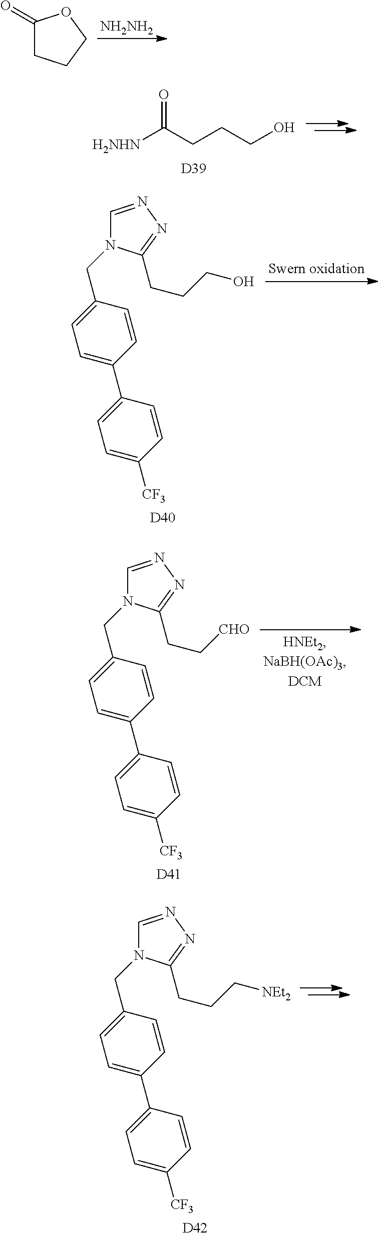

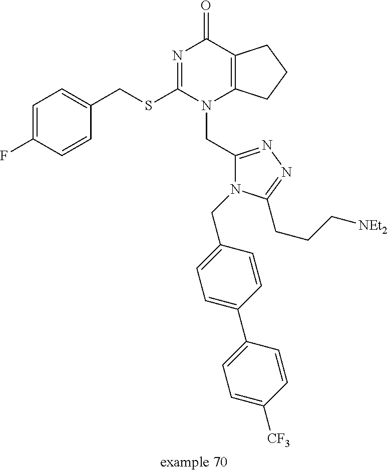

### Intermediate D39—4-hydroxybutanehydrazide

Following a procedure similar to that described for the preparation of D15 except that dihydrofuran-2(3H)-one was used in place of methyl 2-hydroxyacetate. ¹H-NMR (d₆-DMSO, 300 MHz) δ 1.61 (m, 2H), 2.03 (t, 2H), 3.45 (m, 2H), 4.13 (s, 2H), 4.44 (t, 1H, —OH), 8.90 (s, 1H).

### Intermediate D40—3-(4-((4′-(trifluoromethyl)biphenyl-4-yl)methyl)-4H-1,2,4-triazol-3-yl)propan-1-ol

Following a procedure similar to that described for the preparation of D32 except that D39 was used in place of D29. H-NMR (CDCl₃, 300 MHz) δ 2.06 (m, 2H), 2.85 (t, 2H), 3.74 (t, 2H), 5.18 (s, 2H), 7.23 (d, 2H), 7.62 (d, 2H), 7.68 (m, 4H), 8.14 (s, 1H), 9.86 (s, 1H)

### Intermediate D41—3-(4-((4′-(trifluoromethyl)biphenyl-4-yl)methyl)-4H-1,2,4-triazol-3-yl)propanal

To a solution of oxalyl chloride (205 μl, 1.5 equiv) in DCM (5 ml) placed in a cold trap of −60° C. under nitrogen was added a solution of DMSO (305 μl, 3 equiv) in DCM (5 ml) over 5 min via a constant pressure funnel, followed by addition of a solution of D40 (0.52 g, 1 equiv) in DCM (5 ml) and DMSO (105 μl) over 5 min. The resulting mixture was stirred for 10 min and then a solution of NEt₃ (800 μl, 4 equiv) in DCM (5 ml) was added over 5 min. The reaction was stirred for a further 10 min before it was quenched with saturated NH₄Cl solution. The DCM layer was separated, washed with brine twice, dried over anhydrous NaSO₄ and purified by flash chromatography to give the title compound as a jelly (360 mg). H-NMR (CDCl₃, 300 MHz) δ 2.93 (t, 2H), 3.19 (t, 2H), 5.24 (s, 2H), 7.21 (d, 2H), 7.61 (d, 2H), 7.68 (m, 4H), 8.13 (s, 1H)

### Intermediate D42—N,N-diethyl-3-(4-((4′-(trifluoromethyl)biphenyl-4-yl)methyl)-4H-1,2,4-triazol-3-yl)propan-1-amine

To an ice cold solution of D41 (360 mg, 1 equiv) and diethylamine (115 μl, 1.1 equiv) in DCM (5 ml) was added NaBH(OAc)₃ (318 mg, 1.5 equiv) and the mixture was stirred for 1 h before it was quenched with saturated NaHCO₃ solution. The DCM layer was separated, dried over anhydrous NaSO₄ and purified by flash chromatography to give the title compound as a jelly (270 mg). H-NMR (CDCl₃, 300 MHz) δ 1.12 (t, 6H), 2.13 (m, 2H), 2.78 (m, 8H), 5.19 (s, 2H), 7.22 (d, 2H), 7.60 (d, 2H), 7.68 (m, 4H), 8.14 (s, 1H).

### Example 70

### 1-((5-(3-(diethylamino)propyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

Following a procedure similar to that described for the preparation of example 62 except that D42 was used in place of D20. ¹H-NMR (CDCl₃, 300 MHz) δ 1.35 (t, 6H), 2.00 (m, 2H), 2.41 (m, 2H), 2.69 (t, 2H), 2.83 (t, 2H), 3.02 (t, 2H), 3.10 (q, 4H), 3.22 (t, 2H), 4.42 (s, 2H), 5.07 (s, 2H), 5.33 (s, 2H), 6.91 (t, 2H), 6.97 (d, 2H), 7.29 (dd, 2H), 7.44 (d, 2H), 7.59 (d, 2H), 7.70 (d, 2H); MS (ESI): 705 (M+H).

### Example 71

### 1-((5-(4-(diethylamino)butyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

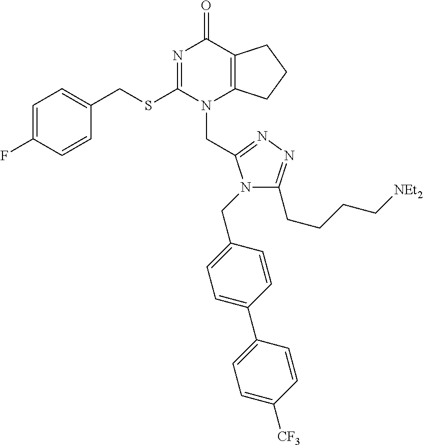

Following a procedure similar to that described for the preparation of example 70 except that tetrahydro-2H-pyran-2-one was used in place of dihydrofuran-2(3H)-one. ¹H-NMR (CDCl₃, 300 MHz) δ 1.23 (t, 6H), 1.89 (m, 6H), 2.63 (t, 2H), 2.79 (m, 6H), 2.89 (q, 4H), 4.42 (s, 2H), 5.08 (s, 2H), 5.24 (s, 2H), 6.88 (d, 2H), 6.91 (t, 2H), 7.30 (dd, 2H), 7.44 (d, 2H), 7.59 (d, 2H), 7.70 (d, 2H); MS (ESI): 719 (M+H)⁺.

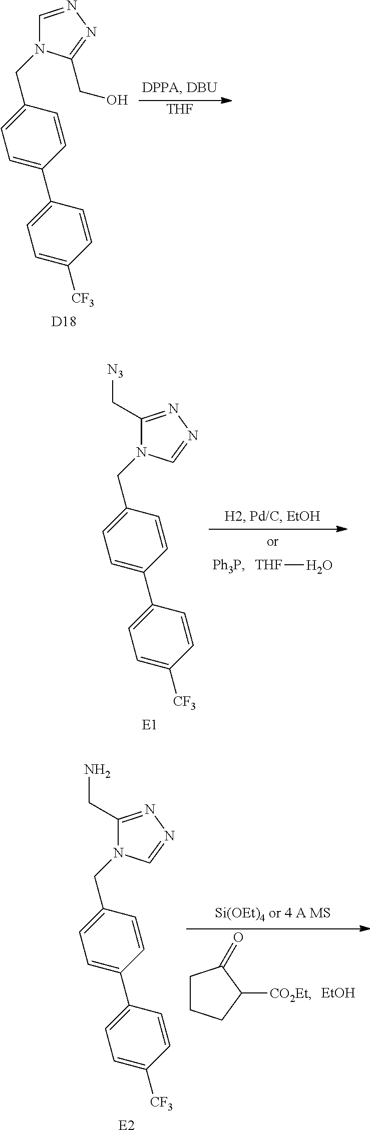

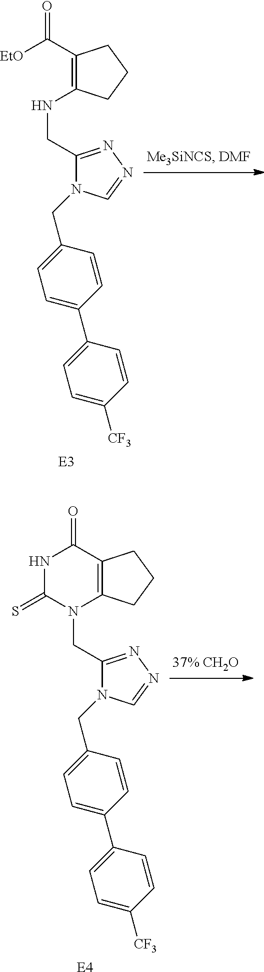 [SMILES: CCOC(=O)C1=C(NCc2nncn2Cc2ccc(-c3ccc(C(F)(F)F)cc3)cc2)CCC1 ; "E3" ; O=c1[nH]c(=S)n(Cc2nncn2Cc2ccc(-c3ccc(C(F)(F)F)cc3)cc2)c2c1CCC2 ; "E4"]

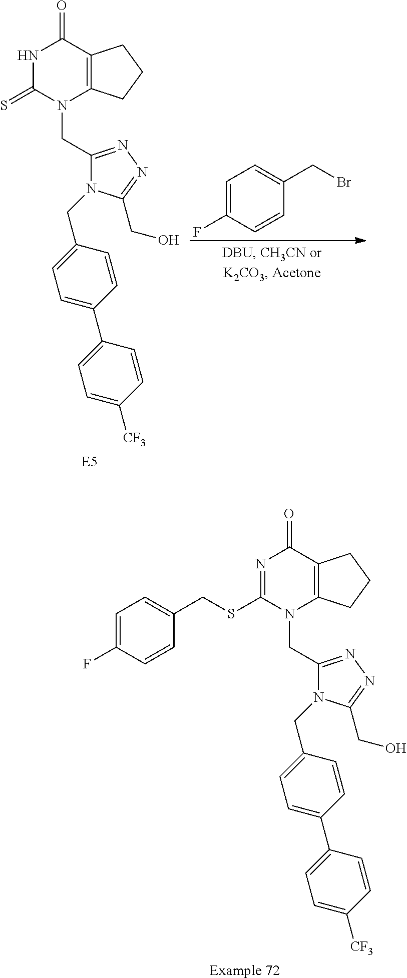

### Intermediate E1—3-(azidomethyl)-4-((4′-(trifluoromethyl)biphenyl-4-yl)methyl)-4H-1,2,4-triazole

A mixture of D18 (9.6 g, 1 equiv), DPPA (7.5 ml, 1.2 equiv) and DBU (5.6 ml, 1.3 equiv) in THF (100 ml) was refluxed for 3 h under nitrogen. TLC detection showed that the reaction was complete. After cooled, the mixture was then poured into saturated NH₄Cl solution and extracted with EA. The combined organic phase was washed with brine twice, dried and then purified by flash chromatography to give the title compound as a jelly (8.7 g). ¹H-NMR (CDCl₃, 300 MHz) δ 4.54 (s, 2H), 5.31 (s, 2H), 7.28 (d, 2H), 7.62 (d, 2H), 7.66 (d, 2H), 7.70 (d, 2H), 8.40 (s, 1H).

### Intermediate E2—(4-((4′-(trifluoromethyl)biphenyl-4-yl)methyl)-4H-1,2,4-triazol-3-yl)methanamine

(Method 1)

To a solution of E1 (10.6 g, 1 equiv) in THF—H₂O (100-10 ml) was added Ph₃P (11.6 g, 1.5 equiv) and the mixture was stirred at room temperature overnight and the reaction was complete. The mixture was then mixed with silica gel, evaporated to dryness and loaded on a short silica column. The column was first eluted with EA to get rid of Ph₃P and Ph₃P═O and then eluted with methanol to afford the target product portion. The methanol solution so collected was evaporated to dryness and the residue was dissolved in a mixed solvent of DCM/MeOH (15:1). Insoluble silica gel was removed by filtration and the filtrate was evaporated to dryness to give the title compound as a solid (8.3 g).

(Method 2)

To a solution of intermediate E1 (8.7 g) in absolute ethanol (100 ml) was added 10% palladium on charcoal (1.3 g) and the mixture was stirred under hydrogen (1 atm.) overnight at room temperature. The mixture was filtered and the filtrate was concentrated to afford the title compound as a solid (8.1 g)

### Intermediate E3—ethyl 2-((4-((4′-(trifluoromethyl)biphenyl-4-yl)methyl)-4H-1,2,4-triazol-3-yl)methylamino)cyclopent-1-enecarboxylate

(Method 1)

To a solution of E2 (2.8 g, 1 equiv) in absolute ethanol (20 ml) were added ethyl 2-oxocyclopentanecarboxylate (1.36 ml, 1.1 equiv) and tetraethyl orthosilicate (3.7 ml, 2 equiv). The mixture was refluxed under nitrogen for 5 h until the completion of the reaction monitored by TLC. Silica gel was added to the solution and the mixture was evaporated to dryness, then purified by column chromatography (DCM/MeOH=25:1) to give the title compound as a solid (3.9 g).

(Method 2) To a solution of E2 (2.63 g, 1 equiv) in absolute ethanol (20 ml) were added ethyl 2-oxocyclopentanecarboxylate (1.2 ml, 1.0 equiv) and 4 Å dried molecular sieve (10 g). The mixture was refluxed for 10 h under nitrogen and then filtered to remove the molecular sieve. The filtrate was concentrated to remove most of the solvent and then stored in the refrigerator for a few hours. The precipitate thus obtained was collected by filtration, dried to give the title compound as a solid (3.4 g). ¹H-NMR (CDCl₃, 300 MHz) δ1.23 (t, 3H, J=7.2), 1.76 (m, 2H), 2.47 (m, 4H), 4.09 (q, 2H, J=7.2), 4.44 (d, 2H, J=6.3), 5.26 (s, 2H), 7.22 (d, 2H, J=8.1), 7.60 (d, 2H, J=8.1), 7.68 (m, 5H), 8.17 (s, 1H).

### Intermediate E4—2-thioxo-1-((4-((4′-(trifluoromethyl)biphenyl-4-yl)methyl)-4H-1,2,4-triazol-3-yl)methyl)-2,3,6,7-tetrahydro-1H-cyclopenta[d]pyrimidin-4(5H)-one

A mixture of E3 (2.0 g, 1 equiv), isothiocyanatotrimethylsilane (2.1 ml, 3.5 equiv) in anhydrous DMF (3 ml) was heated at 140° C. for 4 h under nitrogen. After cooled, the mixture was placed in an ice bath and quenched by addition of saturated NaHCO₃ solution, diluted with EA (30 ml) and water (20 ml), and then stirred for 10 min. The organic layer was separated and the aqueous phase was extracted again with EA (30 ml) once. The combined organic phase was concentrated to about 20 ml and then stored in refrigerator. The precipitate thus obtained was filtered off and dried to give the title compound (1.6 g). ¹H-NMR (d₆-DMSO, 300 MHz) δ1.94 (m, 2H), 2.52 (t, 2H), 2.85 (t, 2H, J=7.5), 5.48 (s, 2H), 5.60 (s, 2H), 7.33 (d, 2H, J=8.1), 7.73 (d, 2H, J=8.4), 7.81 (d, 2H, J=8.4), 7.88 (d, 2H, J=8.7), 8.68 (s, 1H), 12.56 (s, 1H).

### Intermediate E5—1-((5-(hydroxymethyl)-4-((4′-(trifluoromethyl)biphenyl-4-yl)methyl)-4H-1,2,4-triazol-3-yl)methyl)-2-thioxo-2,3,6,7-tetrahydro-1H-cyclopenta[d]pyrimidin-4 (5H)-one

A solution of E4 (1.6 g) in formalin (15 ml, 37%) was refluxed for 8 h until the completion of the reaction monitored by TLC. Then the mixture was cooled and precipitates formed, which was collected by filtration, washed with water several times and dried to give the title compound (1.2 g). ¹H-NMR (d₆-DMSO, 300 MHz) δ1.94 (m, 2H), 2.50 (t, 2H), 2.82 (t, 2H), 4.65 (d, 2H), 4.79 (t, 1H, —OH), 5.50 (s, 2H), 5.55 (s, 2H), 7.26 (d, 2H), 7.72 (d, 2H), 7.81 (d, 2H), 7.88 (d, 2H), 12.56 (s, 1H); MS (ESI): 512(M−H).

### Example 72

### 2-(4-fluorobenzylthio)-1-((5-(hydroxymethyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

(Method 1)

A mixture of E5 (1.2 g, 1 equiv), 1-(bromomethyl)-4-fluorobenzene (0.32 ml, 1.1 equiv), DBU (0.52 ml, 1.5 equiv) and KI (20 mg, 0.05 equiv) in acetonitrile (10 ml) was stirred at room temperature overnight and TLC detection showed that the reaction was complete. Acetonitrile was evaporated under reduced pressure and the residue was diluted with EA (10 ml) and water (10 ml). The resulting mixture was stirred for 0.5 h and the precipitate thus obtained was collected by filtration, washed with water and EA, and then dried to give the title compound (1.16 g).

(Method 2)

A mixture of E5 (1.08 g, 1.1 equiv), 1-(bromomethyl)-4-fluorobenzene (0.29 ml, 1.1 equiv) and anhydrous K₂CO₃ (0.44 g, 1.5 equiv) in acetone (10 ml) was refluxed for 1 h and TLC detection showed that the reaction was complete. Acetone was evaporated under reduced pressure and the residue was diluted with EA (10 ml) and water (10 ml). The resulting mixture was stirred for 0.5 h and the precipitate thus obtained was collected by filtration, washed with water and EA, and then dried to give the title compound (0.63 g). ¹H-NMR (d₆-DMSO, 400 MHz) δ1.90 (m, 2H), 2.54 (t, 2H), 2.67 (t, 2H), 4.27 (s, 2H), 4.64 (s, 2H), 5.14 (s, 2H), 5.44 (s, 2H), 5.80 (vbrs, 1H), 6.99 (t, 2H, J=8.4), 7.28 (d, 2H), 7.31 (dd, 2H), 7.64 (d, 2H), 7.80 (m, 4H).

### Example 73

### 2-(4-fluorobenzylthio)-1-((4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

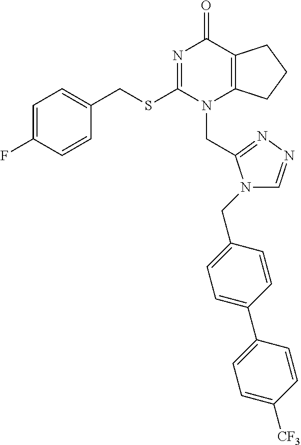 [SMILES: O=c1nc(SCc2ccc(F)cc2)n(Cc2nncn2Cc2ccc(-c3ccc(C(F)(F)F)cc3)cc2)c2c1CCC2]

Following a procedure similar to that described for the preparation of example 72 except that E4 was used in place of E5. ¹H-NMR (d₆-DMSO, 400 MHz) δ1.90 (m, 2H), 2.54 (t, 2H, J=7.2), 2.69 (t, 2H, J=7.2), 4.29 (s, 2H), 5.24 (s, 2H), 5.39 (s, 2H), 7.02 (t, 2H, J=8.4), 7.32 (m, 4H), 7.67 (d, 2H, J=8.8), 7.82 (m, 4H), 8.74 (s, 1H).

### Example 74

### 5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazole-3-carbaldehyde

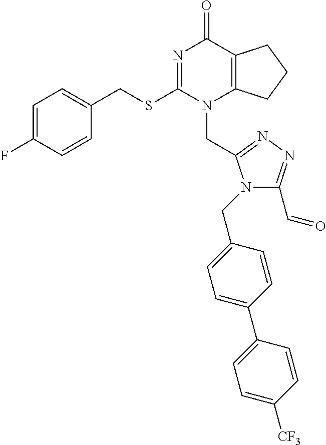 [SMILES: O=Cc1nnc(Cn2c(SCc3ccc(F)cc3)nc(=O)c3c2CCC3)n1Cc1ccc(-c2ccc(C(F)(F)F)cc2)cc1]

To a solution of the compound of Example 72 (700 mg, 1 equiv) in dioxane (10 ml) was added activated MnO₂ (0.6 g, 6 equiv) and the mixture was stirred at 70-80° C. for 3 h. MnO₂ was then removed by filtration and the filtrate was purified by column chromatography (DCM/MeOH=20:1) to give the title compound as a solid (540 mg). ¹H-NMR (CDCl₃, 300 MHz) δ1.99 (m, 2H), 2.71 (m, 4H), 4.42 (s, 2H), 5.13 (s, 2H), 5.73 (s, 2H), 6.90 (m, 2H), 7.03 (d, 2H), 7.28 (dd, 2H), 7.47 (d, 2H), 7.59 (d, 2H), 7.70 (d, 2H).

### Example 75

### 1-((5-((dimethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

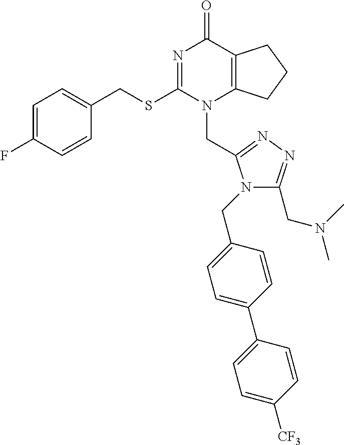 [SMILES: CN(C)Cc1nnc(Cn2c(SCc3ccc(F)cc3)nc(=O)c3c2CCC3)n1Cc1ccc(-c2ccc(C(F)(F)F)cc2)cc1]

A mixture of the compound of Example 74 (400 mg, 1 equiv), dimethylamine hydrochloride (105 mg, 2 equiv), DIPEA (215 μl, 2 equiv) and NaHB(OAc)₃ (205 mg, 1.5 equiv) in DCM (8 ml) was stirred at room temperature. After the completion of the reaction monitored by TLC, the mixture was transferred into a separating funnel, washed with brine three times, dried and purified by column chromatography (DCM/MeOH=10:1) to give the title compound as a solid (280 mg). ¹H-NMR (CDCl₃, 300 MHz) δ1.96 (m, 2H), 2.32 (s, 6H), 2.63 (t, 2H, J=7.2), 2.75 (t, 2H, J=7.2), 3.70 (s, 2H), 4.44 (s, 2H), 5.03 (s, 2H), 5.49 (s, 2H), 6.93 (m, 4H), 7.31 (dd, 2H, J=8.4, 5.4), 7.45 (d, 2H, J=8.4), 7.61 (d, 2H, J=8.1), 7.70 (d, 2H, J=8.1); MS (ESI): 649 (M+H).

### Example 76

### N-(4-fluorobenzyl)-1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4 H-1,2,4-triazol-3-yl)-N,N-dimethylmethanaminium bromide

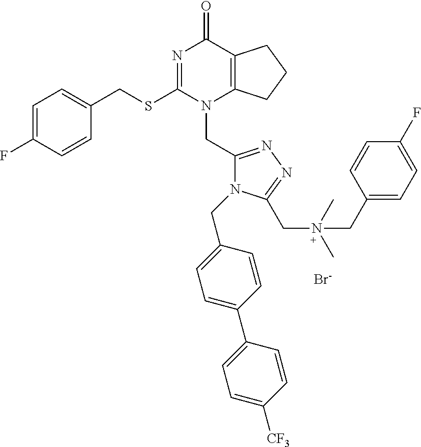

Following a procedure similar to that described for the preparation of example 47 except that example 75 was used in place of example 46. ¹H-NMR (d₆-DMSO, 300 MHz) δ1.92 (m, 2H), 2.55 (t, 2H, J=6.6), 2.75 (t, 2H, J=6.6), 3.08 (s, 6H), 4.29 (s, 2H), 4.48 (s, 2H), 4.89 (s, 2H), 5.20 (s, 2H), 5.64 (s, 2H), 6.99 (t, 2H, J=8.4), 7.21 (d, 2H, J=7.8), 7.30 (t, 2H, J=7.8), 7.38 (t, 2H, J=8.7), 7.65 (d, 2H, J=8.4), 7.73 (t, 2H, J=7.8), 7.81 (m, 4H); MS (ESI): 757 (M-Br).

### Example 77

### 1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)-N,N,N-trimethylmethanaminium iodide

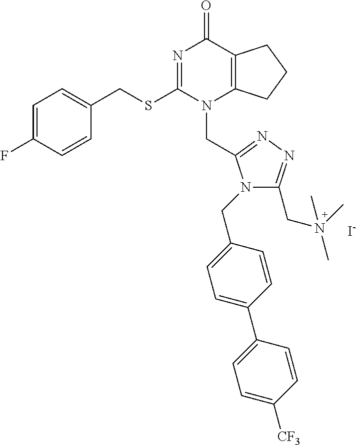

To a solution of the compound of example 75 (65 mg, 1 equiv) in DCM (3 ml) was added CH₃I (19 μl, 3 equiv) and the plug was plug was screwed tightly. The mixture was stirred at room temperature overnight. Solvent was evaporated under reduced pressure and the residue was recrystallized from acetone to give the title compound as a light yellow solid (40 mg). ¹H-NMR (d₆-DMSO, 300 MHz) δ1.91 (m, 2H), 2.54 (t, 2H), 2.71 (t, 2H, J=6.9), 3.21 (s, 9H), 4.28 (s, 2H), 4.93 (s, 2H), 5.14 (s, 2H), 5.56 (s, 2H), 6.99 (t, 2H, J=8.7), 7.19 (d, 2H, J=7.8), 7.30 (dd, 2H, J=8.1, 5.7), 7.66 (d, 2H, J=8.1), 7.81 (m, 4H).

### Example 78

### 1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)-N,N,N-trimethylmethanaminium bromide

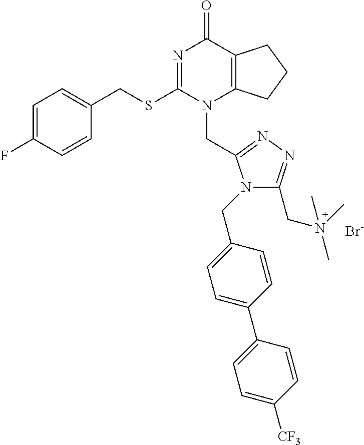

To solution of the compound of Example 75 (90 mg) in acetone (2 ml) in a flask sealed with rubber plug and placed in a cold trap at −5° C. was added CH₃Br (0.5 ml) dropwise with an injector. The mixture was stirred at room temperature overnight and the solid thus formed was collected by filtration, washed with acetone, and then dried in vacuo to give the title compound as a white solid (70 mg).

### Example 79

### 1-((5-((diethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

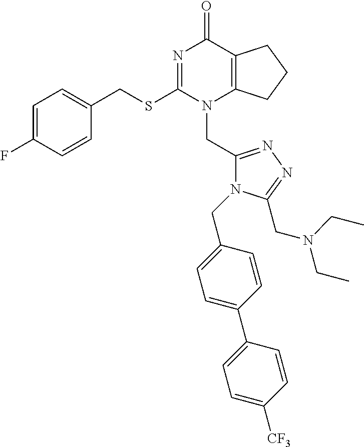 [SMILES: CCN(CC)Cc1nnc(Cn2c(SCc3ccc(F)cc3)nc(=O)c3c2CCC3)n1Cc1ccc(-c2ccc(C(F)(F)F)cc2)cc1]

A mixture of the compound of Example 74 (200 mg, 1 equiv), diethylamine (40 μl, 1.2 equiv) and NaHB(OAc)₃ (103 mg, 1.5 equiv) in DCM (5 ml) was stirred at room temperature. After the completion of the reaction monitored by TLC, the mixture was transferred into a separating funnel, washed with brine three times, dried and purified by column chromatography (DCM/MeOH=15:1) to give the title compound as a colloidal solid (160 mg). ¹H-NMR (CDCl₃, 300 MHz) δ0.99 (t, 6H, J=7.2), 1.97 (m, 2H), 2.57-2.65 (m, 6H), 2.77 (t, 2H, J=7.2), 3.82 (s, 2H), 4.44 (s, 2H), 5.02 (s, 2H), 5.53 (s, 2H), 6.93 (m, 4H), 7.30 (m, 2H), 7.45 (d, 2H, J=8.1), 7.63 (d, 2H, J=8.4), 7.70 (d, 2H, J=8.1); MS (ESI): 677 (M+H).

### Example 80

### 1-((5-((diethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one hydrochloride

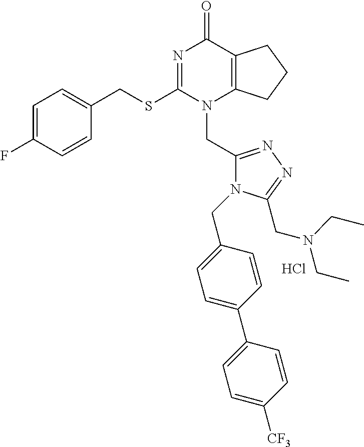 [SMILES: CCN(CC)Cc1nnc(Cn2c(SCc3ccc(F)cc3)nc(=O)c3c2CCC3)n1Cc1ccc(-c2ccc(C(F)(F)F)cc2)cc1 ; Cl]

To an ice cold solution of the compound of example 79 (105 mg, 1 equiv) in isopropanol (2 ml) was added concd. HCl (14 μl, about 1 equiv), and the mixture was stirred for 0.5 h. Solvent was then evaporated under reduced pressure and to the residue was added DCM (3 ml) and the residue was smashed by ultrasonic. The solid thus formed was collected by filtration, dried in vacuo to give the title compound as a white solid (60 mg). ¹H-NMR (d₆-DMSO, 300 MHz) δ1.23 (m, 6H), 1.89 (m, 2H), 2.55 (t, 2H, J=7.2), 2.72 (t, 2H, J=7.2), 3.26 (m, 4H), 4.31 (s, 2H), 4.56 (s, 2H), 5.23 (s, 2H), 5.59 (s, 2H), 7.02 (t, 2H, J=8.7), 7.22 (d, 2H, J=8.1), 7.33 (dd, 2H, J=8.7, 5.4), 7.67 (d, 2H, J=8.1), 7.82 (m, 4H), 11.06 (s, 1H).

### Example 81

### 1-((5-(((4-fluorobenzyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

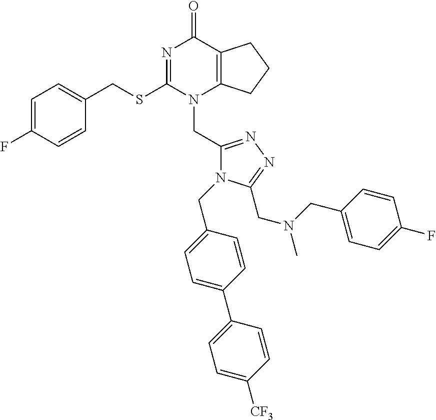 [SMILES: CN(Cc1ccc(F)cc1)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that 1-(4-fluorophenyl)-N-methylmethanamine was used instead of diethylamine ¹H-NMR (CDCl₃, 300 MHz) δ1.95 (m, 2H), 2.34 (s, 3H), 2.62 (t, 2H, J=7.2), 2.73 (t, 2H, J=7.2), 3.61 (s, 2H), 3.75 (s, 2H), 4.44 (s, 2H), 5.01 (s, 2H), 5.31 (s, 2H), 6.80 (d, 2H, J=7.8), 6.93 (m, 4H), 7.18 (m, 2H), 7.30 (dd, 2H, J=8.1, 5.7), 7.41 (d, 2H, J=7.8), 7.62 (d, 2H, J=8.1), 7.73 (d, 2H, J=8.4); MS (ESI): 743 (M+H).

### Example 82

### 2-(4-fluorobenzylthio)-1-((5-((isopropyl(methyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

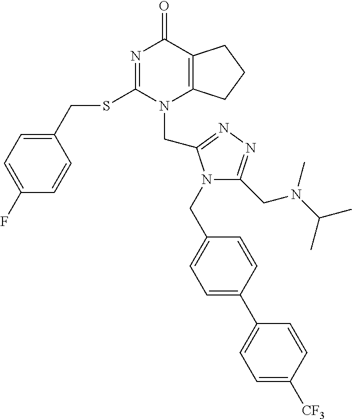 [SMILES: CC(C)N(C)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that N-methylpropan-2-amine was used instead of diethylamine. ¹H-NMR (CDCl₃, 400 MHz) δ 1.01 (d, 6H, J=6.8), 1.96 (m, 2H), 2.17 (s, 3H), 2.63 (t, 2H), 2.75 (t, 2H), 2.87 (m, 1H), 3.79 (s, 2H), 4.44 (s, 2H), 5.02 (s, 2H), 5.48 (s, 2H), 6.93 (m, 4H), 7.30 (dd, 2H), 7.45 (d, 2H), 7.61 (d, 2H, J=8.0), 7.70 (d, 2H, J=8.4).

### Example 83

### (±)-1-((5-(((1-ethylpyrrolidin-2-yl)methylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

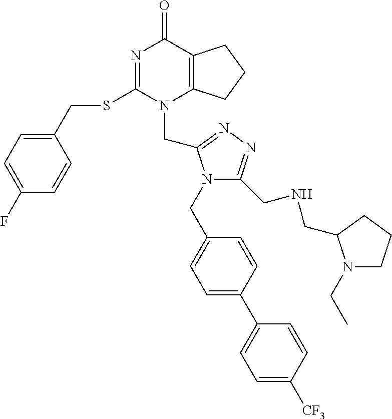 [SMILES: CCN1CCCC1CNCc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that (±)-(1-ethylpyrrolidin-2-yl)methanamine was used instead of diethylamine ¹H-NMR (CDCl₃, 400 MHz) δ 1.30 (t, 3H, J=7.2), 1.86 (m, 1H), 2.00 (m, 3H), 2.10 (m, 2H), 2.69 (t, 2H, J=7.2), 2.83 (m, 4H), 3.13 (q, 2H), 3.37 (m, 2H), 3.77 (m, 1H), 4.09 (s, 2H), 4.40 (s, 2H), 5.02/5.08 (2×d, 2H, J=17.6), 5.44/5.66 (2×d, 2H J=16.8), 6.89 (t, 2H, J=8.8), 7.02 (d, 2H, J=8.0), 7.28 (m, 2H), 7.43 (d, 2H, J=7.6), 7.57 (d, 2H, J=8.0), 7.69 (d, 2H, J=8.0).

### Example 84

### 1-((5-(((2S,6R)-2,6-dimethylmorpholino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

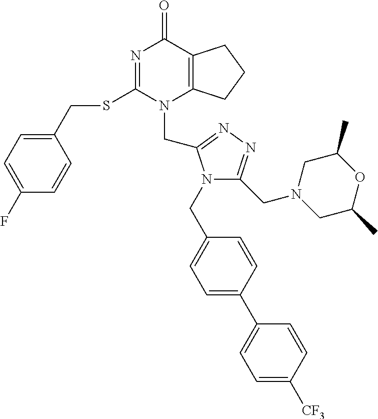 [SMILES: C[C@@H]1CN(Cc2nnc(Cn3c(SCc4ccc(F)cc4)nc(=O)c4c3CCC4)n2Cc2ccc(-c3ccc(C(F)(F)F)cc3)cc2)C[C@H](C)O1]

Following a procedure similar to that described for the preparation of example 79 except that (2S,6R)-2,6-dimethylmorpholine was used instead of diethylamine ¹H-NMR (CDCl₃, 400 MHz) δ 1.11 (d, 6H, J=6.0), 1.94 (m, 4H), 2.62 (m, 4H), 2.75 (t, 2H, J=7.2), 3.48 (m, 2H), 3.70 (s, 2H), 4.45 (s, 2H), 5.05 (s, 2H), 5.42 (s, 2H), 6.92 (m, 4H), 7.32 (dd, 2H, J=8.8, 5.6), 7.45 (d, 2H, J=8.0), 7.62 (d, 2H, J=8.0), 7.71 (d, 2H, J=8.0).

### Example 85

### methyl-2-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methylamino)-2-methylpropanoate

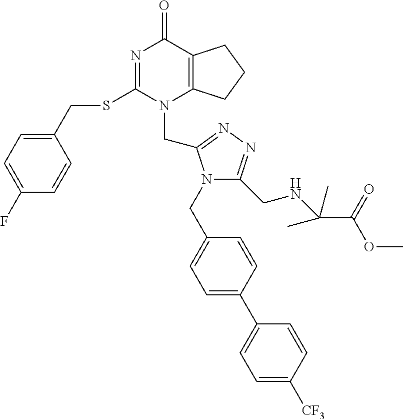 [SMILES: COC(=O)C(C)(C)NCc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 75 except that methyl-2-amino-2-methylpropanoate hydrochloride was used instead of dimethylamine hydrochloride. ¹H-NMR (CDCl₃, 400 MHz) δ1.33 (s, 6H), 1.94 (m, 2H), 2.62 (t, 2H), 2.71 (t, 2H), 3.68 (s, 3H), 3.94 (s, 2H), 4.45 (s, 2H), 5.03 (s, 2H), 5.45 (s, 2H), 6.94 (m, 4H), 7.32 (dd, 2H), 7.45 (d, 2H), 7.62 (d, 2H), 7.71 (d, 2H).

### Example 86

### 1-((5-((4-ethylpiperazin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

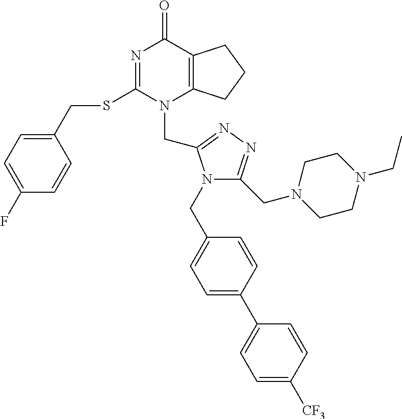 [SMILES: CCN1CCN(Cc2nnc(Cn3c(SCc4ccc(F)cc4)nc(=O)c4c3CCC4)n2Cc2ccc(-c3ccc(C(F)(F)F)cc3)cc2)CC1]

Following a procedure similar to that described for the preparation of example 79 except that 1-ethylpiperazine was used instead of diethylamine ¹H-NMR (CDCl₃, 300 MHz) δ 1.07 (t, 3H, J=7.2), 1.96 (m, 4H), 2.39 (m, 4H), 2.54 (m, 4H), 2.64 (t, 2H, J=7.2), 2.76 (t, 2H, J=7.2), 3.72 (s, 2H), 4.45 (s, 2H), 5.04 (s, 2H), 5.42 (s, 2H), 6.94 (m, 4H), 7.31 (dd, 2H), 7.46 (d, 2H, J=8.1), 7.62 (d, 2H), 7.71 (d, 2H).

### Example 87

### 2-(4-fluorobenzylthio)-1-((5-((2-methoxyethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

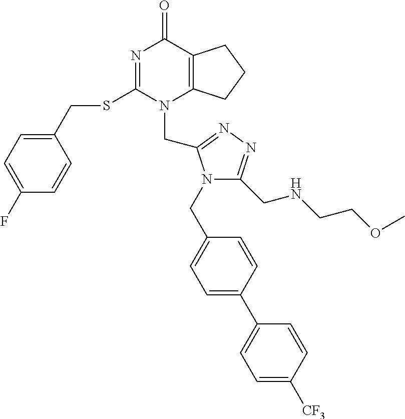 [SMILES: COCCNCc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that 2-methoxyethylamine was used instead of diethylamine. ¹H-NMR (CDCl₃, 300 MHz) δ1.95 (m, 2H), 2.50 (vbrs, 1H), 2.62 (t, 2H), 2.74 (t, 2H), 2.91 (t, 2H), 3.31 (s, 3H), 3.51 (t, 2H), 4.09 (s, 2H), 4.43 (s, 2H), 5.06 (s, 2H), 5.45 (s, 2H), 6.93 (m, 4H), 7.31 (dd, 2H), 7.44 (d, 2H), 7.60 (d, 2H), 7.70 (d, 2H).

### Example 88

### methyl-2-(4-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)piperazin-1-yl)-2-methylpropanoate

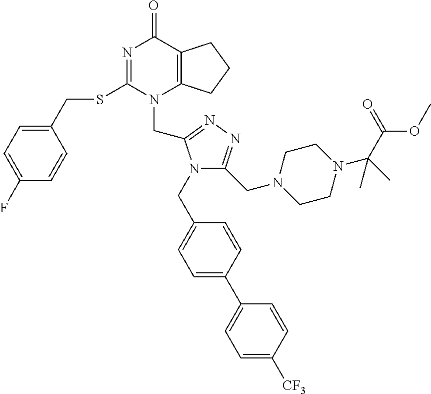 [SMILES: COC(=O)C(C)(C)N1CCN(Cc2nnc(Cn3c(SCc4ccc(F)cc4)nc(=O)c4c3CCC4)n2Cc2ccc(-c3ccc(C(F)(F)F)cc3)cc2)CC1]

Following a procedure similar to that described for the preparation of example 75 except that M20 was used instead of dimethylamine hydrochloride. ¹H-NMR (CDCl₃, 400 MHz) δ1.29 (s, 6H), 1.96 (m, 2H), 2.53 (m, 8H), 2.63 (t, 2H), 2.75 (t, 2H), 3.64 (s, 3H), 3.71 (s, 2H), 4.44 (s, 2H), 5.03 (s, 2H), 5.42 (s, 2H), 6.94 (m, 4H), 7.31 (dd, 2H), 7.45 (d, 2H), 7.62 (d, 2H), 7.70 (d, 2H).

### Example 89

### 2-(4-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)piperazin-1-yl)-2-methylpropanoic acid

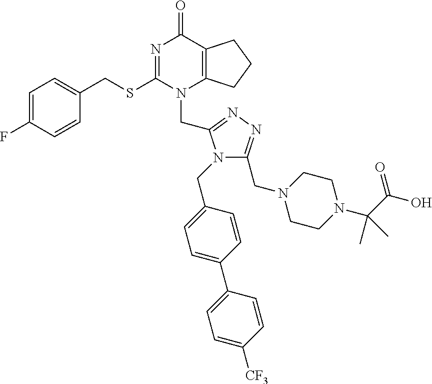 [SMILES: CC(C)(C(=O)O)N1CCN(Cc2nnc(Cn3c(SCc4ccc(F)cc4)nc(=O)c4c3CCC4)n2Cc2ccc(-c3ccc(C(F)(F)F)cc3)cc2)CC1]

Following a procedure similar to that described for the preparation of example 63 except that example 88 was used instead of example 62. MS (ESI): 774 (M−H).

### Example 90

### 1-((5-(((2-(dimethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

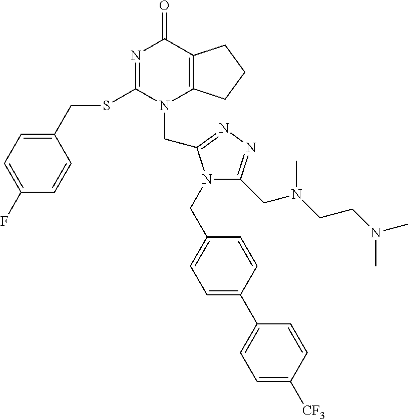 [SMILES: CN(C)CCN(C)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that N,N,N′-trimethylethylenediamine was used instead of diethylamine ¹H-NMR (CDCl₃, 300 MHz) δ1.93 (m, 2H), 2.07 (s, 6H), 2.24 (s, 3H), 2.33 (t, 2H), 2.53 (t, 2H), 2.59 (t, 2H), 2.70 (t, 2H), 3.78 (s, 2H), 4.45 (s, 2H), 5.02 (s, 2H), 5.57 (s, 2H), 6.87 (d, 2H), 6.94 (t, 2H), 7.32 (dd, 2H), 7.43 (d, 2H), 7.62 (d, 2H), 7.70 (d, 2H).

### Example 91a

### 1-((5-(((2-(diethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

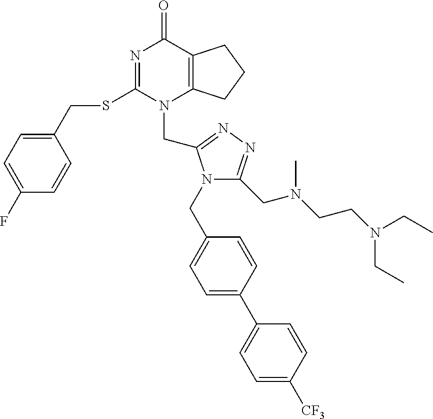 [SMILES: CCN(CC)CCN(C)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that N,N-Diethyl-N′-methylethylenediamine was used instead of diethylamine. ¹H-NMR (CDCl₃, 300 MHz) δ 1.28 (t, 6H), 2.03 (m, 2H), 2.41 (s, 3H), 2.74 (t, 2H), 2.95 (t, 2H), 3.05 (m, 8H), 3.90 (s, 2H), 4.37 (s, 2H), 5.15 (s, 2H), 5.62 (s, 2H), 6.84 (t, 2H), 7.06 (d, 2H), 7.23 (dd, 2H), 7.40 (d, 2H), 7.55 (d, 2H), 7.70 (d, 2H).

### Example 91b

### 1-((5-(((2-(diethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one tartrate

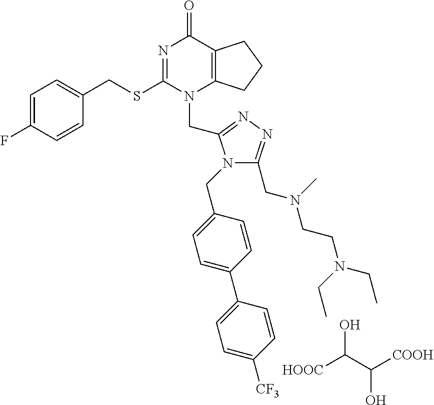

To a solution of 91a (76 mg) in methanol (2 ml) was added tartaric acid (15 mg, 1 equiv) at room temperature. The reaction was stirred for 5 min and then evaporated to dryness under reduced pressure and dried under vacuo to give 91b as a white solid. ¹H-NMR (d₆-DMSO, 300 MHz) δ 1.09 (t, 6H, J=7.2), 1.91 (m, 2H), 2.23 (s, 3H), 2.54 (t, 2H, J=6.9), 2.70 (t, 2H, J=6.9), 2.75 (t, 2H, J=5.7), 2.99 (q, 4H, J=7.2), 3.06 (t, 2H, J=6.0), 3.77 (s, 2H), 4.26 (s, 2H), 4.30 (s, 2H), 5.17 (s, 2H), 5.45 (s, 2H), 7.02 (t, 2H, J=8.7), 7.19 (d, 2H, J=8.1), 7.23 (dd, 2H, J=8.4, 6.0), 7.65 (d, 2H, J=8.4), 7.81 (m, 4H).

### Example 92

### 1-((5-(((3-(dimethylamino)propyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

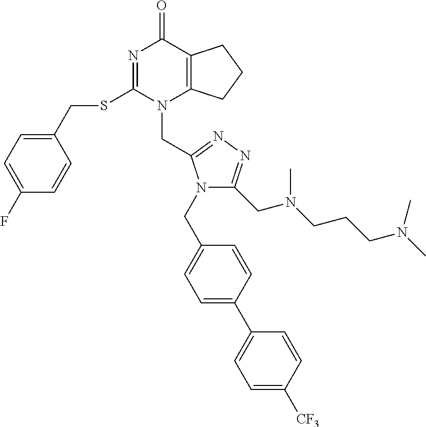 [SMILES: CN(C)CCCN(C)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that N,N,N′-trimethyl-1,3-propanediamine was used instead of diethylamine ¹H-NMR (CDCl₃, 300 MHz) δ1.89 (m, 2H), 2.00 (m, 2H), 2.39 (s, 3H), 2.64 (s, 6H), 2.70 (m, 4H), 2.89 (m, 4H), 3.86 (s, 2H), 4.39 (s, 2H), 5.17 (s, 2H), 5.58 (s, 2H), 6.87 (t, 2H), 7.04 (d, 2H), 7.27 (m, 2H), 7.42 (d, 2H), 7.58 (d, 2H), 7.70 (d, 2H).

### Example 93

### 2-(4-fluorobenzylthio)-1-((5-((2-morpholinoethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

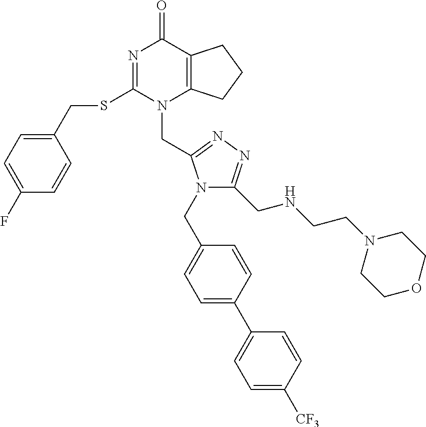 [SMILES: O=c1nc(SCc2ccc(F)cc2)n(Cc2nnc(CNCCN3CCOCC3)n2Cc2ccc(-c3ccc(C(F)(F)F)cc3)cc2)c2c1CCC2]

Following a procedure similar to that described for the preparation of example 79 except that 4-(2-aminoethyl)morpholine was used instead of diethylamine ¹H-NMR (CDCl₃, 300 MHz) δ 1.94 (m, 2H), 2.50 (m, 6H), 2.61 (t, 2H), 2.72 (t, 2H), 2.79 (t, 2H), 3.70 (t, 4H), 4.03 (s, 2H), 4.46 (s, 2H), 5.04 (s, 2H), 5.45 (s, 2H), 6.92 (m, 4H), 7.33 (m, 2H), 7.44 (d, 2H), 7.61 (d, 2H), 7.71 (d, 2H).

### Example 94

### (R)-1-((5-((3-(dimethylamino)pyrrolidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

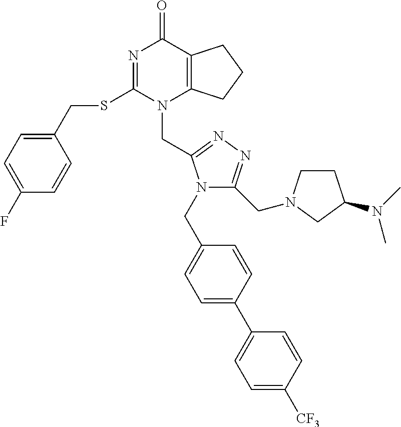 [SMILES: CN(C)[C@@H]1CCN(Cc2nnc(Cn3c(SCc4ccc(F)cc4)nc(=O)c4c3CCC4)n2Cc2ccc(-c3ccc(C(F)(F)F)cc3)cc2)C1]

Following a procedure similar to that described for the preparation of example 79 except that (R)—N,N-dimethylpyrrolidin-3-amine was used instead of diethylamine. ¹H-NMR (CDCl₃, 400 MHz) δ 1.72 (m, 1H), 1.95 (m, 3H), 2.17 (s, 6H), 2.50 (m, 1H), 2.61 (m, 4H), 2.74 (m, 4H), 3.83 (s, 2H), 4.46 (s, 2H), 5.05 (s, 2H), 5.43 (2×d, 2H), 6.88 (d, 2H), 6.95 (t, 2H), 7.33 (dd, 2H), 7.44 (d, 2H), 7.63 (d, 2H), 7.71 (d, 2H).

### Example 95

### (S)-1-((5-((3-(dimethylamino)pyrrolidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

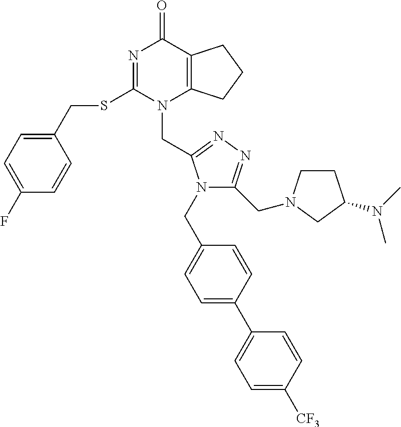 [SMILES: CN(C)[C@H]1CCN(Cc2nnc(Cn3c(SCc4ccc(F)cc4)nc(=O)c4c3CCC4)n2Cc2ccc(-c3ccc(C(F)(F)F)cc3)cc2)C1]

Following a procedure similar to that described for the preparation of example 79 except that (S)—N,N-dimethylpyrrolidin-3-amine was used instead of diethylamine ¹H-NMR (CDCl₃, 400 MHz) δ 1.70 (m, 1H), 1.94 (m, 3H), 2.17 (s, 6H), 2.49 (m, 1H), 2.61 (m, 4H), 2.73 (m, 4H), 3.83 (s, 2H), 4.46 (s, 2H), 5.04 (s, 2H), 5.43 (2×d, 2H), 6.88 (d, 2H), 6.95 (t, 2H), 7.33 (dd, 2H), 7.44 (d, 2H), 7.62 (d, 2H), 7.71 (d, 2H).

### Example 96

### 2-(4-fluorobenzylthio)-1-((5-(2-(piperidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

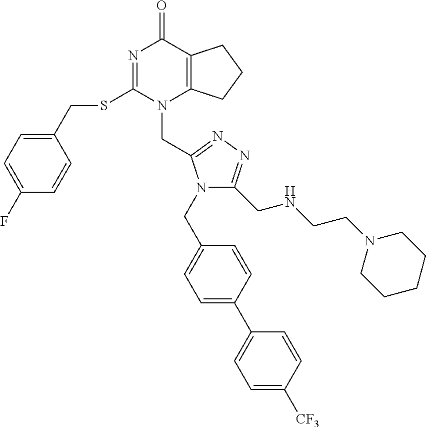 [SMILES: O=c1nc(SCc2ccc(F)cc2)n(Cc2nnc(CNCCN3CCCCC3)n2Cc2ccc(-c3ccc(C(F)(F)F)cc3)cc2)c2c1CCC2]

Following a procedure similar to that described for the preparation of example 79 except that N-(2-aminoethyl)piperidine was used instead of diethylamine ¹H-NMR (CDCl₃, 300 MHz) δ1.40 (m, 2H), 1.51 (m, 4H), 1.93 (m, 2H), 2.03 (t, 2H), 2.31 (t, 2H), 2.38 (t, 2H), 2.59 (t, 2H), 2.71 (t, 4H), 4.02 (s, 2H), 4.46 (s, 2H), 5.04 (s, 2H), 5.46 (s, 2H), 6.90 (d, 2H), 6.95 (t, 2H), 7.33 (dd, 2H), 7.43 (d, 2H), 7.62 (d, 2H), 7.71 (d, 2H).

### Example 97

### 2-(4-fluorobenzylthio)-1-((5-(2-(pyrrolidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

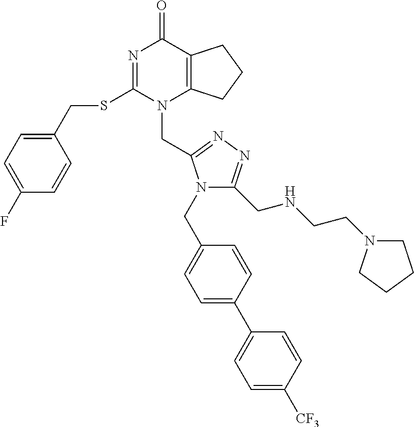 [SMILES: O=c1nc(SCc2ccc(F)cc2)n(Cc2nnc(CNCCN3CCCC3)n2Cc2ccc(-c3ccc(C(F)(F)F)cc3)cc2)c2c1CCC2]

Following a procedure similar to that described for the preparation of example 79 except that 2-(pyrrolidin-1-yl)ethanamine was used instead of diethylamine ¹H-NMR (CDCl₃, 300 MHz) δ1.73 (m, 4H), 1.94 (m, 2H), 2.47 (m, 4H), 2.58 (m, 4H), 2.73 (m, 4H), 4.02 (s, 2H), 4.46 (s, 2H), 5.04 (s, 2H), 5.46 (s, 2H), 6.90 (d, 2H), 6.94 (t, 2H), 7.33 (dd, 2H), 7.43 (d, 2H), 7.62 (d, 2H), 7.71 (d, 2H).

### Example 98

### 1-((5-((2-(diisopropylamino)ethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

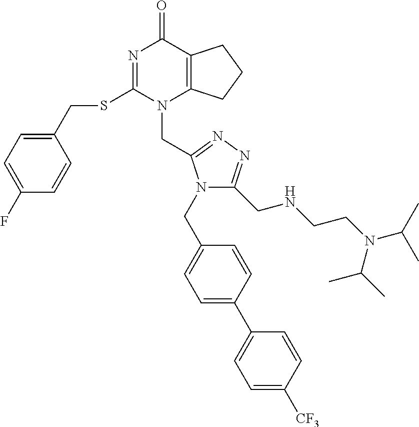 [SMILES: CC(C)N(CCNCc1nnc(Cn2c(SCc3ccc(F)cc3)nc(=O)c3c2CCC3)n1Cc1ccc(-c2ccc(C(F)(F)F)cc2)cc1)C(C)C]

Following a procedure similar to that described for the preparation of example 79 except that N,N-di(propan-2-yl)ethane-1,2-diamine was used instead of diethylamine ¹H-NMR (CDCl₃, 300 MHz) δ 1.11 (d, 12H), 1.96 (m, 2H), 2.64 (t, 2H), 2.75 (m, 6H), 3.17 (m, 2H), 4.01 (s, 2H), 4.43 (s, 2H), 5.04 (s, 2H), 5.47 (s, 2H), 6.94 (m, 4H), 7.31 (dd, 2H), 7.43 (d, 2H), 7.60 (d, 2H), 7.70 (d, 2H).

### Example 99

### 1-((5-((2-(diethylamino)ethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

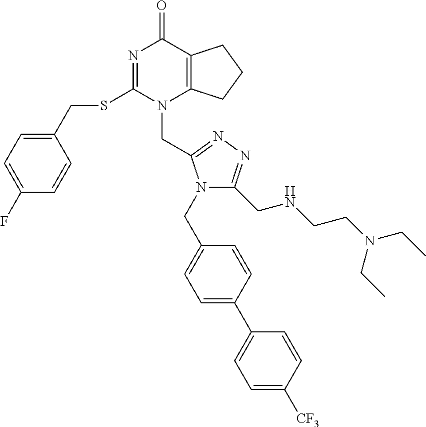 [SMILES: CCN(CC)CCNCc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that N,N-Diethylethylenediamine was used instead of diethylamine. ¹H-NMR (CDCl₃, 300 MHz) δ 0.96 (t, 6H), 1.94 (m, 2H), 2.49 (m, 6H), 2.66 (m, 6H), 4.01 (s, 2H), 4.46 (s, 2H), 5.03 (s, 2H), 5.46 (s, 2H), 6.94 (m, 4H), 7.32 (dd, 2H), 7.44 (d, 2H), 7.61 (d, 2H), 7.71 (d, 2H).

### Example 100

### 2-(4-fluorobenzylthio)-1-((5-((methyl(pyridin-2-ylmethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

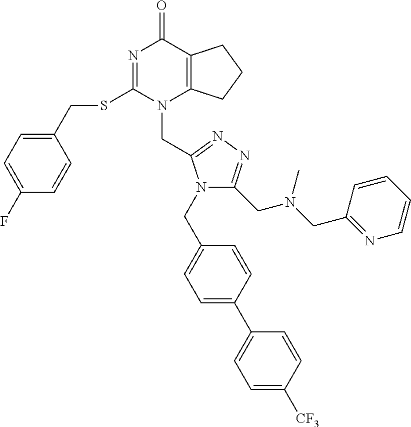 [SMILES: CN(Cc1ccccn1)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that N-methyl-1-(pyridin-2-yl)methanamine was used instead of diethylamine ¹H-NMR (MeOD, 300 MHz) δ 2.04 (m, 2H), 2.26 (s, 3H), 2.67 (t, 2H), 2.82 (t, 2H), 3.73 (s, 2H), 3.84 (s, 2H), 4.36 (s, 2H), 5.25 (s, 2H), 5.49 (s, 2H), 6.87 (t, 2H), 7.04 (d, 2H), 7.20 (m, 1H), 7.29 (m, 3H), 7.51 (d, 2H), 7.57 (d, 1H), 7.62 (m, 1H), 7.72 (d, 2H), 7.75 (d, 2H), 8.36 (d, 1H).

### Example 101

### 1-((5-((cyclopropyl(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

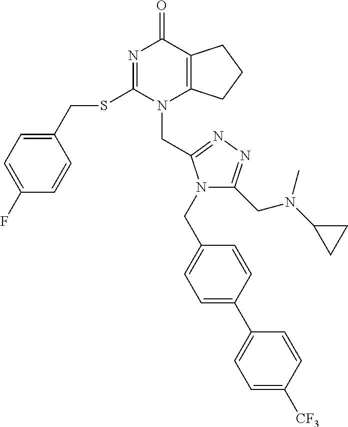 [SMILES: CN(Cc1nnc(Cn2c(SCc3ccc(F)cc3)nc(=O)c3c2CCC3)n1Cc1ccc(-c2ccc(C(F)(F)F)cc2)cc1)C1CC1]

Following a procedure similar to that described for the preparation of example 79 except that N-methylcyclopropanamine was used instead of diethylamine ¹H-NMR (CDCl₃, 400 MHz) δ 0.19 (m, 2H), 0.45 (m, 2H), 1.80 (m, 1H), 1.95 (m, 2H), 2.30 (s, 3H), 2.62 (t, 2H, J=7.2), 2.74 (t, 2H, J=7.2), 3.90 (s, 2H), 4.40 (s, 2H), 5.01 (s, 2H), 5.33 (s, 2H), 6.88 (d, 2H, J=8.0), 6.94 (t, 2H, J=8.8), 7.31 (dd, 2H, J=8.8, 5.2), 7.45 (d, 2H, J=8.4), 7.62 (d, 2H, J=8.0), 7.70 (d, 2H, J=8.4).

### Example 102

### 2-(4-fluorobenzylthio)-1-((5-((2-(2-oxoimidazolidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

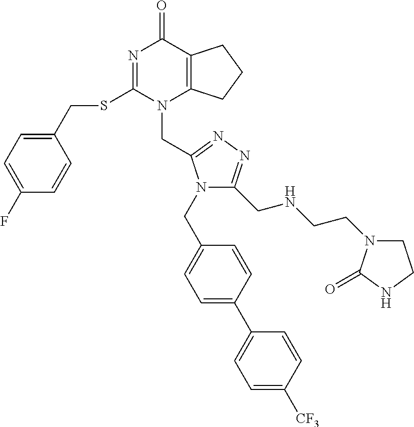 [SMILES: O=C1NCCN1CCNCc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that 1-(2-aminoethyl)imidazolidin-2-one was used instead of diethylamine. ¹H-NMR (CDCl₃, 300 MHz) δ1.94 (m, 2H), 2.64 (t, 2H), 2.69 (t, 2H), 3.40 (t, 2H), 3.45 (t, 2H), 4.43 (s, 2H), 4.61 (s, 2H), 5.03 (s, 2H), 5.54 (s, 2H), 6.33 (s, 1H), 6.85 (t, 2H), 7.05 (d, 2H), 7.22 (dd, 2H), 7.33 (s, 1H), 7.46 (d, 2H), 7.58 (d, 2H), 7.69 (d, 2H).

The following compounds were also prepared by a procedure similar to that described for the preparation of example 79:

| Name | Structure | Reactant |
|---|---|---|
| Example 103: 1-((5-((4-(dimethylami- no)piperidin-1-yl)methyl)- 4-((4′-(trifluoromethyl)bi- phenyl-4-yl)methyl)-4H- 1,2,4-triazol-3-yl)methyl)- 2-(4-fluorobenzylthio)-6,7- dihydro-1-cyclopen- ta[d]pyrimidin-4(5H)-one | 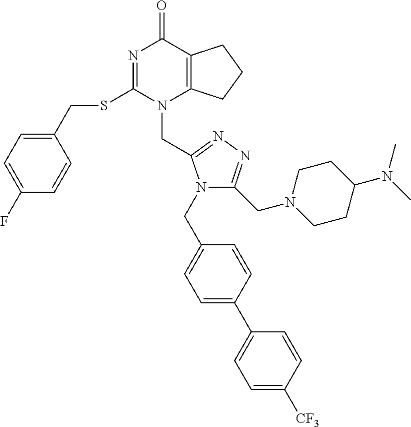 [SMILES: CN(C)C1CCN(Cc2nnc(Cn3c(SCc4ccc(F)cc4)nc(=O)c4c3CCC4)n2Cc2ccc(-c3ccc(C(F)(F)F)cc3)cc2)CC1] | N,N-dimethylpip- eridin-4-amine |
| Example 104: 1-((5-((3,3-difluoropyrrol- idin-1-yl)methyl)-4-((4′- (trifluoromethyl)biphenyl- 4-yl)methyl)-4H-1,2,4-tri- azol-3-yl)methyl)-2-(4- fluorobenzylthio)-6,7-dihy- dro-1H-cyclopenta[d]py- rimidin-4(5H)-one | 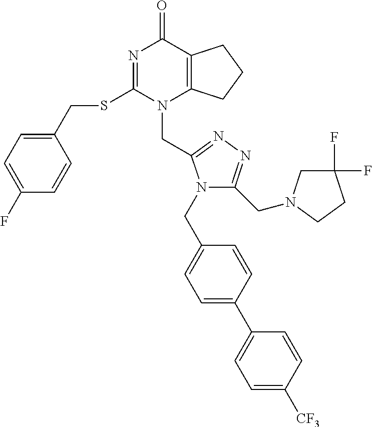 [SMILES: O=c1nc(SCc2ccc(F)cc2)n(Cc2nnc(CN3CCC(F)(F)C3)n2Cc2ccc(-c3ccc(C(F)(F)F)cc3)cc2)c2c1CCC2] | 3,3-difluoropy- rrolidine hydrochloride, DIPEA |
| Example 105: 1-((5-((2-(dimethylami- no)ethylamino)methyl)-4- ((4′-(trifluoromethyl)biphe- nyl-4-yl)methyl)-4H- 1,2,4-triazol-3-yl)methyl)- 2-(4-fluorobenzylthio)-6,7- dihydro-1H-cyclopen- ta[d]pyrimidin-4(5H)-one | 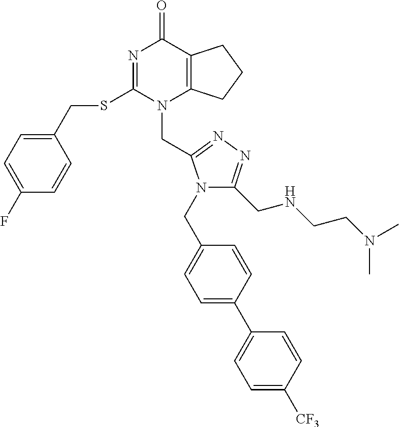 [SMILES: CN(C)CCNCc1nnc(Cn2c(SCc3ccc(F)cc3)nc(=O)c3c2CCC3)n1Cc1ccc(-c2ccc(C(F)(F)F)cc2)cc1] | N,N-dimethyleth- ylenediamine |
| Example 106: (S)-1-((5-(((1-ethylpyrro- lidin-2-yl)methylami- no)methyl)-4-((4′-(trifluoro- methyl)biphenyl-4-yl)meth- yl)-4H-1,2,4-triazol-3- yl)methyl)-2-(4-fluorobenzyl- thio)-6,7-dihydro-1H-cy- clopenta[d]pyrimidin- 4(5H)-one | 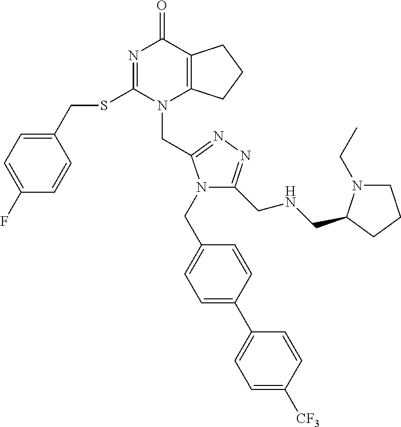 [SMILES: CCN1CCC[C@H]1CNCc1nnc(Cn2c(SCc3ccc(F)cc3)nc(=O)c3c2CCC3)n1Cc1ccc(-c2ccc(C(F)(F)F)cc2)cc1] | (S)-(1-ethylpy- rrolidin-2- yl)methanamine |
| Example 107: 2-(4-fluorobenzylthio)-1- ((5-((4-methylpiperazin- 1-yl)methyl)-4-((4′-(trifluoro- methyl)biphenyl-4- yl)methyl)-4H-1,2,4-triazol- 3-yl)methyl)-6,7-dihydro- 1H-cyclopenta[d]pyrimi- din-4(5H)-one | 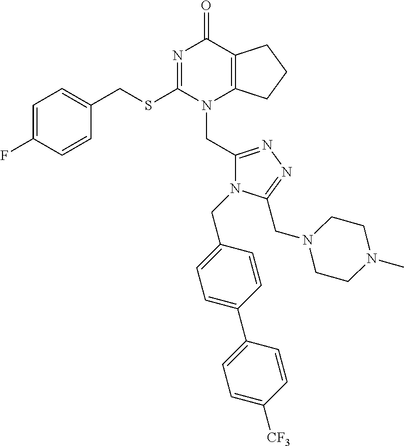 [SMILES: CN1CCN(Cc2nnc(Cn3c(SCc4ccc(F)cc4)nc(=O)c4c3CCC4)n2Cc2ccc(-c3ccc(C(F)(F)F)cc3)cc2)CC1] | 1-methyl- piperazine |

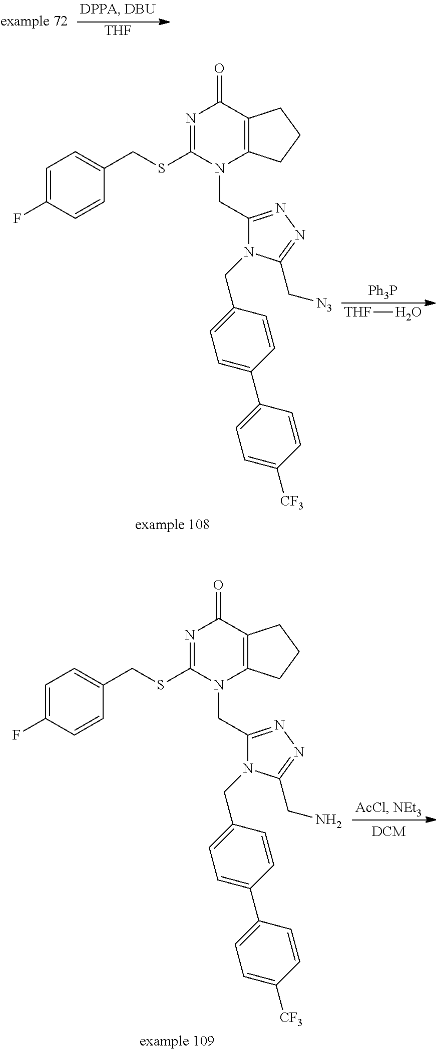

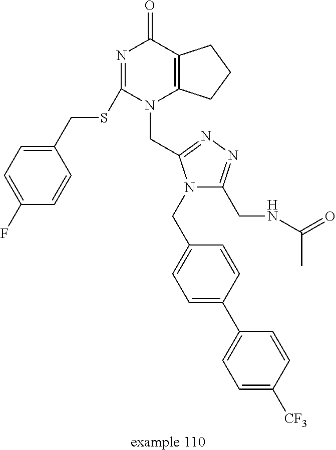 [SMILES: CC(=O)NCc1nnc(Cn2c(SCc3ccc(F)cc3)nc(=O)c3c2CCC3)n1Cc1ccc(-c2ccc(C(F)(F)F)cc2)cc1 ; "example 110"]

### Example 108

1-((5-(azidomethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

Following a procedure similar to that described for the preparation of intermediate E1 except that example 72 was used instead of D18. ¹H-NMR (CDCl₃, 400 MHz) δ1.99 (m, 2H), 2.66 (t, 2H), 2.80 (t, 2H), 4.42 (s, 2H), 4.60 (s, 2H), 5.11 (s, 2H), 5.33 (s, 2H), 6.91 (t, 2H), 6.91 (d, 2H), 7.29 (dd, 2H), 7.47 (d, 2H), 7.60 (d, 2H), 7.71 (d, 2H).

### Example 109

### 1-((5-(aminomethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

Following a procedure similar to that described for the preparation of intermediate E2 except that example 108 was used instead of E1. ¹H-NMR (CDCl₃, 400 MHz) δ1.95 (m, 2H), 2.60 (t, 2H), 2.72 (t, 2H), 4.09 (s, 2H), 4.43 (s, 2H), 5.03 (s, 2H), 5.40 (s, 2H), 6.92 (m, 4H), 7.30 (dd, 2H), 7.44 (d, 2H), 7.60 (d, 2H), 7.70 (d, 2H).

### Example 110

### N-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)acetamide

To the compound of example 109 (20 mg, 1 equiv) in CH₂Cl₂ (2 ml) were added triethylamine (10 μl, 2 equiv) and acetyl chloride (5 μl, 2 equiv) (a CaCl₂ drying tube was used to insulate moisture) and the mixture was stirred at room temperature for 1 h. Then the mixture was transferred to a separating funnel and washed with brine (X3). The organic phase was dried and concentrated. The crude product was purified by preparative TLC to afford 110 (15 mg) as a solid. ¹H-NMR (CDCl₃, 400 MHz) δ2.00 (m, 2H), 2.29 (s, 3H), 2.67 (t, 2H), 2.79 (t, 2H), 4.39 (s, 2H), 4.66 (s, 2H), 5.09 (s, 2H), 5.48 (s, 2H), 6.90 (t, 2H), 6.99 (d, 2H), 7.07 (vbrs, 1H), 7.28 (dd, 2H), 7.46 (d, 2H), 7.58 (d, 2H), 7.70 (d, 2H).

### Example 111

### N-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)ethanesulfonamide

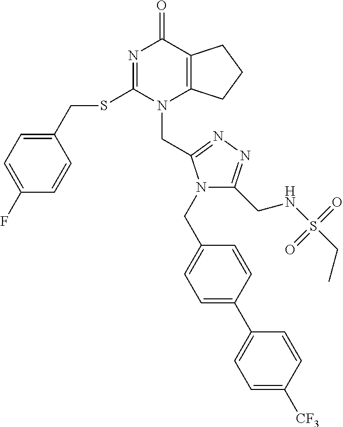 [SMILES: CCS(=O)(=O)NCc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 110 except that ethanesulfonyl chloride was used instead of ethanoyl chloride. ¹H-NMR (CDCl₃, 400 MHz) δ 1.37 (t, 3H), 2.00 (m, 2H), 2.69 (t, 2H), 2.77 (t, 2H), 3.10 (q, 2H), 4.40 (s, 2H), 4.47 (s, 2H), 5.03 (s, 2H), 5.41 (s, 2H), 6.11 (vbrs, 1H), 6.90 (t, 2H), 7.02 (d, 2H), 7.27 (m, 2H), 7.46 (d, 2H), 7.58 (d, 2H), 7.70 (d, 2H).

### Example 112

### 2-(4-fluorobenzylthio)-1-((5-((2-oxoimidazolidin-1-yl)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

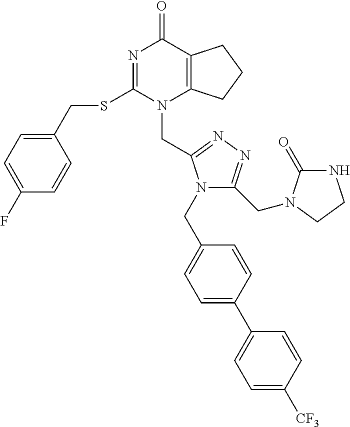 [SMILES: O=C1NCCN1Cc1nnc(Cn2c(SCc3ccc(F)cc3)nc(=O)c3c2CCC3)n1Cc1ccc(-c2ccc(C(F)(F)F)cc2)cc1]

To a mixture of 109 (25 mg) in EtOH (2 ml) were added triethylamine (12 μl) and 2-chloroethyl isocyanate (6 μl). The mixture was stirred over night at room temperature and then concentrated under reduced pressure. The residue was dissolved in CH₂Cl₂ (5 ml), transferred to a separating funnel, washed with brine (X3). The organic phase was dried and concentrated. The crude product was purified by preparative TLC to afford 112 (15 mg) as a solid. ¹H-NMR (CDCl₃, 400 MHz) δ 1.94 (m, 2H), 2.66 (m, 4H), 3.40 (t, 2H), 3.45 (t, 2H), 4.34 (s, 2H), 4.61 (s, 2H), 5.03 (s, 2H), 5.54 (s, 2H), 6.33 (vbrs, 1H), 6.85 (t, 2H), 7.05 (d, 2H), 7.22 (dd, 2H), 7.32 (vbrs, 1H), 7.46 (d, 2H), 7.58 (d, 2H), 7.69 (d, 2H).

### Example 113

### 1-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)-3-methylthiourea

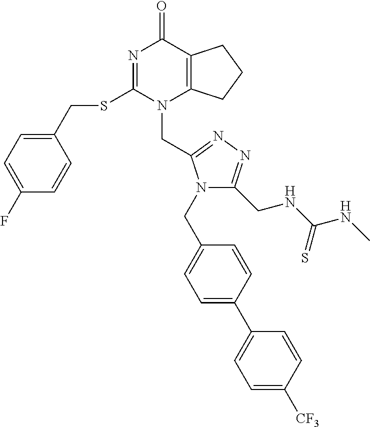 [SMILES: CNC(=S)NCc1nnc(Cn2c(SCc3ccc(F)cc3)nc(=O)c3c2CCC3)n1Cc1ccc(-c2ccc(C(F)(F)F)cc2)cc1]

To a solution of the compound of Example 109 (20 mg) in CH₂Cl₂ (2 ml) was added isothiocyanatomethane (5 mg). The mixture was stirred under reflux for 2 h and then purified by preparative TLC to afford 113 (12 mg) as a solid. ¹H-NMR (CDCl₃, 400 MHz) δ 1.98 (m, 2H), 2.69 (m, 4H), 2.91 (s, 3H), 4.35 (s, 2H), 5.00 (s, 2H), 5.09 (s, 2H), 5.71 (s, 2H), 6.83 (t, 2H), 7.13 (d, 2H), 7.20 (dd, 2H), 7.50 (m, 3H), 7.59 (d, 2H), 7.70 (d, 2H), 9.07 (vbrs, 1H).

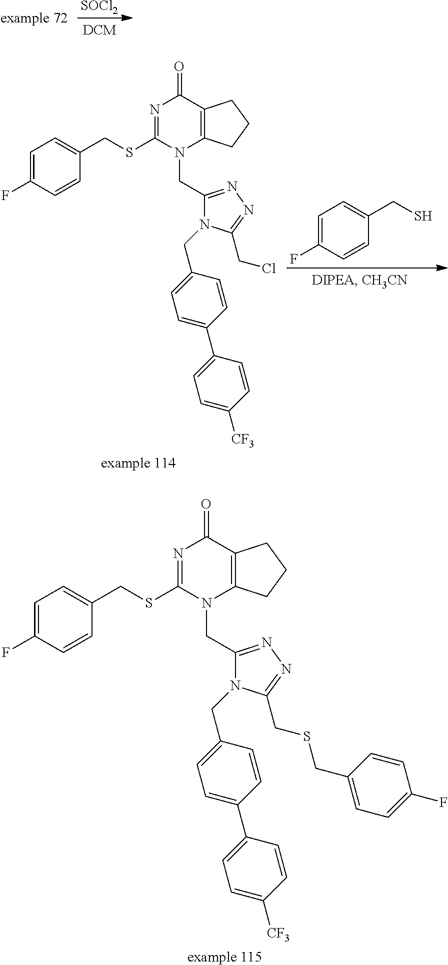

### Example 114

### 1-((5-(chloromethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

To the compound of Example 72 (200 mg, 1 equiv) in dry CH₂Cl₂ (5 ml) in a flask was added DMF (1 ml). CaCl₂ drying tube was used to insulate moisture and the flask was placed in an ice bath. To the mixture was added thionyl chloride (30 μl, 1.2 equiv). The mixture was stirred for 0.5 h before it was quenched with saturated NaHCO₃ solution. The organic phase was separated and washed with brine (X1), dried over Na₂SO₄ and concentrated to afford 114 (180 mg) as a solid which could be used in next step without further purification.

### Example 115

### 2-(4-fluorobenzylthio)-1-((5-((4-fluorobenzylthio)methyl)-4-((4′-(trifluoromethyl)biphenyl-4-yl)methyl)-4H-1,2,4-triazol-3-yl)methyl)-6,7-dihydro-1H-cyclopenta[d ]pyrimidin-4(5H)-one

Following a procedure similar to that described for the preparation of intermediate D36 except that the compound of example 114 was used instead of D19 and “4-fluoro benzyl mercaptan” was used in place of “benzyl mercaptan”. ¹H-NMR (CDCl₃, 300 MHz) δ1.98 (m, 2H), 2.63 (t, 2H), 2.76 (t, 2H), 2.91 (t, 2H), 3.69 (s, 4H), 4.46 (s, 2H), 5.04 (s, 2H), 5.26 (s, 2H), 6.95 (m, 6H), 7.34 (m, 4H), 7.43 (d, 2H), 7.60 (d, 2H), 7.71 (d, 2H).

### Example 116

### (E)-1-((5-((2,2-dimethylhydrazono)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

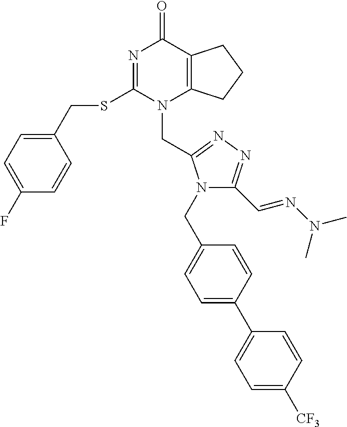 [SMILES: CN(C)/N=C/c1nnc(Cn2c(SCc3ccc(F)cc3)nc(=O)c3c2CCC3)n1Cc1ccc(-c2ccc(C(F)(F)F)cc2)cc1]

A mixture of example 74 (30 mg, 1 equiv), DIPEA (16 μl, 2 equiv), 1,1-dimethylhydrazine monohydrochloride (7 mg, 1.5 equiv) and 4 Å molecular sieve (0.5 g) in dry CH₂Cl₂ (3 ml) was shaked occasionally for 2 h. The mixture was then filtrated to remove the molecular sieve and the filtrate was washed with brine (X3), dried and then purified by preparative TLC to afford example 116 (22 mg) as a solid. ¹H-NMR (CDCl₃, 300 MHz) δ1.90 (m, 2H), 2.57 (t, 2H), 2.65 (t, 2H), 2.97 (s, 6H), 4.45 (s, 2H), 5.08 (s, 2H), 5.71 (s, 2H), 6.93 (t, 2H), 7.96 (d, 2H), 7.32 (dd, 2H), 7.35 (s, 1H), 7.42 (d, 2H), 7.62 (d, 2H), 7.69 (d, 2H); MS (ESI): 662 (M+H).

### Example 117

### (E)-2-(4-fluorobenzylthio)-1-((5-((piperidin-1-ylimino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

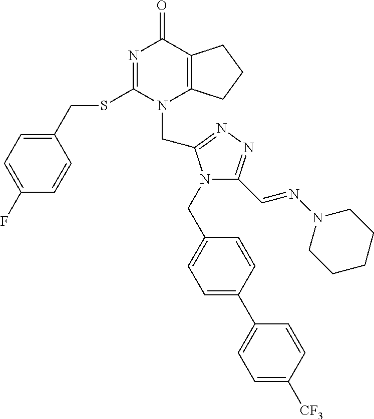 [SMILES: O=c1nc(SCc2ccc(F)cc2)n(Cc2nnc(/C=N/N3CCCCC3)n2Cc2ccc(-c3ccc(C(F)(F)F)cc3)cc2)c2c1CCC2]

Following a procedure similar to that described for the preparation of example 116 except that piperidin-1-amine was used instead of 1,1-dimethylhydrazine monohydrochloride. ¹H-NMR (CDCl₃, 300 MHz) δ1.53 (m, 2H), 1.67 (m, 4H), 1.91 (m, 2H), 2.58 (t, 2H), 2.65 (t, 2H), 3.11 (t, 4H), 4.45 (s, 2H), 5.08 (s, 2H), 5.74 (s, 2H), 6.93 (t, 2H), 7.97 (d, 2H), 7.31 (dd, 2H), 7.42 (d, 2H), 7.62 (d, 2H), 7.68 (s, 1H), 7.69 (d, 2H).

The following compounds were also prepared by a procedure similar to that described for the preparation of example 116:

| Name | Structure | Reactant |
|---|---|---|
| Example 118: (E)-1-((5-((2-tert-but- ylhydrazono)methyl)-4- ((4′-(trifluoromethyl)bi- phenyl-4-yl)methyl)- 4H-1,2,4-triazol-3- yl)methyl)-2-(4-fluoro- benzylthio)-6,7-dihydro- 1H-cyclopenta[d]py- rimidin-4(5H)-one | 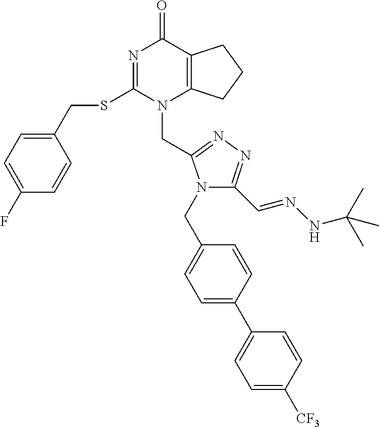 [SMILES: CC(C)(C)N/N=C/c1nnc(Cn2c(SCc3ccc(F)cc3)nc(=O)c3c2CCC3)n1Cc1ccc(-c2ccc(C(F)(F)F)cc2)cc1] | tert-Butylhydrazine monohydrochloride, DIPEA |

### Example 119

### 1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)vinyl acetate

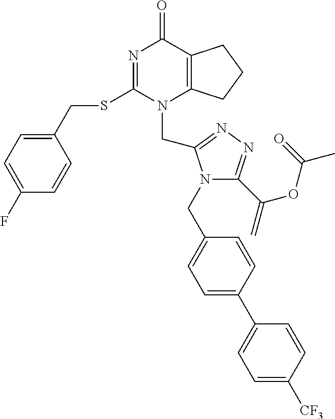 [SMILES: C=C(OC(C)=O)c1nnc(Cn2c(SCc3ccc(F)cc3)nc(=O)c3c2CCC3)n1Cc1ccc(-c2ccc(C(F)(F)F)cc2)cc1]

To a mixture of example 73 (920 mg, 1 equiv) and triethylamine (0.65 ml, 3 equiv) in CH₂Cl₂ (10 ml) placed in an ice bath was added acetyl chloride (0.28 ml, 2.5 equiv) slowly in a nitrogen atmosphere. The mixture was stirred for 2 h before it was transferred to a separating funnel and washed with brine (X3). The organic phase was dried and concentrated. The crude product was purified by flash chromatography to afford example 119 (0.78 g) as a jelly. ¹H-NMR (CDCl₃, 400 MHz) δ 1.99 (m, 2H), 2.07 (s, 3H), 2.66 (t, 2H), 2.77 (t, 2H), 4.42 (s, 2H), 5.03 (s, 2H), 5.42 (s, 2H), 5.43 (d, 1H, J=2.4), 5.49 (d, 1H, J=2.8), 6.95 (m, 4H), 7.31 (dd, 2H, J=8.8, 5.6), 7.47 (d, 2H, J=8.0), 7.61 (d, 2H), 7.71 (d, 2H).

### Example 120

### 2-(4-fluorobenzylthio)-1-((5-(1-hydroxyethyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

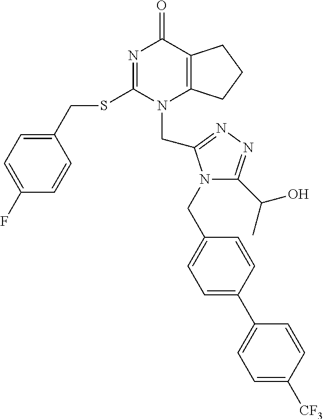 [SMILES: CC(O)c1nnc(Cn2c(SCc3ccc(F)cc3)nc(=O)c3c2CCC3)n1Cc1ccc(-c2ccc(C(F)(F)F)cc2)cc1]

(Method 1)

To a solution of the compound of example 74 (200 mg, 1 equiv) in sufficiently dried THF (3 ml) placed in an ice bath was added methylmagnesium bromide solution (0.36 ml, 1 mol/L in THF, 1.1 equiv) in a nitrogen atmosphere. The mixture was stirred for 2 h before it was quenched with saturated aqueous NH₄Cl solution and extracted with EA. The combined organic phase was dried and then concentrated. The crude product was purified by column chromatography (CH₂Cl₂/CH₃OH=15/1) to afford example 120 (100 mg) as a solid.

(Method 2)

To a solution of the compound of example 119 (760 mg) and NaBH₄ (85 mg) in EtOH (5 ml) in an ice bath was stirred for 1 h and then evaporated to remove the solvent. The residue was diluted with water, placed in an ice bath followed by addition of concd. HCl under stirring until no more bubbles was generated. Then the mixture was quenched with saturated aqueous NaHCO₃ solution and extracted with DCM (X3). The combined organic phase was dried over Na₂SO₄ and concentrated. The crude product was purified by flash chromatography to afford example 120 (550 mg) as a solid. ¹H-NMR (d₆-DMSO, 300 MHz) δ 1.53 (d, 3H), 1.90 (m, 2H), 2.54 (t, 2H), 2.67 (t, 2H), 4.27 (s, 2H), 4.91 (m, 1H), 5.10 (2×d, 2H), 5.48 (s, 2H), 6.99 (t, 2H), 7.24 (d, 2H), 7.31 (t, 2H), 7.63 (d, 2H), 7.80 (m, 4H).

### Example 121

### 1-((5-acetyl-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

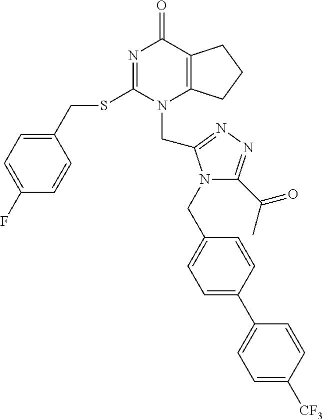 [SMILES: CC(=O)c1nnc(Cn2c(SCc3ccc(F)cc3)nc(=O)c3c2CCC3)n1Cc1ccc(-c2ccc(C(F)(F)F)cc2)cc1]

(Method 1)

To a solution of the compound of example 119 (0.5 g) in isopropanol (10 ml) was added 6N HCl (1 ml) and the mixture was stirred at room temperature over night. Then the mixture was concentrated to remove the solvent. The residue was dissolved in CH₂Cl₂ (10 ml), washed with brine (X3), dried and concentrated. The crude product was purified by flash chromatography to afford example 121 (230 mg) as a solid.

(Method 2)

To a solution of example 120 (90 mg, 1 equiv) in 1,4-dioxane (5 ml) was added activated MnO₂ (123 mg, 10 equiv). The mixture was stirred for 2 h at 70° C. and then filtered to remove MnO₂. The filtrate was evaporated to give a crude product which was then purified by flash chromatography to afford example 121 (50 mg) as a solid. ¹H-NMR (CDCl₃, 400 MHz) δ 1.99 (m, 2H), 2.69 (m, 4H), 2.83 (s, 3H), 4.43 (s, 2H), 5.08 (s, 2H), 5.73 (s, 2H), 6.91 (t, 2H), 7.00 (d, 2H), 7.27 (dd, 2H), 7.46 (d, 2H), 7.59 (d, 2H), 7.70 (d, 2H).

### Example 122

### N-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)-2-hydroxy-N,N-dimethylethanaminium chloride

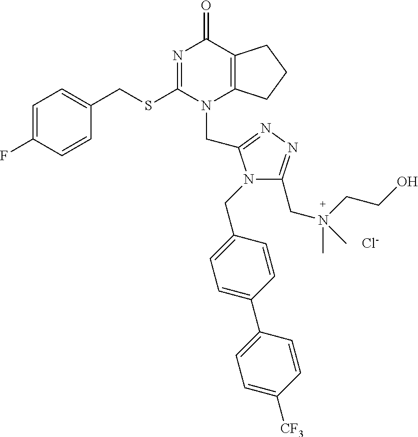

The mixture of 114 (50 mg), Cs₂CO₃ (50 mg) and N,N-Dimethylethanolamine (10 μl) in acetonitrile (2 ml) was heated under reflux for 2 h and the reaction was complete. The mixture was filtered to remove the insoluble materials and the solvent was evaporated under reduced pressure to give a residue which was crystallized from acetone to afford example 122 as a white solid. ¹H-NMR (CD₃OD, 400 MHz) δ 2.08 (m, 2H), 2.71 (t, 2H), 2.91 (t, 2H), 3.39 (s, 6H), 3.77 (t, 2H), 4.08 (t, 2H), 4.36 (s, 2H), 5.09 (s, 2H), 5.36 (s, 2H), 5.62 (s, 2H), 6.87 (t, 2H), 7.20 (d, 2H), 7.29 (dd, 2H), 7.59 (d, 2H), 7.71 (d, 2H), 7.75 (d, 2H).

### Example 123

### 2-(4-fluorobenzylthio)-1-((5-((2-hydroxyethoxy)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

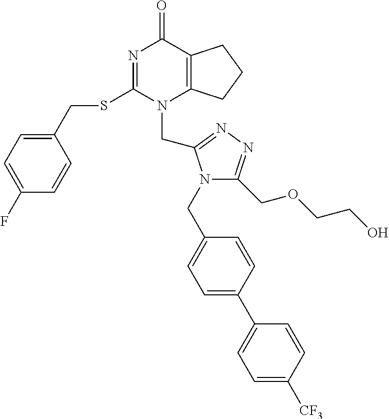 [SMILES: O=c1nc(SCc2ccc(F)cc2)n(Cc2nnc(COCCO)n2Cc2ccc(-c3ccc(C(F)(F)F)cc3)cc2)c2c1CCC2]

To an ice cooled suspension of NaH (55-65% suspended in mineral oil, 24 mg, 1.5 equiv) in anhydrous DMF (2 ml) was added ethylene glycol (42 μl, 2 equiv) under a nitrogen atmosphere. The mixture was stirred for 0.5 h, followed by addition of a solution of the compound of example 114 (250 mg) in DMF (0.5 ml) over 2 min. The resulting solution was stirred for 15 min before it was quenched with saturated aqueous NH₄Cl solution. The mixture was extracted with DCM (X2) and the combined organic phase was washed with brine (X2), dried over Na₂SO₄ and concentrated. The crude product was purified by flash chromatography to afford example 123 (80 mg) as a solid. ¹H-NMR (CDCl₃, 300 MHz) δ1.96 (m, 2H), 2.63 (t, 2H), 2.73 (t, 2H), 3.67 (m, 4H), 4.42 (s, 2H), 4.78 (s, 2H), 5.03 (s, 2H), 5.36 (s, 2H), 6.91 (t, 2H), 6.98 (d, 2H), 7.29 (dd, 2H), 7.45 (d, 2H), 7.60 (d, 2H), 7.70 (d, 2H).

### Example 124

### 2-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methoxy)acetaldehyde

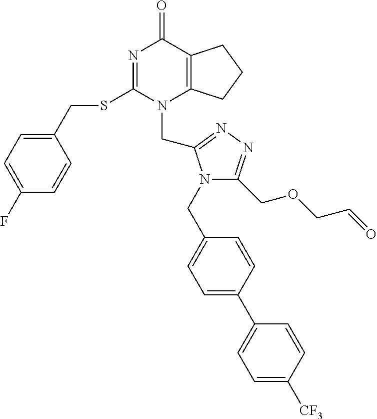 [SMILES: O=CCOCc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of intermediate D41 except that the compound of example 123 was used instead of D40.

### Example 125

### 1-((5-((2-(diethylamino)ethoxy)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

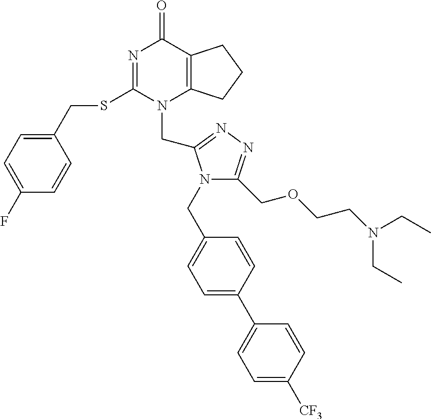 [SMILES: CCN(CC)CCOCc1nnc(Cn2c(SCc3ccc(F)cc3)nc(=O)c3c2CCC3)n1Cc1ccc(-c2ccc(C(F)(F)F)cc2)cc1]

To a mixture of the compound of example 124 (20 mg) and diethylamine (5 μl) in CH₂Cl₂ (2 ml) was added NaBH(OAc)₃ (10 mg) and the mixture was stirred for 0.5 h at room temperature. Then the solution was washed with saturated aqueous NaHCO₃ (X2) solution, dried over Na₂SO₄ and concentrated. The crude product was purified by preparative TLC (spread with DCM/MeOH=5/1) to afford example 125 (10 mg) as a solid. ¹H-NMR (CDCl₃, 300 MHz) δ 1.02 (t, 6H), 1.96 (m, 2H), 2.59-2.78 (m, 10H), 3.68 (t, 2H), 4.42 (s, 2H), 4.75 (s, 2H), 5.04 (s, 2H), 5.42 (s, 2H), 6.91 (t, 2H), 6.97 (d, 2H), 7.29 (dd, 2H), 7.43 (d, 2H), 7.59 (d, 2H), 7.70 (d, 2H); MS (ESI): 721 (M+H).

### Example 126

### 1-((5-(n-butoxymethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

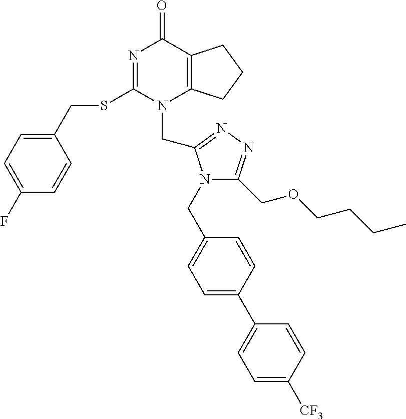 [SMILES: CCCCOCc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 123 except that 1-butanol was used instead of ethylene glycol. ¹H-NMR (CDCl₃, 400 MHz) δ 0.86 (t, 3H), 1.29 (m, 2H), 1.41 (m, 2H), 2.06 (m, 2H), 2.63 (t, 2H), 3.11 (t, 2H), 3.60 (s, 2H), 3.69 (s, 2H), 5.10 (s, 2H), 5.53 (s, 2H), 6.89 (d, 2H), 6.98 (t, 2H), 7.35 (dd, 2H), 7.46 (d, 2H), 7.60 (d, 2H), 7.69 (d, 2H).

### Example 127

### 1-((5-(1-chloroethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

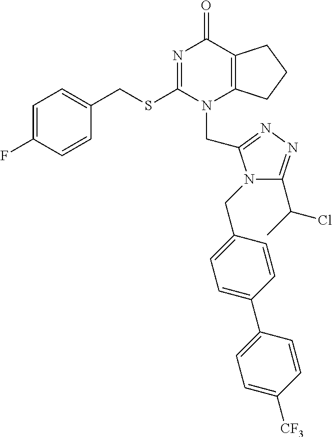 [SMILES: CC(Cl)c1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 114 except that example 120 was used instead of example 72.

### Example 128

### 1-((5-(1-(2-(dimethylamino)ethylamino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

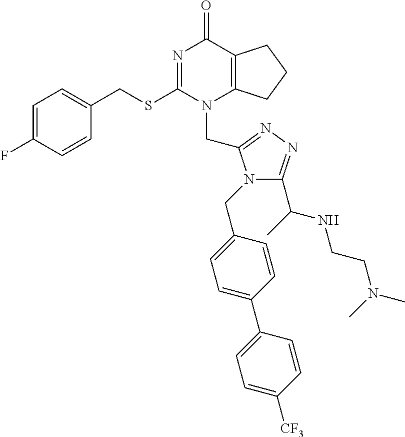 [SMILES: CC(NCCN(C)C)c1nnc(Cn2c(SCc3ccc(F)cc3)nc(=O)c3c2CCC3)n1Cc1ccc(-c2ccc(C(F)(F)F)cc2)cc1]

The mixture of the compound of Example 127 (50 mg, 1 equiv), N,N-Dimethylethylenediamine (18 μl, 2.1 equiv) and KI (1 mg) in acetonitrile (2 ml) was heated under reflux until the completion of the reaction monitored by TLC. Then solvent was removed under reduced pressure and the residue was dissolved in CH₂Cl₂, washed with brine (X3), dried over Na₂SO₄ and concentrated. The crude product was purified by preparative TLC (spread with DCM/MeOH=5/1) to afford example 128 (35 mg) as a solid. ¹H-NMR (CDCl₃, 400 MHz) δ1.53 (d, 3H), 1.98 (m, 2H), 2.24 (s, 6H), 2.37 (m, 2H), 2.55-2.78 (m, 6H), 4.11 (q, 1H), 4.43 (s, 2H), 4.98 (s, 2H), 5.53 (2×d, 2H), 6.92 (m, 4H), 7.29 (dd, 2H), 7.45 (d, 2H), 7.60 (d, 2H), 7.70 (d, 2H).

### Example 129

### 1-((5-(1-((2-(dimethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

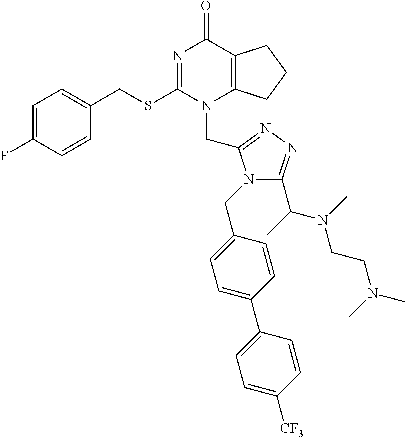 [SMILES: CC(c1nnc(Cn2c(SCc3ccc(F)cc3)nc(=O)c3c2CCC3)n1Cc1ccc(-c2ccc(C(F)(F)F)cc2)cc1)N(C)CCN(C)C]

Following a procedure similar to that described for the preparation of example 128 except that N,N,N′-trimethylethylenediamine was used instead of N,N-Dimethylethylenediamine. ¹H-NMR (CDCl₃, 400 MHz) δ1.48 (d, 3H), 2.01 (m, 2H), 2.35 (s, 3H), 2.45 (s, 6H), 2.59-3.00 (m, 8H), 4.01 (q, 1H), 4.40 (2×d, 2H), 5.00 (d, 1H), 5.26 (d, 1H), 5.56 (d, 1H), 5.74 (d, 1H), 6.87 (t, 2H), 6.99 (d, 2H), 7.26 (m, 2H), 7.40 (d, 2H), 7.56 (d, 2H), 7.69 (d, 2H).

### Example 130

### 1-((5-(1-(2-(diethylamino)ethylamino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

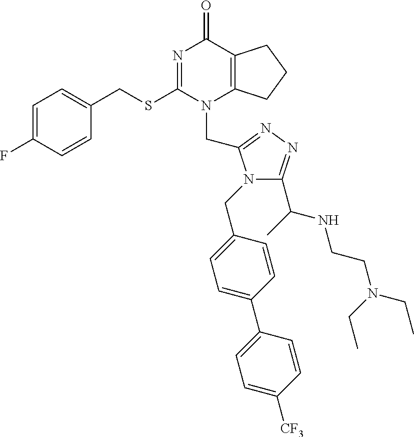 [SMILES: CCN(CC)CCNC(C)c1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 128 except that N,N-diethylethylenediamine was used instead of N,N-Dimethylethylenediamine. ¹H-NMR (CDCl₃, 300 MHz) δ1.10 (t, 6H), 1.53 (d, 3H), 1.99 (m, 2H), 2.60-2.85 (m, 12H), 4.21 (q, 1H), 4.43 (s, 2H), 4.98 (s, 2H), 5.57 (2×d, 2H), 6.93 (m, 4H), 7.30 (dd, 2H), 7.45 (d, 2H), 7.60 (d, 2H), 7.70 (d, 2H).

### Example 131a

### 1-((5-(1-((2-(diethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

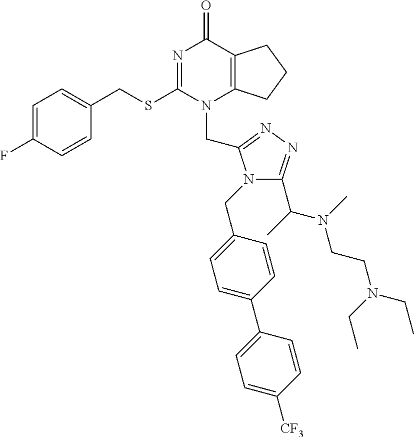 [SMILES: CCN(CC)CCN(C)C(C)c1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 128 except that N,N-diethyl-N-methylethylenediamine was used instead of N,N-Dimethylethylenediamine. ¹H-NMR (CDCl₃, 300 MHz) δ1.25 (m, 6H), 1.48 (d, 3H), 2.02 (m, 2H), 2.35 (s, 3H), 2.74 (t, 2H), 2.85-3.03 (m, 10H), 4.10 (q, 1H), 4.38 (2×d, 2H), 5.03 (d, 1H), 5.21 (d, 1H), 5.65 (2×d, 2H), 6.85 (t, 2H), 7.02 (d, 2H), 7.25 (dd, 2H), 7.39 (d, 2H), 7.55 (d, 2H), 7.69 (d, 2H).

### Example 131b

### 1-((5-(1-((2-(diethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one tartrate

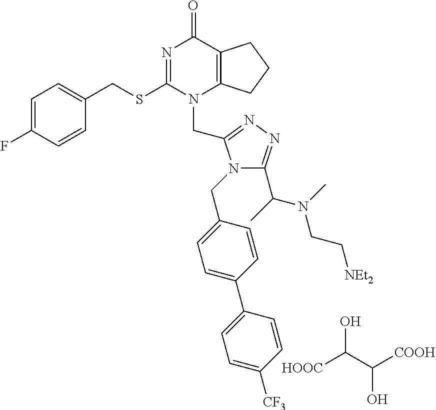

Following a procedure similar to that described for the preparation of example 91b except that example 131a was used instead of example 91a. ¹H-NMR (d₆-DMSO, 300 MHz) δ1.06 (t, 6H, J=6.9), 1.35 (d, 3H, J=6.0), 1.91 (m, 2H), 2.17 (s, 3H), 2.54 (t, 2H, J=7.2), 2.71 (m, 4H), 2.91 (m, 6H), 4.14 (q, 1H, J=6.0), 4.23 (s, 2H), 4.31 (s, 2H), 5.09/5.21 (2×d, 2H, J=12.9), 5.44/5.52 (2×d, 2H, J=16.5), 7.03 (t, 2H, J=8.4), 7.15 (d, 2H, J=7.8), 7.34 (dd, 2H, J=8.4, 6.0), 7.65 (d, 2H, J=7.8), 7.81 (m, 4H).

### Example 132

### 1-((5-(1-((3-(dimethylamino)propyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

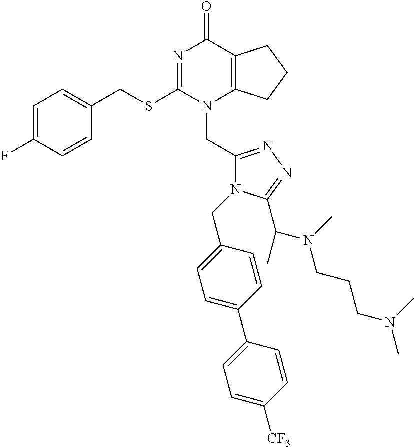 [SMILES: CC(c1nnc(Cn2c(SCc3ccc(F)cc3)nc(=O)c3c2CCC3)n1Cc1ccc(-c2ccc(C(F)(F)F)cc2)cc1)N(C)CCCN(C)C]

Following a procedure similar to that described for the preparation of example 128 except that N,N,N′-trimethyl-1,3-propanediamine was used instead of N,N-dimethylethylenediamine ¹H-NMR (CDCl₃, 300 MHz) δ1.48 (d, 3H), 1.75 (m, 2H), 2.01 (m, 2H), 2.30 (s, 3H), 2.59 (m, 9H), 2.70 (t, 2H), 2.85 (m, 3H), 4.05 (q, 1H), 4.36 (2×d, 2H), 5.00 (d, 1H), 5.38 (d, 1H), 5.50 (d, 1H), 5.67 (d, 1H), 6.84 (t, 2H), 7.03 (d, 2H), 7.23 (m, 2H), 7.40 (d, 2H), 7.55 (d, 2H), 7.69 (d, 2H).

### Example 133

### 1-((5-(((2-(dimethylamino)ethyl)(ethyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one

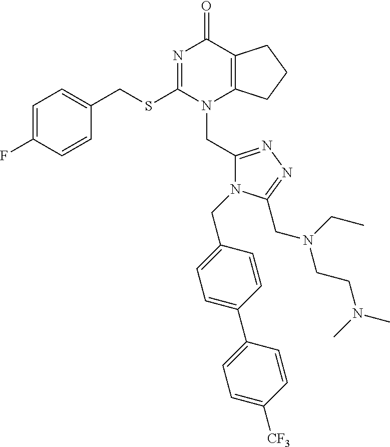 [SMILES: CCN(CCN(C)C)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that N,N-dimethyl-N-ethylethylenediamine was used instead of diethylamine ¹H-NMR (CDCl₃, 300 MHz) δ1.11 (t, 3H), 2.03 (m, 2H), 2.61 (s, 6H), 2.69 (q, 2H), 2.75 (t, 2H), 2.97 (m, 6H), 3.77 (s, 2H), 4.39 (s, 2H), 5.18 (s, 2H), 5.58 (s, 2H), 6.86 (t, 2H), 7.03 (d, 2H), 7.25 (m, 2H), 7.40 (d, 2H), 7.56 (d, 2H), 7.70 (d, 2H).

### Example 134

### ethyl-2-((2-(diethylamino)ethyl)((3-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-5-yl)methyl)amino)acetate

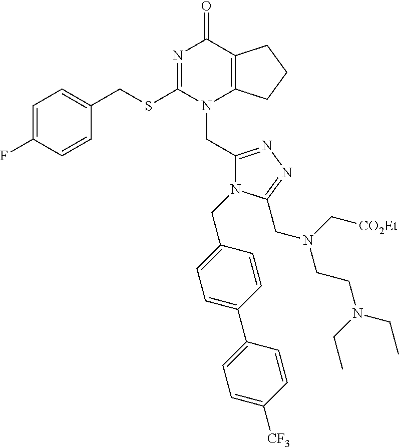 [SMILES: CCOC(=O)CN(CCN(CC)CC)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that ethyl-2-(2-(diethylamino)ethylamino)acetate was used instead of diethylamine ¹H-NMR (CDCl₃, 300 MHz) δ1.22 (m, 3H), 1.30 (m, 6H), 2.02 (m, 2H), 2.73 (t, 2H), 2.95 (t, 2H), 3.09 (m, 6H), 3.25 (t, 2H), 3.56 (s, 2H), 4.12 (q, 2H), 4.19 (s, 2H), 4.35 (s, 2H), 5.20 (s, 2H), 5.63 (s, 2H), 6.84 (t, 2H), 7.09 (d, 2H), 7.22 (dd, 2H), 7.41 (d, 2H), 7.55 (d, 2H), 7.70 (d, 2H).

### Example 135

### 2-((2-(diethylamino)ethyl)((3-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-5-yl)methyl)amino)acetic acid

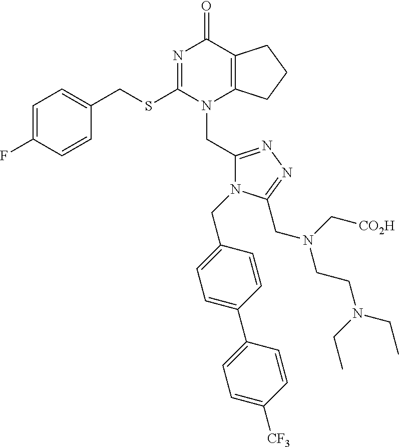 [SMILES: CCN(CC)CCN(CC(=O)O)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 63 except that example 134 was used instead of example 62. ¹H-NMR (d₆-DMSO, 300 MHz) δ0.98 (t, 6H), 1.91 (m, 2H), 2.54 (t, 2H), 2.65-2.75 (m, 10H), 3.13 (s, 2H), 3.93 (s, 2H), 4.30 (s, 2H), 5.16 (s, 2H), 5.48 (s, 2H), 7.03 (t, 2H), 7.19 (d, 2H), 7.33 (dd, 2H), 7.63 (d, 2H), 7.90 (m, 4H).

### Example 136

### 2-(4-fluorobenzylthio)-1-((5-((methyl(2-(pyrrolidin-1-yl)ethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

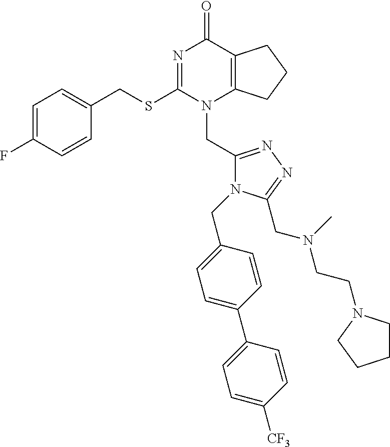 [SMILES: CN(CCN1CCCC1)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that N-methyl-2-(pyrrolidin-1-yl)ethanamine was used instead of diethylamine ¹H-NMR (CDCl₃, 300 MHz) δ2.00 (m, 6H), 2.37 (s, 3H), 2.73 (t, 2H), 2.91 (t, 2H), 3.07 (t, 2H), 3.77 (s, 2H), 4.39 (s, 2H), 5.14 (s, 2H), 5.59 (s, 2H), 6.86 (t, 2H), 7.02 (d, 2H), 7.26 (dd, 2H), 7.40 (d, 2H), 7.56 (d, 2H), 7.70 (d, 2H).

### Example 137

### N-(2-(diethylamino)ethyl)-2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxamide

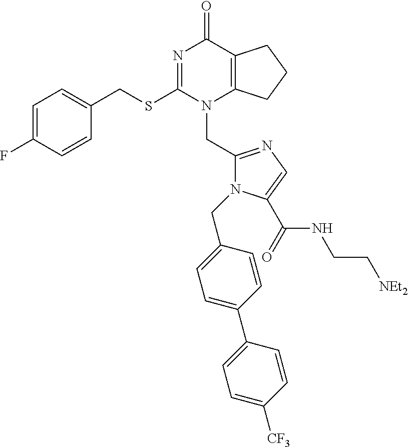

Following a procedure similar to that described for the preparation of example 51 except that N,N-diethylethylenediamine was used instead of dimethylamine hydrochloride. ¹H-NMR (CDCl₃, 300 MHz) δ 1.38 (t, 6H), 1.96 (m, 2H), 2.66 (t, 4H), 3.17 (m, 6H), 3.78 (m, 2H), 4.42 (s, 2H), 4.96 (s, 2H), 5.74 (s, 2H), 6.90 (t, 2H), 7.01 (d, 2H), 7.29 (dd, 2H), 7.43 (d, 2H), 7.58 (d, 2H), 7.68 (d, 2H), 8.03 (s, 1H), 8.95 (t, 1H, —CONH—).

### Example 138

### N-(2-(diethylamino)ethyl)-2-((2-(4-fluorobenzylthio)-4-oxo-4,5,6,7-tetrahydro-1H-cyclopenta[d]pyrimidin-1-yl)methyl)-N-methyl-1-((4′-(trifluoromethyl)biphenyl-4-yl)-methyl)-1H-imidazole-5-carboxamide

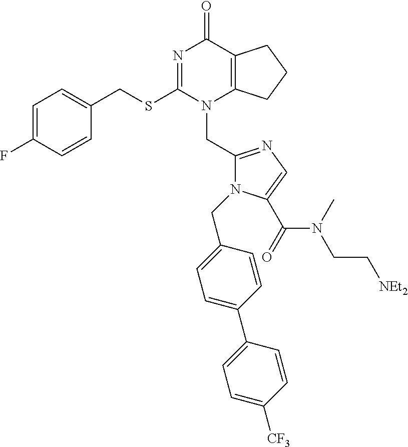

Following a procedure similar to that described for the preparation of example 51 except that N,N-diethyl-N-methylethylenediamine was used instead of dimethylamine hydrochloride. ¹H-NMR (CDCl₃, 300 MHz) δ 1.20 (t, 6H), 1.99 (m, 2H), 2.69 (m, 6H), 2.89 (m, 4H), 3.22 (s, 3H), 3.70 (t, 3H), 4.36 (s, 2H), 4.92 (s, 2H), 5.43 (s, 2H), 6.84 (t, 2H), 7.08 (d, 2H), 7.22 (dd, 2H), 7.26 (s, 1H), 7.43 (d, 2H), 7.55 (d, 2H), 7.65 (d, 2H).

### Example 139

### 1-((5-(((2-(diethylamino)ethyl)(methyl)amino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one

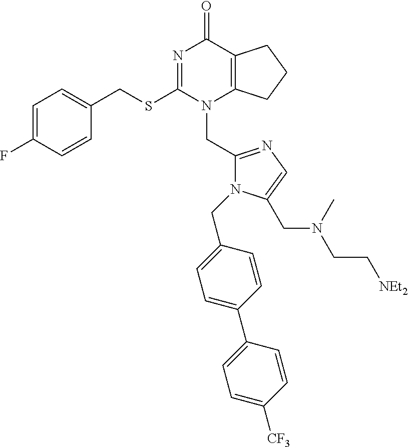

Following a procedure similar to that described for the preparation of example 45 except that N,N-diethyl-N-methylethylenediamine was used instead of 1-benzylpiperazine. ¹H-NMR (CDCl₃, 300 MHz) δ 1.08 (t, 6H), 1.98 (m, 2H), 2.21 (s, 3H), 2.67-2.85 (m, 12H), 3.47 (s, 2H), 4.42 (s, 2H), 4.95 (s, 2H), 5.46 (s, 2H), 6.89 (t, 2H), 6.95 (d, 2H), 6.96 (s, 1H), 7.27 (dd, 2H), 7.42 (d, 2H), 7.58 (d, 2H), 7.69 (d, 2H).

### Example 140

### 2-(4-fluorobenzylthio)-1-((5-((methyl(2-(piperidin-1-yl)ethyl)amino)methyl)-1-((4′-(trifluoromethyl)biphenyl-4-yl)methyl)-1H-imidazol-2-yl)methyl)-6,7-dihydro-1 H-cyclopenta[d]pyrimidin-4(5H)-one

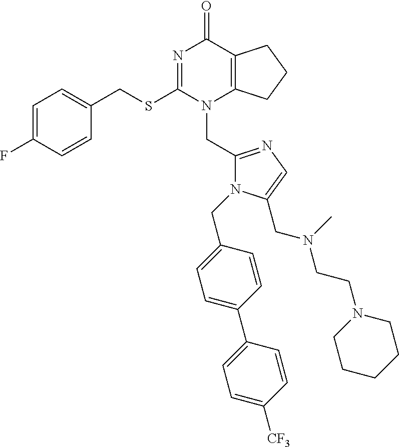 [SMILES: CN(CCN1CCCCC1)Cc1cnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 45 except that N-methyl-2-(piperidin-1-yl)ethanamine was used instead of 1-benzylpiperazine. ¹H-NMR (CDCl₃, 300 MHz) δ 1.37 (m, 2H), 1.48 (m, 4H), 1.95 (m, 2H), 2.16 (s, 3H), 2.33 (m, 4H), 2.40 (t, 2H), 2.54 (t, 2H), 2.64 (t, 2H), 2.68 (t, 2H), 3.46 (s, 2H), 4.43 (s, 2H), 4.92 (s, 2H), 5.44 (s, 2H), 6.92 (m, 5H), 7.31 (dd, 2H), 7.42 (d, 2H), 7.60 (d, 2H), 7.69 (d, 2H).

### Example 141

### 2-(4-fluorobenzylthio)-1-((5-(3-(pyrrolidin-1-yl)propyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

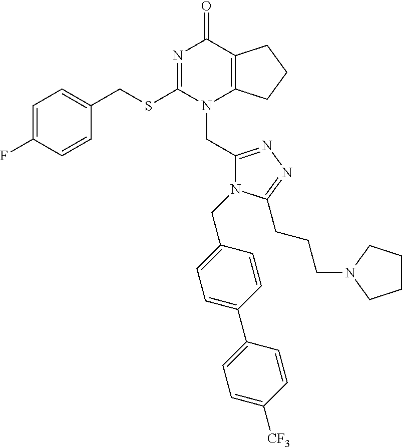 [SMILES: O=c1nc(SCc2ccc(F)cc2)n(Cc2nnc(CCCN3CCCC3)n2Cc2ccc(-c3ccc(C(F)(F)F)cc3)cc2)c2c1CCC2]

Following a procedure similar to that described for the preparation of example 70 except that pyrrolidine was used instead of diethylamine. ¹H-NMR (CDCl₃, 400 MHz) δ 1.91 (m, 4H), 1.96 (m, 2H), 2.21 (m, 2H), 2.62 (t, 2H), 2.79 (m, 6H), 2.86 (t, 2H), 2.92 (t, 2H), 4.45 (s, 2H), 5.07 (s, 2H), 5.27 (s, 2H), 6.87 (d, 2H), 6.94 (t, 2H), 7.33 (dd, 2H), 7.43 (d, 2H), 7.60 (d, 2H), 7.70 (d, 2H).

### Example 142

### 2-(4-fluorobenzylthio)-1-((5-(3-(piperidin-1-yl)propyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

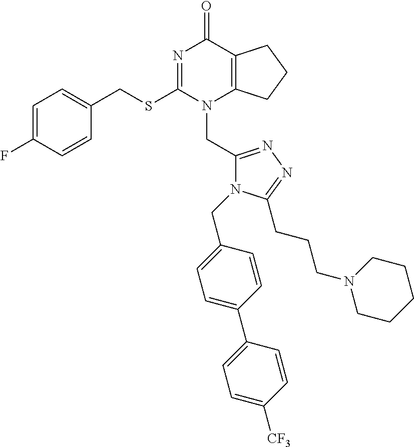 [SMILES: O=c1nc(SCc2ccc(F)cc2)n(Cc2nnc(CCCN3CCCCC3)n2Cc2ccc(-c3ccc(C(F)(F)F)cc3)cc2)c2c1CCC2]

Following a procedure similar to that described for the preparation of example 70 except that piperidine was used instead of diethylamine ¹H-NMR (CDCl₃, 300 MHz) δ 1.46 (m, 2H), 1.61 (m, 4H), 1.94 (m, 2H), 2.10 (m, 2H), 2.47 (m, 4H), 2.54 (t, 2H), 2.59 (t, 2H), 2.75 (t, 2H), 2.83 (t, 2H), 4.46 (s, 2H), 5.08 (s, 2H), 5.27 (s, 2H), 6.83 (d, 2H), 6.95 (t, 2H), 7.33 (dd, 2H), 7.43 (d, 2H), 7.61 (d, 2H), 7.70 (d, 2H).

### Example 143

### 2-(4-fluorobenzylthio)-1-((5-(4-(pyrrolidin-1-yl)butyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

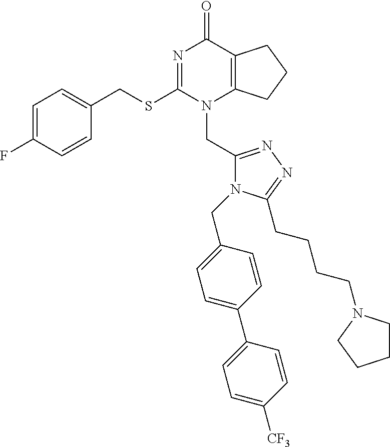 [SMILES: O=c1nc(SCc2ccc(F)cc2)n(Cc2nnc(CCCCN3CCCC3)n2Cc2ccc(-c3ccc(C(F)(F)F)cc3)cc2)c2c1CCC2]

Following a procedure similar to that described for the preparation of example 71 except that pyrrolidine was used instead of diethylamine. ¹H-NMR (CDCl₃, 300 MHz) δ 1.70 (m, 2H), 1.80 (m, 4H), 1.91 (m, 4H), 2.58 (m, 8H), 2.76 (m, 4H), 4.46 (s, 2H), 5.07 (s, 2H), 5.20 (s, 2H), 6.82 (d, 2H), 6.95 (t, 2H), 7.34 (dd, 2H), 7.44 (d, 2H), 7.62 (d, 2H), 7.70 (d, 2H).

### Example 144

### 2-(4-fluorobenzylthio)-1-((5-(4-(piperidin-1-yl)butyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

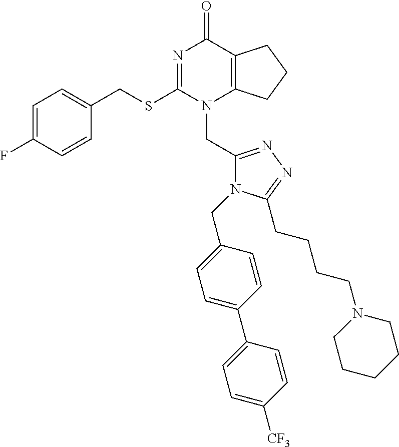 [SMILES: O=c1nc(SCc2ccc(F)cc2)n(Cc2nnc(CCCCN3CCCCC3)n2Cc2ccc(-c3ccc(C(F)(F)F)cc3)cc2)c2c1CCC2]

Following a procedure similar to that described for the preparation of example 71 except that piperidine was used instead of diethylamine ¹H-NMR (CDCl₃, 300 MHz) δ 1.80 (m, 2H), 2.00 (m, 2H), 2.18 (m, 6H), 2.58 (t, 2H), 2.66 (t, 2H), 2.85 (m, 4H), 2.95 (m, 2H), 3.50 (m, 2H), 4.40 (s, 2H), 5.11 (s, 2H), 5.29 (s, 2H), 6.89 (t, 2H), 6.95 (d, 2H), 7.28 (dd, 2H), 7.44 (d, 2H), 7.59 (d, 2H), 7.69 (d, 2H).

### Example 145

### 2-(4-fluorobenzylthio)-1-((5-((methyl(2-(piperidin-1-yl)ethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

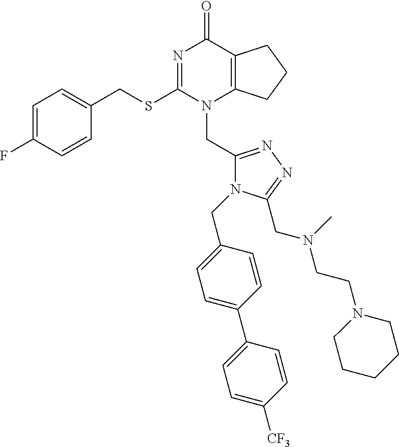 [SMILES: CN(CCN1CCCCC1)Cc1nnc(Cn2c(SCc3ccc(F)cc3)nc(=O)c3c2CCC3)n1Cc1ccc(-c2ccc(C(F)(F)F)cc2)cc1]

Following a procedure similar to that described for the preparation of example 79 except that N-methyl-2-(piperidin-1-yl)ethanamine was used instead of diethylamine ¹H-NMR (CDCl₃, 300 MHz) δ 1.47 (m, 2H), 1.66 (m, 4H), 1.98 (m, 2H), 2.29 (s, 3H), 2.67 (m, 4H), 2.80 (m, 8H), 3.82 (s, 2H), 4.40 (s, 2H), 5.07 (s, 2H), 5.57 (s, 2H), 6.88 (t, 2H), 6.98 (d, 2H), 7.29 (m, 2H), 7.41 (d, 2H), 7.58 (d, 2H), 7.70 (d, 2H).

### Example 146

### 1-((5-(((2-(diethylamino)ethyl)(methyl)amino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one tartrate

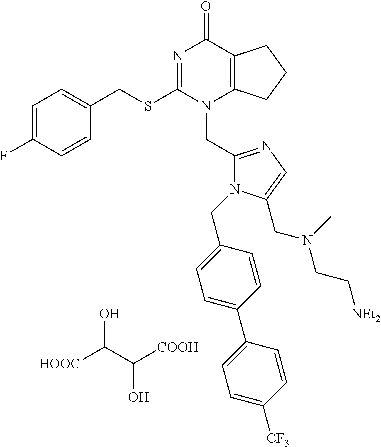

Following a procedure similar to that described for the preparation of example 91b except that example 139 was used instead of example 91a. ¹H-NMR (d₆-DMSO, 400 MHz) δ 1.04 (t, 6H), 1.88 (m, 2H), 2.12 (s, 3H), 2.53 (t, 2H), 2.59 (t, 2H), 2.67 (t, 2H), 2.90 (q, 4H), 2.99 (t, 2H), 3.43 (s, 2H), 4.20 (s, 2H), 4.29 (s, 2H), 5.05 (s, 2H), 5.38 (s, 2H), 6.86 (s, 1H), 7.02 (d, 2H), 7.07 (t, 2H), 7.33 (dd, 2H), 7.63 (d, 2H), 7.79 (m, 4H).

### Example 147

### 2-(4-fluorobenzylthio)-1-((5-((methyl(2-(piperidin-1-yl)ethyl)amino)methyl)-1-((4′-(trifluoromethyl)biphenyl-4-yl)methyl)-1H-imidazol-2-yl)methyl)-6,7-dihydro-1H-cyclopenta[d]pyrimidin-4(5H)-one tartrate

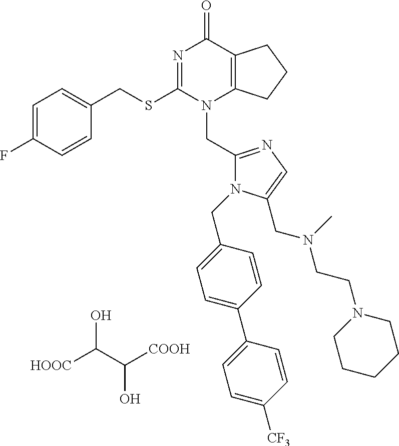 [SMILES: CN(CCN1CCCCC1)Cc1cnc(Cn2c(SCc3ccc(F)cc3)nc(=O)c3c2CCC3)n1Cc1ccc(-c2ccc(C(F)(F)F)cc2)cc1 ; O=C(O)C(O)C(O)C(=O)O]

Following a procedure similar to that described for the preparation of example 91b except that example 140 was used instead of example 91a. ¹H-NMR (d₆-DMSO, 300 MHz) δ 1.36 (m, 2H), 1.46 (m, 4H), 1.89 (m, 2H), 2.11 (s, 3H), 2.53 (m, 4H), 2.66 (m, 8H), 3.42 (s, 2H), 4.08 (s, 2H), 4.30 (s, 2H), 5.04 (s, 2H), 5.43 (s, 2H), 6.86 (s, 1H), 7.04 (t, 2H), 7.10 (d, 2H), 7.34 (dd, 2H), 7.64 (d, 2H), 7.81 (m, 2H).

### Example 148

### 1-((5-(3-(diethylamino)propyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one tartrate

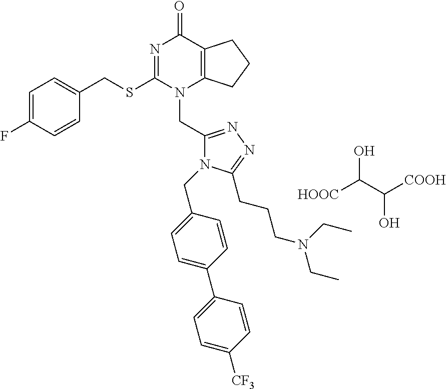

Following a procedure similar to that described for the preparation of example 91b except that example 70 was used instead of example 91a. ¹H-NMR (d₆-DMSO, 300 MHz) δ 1.09 (t, 6H), 1.92 (m, 4H), 2.55 (t, 2H), 2.74 (q, 4H), 2.93 (m, 6H), 4.08 (s, 2H), 4.33 (s, 2H), 5.23 (s, 2H), 5.36 (s, 2H), 7.04 (t, 2H), 7.16 (d, 2H), 7.36 (dd, 2H), 7.65 (d, 2H), 7.81 (m, 4H).

### Example 149

### 2-(4-fluorobenzylthio)-1-((5-(3-(piperidin-1-yl)propyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one tartrate

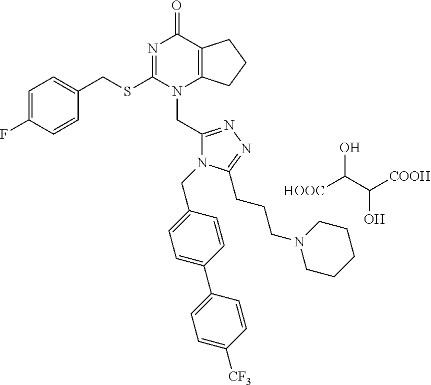

Following a procedure similar to that described for the preparation of example 91b except that example 142 was used instead of example 91a. ¹H-NMR (d₆-DMSO, 300 MHz) δ 1.45 (m, 2H), 1.62 (m, 4H), 1.95 (m, 4H), 2.55 (t, 2H), 2.73 (m, 4H), 2.83 (m, 6H), 4.13 (s, 2H), 4.33 (s, 2H), 5.23 (s, 2H), 5.36 (s, 2H), 7.04 (t, 2H), 7.15 (d, 2H), 7.36 (dd, 2H), 7.65 (d, 2H), 7.81 (d, 2H).

### Example 150

### 2-(4-fluorobenzylthio)-1-((5-(4-(piperidin-1-yl)butyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one tartrate

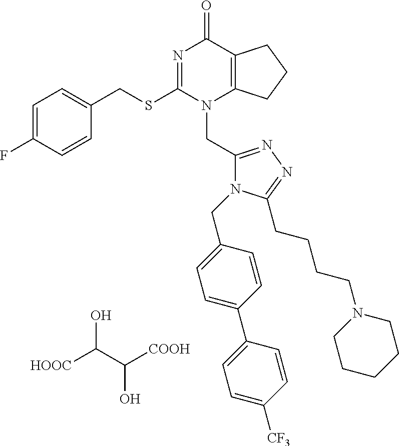 [SMILES: O=c1nc(SCc2ccc(F)cc2)n(Cc2nnc(CCCCN3CCCCC3)n2Cc2ccc(-c3ccc(C(F)(F)F)cc3)cc2)c2c1CCC2 ; O=C(O)C(O)C(O)C(=O)O]

Following a procedure similar to that described for the preparation of example 91b except that example 144 was used instead of example 91a. ¹H-NMR (d₆-DMSO, 400 MHz) δ 1.49 (m, 2H), 1.71 (m, 8H), 1.91 (m, 2H), 2.55 (t, 2H), 2.72 (m, 4H), 2.80-3.20 (m, 6H), 4.25 (s, 2H), 4.32 (s, 2H), 5.21 (s, 2H), 5.37 (s, 2H), 7.04 (t, 2H), 7.14 (d, 2H), 7.36 (dd, 2H), 7.68 (d, 2H), 7.81 (m, 4H).

### Example 151

### 1-((5-(((2-(diethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one hydrochloride

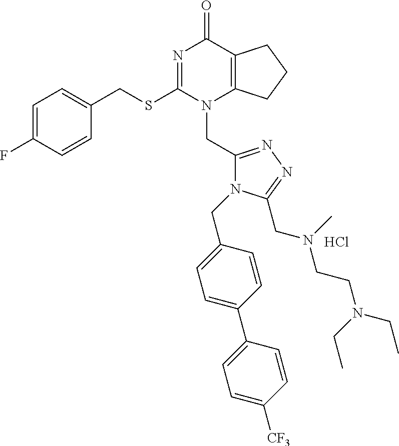 [SMILES: CCN(CC)CCN(C)Cc1nnc(Cn2c(SCc3ccc(F)cc3)nc(=O)c3c2CCC3)n1Cc1ccc(-c2ccc(C(F)(F)F)cc2)cc1 ; Cl]

Following a procedure similar to that described for the preparation of example 80 except that example 91a was used instead of example 79. ¹H-NMR (d₆-DMSO, 300 MHz) δ 1.17 (t, 6H), 1.91 (m, 2H), 2.55 (m, 4H), 2.71 (t, 2H), 3.08 (q, 4H), 3.28 (s, 3H), 3.45 (t, 2H), 4.21 (s, 2H), 4.30 (s, 2H), 5.19 (s, 2H), 5.53 (s, 2H), 7.01 (t, 2H), 7.21 (d, 2H), 7.32 (dd, 2H), 7.66 (d, 2H), 7.81 (m, 4H).

### Example 152

### 2-(4-fluorobenzylthio)-1-((5-((methyl(2-(pyrrolidin-1-yl)ethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one tartrate

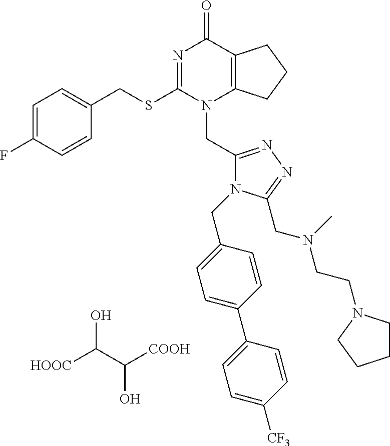 [SMILES: CN(CCN1CCCC1)Cc1nnc(Cn2c(SCc3ccc(F)cc3)nc(=O)c3c2CCC3)n1Cc1ccc(-c2ccc(C(F)(F)F)cc2)cc1 ; O=C(O)C(O)C(O)C(=O)O]

Following a procedure similar to that described for the preparation of example 91b except that example 136 was used instead of example 91a. ¹H-NMR (d₆-DMSO, 400 MHz) δ 1.78 (m, 4H), 1.91 (m, 2H), 2.20 (s, 3H), 2.55 (t, 2H), 2.70 (m, 4H), 2.99 (m, 4H), 3.10 (t, 2H), 3.73 (s, 2H), 4.18 (s, 2H), 4.31 (s, 2H), 5.17 (s, 2H), 5.48 (s, 2H), 7.03 (t, 2H), 7.19 (d, 2H), 7.34 (dd, 2H), 7.66 (d, 2H), 7.81 (m, 4H).

### Example 153

### 2-(4-fluorobenzylthio)-1-((5-((methyl(2-(piperidin-1-yl)ethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one

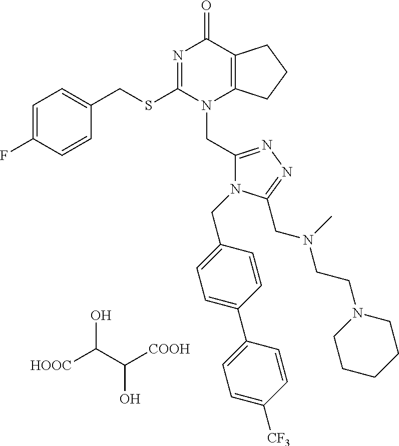 [SMILES: CN(CCN1CCCCC1)Cc1nnc(Cn2c(SCc3ccc(F)cc3)nc(=O)c3c2CCC3)n1Cc1ccc(-c2ccc(C(F)(F)F)cc2)cc1 ; O=C(O)C(O)C(O)C(=O)O]

Following a procedure similar to that described for the preparation of example 91b except that example 145 was used instead of example 91a. ¹H-NMR (d₆-DMSO, 400 MHz) δ 1.38 (m, 2H), 1.54 (m, 4H), 1.90 (m, 2H), 2.20 (s, 3H), 2.53 (t, 2H), 2.69 (t, 2H), 2.79 (t, 2H), 3.05 (m, 6H), 3.74 (s, 2H), 4.15 (s, 2H), 4.30 (s, 2H), 5.13 (s, 2H), 5.49 (s, 2H), 7.01 (d, 2H), 7.19 (t, 2H), 7.32 (m, 2H), 7.65 (d, 2H), 7.79 (d, 2H), 7.82 (d, 2H).

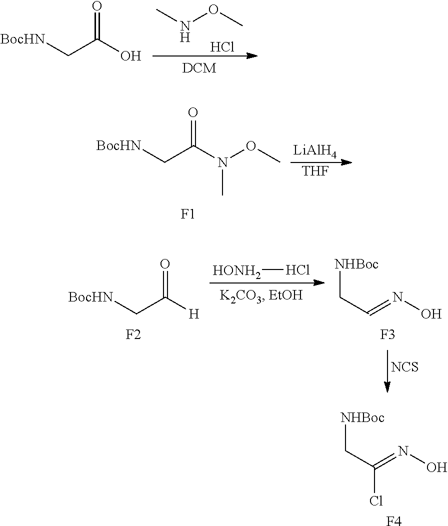

A mixture of Boc-glycine (8.75 g, 50 mmol, 1 equiv), EDCI (11.5 g, 60 mmol) and HOBt (7.43 g, 55 mmol) in DCM (100 ml) was stirred in an ice bath for 15 min to give a clear solution. Then N,O-Dimethylhydroxylamine hydrochloride (5.1 g, 52.5 mmol) and triethylamine (5.85 ml, 55 mmol) were added and the mixture was stirred for 2 h at room temperature. Water (100 ml) was added and the solution was stirred tempestuously. The precipitate so formed was filtered off and the filtrate was transferred into a separating funnel. The organic phase was separated and the aqueous phase was extracted with CH₂Cl₂ once again. The combined organic phase was washed with brine (X2), dried over MgSO₄ and concentrated. The residue was recrystallized from PE-EA to give F1 (6.0 g) as a crystal.

To an ice cooled suspension of LiAlH₄ (2.13 g, 56 mmol) in dry THF (20 ml) was added F1 (12.2 g, 56 mmol) in small batches to avoid drastic reaction. After the addition, the mixture was stirred for a further 1 h. Then the reaction was quenched cautiously by addition of saturated Na₂CO₃ solution dropwise until no gas bubbles were generated, followed by addition of water dropwise with vigorous stirring. The solid so formed was filtered off under reduced pressure and washed with THF. The filtrate was concentrated to afford F2 (9.6 g) as a jelly which could be used in next step without further purification.

A mixture of F2 (9.6 g, 60 mmol, 1 equiv), hydroxylamine hydrochloride (4.6 g, 1.1 equiv) and K₂CO₃ (12.5 g, 1.5 equiv) in EtOH (100 ml) was stirred over night at room temperature. Then the mixture was concentrated under reduced pressure to remove the solvent and the residue was diluted with water, extracted with DCM (X3). The combined organic phase was washed with brine, dried over MgSO₄. After MgSO₄ was filtered off, the filtrate was concentrated to afford F3 (7.7 g) as a jelly. ¹H-NMR (CDCl₃, 300 MHz) δ 1.45 (s, 9H), 3.91/4.04 (2×t, 2H, J=5.1, —CH₂—), 4.93 (vbrs, 1H, —NH—), 7.45/6.79 (2×t, 2H, J=4.8, —CH═), 7.72/8.04 (2×s, 1H, —OH).

To a solution of F3 (7.7 g, 44.25 mmol, 1 equiv) in dry DMF (40 ml) was added NCS (5.9 g, 1 equiv) and the mixture was stirred for 3 h at room temperature. EA (100 ml) was added and the mixture was transferred to a separating funnel, washed with brine (X5). The organic phase was dried over MgSO₄. After MgSO₄ was filtered off, the filtrate was concentrated to afford F4 (8.7 g) as a jelly.

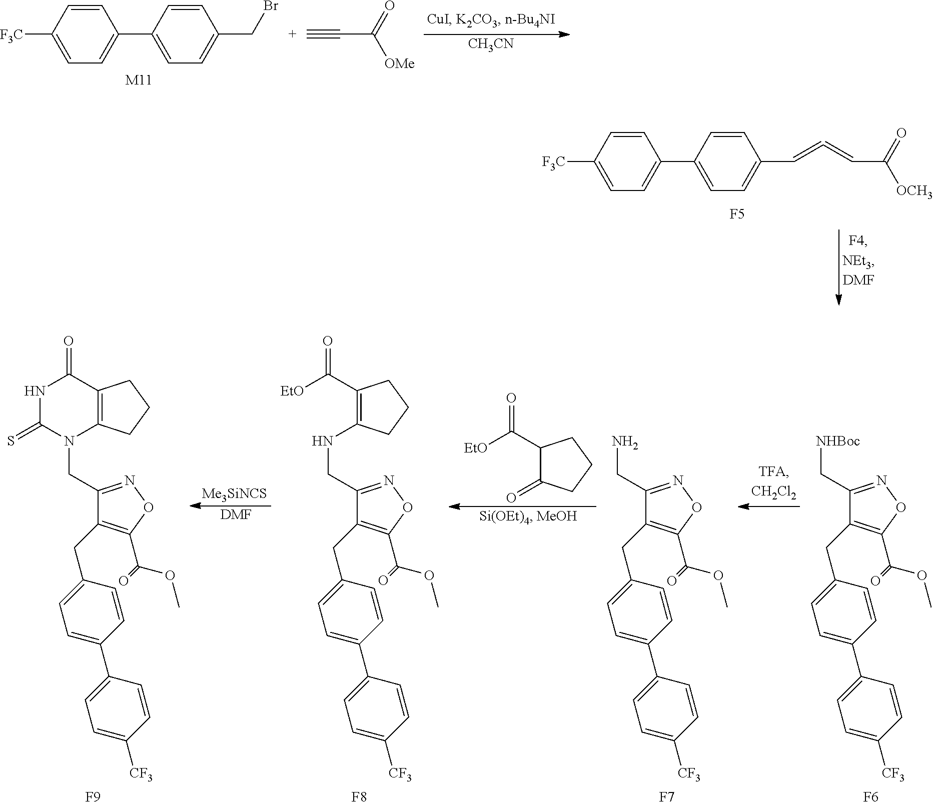

### Intermediate F5—methyl 4-(4′-(trifluoromethyl)biphenyl-4-yl)buta-2,3-dienoate

A mixture of M11 (3.66 g, 11.62 mmol, 1 equiv), methyl propiolate (1.93 ml, 23.24 mmol, 2 equiv), CuI (2.43 g, 12.78 mmol, 1.1 equiv), K₂CO₃ (3.21 g, 23.24 mmol, 2 equiv) and tetran-butylammonium iodide (4.29 g, 11.62 mmol, 1 equiv) in acetonitrile (30 ml) were stirred for 4 h at 40° C. under a nitrogen atmosphere. Then the mixture was concentrated under reduced pressure to remove the solvent and the residue was diluted with water, extracted with EA (X2). The combined organic phase was dried over Na₂SO₄ and concentrated. The crude product was purified by column chromatography to afford F5 (1.63 g) as a white solid. M11 (1.19 g) was recycled. ¹H-NMR (CDCl₃, 400 MHz) δ3.78 (s, 3H), 6.08 (d, 1H, J=6.4), 6.68 (d, 1H, J=6.4), 7.41 (d, 2H, J=8.4), 7.58 (d, 2H, J=8.4), 7.69 (m, 4H). Intermediate F6—methyl 3-((tert-butoxycarbonylamino)methyl)-4-((4′-(trifluoromethyl)biphenyl-4-yl)methyl)isoxazole-5-carboxylate

To a two-neck flask containing dry DMF (10 ml) were added F5 (1.02 g, 3.2 mmol, 1 equiv) and triethylamine (0.67 ml, 4.8 mmol, 1.5 equiv) and the mixture was placed in an oil bath of 70° C. under a nitrogen atmosphere. Then a solution of F4 (0.95 g, 4.2 mmol) in DMF (10 ml) was added dropwise over 2 h through a constant pressure drop funnel. After the addition, the mixture was stirred for a further 1 h before it was cooled and diluted with EA (100 ml). The mixture was transferred to a separating funnel, washed with brine several times, dried over Na₂SO₄ and concentrated. The residue was recrystallized from PE-EA to give F6 (0.53 g) as a white solid. F5 (0.43 g) was recycled by column chromatography. ¹H-NMR (CDCl₃, 400 MHz) δ1.41 (s, 9H), 3.99 (s, 3H), 4.21 (s, 2H), 4.32 (d, 2H, J=5.4), 4.98 (vbrs, 1H), 7.28 (d, 2H, J=7.8), 7.51 (d, 2H, J=8.1), 7.66 (m, 4H).

### Intermediate F8—methyl 3-((2-(ethoxycarbonyl)cyclopent-1-enylamino)methyl)-4-((4′-(trifluoromethyl)biphenyl-4-yl)methyl)isoxazole-5-carboxylate

To a solution of F6 (0.64 g) in DCM (5 ml) was added CF₃COOH (1 ml) and the mixture was stirred for 2 h at room temperature. Then the mixture was concentrated to remove the solvent and the residue was quenched with saturated aqueous NaHCO₃ solution. The resulting mixture was extracted with DCM and the organic phase was washed with brine, dried over MgSO₄ and concentrated to afford Intermediate F7 (0.51 g) as a jelly.

To a solution of F7 (0.51 g, 1 equiv) in anhydrous MeOH (10 ml) was added ethyl 2-oxocyclopentanecarboxylate (0.2 ml, 1 equiv) and tetraethyl orthosilicate (0.58 ml, 2 equiv). The resulting mixture was refluxed for 5 h and then concentrated. The crude product was purified by flash chromatography to afford F8 (0.52 g) as a jelly. ¹H-NMR (CDCl₃, 300 MHz) δ1.24 (t, 3H, J=6.9) 1.74 (m, 2H), 2.39 (t, 2H, J=7.2), 2.45 (t, 2H, J=7.2), 4.00 (s, 3H), 4.11 (q, 2H, J=6.9), 4.21 (d, 2H, J=6.3), 4.26 (s, 2H), 7.25 (d, 2H, J=7.8), 7.52 (d, 2H, J=8.1), 7.59 (t, 1H, —NH—), 7.67 (m, 4H).

### Intermediate F9—methyl 3-((4-oxo-2-thioxo-2,3,4,5,6,7-hexahydro-1H-cyclopenta[d]pyrimidin-1-yl)methyl)-4-((4′-(trifluoromethyl)biphenyl-4-yl)methyl)isoxazole-5-carboxylate

To a solution of F8 (0.52 g, 1 equiv) in dry DMF (1 ml) was added Me₃SiNCS (0.56 ml, 4 equiv). The mixture was stirred for 4 h at 140° C. before it was quenched with saturated aqueous NaHCO₃ solution in an ice bath. The resulting mixture was extracted with EA (X2) and the combined organic phase was washed with brine, dried and concentrated. The crude product was purified by column chromatography to afford F9 (0.18 g) as a solid. ¹H-NMR (d₆-DMSO, 300 MHz) δ1.86 (m, 2H), 2.41 (t, 2H, J=7.2), 2.69 (t, 2H, J=7.2), 3.92 (s, 3H), 4.28 (s, 2H), 5.57 (s, 2H), 7.29 (d, 2H, J=8.1), 7.61 (d, 2H, J=8.4), 7.80 (d, 2H, J=8.4), 7.85 (d, 2H, J=8.4), 12.57 (s, 1H).

### Example 154

### methyl-3-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)isoxazole-5-carboxylate

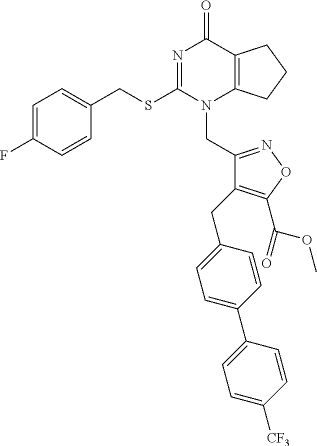 [SMILES: COC(=O)c1onc(Cn2c(SCc3ccc(F)cc3)nc(=O)c3c2CCC3)c1Cc1ccc(-c2ccc(C(F)(F)F)cc2)cc1]

A mixture of F9 (0.15 g, 1 equiv), 4-fluorobenzyl bromide (38 μl, 1.1 equiv) and DBU (50 μl, 1.2 equiv) in acetonitrile (3 ml) was stirred at room temperature for 1 h and then concentrated to remove the solvent. The residue was dissolved in DCM, washed with water, dried and concentrated. The crude product was purified by column chromatography to afford 154 (0.15 g) as a solid. ¹H-NMR (CDCl₃, 300 MHz) δ 1.93 (m, 2H), 2.58 (t, 2H, J=7.5), 2.66 (t, 2H, J=7.5), 4.02 (s, 3H), 4.22 (s, 2H), 4.43 (s, 2H), 4.86 (s, 2H), 6.92 (t, 2H, J=8.7), 7.10 (d, 2H, J=8.1), 7.28 (dd, 2H, J=9.0, 5.4), 7.44 (d, 2H, J=8.1), 7.61 (d, 2H, J=8.1), 7.70 (d, 2H, J=8.4).

### Example 155

### 2-(4-fluorobenzylthio)-1-((5-(hydroxymethyl)-4-((4′-(trifluoromethyl)biphenyl-4-yl)methyl)isoxazol-3-yl)methyl)-6,7-dihydro-1H-cyclopenta[d]pyrimidin-4(5H)-one

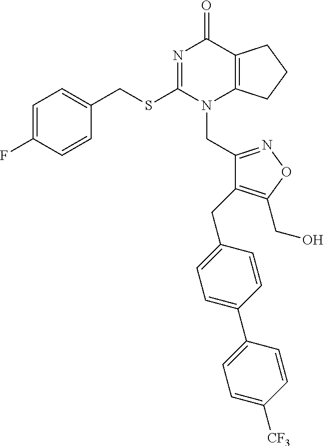 [SMILES: O=c1nc(SCc2ccc(F)cc2)n(Cc2noc(CO)c2Cc2ccc(-c3ccc(C(F)(F)F)cc3)cc2)c2c1CCC2]

To a solution of the compound of Example 154 (0.15 g, 1 equiv) in MeOH (3 ml) was added NaBH₄ (10 mgX4) portion-wise over a period of 0.5 h. Then the solution was evaporated to dryness, diluted with water and extracted with DCM (X2). The combined organic phase was washed with brine (X1), dried over Na₂SO₄ and concentrated. The crude product was purified by column chromatography to afford 155 (100 mg) as a white solid. ¹H-NMR (CDCl₃, 400 MHz) δ1.91 (m, 2H), 2.60 (m, 4H), 2.95 (s, 1H), 3.98 (s, 2H), 4.41 (s, 2H), 4.81 (s, 2H), 4.84 (s, 2H), 6.91 (t, 2H, J=8.4), 7.10 (d, 2H, J=8.0), 7.29 (m, 2H), 7.43 (d, 2H, J=8.0), 7.61 (d, 2H, J=7.6), 7.69 (d, 2H, J=8.0).

### Example 156

### 1-((5-(chloromethyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one

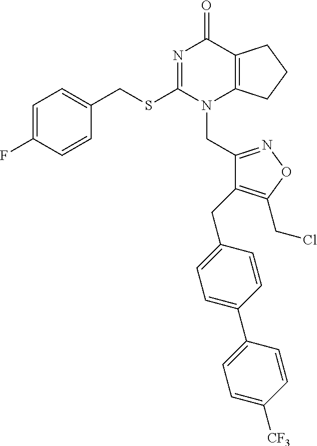 [SMILES: O=c1nc(SCc2ccc(F)cc2)n(Cc2noc(CCl)c2Cc2ccc(-c3ccc(C(F)(F)F)cc3)cc2)c2c1CCC2]

To a solution of the compound of Example 155 (90 mg, 1 equiv) in DCM (2 ml) was added thionyl chloride (12 μl, 1.2 equiv) in an ice bath. The mixture was stirred for 1 h before it was quenched with saturated aqueous NaHCO₃ solution. The organic phase was separated, dried over MgSO₄ and concentrated to afford 156 (90 mg) as a solid. ¹H-NMR (CDCl₃, 400 MHz) δ1.94 (m, 2H), 2.64 (m, 4H), 3.87 (s, 2H), 4.45 (s, 2H), 4.63 (s, 2H), 4.84 (s, 2H), 6.93 (t, 2H, J=8.4), 7.10 (d, 2H, J=8.4), 7.31 (dd, 2H, J=8.8, 5.2), 7.46 (d, 2H, J=8.4), 7.63 (d, 2H, J=8.4), 7.70 (d, 2H, J=8.4).

### Example 157

### 1-((5-((diethylamino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one

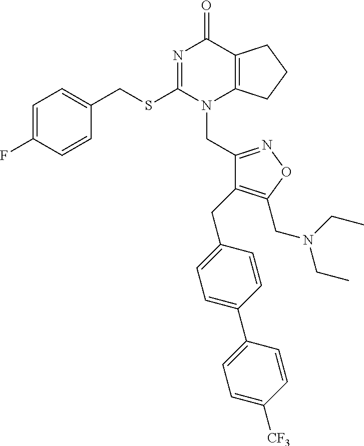 [SMILES: CCN(CC)Cc1onc(Cn2c(SCc3ccc(F)cc3)nc(=O)c3c2CCC3)c1Cc1ccc(-c2ccc(C(F)(F)F)cc2)cc1]

A mixture of the compound of Example 156 (53 mg, 0.083 mmol, 1 equiv) and diethylamine (11 μl, 1.3 equiv), K₂CO₃ (23 mg, 2 equiv) and KI (1 mg, cat.) in acetonitrile (2 ml) was stirred under reflux for 1 h, and then filtered to remove the insoluble salts and concentrated. The crude product was purified by column chromatography to afford 157 (50 mg) as a solid. ¹H-NMR (CDCl₃, 400 MHz) δ 1.07 (t, 6H, J=7.2), 1.94 (m, 2H), 2.62 (m, 8H), 3.81 (s, 2H), 3.91 (s, 2H), 4.43 (s, 2H), 4.81 (s, 2H), 6.92 (t, 2H, J=8.4), 7.08 (d, 2H, J=8.0), 7.30 (dd, 2H, J=8.0, 5.6), 7.43 (d, 2H, J=8.0), 7.62 (d, 2H, J=8.0), 7.69 (d, 2H, J=8.0); MS (ESI): 677 (M+H).

### Example 158

### 1-((5-(((2-(dimethylamino)ethyl)(methyl)amino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one

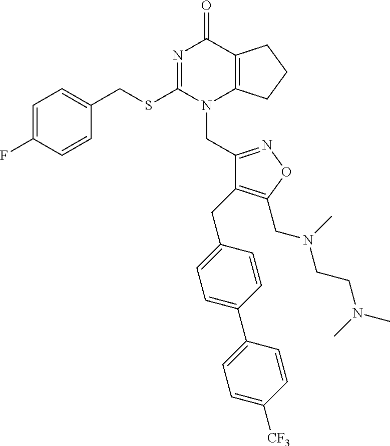 [SMILES: CN(C)CCN(C)Cc1onc(Cn2c(SCc3ccc(F)cc3)nc(=O)c3c2CCC3)c1Cc1ccc(-c2ccc(C(F)(F)F)cc2)cc1]

Following a procedure similar to that described for the preparation of example 157 except that N,N,N′-trimethylethylenediamine was used instead of diethylamine ¹H-NMR (CDCl₃, 400 MHz) δ1.98 (m, 2H), 2.35 (s, 3H), 2.60 (s, 6H), 2.70 (m, 4H), 2.89 (m, 4H), 3.80 (s, 2H), 3.95 (s, 2H), 4.41 (s, 2H), 4.86 (s, 2H), 6.89 (t, 2H, J=8.4), 7.11 (d, 2H, J=8.0), 7.27 (m, 2H), 7.42 (d, 2H, J=8.4), 7.60 (d, 2H, J=8.4), 7.69 (d, 2H, J=8.0).

### Example 159

### 1-((5-(((2-(diethylamino)ethyl)(methyl)amino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one

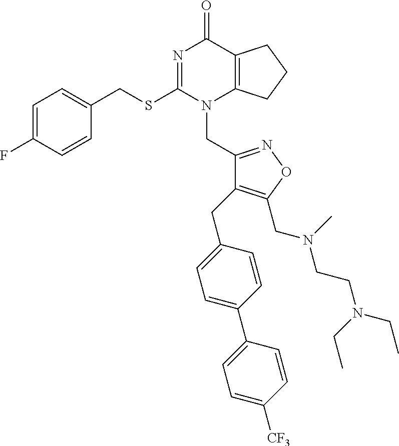 [SMILES: CCN(CC)CCN(C)Cc1onc(Cn2c(SCc3ccc(F)cc3)nc(=O)c3c2CCC3)c1Cc1ccc(-c2ccc(C(F)(F)F)cc2)cc1]

Following a procedure similar to that described for the preparation of example 157 except that N,N-diethyl-N′-methylethylenediamine was used instead of diethylamine. ¹H-NMR (CDCl₃, 300 MHz) δ1.25 (t, 6H, J=7.2), 1.97 (m, 2H), 2.34 (s, 3H), 2.70 (m, 4H), 2.94-3.03 (m, 8H), 3.80 (s, 2H), 3.95 (s, 2H), 4.40 (s, 2H), 4.86 (s, 2H), 6.88 (t, 2H, J=8.7), 7.10 (d, 2H, J=7.8), 7.27 (m, 2H), 7.42 (d, 2H, J=8.1), 7.59 (d, 2H, J=8.1), 7.69 (d, 2H, J=7.8).

### Example 160

### 1-((5-(((2-(diethylamino)ethyl)(methyl)amino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one single-tartrate

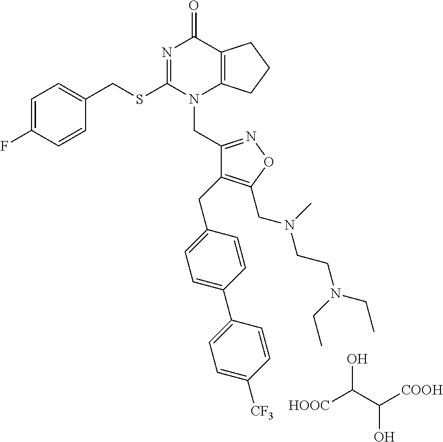

Following a procedure similar to that described for the preparation of example 91b except that example 159 was used instead of example 91a. ¹H-NMR (d₆-DMSO, 300 MHz) δ1.14 (t, 6H), 1.87 (m, 2H), 2.22 (s, 3H), 2.67 (t, 2H), 2.75 (t, 2H), 3.05-3.13 (m, 8H), 3.85 (s, 2H), 3.95 (s, 2H), 4.24 (s, 2H), 4.32 (s, 2H), 5.03 (s, 2H), 7.04 (t, 2H), 7.22 (d, 2H), 7.35 (m, 2H), 7.60 (d, 2H), 7.80 (m, 4H).

### Example 161

### 2-(4-fluorobenzylthio)-1-((5-((methyl(2-(piperidin-1-yl)ethyl)amino)methyl)-4-((4′-(trifluoromethyl)biphenyl-4-yl)methyl)isoxazol-3-yl)methyl)-6,7-dihydro-1H-cyclopenta[d]pyrimidin-4(5H)-one

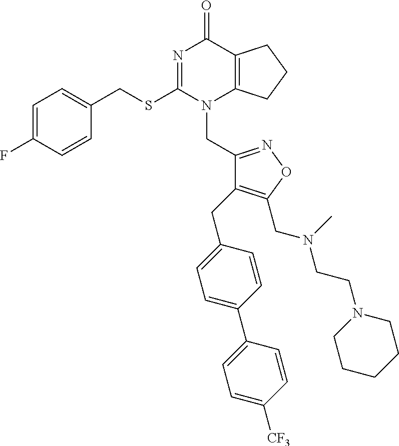 [SMILES: CN(CCN1CCCCC1)Cc1onc(Cn2c(SCc3ccc(F)cc3)nc(=O)c3c2CCC3)c1Cc1ccc(-c2ccc(C(F)(F)F)cc2)cc1]

Following a procedure similar to that described for the preparation of example 157 except that N-methyl-2-(piperidin-1-yl)ethanamine was used instead of diethylamine ¹H-NMR (CDCl₃, 400 MHz) δ1.55 (m, 2H), 1.81 (m, 4H), 1.97 (m, 2H), 2.34 (s, 3H), 2.68 (t, 2H, J=7.2), 2.74 (t, 2H, J=7.2), 2.82 (m, 4H), 2.90 (m, 4H), 3.78 (s, 2H), 3.95 (s, 2H), 4.40 (s, 2H), 4.88 (s, 2H), 6.88 (t, 2H, J=8.4), 7.11 (d, 2H, J=8.0), 7.26 (m, 2H), 7.41 (d, 2H, J=8.1), 7.59 (d, 2H, J=8.0), 7.69 (d, 2H, J=8.4).

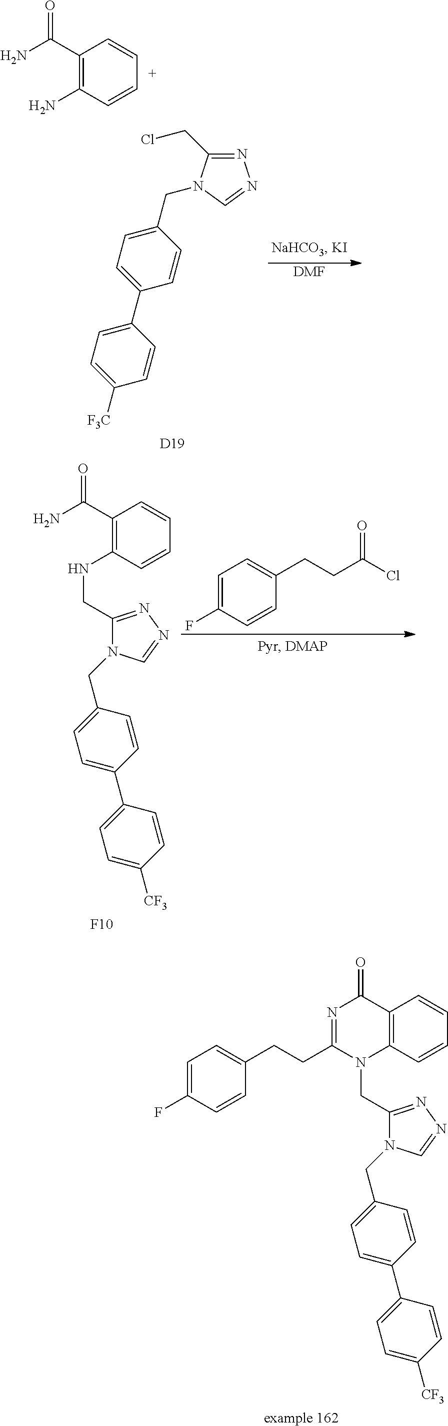

### Example 162

### 2-(4-fluorophenethyl)-1-((4-((4′-(trifluoromethyl)biphenyl-4-yl)methyl)-4H-1,2,4-triazol-3-yl)methyl)quinazolin-4(1H)-one

The mixture of 2-aminobenzamide (320 mg, 2.3 mmol, 1 equiv), D19 (1 g, 1.1 equiv), NaHCO₃ (580 mg, 3 equiv) and KI (38 mg, 0.1 equiv) in DMF (10 ml) was heated for 2 h at 80° C. Then it was poured into water and extracted with EA. The organic phase was dried and concentrated. The crude product was purified by column chromatography to afford F10 (380 mg) as a solid.

The mixture of F10 (370 mg, 0.82 mmol, 1 equiv), 3-(4-fluorophenyl)propanoyl chloride (185 mg, 1.2 equiv), pyridine (132 μl, 2 equiv) and DMAP (4 mg) in dry 1,4-dioxacyclohexane (5 ml) was refluxed over night and then mixed with silica gel and concentrated to remove the solvent. The crude product was purified by column chromatography to afford 161 (50 mg) as a solid. ¹H-NMR (CDCl₃, 400 MHz) δ3.25 (t, 2H, J=6.8), 3.35 (t, 2H, J=6.8), 5.30 (s, 2H), 5.32 (s, 2H), 6.93 (t, 2H, J=8.8), 7.20 (dd, 2H, J=8.4, 5.6), 7.31 (d, 2H, J=8.0), 7.44 (t, 1H, J=7.2), 7.56 (d, 2H, J=8.4), 7.65 (m, 4H), 7.70 (d, 2H, J=8.4), 8.06 (s, 1H), 8.37 (d, 1H, J=8.0); MS (ESI): 584 (M+H).

### Pharmacological Examples

### 1. Test of In Vitro Inhibitory Activities of the Compounds Towards the Enzyme Lp-PLA₂ in Rabbit Serum

1.1 Test method

The activities of compounds were measured using [³H] labeled platelet activating factor ([³H]PAF, Perkinelmer, Lot NET910) as the substrate. The reaction was carried out in a system containing 50 mmol/L HEPES and 150 mmol/L NaCl (pH 7.4) in a total volume of 200 μl. First, 10 μL rabbit serum and 10 μl DMSO solution of the test compound were added to the system and preincubated for 10 min at 37° C. Then 20 nM [³H]PAF was added as listed in table 1 to start the reaction and incubated for a further 10 min at 37° C. After that, 600 μl of CHCl₃/MeOH (2:1) was added and the resulting mixture was vortex mixed and the reaction was stopped. After settled for a while, the mixture was centrifugated at 12000×g for 15 min. The supernatant was transferred to a new tube and 200 μl of CHCl₃ was added. The mixture was mixed by vortexing and then settled or centrifugated for 2 min. A 100 μl portion of the supernatant was then collected for radioactivity intensity measurement.

TABLE 1
reaction system lists

| | blank tube(μl) | control tube(μl) | compound tube(μl) |
|---|---|---|---|
| reaction buffer | 180 | 170 | 170 |
| test compound | | | 10 |
| DMSO (solvent) | 10 | 10 | |
| substrate | 10 | 10 | 10 |
| ([³H] PAF) | | | |
| enzyme source | | 10 | 10 |
| (rabbit serum) | | | |

The inhibition rate was determined by the following equation:

Inhibitory rate (%)=1−(DPM_{compound tube}−DPM_{blank tube})/(DPM_{control tube}−DPM_{blank tube})*100%

(Note: DPM is radioactivity intensity unit.)

### 1.2 Test Results are Listed in Table 2.

TABLE 2
inhibitory activities of part of the compounds towards the
enzyme Lp-PLA₂ in rabbit serum at different concentration

| | inhibition, % | | |
|---|---|---|---|
| compound | 10 μM | 1 μM | 100 nM |
| Example 1 | 62.6 | 10.0 | NT |
| Example 2 | 82.7 | 26.6 | NT |
| Example 3 | 12.5 | NT | NT |
| Example 6 | 74.0 | 16.2 | NT |
| Example 7 | 60.3 | 25.0 | NT |
| Example 18 | 41.9 | 7.4 | NT |
| Example 19 | 76.8 | 29 | NT |
| Example 20 | 86.6 | 31 | NT |
| Example 21 | 51.2 | — | NT |
| Example 22 | 63.0 | — | NT |
| Example 23 | 82.4 | 33 | NT |
| Example 24 | 78.5 | 16.3 | NT |
| Example 25 | 74.3 | 23 | NT |
| Example 26 | 7.4 | NT | NT |
| Example 28 | 96.5 | 79.6 | 16.9 |
| Example 29 | NT | 64.9 | 12.6 |
| Example 30 | 87.5 | 42.3 | — |
| Example 32 | NT | 50.0 | 2.1 |
| Example 34 | NT | 14.6 | NT |
| Example 38 | 20.4 | 16.0 | NT |
| Example 40 | NT | 90.7 | 53.1 |
| Example 41 | NT | 64.9 | 12.6 |
| Example 42 | NT | 97.7 | 88.3 |
| Example 43 | NT | NT | 78.6 |
| Example 44 | NT | NT | 89.1 |
| Example 45 | NT | NT | 87 |
| Example 46 | NT | 97.2 | 87.1 |
| Example 47 | NT | 98.6 | 96.9 |
| Example 49 | NT | NT | 96.6 |
| Example 50 | NT | NT | 57 |
| Example 52 | NT | NT | 90 |
| Example 53 | NT | 90.1 | 43.2 |
| Example 54 | NT | 88.8 | 35.9 |
| Example 55 | 33.6 | 21 | NT |
| Example 57 | 72.4 | 13 | NT |
| Example 58 | 94.8 | 32.0 | NT |
| Example 59 | 9.3 | — | NT |
| Example 60 | 40.0 | — | NT |
| Example 61 | NT | NT | 61.0 |
| Example 62 | NT | NT | 81.9 |
| Example 62 | NT | NT | 73.8 |
| Example 64 | NT | NT | 78.9 |
| Example 65 | NT | NT | 60.0 |
| Example 66 | NT | NT | 88 |
| Example 67 | NT | NT | 91 |
| Example 68 | NT | 87 | 34 |
| Example 69 | NT | NT | 89 |
| Example 70 | NT | NT | 96 |
| Example 72 | NT | NT | 73 |
| Example 75 | NT | NT | 88.3 |
| Example 76 | NT | NT | 97.0 |
| Example 77 | NT | NT | 93 |
| Example 79 | NT | NT | 85.3 |
| Example 81 | NT | NT | 90 |
| Example 82 | NT | NT | 85 |
| Example 83 | NT | NT | 93 |
| Example 84 | NT | NT | 76 |
| Example 85 | NT | NT | 77 |
| Example 86 | NT | NT | 90 |
| Example 87 | NT | NT | 75 |
| Example 88 | NT | NT | 67 |
| Example 89 | NT | NT | 13.4 |
| Example 90 | NT | NT | 96 |
| Example 91a | NT | NT | 98 |
| Example 92 | NT | NT | 98 |
| Example 93 | NT | NT | 98 |
| Example 94 | NT | NT | 98 |
| Example 95 | NT | NT | 99 |
| Example 96 | NT | NT | 97 |
| Example 97 | NT | NT | 98 |
| Example 98 | NT | NT | 97 |
| Example 99 | NT | NT | 97 |
| Example 100 | NT | NT | 91 |
| Example 101 | NT | NT | 86 |
| Example 102 | NT | NT | 97 |
| Example 103 | NT | NT | 82 |
| Example 104 | NT | NT | 67 |
| Example 105 | NT | NT | 98 |
| Example 106 | NT | NT | 96 |
| Example 107 | NT | NT | 68 |
| Example 110 | NT | NT | 67 |
| Example 111 | NT | NT | 75 |
| Example 112 | NT | NT | 63 |
| Example 113 | NT | NT | 64 |
| Example 115 | NT | NT | 78 |
| Example 116 | NT | NT | 94 |
| Example 117 | NT | NT | 80 |
| Example 118 | NT | NT | 68 |
| Example 119 | NT | NT | 93 |
| Example 120 | NT | NT | 90 |
| Example 122 | NT | NT | 95 |
| Example 123 | NT | NT | 88 |
| Example 125 | NT | NT | 92 |
| Example 128 | NT | NT | 94 |
| Example 129 | NT | NT | 96 |
| Example 130 | NT | NT | 94 |
| Example 131a | NT | NT | 96 |
| Example 132 | NT | NT | 96 |
| Example 133 | NT | NT | 97 |
| Example 135 | NT | NT | 83 |
| Example 157 | NT | NT | 89 |
| Example 158 | NT | NT | 98 |
| Example 159 | NT | NT | 97 |
| Example 161 | NT | NT | 99 |

Note:
“—”, inactive;
“NT”, not tested.

### 2. Test of In Vitro Inhibitory Activities of the Compounds Towards the Enzyme Lp-PLA₂ in Human Serum

### 2.1 Test Method

The activities of compounds were measured using [³H] labeled platelet activating factor ([³H]PAF, Perkinelmer, Lot NET910) as the substrate. The reaction was carried out in a system containing 50 mmol/l HEPES and 150 mmol/l NaCl (pH 7.4) in a total volume of 200 μl. First, 10 μL human plasma and 10 μl DMSO solution of the test compound were added to the system and preincubated for 10 min at 37° C. Then 20 nM [³H]PAF was added as listed in table 1 to start the reaction and incubated for a further 10 min at 37° C. After that, 600 μl of CHCl₃/MeOH (2:1) was added and the resulting mixture was mixed with vortex. After settled for a while, the mixture was centrifugated at 12000×g for 15 min. The supernatant was transferred to a new tube and 200 μl of CHCl₃ was added and mixed by vortexing. The mixture was settled or centrifugated for 2 min. A 100 μl portion of the supernatant was then collected for radioactivity intensity measurement.

### 2.2 Test Results are Listed in Table3.

TABLE 3
inhibitory activities of part of the compounds towards the
enzyme Lp-PLA₂ in human serum at different concentration

inhibition, %

| compound | 100 nM | 10 nM |
|---|---|---|
| Example 42 | 92 | 75 |
| Example 43 | 92 | 67 |
| Example 44 | 95 | 69 |
| Example 45 | 90 | 56 |
| Example 46 | 89 | 75 |
| Example 47 | 99 | 93 |
| Example 49 | 98 | 97 |
| Example 52 | 99 | 97 |
| Example 61 | 91 | 59 |
| Example 62 | 96 | 83 |
| Example 62 | 94 | 76 |
| Example 64 | 93 | 73 |
| Example 65 | 96 | 82 |
| Example 66 | 98 | 88 |
| Example 67 | 98 | 92 |
| Example 68 | 97 | 93 |
| Example 69 | 97 | 87 |
| Example 70 | 97 | 91 |
| Example 72 | 91 | 65 |
| Example 75 | 97 | 85 |
| Example 76 | 100 | 100 |
| Example 77 | 98 | 97 |
| Example 79 | 93 | 68 |
| Example 81 | 98 | 93 |
| Example 82 | 98 | 77 |
| Example 83 | 95 | 81 |
| Example 84 | 91 | 69 |
| Example 85 | 91 | 59 |
| Example 86 | 96 | 82 |
| Example 87 | 87 | 76 |
| Example 88 | 91 | 59 |
| Example 90 | 100 | 95 |
| Example 91a | NT | 94 |
| Example 92 | NT | 93 |
| Example 93 | NT | 85 |
| Example 94 | NT | 89 |
| Example 95 | NT | 86 |
| Example 96 | NT | 78 |
| Example 97 | NT | 81 |
| Example 98 | NT | 82 |
| Example 99 | NT | 81 |
| Example 100 | 98 | 91 |
| Example 101 | 96 | 77 |
| Example 102 | NT | 77 |
| Example 103 | 98 | 71 |
| Example 104 | 86 | 53 |
| Example 105 | NT | 72 |
| Example 106 | NT | 80 |
| Example 107 | 91 | 70 |
| Example 110 | 83 | 67 |
| Example 111 | 92 | 68 |
| Example 112 | 87 | 56 |
| Example 113 | 91 | 56 |
| Example 115 | NT | 77 |
| Example 116 | 100 | 88 |
| Example 117 | 92 | 67 |
| Example 118 | 88 | 58 |
| Example 119 | 98 | 94 |
| Example 120 | 98 | 89 |
| Example 122 | NT | 92 |
| Example 123 | NT | 76 |
| Example 125 | NT | 76 |
| Example 128 | NT | 90 |
| Example 129 | NT | 94 |
| Example 130 | NT | 92 |
| Example 131a | NT | 92 |
| Example 132 | NT | 93 |
| Example 133 | NT | 92 |
| Example 135 | NT | 66 |
| Example 157 | NT | 96 |
| Example 158 | NT | 98 |
| Example 159 | NT | 100 |
| Example 161 | NT | 100 |

Note:
“NT”, not tested.

### 3. Analysis on the Pharmacological Results

From the in vitro activity test results, it can be seen that most compounds have a relatively good Lp-PLA₂ inhibitory activity. The inhibitory rate of compounds 47, 49, 52, 67, 70, 76, 77, 81, 83, 86, 90-100, 106, 116, 119, 120, 122, 128, 133, 158, 159, 161 at 100 nM towards the rabbit serum derived Lp-PLA₂ enzyme are all greater than 90%, and at 10 nM towards the human serum derived Lp-PLA₂ enzyme are all greater than 80%. It can be seen that the compounds of formula (I), (II) or (III) of the present application are potent Lp-PLA₂ inhibitors.

All literatures mentioned in the present application are incorporated by reference herein, as though individually incorporated by reference. Additionally, it should be understood that after reading the description above, many variations and modifications may be made by the skilled in the art, and these equivalents also fall within the scope as defined by the appended claims.

1. An azole heterocyclic compound of Formula (I), (II) or (III), cis-trans isomers, enantiomers, diastereoisomers, racemes, solvates, hydrates, or pharmaceutically acceptable salts thereof: 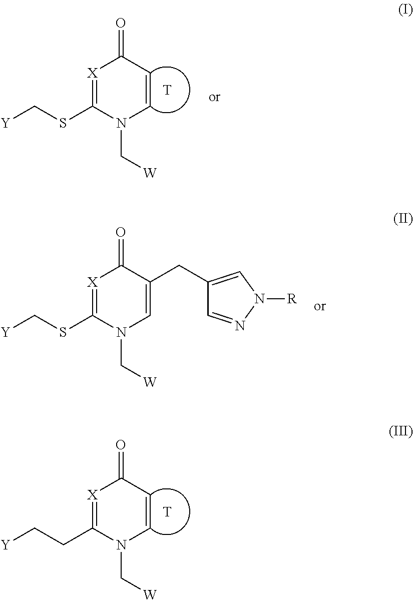
in which:
T is 4 to 6-membered aliphatic ring or benzene ring;
R is C₁-C₆ alkyl;
X is CH or N;
Y is phenyl group, optionally substituted by one or more substituents selected from halogen, C₁-C₆ alkoxyl, C₁-C₆ alkyl or halogenated C₁-C₆ alkyl;
W is selected from 12 structures of Formulae (a-1) as follows: 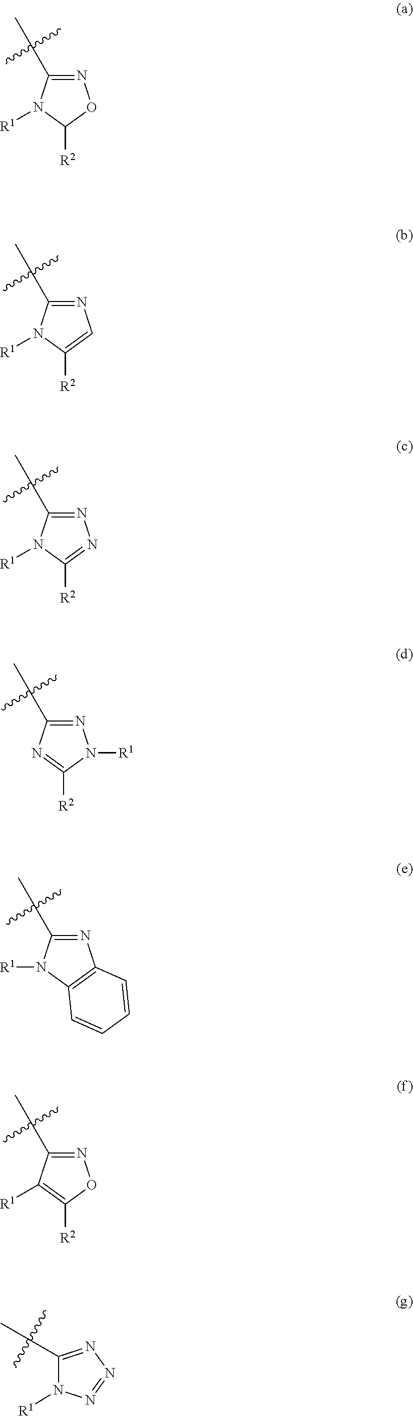 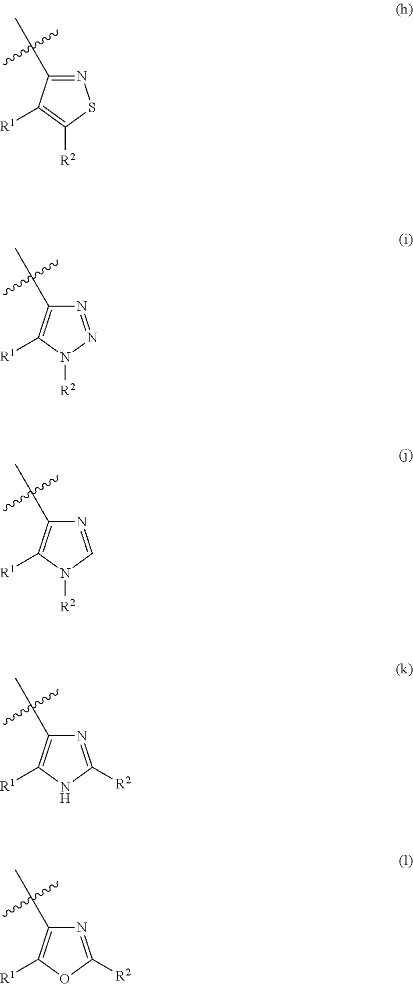
R¹ is selected from H, 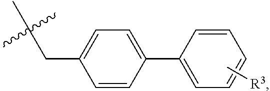
C₃-C₁₂ alkenyl, C₁-C₁₂ alkyl, —NR⁴R⁵ substituted C₂-C₄ alkyl, benzyl or piperidyl which is optionally substituted by —COOR⁴;
R² is selected from H, 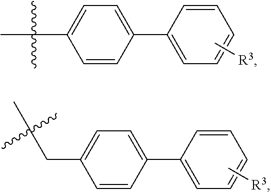
COR⁴, —COOR⁴, —CONR⁴R⁵, —CH═NNR⁴R⁵, —C(═CH₂)—OC(═O)R⁴, C₁-C₁₂ alkyl, C₃-C₇ cycloalkyl, phenyl, wherein alkyl, cycloalkyl and phenyl are optionally substituted by halogen, —NR⁴R⁵, —OR⁴, —SR⁴, —SO₂R⁴, —NHCOR⁴, —NHSO₂R⁴, —NHCSNHR⁴, 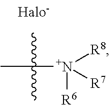
—N₃ or phenyl;
R³, optionally at ortho-, meta- or para-position of the benzene ring, is selected from H, halogen, C₁-C₆ alkyl or partially or fully halogenated C₁-C₆ alkyl;
R⁴ and R⁵ are independently selected from H, C₃-C₇ cycloalkyl, straight or branched C₁-C₆ alkyl, wherein alkyl and cycloalkyl are optionally substituted by —COOR⁹, —NR⁹R¹⁰, —OR⁹, —COR⁹, phenyl, benzyl, aromatic or nonaromatic heterocycle, wherein phenyl, benzyl, aromatic and nonaromatic heterocycle are optionally further substituted by halogen or C₁-C₆ alkyl; or
R⁴ and R⁵ together with the N-atom to which they are attached form 5 to 8-membered nonaromatic heterocycle which may contain another heteroatom selected from the group consisting of N, O and S, and is optionally substituted by halogen, C₁-C₆ alkyl, —NR¹¹R¹², —OR¹¹, ═O, or benzyl, wherein C₁-C₆ alkyl is optionally substituted by —COOR⁴;
R⁶, R⁷ and R⁸ are independently selected from C₁-C₆ alkyl, hydroxyl substituted C₂-C₄ alkyl or benzyl, wherein benzyl is optionally substituted by halogen or C₁-C₆ alkyl;
R⁹ and R¹⁰ are independently selected from H, C₁-C₆ alkyl; or
R⁹, R¹⁰ together with the N-atom to which they are attached form 5 to 8-membered nonaromatic heterocycle which may contain another heteroatom selected from the group consisting of N, O and S;
R¹¹ and R¹² are independently selected from H, C₁-C₆ alkyl;
Halo is an abbreviation of halogen.

2. The azole heterocyclic compound, cis-trans isomers, enantiomers, diastereoisomers, racemes, solvates, hydrates, or pharmaceutically acceptable salts thereof as claimed in claim 1, wherein in Formula (I) or (III), T is 5-membered aliphatic ring or benzene ring; X is CH or N.

3. The azole heterocyclic compound, cis-trans isomers, enantiomers, diastereoisomers, racemes, solvates, hydrates, or pharmaceutically acceptable salts thereof as claimed in claim 1, wherein in Formula (I), when T is 5-membered aliphatic ring, X is N; when T is benzene ring, X is CH.

4. The azole heterocyclic compound, cis-trans isomers, enantiomers, diastereoisomers, racemes, solvates, hydrates, or pharmaceutically acceptable salts thereof as claimed in claim 1, wherein in Formula (I), (II) or (III), Y is phenyl substituted by fluorine atoms.

5. The azole heterocyclic compound, cis-trans isomers, enantiomers, diastereoisomers, racemes, solvates, hydrates, or pharmaceutically acceptable salts thereof as claimed in claim 1, wherein in Formula (I), (II) or (III), Y is 4-fluorophenyl or 2,3-difluorophenyl.

6. The azole heterocyclic compound, cis-trans isomers, enantiomers, diastereoisomers, racemes, solvates, hydrates, or pharmaceutically acceptable salts thereof as claimed in claim 1, wherein in Formula (I), (II) or (III), W is selected from 6 structures of Formulae (a-f) as follows: 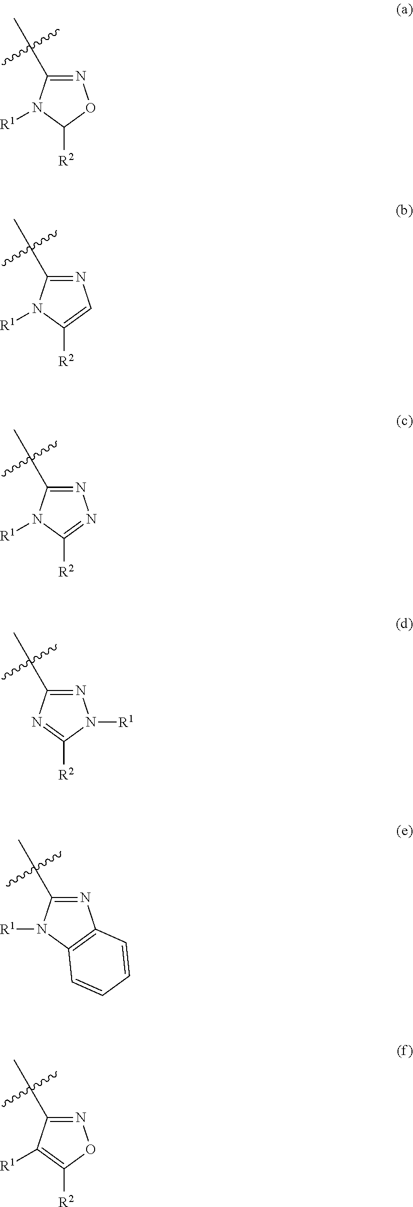
in which R¹ and R² are as defined in claim 1.

7. The azole heterocyclic compound, cis-trans isomers, enantiomers, diastereoisomers, racemes, solvates, hydrates, or pharmaceutically acceptable salts thereof as claimed in claim 6, wherein in Formula (I), (II) or (III), R¹ is 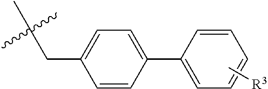
or —NR⁴R⁵ substituted C₂-C₄ alkyl.

8. The azole heterocyclic compound, cis-trans isomers, enantiomers, diastereoisomers, racemes, solvates, hydrates, or pharmaceutically acceptable salts thereof as claimed in claim 6, wherein in Formula (I), (II) or (III), R¹ is (4-(trifluoromethyl)biphenyl-4-yl)methyl.

9. The azole heterocyclic compound, cis-trans isomers, enantiomers, diastereoisomers, racemes, solvates, hydrates, or pharmaceutically acceptable salts thereof as claimed in claim 6, wherein in Formula (I), (II) or (III), R² is —COR⁴, —CONR⁴R⁵, C₁-C₅ alkyl or C₃-C₅ cycloalkyl, wherein alkyl and cycloalkyl are optionally substituted by —NR⁴R⁵, —OR⁴, —SR⁴, —SO₂R⁴, ═NNR⁴R⁵, —NHCOR⁴, —NHSO₂R⁴, —NHCSNHR⁴ or

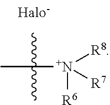

10. The azole heterocyclic compound, cis-trans isomers, enantiomers, diastereoisomers, racemes, solvates, hydrates, or pharmaceutically acceptable salts thereof as claimed in claim 6, wherein in Formula (I), (II) or (III), R² is —COR⁴, —CONR⁴R⁵, cyclopropyl or C₁-C₅ alkyl, wherein alkyl is substituted by —NR⁴R⁵, —OR⁴, —SR⁴, —SO₂R⁴, ═NNR⁴R⁵, —NHCOR⁴, —NHSO₂R⁴, —NHCSNHR⁴ or

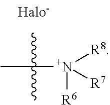

11. The azole heterocyclic compound, cis-trans isomers, enantiomers, diastereoisomers, racemes, solvates, hydrates, or pharmaceutically acceptable salts thereof as claimed in claim 6, wherein in Formula (I), (II) or (III), R² is —CONR⁴R⁵, cyclopropyl or C₁-C₅ alkyl, wherein C₁-C₅ alkyl is substituted by —NR⁴R⁵, —OR⁴, —SR⁴, ═NNR⁴R⁵, or

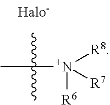

12. The azole heterocyclic compound, cis-trans isomers, enantiomers, diastereoisomers, racemes, solvates, hydrates, or pharmaceutically acceptable salts thereof as claimed in claim 6, wherein C₁-C₅R² is selected from dimethylcarbamoyl, 2-(diethylamino)ethylcarbamoyl, (2-(diethylamino)ethyl)(methyl)carbamoyl, (dimethylamino)methyl, (diethylamino)methyl, pyrrolidin-1-ylmethyl, ((4-fluorobenzyl)(methyl)amino)methyl, isopropyl, cyclopropyl, 3-(diethylamino)propyl, 4-(diethylamino)butyl, hydroxymethyl, 1-hydroxyethyl, (4-fluorobenzylthio)methyl, (isopropyl(methyl)amino)methyl, ((1-ethylpyrrolidin-2-yl)methylamino)methyl, (4-ethylpiperazin-1-yl)methyl, ((2-(dimethylamino)ethylmethyl)amino)methyl, ((2-(diethylamino)ethyl)(methyl)amino)methyl, (((2-(dimethylamino)ethyl)(ethyl)amino)methyl, (((3-(dimethylamino)propyl)(methyl)amino)methyl), (methyl(pyridin-2-ylmethyl)amino)methyl, (4-(dimethylamino)piperidin-1-yl)methyl, (2,2-dimethylhydrazono)methyl, (2-hydroxyethoxy)methyl, (2-(diethylamino)ethoxy)methyl, 1-(2-(dimethylamino)ethylamino)ethyl, 1-((2-(dimethylamino)ethyl)(methyl)amino)ethyl, 1-(2-(diethylamino)ethylamino)ethyl, 1-((2-(diethylamino)ethyl)(methyl)amino)ethyl, 1-((3-(dimethylamino)propyl)(methyl)amino)ethyl, ((methyl(2-(pyrrolidin-1-yl)ethyl)amino)methyl, ((methyl(2-(piperidin-1-yl)ethyl)amino)methyl, 3-(pyrrolidin-1-yl)propyl, 3-(piperidin-1-yl)propyl, 4-(pyrrolidin-1-yl)butyl, 4-(piperidin-1-yl)butyl,

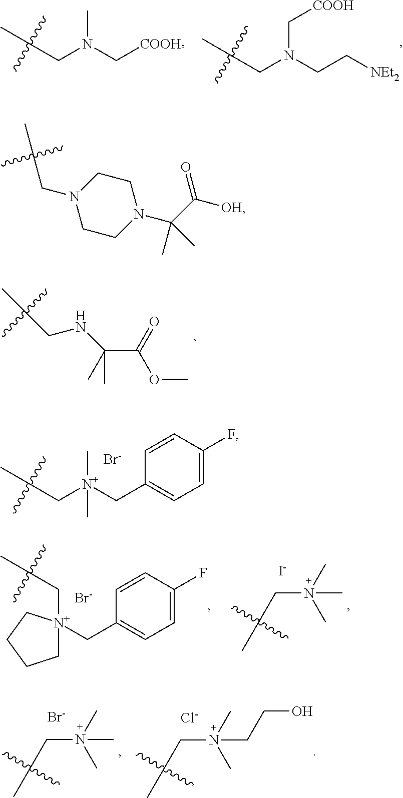

13. The azole heterocyclic compound, cis-trans isomers, enantiomers, diastereoisomers, racemes, solvates, hydrates, or pharmaceutically acceptable salts thereof as claimed in claim 1 having the structure of Formula (IA)-(IG), (IIA) or (IIIA): 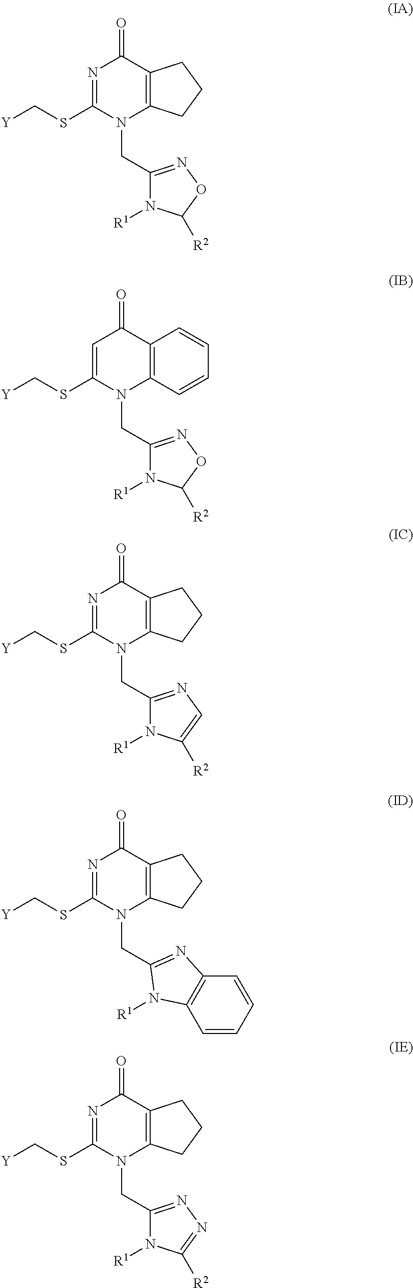 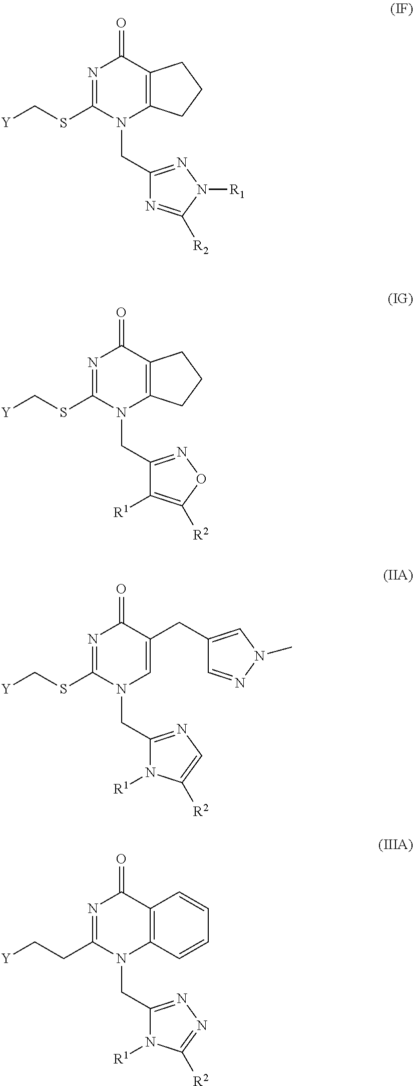
in which Y, R¹ and R² are as defined in claim 1.

14. The azole heterocyclic compound, cis-trans isomers, enantiomers, diastereoisomers, racemes, solvates, hydrates, or pharmaceutically acceptable salts thereof as claimed in claim 1, wherein the compound is selected from:
2-(4-fluorobenzylthio)-1-((5-n-heptyl-4,5-dihydro-1,2,4-oxadiazol-3-yl)methyl)-6,7-dihydro-1H-cyclopenta[d]pyrimidin-4(5H)-one;
1-((5-n-decyl-4,5-dihydro-1,2,4-oxadiazol-3-yl)methyl)-2-(4-fluorobenzylthio)-6,7-dihydro-1H-cyclopenta[d]pyrimidin-4(5H)-one;
2-(4-fluorobenzylthio)-1-((5-n-heptyl-4,5-dihydro-1,2,4-oxadiazol-3-yl)methyl)quinolin-4(1H)-one;
2-(4-fluorobenzylthio)-1-((5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one; (E)-2-(4-fluorobenzylthio)-1-((5-n-heptyl-4-(n-oct-1-enyl)-4,5-dihydro-1,2,4-oxadiazol-3-yl)methyl)-6,7-dihydro-1H-cyclopenta[d]pyrimidin-4(5H)-one;
2-(2,3-difluorobenzylthio)-1-((4-(2-morpholinoethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one;
ethyl 4-(3-((2-(2,3-difluorobenzylthio)-4-oxo-4,5,6,7-tetrahydro-1H-cyclopenta[d]pyrimidin-1-yl)methyl)-5-(4′-(trifluoromethyl)biphenyl-4-yl)-1,2,4-oxadiazol-4(5H)-yl)piperidine-1-carboxylate;
ethyl 4-(3-((2-(4-fluorobenzylthio)-4-oxo-4,5,6,7-tetrahydro-1H-cyclopenta[d]pyrimidin-1-yl)methyl)-5-(4′-(trifluoromethyl)biphenyl-4-yl)-1,2,4-oxadiazol-4(5H)-yl)piperidine-1-carboxylate;
2-(4-fluorobenzylthio)-1-((4-(2-(piperidin-1-yl)ethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one;
1-((4-(2-diethylamino)ethyl)-5-(4′-(trifluoromethyl)biphenyl-4-yl)-4,5-dihydro-1,2,4-oxadiazol-3-yl)methyl)-2-(4-fluorobenzylthio)-6,7-dihydro-1H-cyclopenta[d]pyrimidin-4(5H)-one;
2-(2,3-difluorobenzylthio)-1-((4-(2-(piperidin-1-yl)ethyl)-5-(4′-(trifluoromethyl)biphenyl-4-yl)-4,5-dihydro-1,2,4-oxadiazol-3-yl)methyl)-6,7-dihydro-1H-cyclopenta[d]pyrimidin-4(5H)-one;
2-(4-fluorobenzylthio)-1-((5-phenyl-1H-imidazol-2-yl)methyl)-6,7-dihydro-1H-cyclopenta[d]pyrimidin-4(5H)-one;
1-((1H-tetrazol-5-yl)methyl)-2-(4-fluorobenzylthio)-6,7-dihydro-1H-cyclopenta[d]pyrimidin-4(5H)-one;
2-(4-fluorobenzylthio)-1-((1-((4′-(trifluoromethyl)biphenyl-4-yl)methyl)-1H-imidazol-2-yl)methyl)-6,7-dihydro-1H-cyclopenta[d]pyrimidin-4(5H)-one;
1-((1-n-dodecyl-1H-imidazol-2-yl)methyl)-2-(4-fluorobenzylthio)-6,7-dihydro-1H-cyclopenta[d]pyrimidin-4(5H)-one;
1-((1-n-butyl-1H-imidazol-2-yl)methyl)-2-(4-fluorobenzylthio)-6,7-dihydro-1H-cyclopenta[d]pyrimidin-4(5H)-one;
1-((1-((4′-chlorobiphenyl-4-yl)methyl)-1H-imidazol-2-yl)methyl)-2-(4-fluorobenzylthio)-6,7-dihydro-1H-cyclopenta[d]pyrimidin-4(5H)-one;
1-((1-(biphenyl-4-ylmethyl)-1H-imidazol-2-yl)methyl)-2-(4-fluorobenzylthio)-6,7-dihydro-1H-cyclopenta[d]pyrimidin-4(5H)-one;
1-((1-n-butyl-5-(4-chlorophenyl)-1H-imidazol-2-yl)methyl)-2-(4-fluorobenzylthio)-6,7-dihydro-1H-cyclopenta[d]pyrimidin-4(5H)-one;
1-((1-n-butyl-5-(4-chlorophenyl)-1H-imidazol-2-yl)methyl)-2-(2-nitrobenzylthio)-6,7-dihydro-1H-cyclopenta[d]pyrimidin-4(5H)-one;
1-((5-n-decyl-1-(2-(diethylamino)ethyl)-1H-imidazol-2-yl)methyl)-2-(4-fluorobenzylthio)-6,7-dihydro-1H-cyclopenta[d]pyrimidin-4(5H)-one;
1-((1-(2-(diethylamino)ethyl)-5-(4′-(trifluoromethyl)biphenyl-4-yl)-1H-imidazol-2-yl)methyl)-2-(4-fluorobenzylthio)-6,7-dihydro-1H-cyclopenta[d]pyrimidin-4(5H)-one;
1-((1-benzyl-5-((diethylamino)methyl)-1H-imidazol-2-yl)methyl)-2-(4-fluorobenzylthio)-6,7-dihydro-1H-cyclopenta[d]pyrimidin-4(5H)-one;
2-(4-fluorobenzylthio)-1-((5-methyl-1-((4′-(trifluoromethyl)biphenyl-4-yl)methyl)-1H-imidazol-2-yl)methyl)-6,7-dihydro-1H-cyclopenta[d]pyrimidin-4(5H)-one;
1-((5-((diethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
2-(4-fluorobenzylthio)-1-((5-(pyrrolidin-1-ylmethyl)-1-((4′-(trifluoromethyl)biphenyl-4-yl)methyl)-1H-imidazol-2-yl)methyl)-6,7-dihydro-1H-cyclopenta[d]pyrimidin-4(5H)-one;
1-(5-(((4-fluorobenzyl)(methyl)amino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
1-((5-((4-benzylpiperazin-1-yl)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
1-((5-((dimethylamino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
N-(4-fluorobenzyl)-1-(2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazol-5-yl)-N,N-dimethylmthanamninium bromide;
1-((5-((dimethylamino)methyl)-1-(4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
1-(4-fluorobenzyl)-1-((2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazol-5-yl)methyl)pyrrolidinium bromide;
ethyl 2-((2-(4-fluorobenzylthio)-4-oxo-4,5,6,7-tetrahydro-1H-cyclopenta[d]pyrimidin-1-yl)methyl)-1-(4′-(trifluoromethyl)biphenyl-4-yl)methyl)-1H-imidazole-5-carboxylate;
2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxylic acid;
2-((2-(4-fluorobenzylthio)-4-oxo-4,5,6,7-tetrahydro-1H-cyclopenta[d]pyrimidin-1-yl)methyl)-N,N-dimethyl-1-((4′-(trifluoromethyl)biphenyl-4-yl)methyl)-1H-imidazole-5-carboxamide;
2-(4-fluorobenzylthio)-1-((1-((4′-(trifluoromethyl)biphenyl-4-yl)methyl)-1H-benzo[d]imidazol-2-yl)methyl)-6,7-dihydro-1H-cyclopenta[d]pyrimidin-4(5H)-one;
1-((4-ethyl-5-(4′-(trifluoromethyl)biphenyl-4-yl)-4H-1,2,4-triazol-3-yl)methyl)-2-(4-fluorobenzylthio)-6,7-dihydro-1H-cyclopenta[d]pyrimidin-4(5H)-one;
1-((4-ethyl-5-(4′-(trifluoromethyl)biphenyl-4-yl)-4H-1,2,4-triazol-3-yl)methyl)-2-(4-methoxybenzylthio)-6,7-dihydro-1H-cyclopenta[d]pyrimidin-4(5H)-one;
2-(2,3-difluorobenzylthio)-1-((4-ethyl-5-(4′-(trifluoromethyl)biphenyl-4-yl)-4H-1,2,4-triazol-3-yl)methyl)-6,7-dihydro-1H-cyclopenta[d]pyrimidin-4(5H)-one;
1-((4-ethyl-5-(3-phenylpropyl)-4H-1,2,4-triazol-3-yl)methyl)-2-(4-fluorobenzylthio)-6,7-dihydro-1H-cyclopenta[d]pyrimidin-4(5H)-one;
1-((4-ethyl-5-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((1-((4′-(trifluoromethyl)biphenyl-4-yl)methyl)-1H-1,2,4-triazol-3-yl)methyl)-6,7-dihydro-1H-cyclopenta[d]pyrimidin-4(5H)-one;
methyl 2-(((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)(methyl)amino)acetate;
2-(((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)(methyl)amino)acetic acid;
2-(4-fluorobenzylthio)-1-((5-methyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-n-propyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-isopropyl-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-cyclopropyl-4-((4′-(rifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(benzylsulfonylmethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(3-(diethylamino)propyl)-4-((4′-trifluoromethyl)biphenyl-4-yl)methyl)-4H-1,2,4-triazol-3-yl)methyl)-2-(4-fluorobenzyltbio)-6,7-dihydro-1H-cyclopenta[d]pyrimidin-4(5H)-one;
1-((5-(4-(diethylamino)butyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-(hydroxymethyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((4-((4′-(trifluoromethyl)biphenyl-4-yl)methy)-4H-1,2,4-triazol-3-yl)methyl)-6,7-dihydro-1H-cyclopenta[d]pyrimidin-4(5H)-one;
5-((2-(4-fluorobenzylthio)-4-oxo-4,5,6,7-tetrahydro-1H-cyclopenta[d]pyrimidin-1-yl)methyl)-4-((4′-(rifluoromethyl)biphenyl-4-yl)methyl)-4H-1,2,4-triazole-3-carbaldehyde;
1-((5-((dimethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
N-(4-fluorobenzyl)-1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)-N,N-dimethylmethanaminium bromide;
1-(5-((2-(4-fluorobenzylthio)-4-oxo-4,5,6,7-tetrahydro-1H-cyclopenta[d]pyrimidin-1-yl)methyl)-4-((4-(trifluoromethyl)biphenyl-4-yl)methyl-4H-1,2,4-triazol-3-yl)-N,N,N-trimethylmethanaminium iodide;
1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)-N,N,N-trimethylmethanaminium bromide;
1-((5-((diethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((4-fluorobenzyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((isopropyl(methyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
(±)1-((5-(((1-ethylpyrrolidin-2-yl)methylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((2S,6R)-2,6-dimethylmorpholino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
methyl-2-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methylamino)-2-methylpropanoate;
1-((5-((4-ethylpiperazin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-methoxyethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
methyl-2-(4-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)piperazin-1-yl)-2-methylpropanoate;
2-(4-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)piperazin-1-yl)-2-methylpropanoic acid;
1-((5-(((2-(dimethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((2-(diethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((2-(diethylamino)ethyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one tartrate;
1-((5-(((3-(dimethylamino)propyl)(methyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-morpholinoethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
(R)-1-((5-((3-(dimethylamino)pyrrolidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(S)-1-((5-((3-(dimethylamino)pyrrolidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-(piperidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((2-(pyrrolidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((2-(diisopropylamino)ethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-((2-(diethylamino)ethylamino)methyl)-((4′-trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((4-methylpiperazin-1-yl)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((methyl(pyridin-2-ylmethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((cyclopropyl(methyl)amino)methyl)-4-((4′-(trifluoromethyl)biphenyl-4-yl)methyl)-4H-1,2,4-triazol-3-yl)methy)-2-(4-fluorobenzylthio)-6,7-dihydro-1H-cyclopenta[d]pyrimidin-4(5H)-one;
1-((5-((4-(dimethylamino)piperidin-1-yl)methyl)-4-((4′-(trifluoromethyl)biphenyl-4-yl)methyl)-4H-1,2,4-triazol-3-yl)methyl)-2-(4-fluorobenzylthio)-6,7-dihydro-1-cyclopenta[d]pyrimidin-4(5H)-one;
1-((5-((3,3-difluoropyrrolidin-1-yl)methyl)-4-((4′-(trifluoromethyl)biphenyl-4-yl)methyl)-4H-1,2,4-triazol-3-yl)methy)-2-(4-fluorobenzylthio)-6,7-dihydro-1H-cyclopenta[d]pyrimidin-4(5H)-one;
1-((5-((2-(dimethylamino)ethylamino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one; (S)-1-((5-(((1-ethylpyrrolidin-2-yl)methylamino)methyl)-4-((4-(trifluoromethyl)biphenyl-4-yl)methyl-1H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((2-(2-oxoimidazolidin-1-yl)ethylamino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-(azidomethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(aminomethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
N-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)ethanesulfonamide;
2-(4-fluorobenzylthio)-1-(5-((2-oxoimidazolidin-1-yl)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)-3-methylthiourea;
1-((5-(chloromethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
2-(4-fluorobenzylthio)-1-((5-((4-fluorobenzylthio)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
(E)-1-((5-((2,2-dimethylhydrazono)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(E)-1-((5-((2-tert-butylhydrazono)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
(E)-2-(4-fluorobenzylthio)-1-((5-((piperidin-1-ylimino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-(5-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-3-yl)vinyl acetate;
2-(4-fluorobenzylthio)-1-((5-(1-hydroxyethyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-acetyl-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
N-((5-((2-(4-fluorobenzylthio)-4-oxo-4,5,67-tetrahydro-1H-cyclopenta[d]pyrimidin-1-yl)methyl)-4-((4′-(trifluoromethyl)biphenyl-4-yl)methyl)-4H-1,2,4-triazol-3-yl)methyl)-2-hydroxy-N,N-dimethylethanaminium chloride;
2-(4-fluorobenzylthio)-1-((5-((2-hydroxyethoxy)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
1-((5-((2-(diethylamino)ethoxy)methyl)-4-((4′-(trifluoromethyl)biphenyl-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(n-butoxymethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-(2-(dimethylamino)ethylamino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-((2-(dimethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-(2-(diethylamino)ethylamino)ethyl)-4-((4′-(rifluoromethyl)biphenyl-4-yl)methyl)-4H-1,2,4-triazol-3-yl)methyl-2-(4-fluorobenzylthio)-6,7-dihydro-1H-cyclopenta[d]pyrimidin-4(5H)-one;
1-((5-(1-((2-(diethylamino)ethyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(1-((2-(diethylamino)methyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one tartrate;
1-((5-(1-((3-(dimethylamino)propyl)(methyl)amino)ethyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
1-((5-(((2-(dimethylamino)ethyl)(ethyl)amino)methyl)-4-((4′-(trifluoromethyl)biphenyl-4-yl)methyl)-4H-1,2,4-triazol-3-yl)methyl)-2-(4-fluorobenzylthio)-6,7-dihydro-1H-cyclopenta[d]pyrimidin-4(5H)-one;
ethyl-2-((2-(diethylamino)ethyl)((3-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)-4H-1,2,4-triazol-5-yl)methyl)amino)acetate;
2-((2-(diethylamino)ethyl)((3-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methy)-4H-1,2,4-triazol-5-yl)methyl)amino)acetic acid;
N-(2-(diethylamino)ethyl)-2-((2-(4-fluorobenzylthio)-4-oxo-4,5,6,7-tetrahydro-1H-cyclopenta[d]pyrimidin-1-yl)methyl)-1-((4′-(trifluoromethyl)biphenyl-4-yl)methyl)-1H-imidazole-5-carboxamide;
N-(2-(diethylamino)ethyl)-2-((2-(4-fluorobenzylthio)-4-oxo-5,6,7-tetrahydro-1H-cyclopenta[d]pyrimidin-1-yl)methyl)-N-methyl-1-((4′-(trifluoromethyl)biphenyl-4-yl)methyl)-1H-imidazole-5-carboxamide;
1-((5-(((2-(diethylamino)ethyl)(methyl)amino)methyl)-1-((4′-(trifluoromethyl)biphenyl-4-yl)methyl)-1H-imidazol-2-yl)methyl)-2-(4-fluorobenzylthio)-6,7-dihydro-1H-cyclopenta[d]pyrimidin-4(5H)-one;
2-(4-fluorobenzylthio)-1-((5-((methyl(2-(pyrrolidin-1-yl)ethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((methyl(2-(piperidin-1-yl)ethyl)amino)methyl)-4-((4′-(trifluoromethyl)biphenyl-4-yl)methyl)-1H-imidazol-2-yl)methyl)-6,7-dihydro-1H-cyclopenta[d]pyrimidin-4(5H)-one;
2-(4-fluorobenzylthio)-1-((5-(3-(pyrrolidin-1-yl)propyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-(3-(piperidin-1-yl)propyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-(4-(pyrrolidin-1-yl)butyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-(4-(piperidin-1-yl)butyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
2-(4-fluorobenzylthio)-1-((5-((methyl(2-(piperidin-1-yl)ethyl)amino)methyl)-4-((4′-(trifluoromethyl)biphenyl-4-yl)methyl)-4H-1,2,4-triazol-3-yl)methyl)-6,7-dihydro-1H-cyclopenta[d]pyrimidin-4(5H)-one;
methyl-3-((2-(4-fluorobenzylthio)-4-oxo-4,5,6,7-tetrahydro-1H-cyclopenta[d]pyrimidin-1-yl)methyl)-4-((4′-(trifluoromethyl)biphenyl-4-yl)methyl)isoxazole-5-carboxylate;
2-(4-fluorobenzylthio)-1-((5-(hydroxymethyl)-4-((4′-(trifluoromethyl)biphenyl-4-yl)methyl)isoxazol-3-yl)methyl)-6,7-dihydro-1H-cyclopenta[d]pyrimidin-4(5H)-one;
1-((5-(chloromethyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
1-((5-((diethylamino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
1-((5-(((2-(dimethylamino)ethyl)(methyl)amino)methyl)-4-((4′-(trifluoromethyl)biphenyl-4-yl)methyl)isoxazol-3-yl)methyl)-2-(4-fluorobenzylthio)-6,7-dihydro-1H-cyclopenta[d]pyrimidin-4(5H)-one;
1-((5-(((2-(diethylamino)ethyl)(methyl)amino)methyl)-4-((4′-(trifluoromethyl)biphenyl-4-yl)methy)isoxazol-3-yl)methyl)-2-(4-fluorobenzylthio)-6,7-dihydro-1H-cyclopenta[d]pyrimidin-4(5H)-one;
2-(4-fluorobenzylthio)-1-((5-((methyl(2-(piperidin-1-yl)ethyl)amino)methyl)-4-((4′-(trifluoromethyl)biphenyl-4-yl)methyl)isoxazol-3-yl)methyl)-6,7-dihydro-1H-cyclopenta[d]pyrimidin-4(5H)-one;
2-(4-fluorophenethyl)-1-((4-((4′-(trifluoromethyl)biphenyl-4-yl)methyl)-4H-1,2,4-triazol-3-yl)methyl)quinazolin-4(1H)-one.

15. A pharmaceutical composition comprising a therapeutically effective amount of one or more of the azole heterocyclic compounds, cis-trans isomers, enantiomers, diastereoisomers, racemes, solvates, hydrates, or pharmaceutically acceptable salts thereof of claim 1, and a pharmaceutically acceptable auxiliary.

16. The pharmaceutical composition as claimed in claim 15, further comprising one or more agents selected from the group consisting of anti-hyperlipidaemic, anti-atherosclerotic, anti-diabetic, anti-anginal, antiinflammatory, anti-hypertension agents and agents for lowering Lp(a).

17. A use of the azole heterocyclic compounds, cis-trans isomers, enantiomers, diastereoisomers, racemes, solvates, hydrates, or pharmaceutically acceptable salts thereof of claim 1, in manufacturing a medicament as Lp-PLA₂ inhibitor.

18. A use of the azole heterocyclic compounds, cis-trans isomers, enantiomers, diastereoisomers, racemes, solvates, hydrates, or pharmaceutically acceptable salts thereof of claim 1, in manufacturing a medicament for preventing, curing or ameliorating diseases associated with activity of the enzyme Lp-PLA₂.

19. The use as claimed in claim 18, wherein the diseases include atherosclerosis, stroke, coronary heart disease, diabetes, asthma, psoriasis, rheumatoid arthritis, or acute and chronic inflammation.

20. A method of preventing, curing or ameliorating a disease associated with activity of the enzyme Lp-PLA₂, wherein the method involves administering the compounds, cis-trans isomers, enantiomers, diastereoisomers, racemes, solvates, hydrates, or pharmaceutically acceptable salts thereof of claim 1.

21. The method as claimed in claim 20, wherein the diseases include atherosclerosis, stroke, coronary heart disease, diabetes, asthma, psoriasis, rheumatoid arthritis, or acute and chronic inflammation.

22. A preparation method of the azole heterocyclic compound of Formula (I), cis-trans isomers, enantiomers, diastereoisomers, racemes, solvates, hydrates, or pharmaceutically acceptable salts thereof, wherein one of the processes illustrated by synthetic route (1-4) as follows is employed: 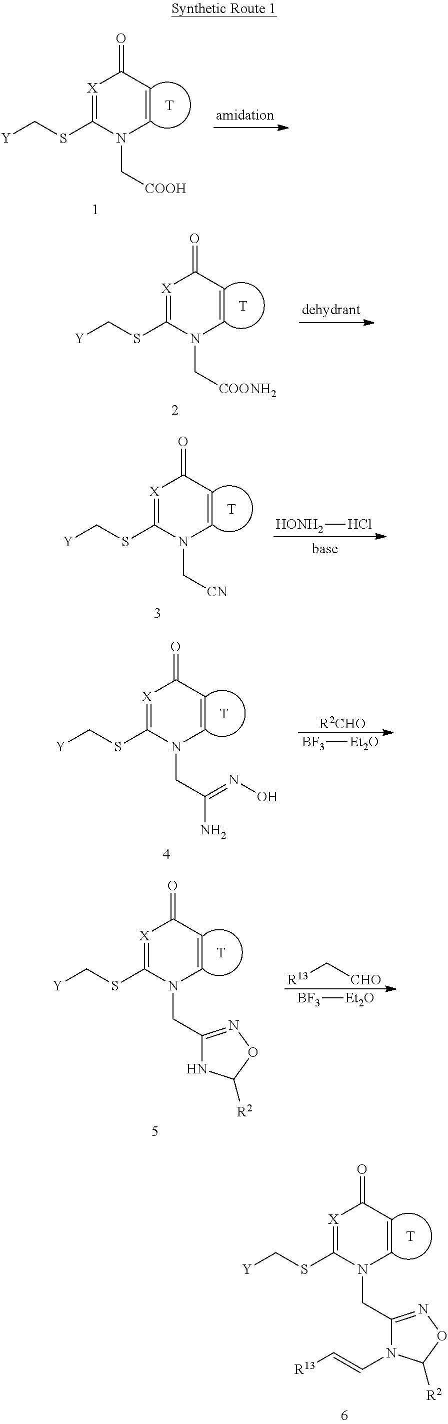
when W is Formula (a) meanwhile R¹ is alkenyl in the compound of Formula (I), the compound of Formula (I) (i.e. compound 6) can be prepared according to the method illustrated by synthetic route 1, in which: R¹³ is C₁-C₁₀ alkyl; T, X, Y and R² are as defined in claim 1;
compound 1 is converted to compound 2 by amidation which is then dehydrated to give compound 3; compound 3 reacts with hydroxylamine hydrochloride in the presence of a base to produce compound 4; catalyzed by boron trifluoride-diethyl ether complex, the reaction of compound 4 with R²CHO yields compound 5 in an aprotic solvent; compound 5 is then transformed into compound 6 by reaction with R¹³CH₂CHO in the presence of boron trifluoride-diethyl ether complex as a catalyst;
or, 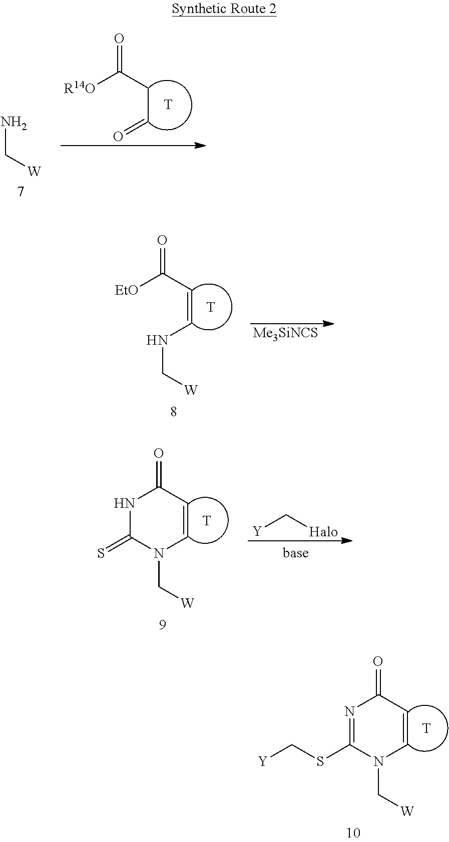
when T is 4 to 6-membered aliphatic ring and X═N, meanwhile R¹ is not alkenyl in the compound of Formula (I), the compound of Formula (I) (i.e. compound 10) can be prepared according to the method illustrated by synthetic route 2, in which: R¹⁴ is methyl or ethyl; Halo, T, W and Y are as defined in claim 1;
in a polar solvent, compound 7 is condensed with a cycloalkanone carboxylate 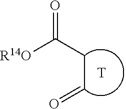
to yield compound 8 in the presence of a dehydrant; compound 8 reacts with Me₃SiNCS to produce compound 9 which is then converted to compound 10 by reaction with 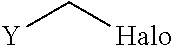
in a polar solvent in the presence of a base;
or, 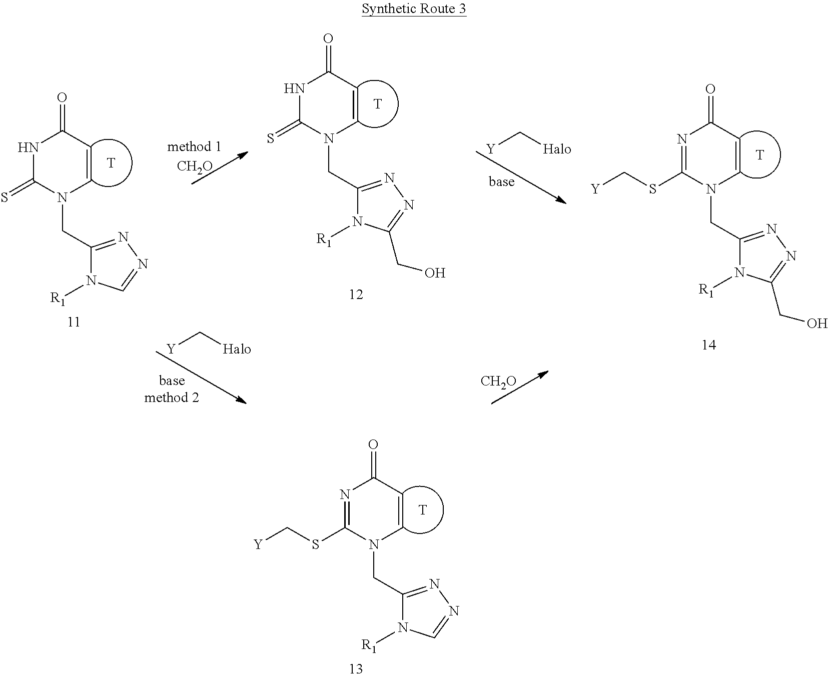
when W is structure (c) meanwhile R² is hydroxymethyl in the above mentioned compound 10, the compound, i.e. compound 14, can be prepared according to either of the methods illustrated by synthetic route 3, in which Halo, T, Y and R¹ are as defined in claim 1;
according to the first method, compound 11 is heated with aqueous formaldehyde to afford compound 12 which then reacts in the presence of a base with 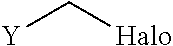
in a polar solvent to give compound 14;
according to the second method, compound 11 first reacts with 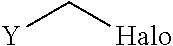
to give compound 13 which is then heated with aqueous formaldehyde to afford compound 14;
or, 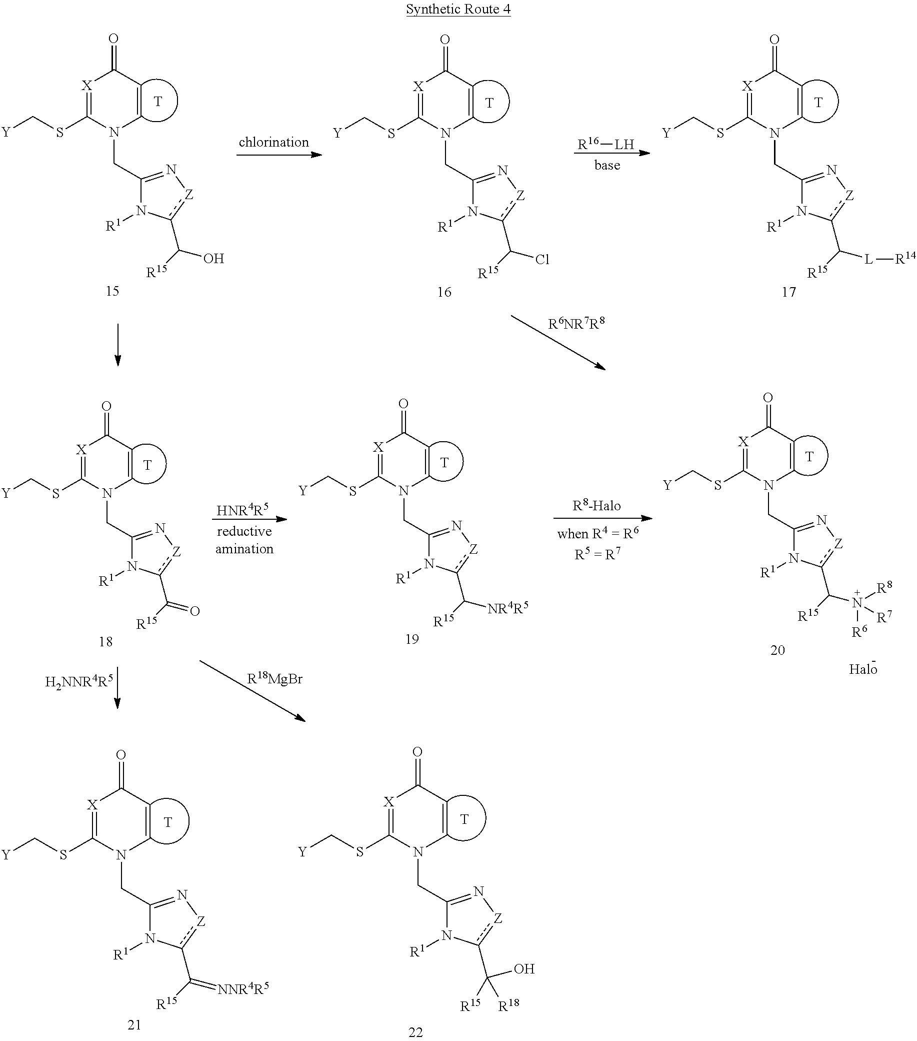
a compound of Formula (I) can be converted into another structure of the compound of Formula (I) by functional transformation, which is illustrated by route 4 as follows:
when R² in Formula (I) is α-hydroxy substituted alkyl, the compound of Formula (I) can be represented by the structure of compound 15, in which R¹⁵ is H or C₁-C₆ alkyl; R¹⁶ is C₁-C₆ alkyl that is optionally substituted by NR⁴R⁵ or phenyl, wherein phenyl is optionally substituted by halogen; R¹⁸ is C₁-C₆ alkyl; L is NR⁴, O or S; Z is CH, N or O; T, X, Y, R¹, R⁴, R⁵, R⁶, R⁷ and R⁸ are as defined in claim 1;
Compound 15 is chlorinated to afford compound 16 which then reacts with R¹⁶LH to give compound 17 in the presence of a base;
Compound 15 is oxidized to afford compound 18 which then reacts with HNR⁴R⁵ to give compound 19 in the presence of a reductant;
the condensation reaction of compound 16 with R⁶NR⁷R⁸ yields compound 20;
the condensation reaction of compound 19 with R⁸-Halo also yields compound 20;
the condensation reaction of compound 18 with H₂NNR⁴R⁵ yields compound 21;
the reaction of compound 18 with R¹⁸BrMg yields compound 22.
